SEQUENCE LISTING (537 sequences)

SEQ ID NO: 1 - protein, 127 aa, synthetic construct
  REGION 1..127: note = alpha-EGFR1
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 2 - protein, 10 aa, synthetic construct
  REGION 1..10: note = alpha-EGFR1 sdCDR1
GRTFSSYAMG

SEQ ID NO: 3 - protein, 16 aa, synthetic construct
  REGION 1..16: note = alpha-EGFR1 sdCDR2
INWSSGSTYYADSVKG

SEQ ID NO: 4 - protein, 18 aa, synthetic construct
  REGION 1..18: note = alpha-EGFR1 sdCDR3
GYQINSGNYNFKDYEYDY

SEQ ID NO: 5 - protein, 124 aa, synthetic construct
  REGION 1..124: note = alpha-EGFR2
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 6 - protein, 10 aa, synthetic construct
  REGION 1..10: note = alpha-EGFR2 sdCDR1
GRTSRSYGMG

SEQ ID NO: 7 - protein, 17 aa, synthetic construct
  REGION 1..17: note = alpha-EGFR2 sdCDR2
GISWRGDSTGYADSVKG

SEQ ID NO: 8 - protein, 15 aa, synthetic construct
  REGION 1..15: note = alpha-EGFR2 sdCDR3
AAGSAWYGTLYEYDY

SEQ ID NO: 9 - protein, 127 aa, synthetic construct
  REGION 1..127: note = h-alpha-EGFR1
EVQLVESGGGLVQPGGSLRLSCAASGRTFSSYAMGWFRQAPGKEREFVVAINWSSGSTYYADSVKGRFTISRDNSKNTLYLQMNSLRAEDTAVYYCAAGYQINSGNYNFKDYEYDYWGQGTLVTVSS

SEQ ID NO: 10 - protein, 10 aa, synthetic construct
  REGION 1..10: note = h-alpha-EGFR1 sdCDR1
GRTFSSYAMG

SEQ ID NO: 11 - protein, 16 aa, synthetic construct
  REGION 1..16: note = h-alpha-EGFR1 sdCDR2
INWSSGSTYYADSVKG

SEQ ID NO: 12 - protein, 18 aa, synthetic construct
  REGION 1..18: note = h-alpha-EGFR1 sdCDR3
GYQINSGNYNFKDYEYDY

SEQ ID NO: 13 - protein, 124 aa, synthetic construct
  REGION 1..124: note = aEGFR2a sdAb
EVQLVESGGGVVRPGGSLRLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSS

SEQ ID NO: 14 - protein, 10 aa, synthetic construct
  REGION 1..10: note = aEGFR2a sdAb sdCDR1
GRTSRSYGMG

SEQ ID NO: 15 - protein, 17 aa, synthetic construct
  REGION 1..17: note = aEGFR2a sdAb sdCDR2
GISWRGDSTGYADSVKG

SEQ ID NO: 16 - protein, 15 aa, synthetic construct
  REGION 1..15: note = aEGFR2a sdAb sdCDR3
AAGSAWYGTLYEYDY

SEQ ID NO: 17 - protein, 124 aa, synthetic construct
  REGION 1..124: note = h-alpha-EGFR2d
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSS

SEQ ID NO: 18 - protein, 10 aa, synthetic construct
  REGION 1..10: note = h-alpha-EGFR2d sdCDR1
GRTSRSYGMG

SEQ ID NO: 19 - protein, 17 aa, synthetic construct
  REGION 1..17: note = h-alpha-EGFR2d sdCDR2
GISWRGDSTGYADSVKG

SEQ ID NO: 20 - protein, 15 aa, synthetic construct
  REGION 1..15: note = h-alpha-EGFR2d sdCDR3
AAGSAWYGTLYEYDY

SEQ ID NO: 21 - protein, 114 aa, synthetic construct
  REGION 1..114: note = alpha-FOLR1 h77-2
QVQLVESGGGLVQPGGSLRLSCAASGFTVSNSVMAWYRQTPGNEREFVAIINSIGITNYADSVKGRFTISRDNSKNTLYLQMNSLRAEDTAVYVCNRNFDRIYWGQGTLVTVSS

SEQ ID NO: 22 - protein, 10 aa, synthetic construct
  REGION 1..10: note = alpha-FOLR1 h77-2 sdCDR1
GFTVSNSVMA

SEQ ID NO: 23 - protein, 16 aa, synthetic construct
  REGION 1..16: note = alpha-FOLR1 h77-2 sdCDR2
IINSIGITNYADSVKG

SEQ ID NO: 24 - protein, 6 aa, synthetic construct
  REGION 1..6: note = alpha-FOLR1 h77-2 sdCDR3
NFDRIY

SEQ ID NO: 25 - protein, 113 aa, synthetic construct
  REGION 1..113: note = alpha-FOLR1 h59.3
QVQLVESGGGLVQPGGSLRLSCAAPGNTFSISAMGWYRQAPGKQREWVAVTHSDYSTNYADSVKGRFTISRDNSKNTLYLQMNSLRAEDTAVYYCKHYGIDYWGQGTLVTVSS

SEQ ID NO: 26 - protein, 10 aa, synthetic construct
  REGION 1..10: note = alpha-FOLR1 h59.3 sdCDR1
GNTFSISAMG

SEQ ID NO: 27 - protein, 16 aa, synthetic construct
  REGION 1..16: note = alpha-FOLR1 h59.3 sdCDR2
VTHSDYSTNYADSVKG

SEQ ID NO: 28 - protein, 5 aa, synthetic construct
  REGION 1..5: note = alpha-FOLR1 h59.3 sdCDR3
YGIDY

SEQ ID NO: 29 - protein, 117 aa, synthetic construct
  REGION 1..117: note = alpha-FOLR1 h22-4
QVQLVESGGGLVQPGGSLRLSCEASGTTFSRDVMGWYRQAPGKQRELVAIISRGGSTNYADSVKGRFTISRDNSKNTLYLQMNSLRAEDTAVYYCNANTATWGRVFWGQGTLVTVSS

SEQ ID NO: 30 - protein, 10 aa, synthetic construct
  REGION 1..10: note = alpha-FOLR1 h22-4 sdCDR1
GTTFSRDVMG

SEQ ID NO: 31 - protein, 16 aa, synthetic construct
  REGION 1..16: note = alpha-FOLR1 h22-4 sdCDR2
IISRGGSTNYADSVKG

SEQ ID NO: 32 - protein, 9 aa, synthetic construct
  REGION 1..9: note = alpha-FOLR1 h22-4 sdCDR3
NTATWGRVF

SEQ ID NO: 33 - protein, 124 aa, synthetic construct
  REGION 1..124: note = alpha-B7H3 hF7
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSS

SEQ ID NO: 34 - protein, 10 aa, synthetic construct
  REGION 1..10: note = alpha-B7H3 hF7 sdCDR1
RRTFHTYHMG

SEQ ID NO: 35 - protein, 17 aa, synthetic construct
  REGION 1..17: note = alpha-B7H3 hF7 sdCDR2
VINWSGGSTVYADSVKG

SEQ ID NO: 36 - protein, 15 aa, synthetic construct
  REGION 1..15: note = alpha-B7H3 hF7 sdCDR3
GGATTQRATEASYDY

SEQ ID NO: 37 - protein, 122 aa, synthetic construct
  REGION 1..122: note = alpha-B7H3 hF12
QVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSS

SEQ ID NO: 38 - protein, 10 aa, synthetic construct
  REGION 1..10: note = alpha-B7H3 hF12 sdCDR1
PRTFSTYSMA

SEQ ID NO: 39 - protein, 17 aa, synthetic construct
  REGION 1..17: note = alpha-B7H3 hF12 sdCDR2
AINWSGGNTSYADSVKG

SEQ ID NO: 40 - protein, 13 aa, synthetic construct
  REGION 1..13: note = alpha-B7H3 hF12 sdCDR3
GGVLAHHNYEYDY

SEQ ID NO: 41 - protein, 122 aa, synthetic construct
  REGION 1..122: note = alpha-B7H3 hF12 (N57Q)
QVQLQESGGGLVQPGGSLRLSCEASPRTFSTYSMAWFRQAPGKERSFVAAINWSGGQTSYADSVKGRFTISRDNSKNTLYLQMNSLRAEDTAVYYCAAGGVLAHHNYEYDYWGQGTLVTVSS

SEQ ID NO: 42 - protein, 10 aa, synthetic construct
  REGION 1..10: note = alpha-B7H3 hF12 (N57Q) sdCDR1
PRTFSTYSMA

SEQ ID NO: 43 - protein, 17 aa, synthetic construct
  REGION 1..17: note = alpha-B7H3 hF12 (N57Q) sdCDR2
AINWSGGQTSYADSVKG

SEQ ID NO: 44 - protein, 13 aa, synthetic construct
  REGION 1..13: note = alpha-B7H3 hF12 (N57Q) sdCDR3
GGVLAHHNYEYDY

SEQ ID NO: 45 - protein, 122 aa, synthetic construct
  REGION 1..122: note = alpha-B7H3 hF12 (N57E)
QVQLQESGGGLVQPGGSLRLSCEASPRTFSTYSMAWFRQAPGKERSFVAAINWSGGETSYADSVKGRFTISRDNSKNTLYLQMNSLRAEDTAVYYCAAGGVLAHHNYEYDYWGQGTLVTVSS

SEQ ID NO: 46 - protein, 10 aa, synthetic construct
  REGION 1..10: note = alpha-B7H3 hF12 (N57E) sdCDR1
PRTFSTYSMA

SEQ ID NO: 47 - protein, 17 aa, synthetic construct
  REGION 1..17: note = alpha-B7H3 hF12 (N57E) sdCDR2
AINWSGGETSYADSVKG

SEQ ID NO: 48 - protein, 13 aa, synthetic construct
  REGION 1..13: note = alpha-B7H3 hF12 (N57E) sdCDR3
GGVLAHHNYEYDY

SEQ ID NO: 49 - protein, 122 aa, synthetic construct
  REGION 1..122: note = alpha-B7H3 hF12 (N57D)
QVQLQESGGGLVQPGGSLRLSCEASPRTFSTYSMAWFRQAPGKERSFVAAINWSGGDTSYADSVKGRFTISRDNSKNTLYLQMNSLRAEDTAVYYCAAGGVLAHHNYEYDYWGQGTLVTVSS

SEQ ID NO: 50 - protein, 10 aa, synthetic construct
  REGION 1..10: note = alpha-B7H3 hF12 (N57D) sdCDR1
PRTFSTYSMA

SEQ ID NO: 51 - protein, 17 aa, synthetic construct
  REGION 1..17: note = alpha-B7H3 hF12 (N57D) sdCDR2
AINWSGGDTSYADSVKG

SEQ ID NO: 52 - protein, 13 aa, synthetic construct
  REGION 1..13: note = alpha-B7H3 hF12 (N57D) sdCDR3
GGVLAHHNYEYDY

SEQ ID NO: 53 - protein, 122 aa, synthetic construct
  REGION 1..122: note = alpha-B7H3 hF12 (S59A)
QVQLQESGGGLVQPGGSLRLSCEASPRTFSTYSMAWFRQAPGKERSFVAAINWSGGNTAYADSVKGRFTISRDNSKNTLYLQMNSLRAEDTAVYYCAAGGVLAHHNYEYDYWGQGTLVTVSS

SEQ ID NO: 54 - protein, 10 aa, synthetic construct
  REGION 1..10: note = alpha-B7H3 hF12 (S59A) sdCDR1
PRTFSTYSMA

SEQ ID NO: 55 - protein, 17 aa, synthetic construct
  REGION 1..17: note = alpha-B7H3 hF12 (S59A) sdCDR2
AINWSGGNTAYADSVKG

SEQ ID NO: 56 - protein, 13 aa, synthetic construct
  REGION 1..13: note = alpha-B7H3 hF12 (S59A) sdCDR3
GGVLAHHNYEYDY

SEQ ID NO: 57 - protein, 122 aa, synthetic construct
  REGION 1..122: note = alpha-B7H3 hF12 (S59Y)
QVQLQESGGGLVQPGGSLRLSCEASPRTFSTYSMAWFRQAPGKERSFVAAINWSGGNTYYADSVKGRFTISRDNSKNTLYLQMNSLRAEDTAVYYCAAGGVLAHHNYEYDYWGQGTLVTVSS

SEQ ID NO: 58 - protein, 10 aa, synthetic construct
  REGION 1..10: note = alpha-B7H3 hF12 (S59Y) sdCDR1
PRTFSTYSMA

SEQ ID NO: 59 - protein, 17 aa, synthetic construct
  REGION 1..17: note = alpha-B7H3 hF12 (S59Y) sdCDR2
AINWSGGNTYYADSVKG

SEQ ID NO: 60 - protein, 13 aa, synthetic construct
  REGION 1..13: note = alpha-B7H3 hF12 (S59Y) sdCDR3
GGVLAHHNYEYDY

SEQ ID NO: 61 - protein, 122 aa, synthetic construct
  REGION 1..122: note = alpha-EpCAM h13
QVQLVESGGGLVQPGGSLTLSCAASGTGSIFSINLMGWYRQAPGKQRELVARITSGDSTVYADSVKGRFTISRDNSKNTLYLQMNSLRPEDTAVYYCNLLLRSSPGATTPYWGQGTLVTVSS

SEQ ID NO: 62 - protein, 12 aa, synthetic construct
  REGION 1..12: note = alpha-EpCAM h13 sdCDR1
GTGSIFSINLMG

SEQ ID NO: 63 - protein, 16 aa, synthetic construct
  REGION 1..16: note = alpha-EpCAM h13 sdCDR2
RITSGDSTVYADSVKG

SEQ ID NO: 64 - protein, 12 aa, synthetic construct
  REGION 1..12: note = alpha-EpCAM h13 sdCDR3
LLRSSPGATTPY

SEQ ID NO: 65 - protein, 114 aa, synthetic construct
  REGION 1..114: note = alpha-EpCAM h23
QVQLVESGGGLVQPGGSLTLSCVISGSFSALWAMRWYRQAPGQQRELVASSRGGTTSYADSVKGRFTISRDNSKNTLYLQMNSLRPEDTAVYYCNAIDGHLAYWGQGTLVTVSS

SEQ ID NO: 66 - protein, 10 aa, synthetic construct
  REGION 1..10: note = alpha-EpCAM h23 sdCDR1
GSFSALWAMR

SEQ ID NO: 67 - protein, 15 aa, synthetic construct
  REGION 1..15: note = alpha-EpCAM h23 sdCDR2
SSRGGTTSYADSVKG

SEQ ID NO: 68 - protein, 7 aa, synthetic construct
  REGION 1..7: note = alpha-EpCAM h23 sdCDR3
IDGHLAY

SEQ ID NO: 69 - protein, 126 aa, synthetic construct
  REGION 1..126: note = acEpCAM hVIB665
QVQLLESGGGLVQPGGSLRLSCAASGRTFSDYDMGWFRQGPGKEREFVAAISWSGGHTNYADSVKGRFTISRDNSKNTLYLQMNSLRAEDTAVYYCAADLRFTGGDTTTPETYDYWGQGTLVTVSS

SEQ ID NO: 70 - protein, 10 aa, synthetic construct
  REGION 1..10: note = acEpCAM hVIB665 sdCDR1
GRTFSDYDMG

SEQ ID NO: 71 - protein, 17 aa, synthetic construct
  REGION 1..17: note = acEpCAM hVIB665 sdCDR2
AISWSGGHTNYADSVKG

SEQ ID NO: 72 - protein, 17 aa, synthetic construct
  REGION 1..17: note = acEpCAM hVIB665 sdCDR3
DLRFTGGDTTTPETYDY

SEQ ID NO: 73 - protein, 126 aa, synthetic construct
  REGION 1..126: note = acEpCAM hVIB666
QVQLVESGGGLVQPGRSLRLSCAASGRTLDNYDMGWFRQGPGKEREFVAAISWSGGSTDYAYSVTGRFTISRDNAKNSLYLQMNSLRAEDTALYYCAADLRFTGGDTMTPETYDYWGQGTLVTVSS

SEQ ID NO: 74 - protein, 10 aa, synthetic construct
  REGION 1..10: note = acEpCAM hVIB666 sdCDR1
GRTLDNYDMG

SEQ ID NO: 75 - protein, 17 aa, synthetic construct
  REGION 1..17: note = acEpCAM hVIB666 sdCDR2
AISWSGGSTDYAYSVTG

SEQ ID NO: 76 - protein, 17 aa, synthetic construct
  REGION 1..17: note = acEpCAM hVIB666 sdCDR3
DLRFTGGDTMTPETYDY

SEQ ID NO: 77 - protein, 129 aa, synthetic construct
  REGION 1..129: note = aTrop2 hVIB557
QVQLLESGGGLVQPGGSLRLSCAASGRTFSSQSMGWFRQAPGKEREFVSAISWTGANPTYADSVKGRFTISRDNSKNTLYLQMNSLRAEDTAVYYCAADTSGGSYYYERATAETSYDYWGQGTLVTVSS

SEQ ID NO: 78 - protein, 10 aa, synthetic construct
  REGION 1..10: note = aTrop2 hVIB557 sdCDR1
GRTFSSQSMG

SEQ ID NO: 79 - protein, 17 aa, synthetic construct
  REGION 1..17: note = aTrop2 hVIB557 sdCDR2
AISWTGANPTYADSVKG

SEQ ID NO: 80 - protein, 20 aa, synthetic construct
  REGION 1..20: note = aTrop2 hVIB557 sdCDR3
DTSGGSYYYERATAETSYDY

SEQ ID NO: 81 - protein, 128 aa, synthetic construct
  REGION 1..128: note = aTrop2 hVIB565
QVQLLESGGGLVQPGGSLRLSCAASGFTFDYYAIGWFRQAPGKEREGVSCISSSHGSTYYADSVKGRFTISRDNSKNTVYLQMNSLRAEDTAVYYCATAGDGGDYHCSGLVDYGMDYWGKGTLVTVSS

SEQ ID NO: 82 - protein, 10 aa, synthetic construct
  REGION 1..10: note = aTrop2 hVIB565 sdCDR1
GFTFDYYAIG

SEQ ID NO: 83 - protein, 17 aa, synthetic construct
  REGION 1..17: note = aTrop2 hVIB565 sdCDR2
CISSSHGSTYYADSVKG

SEQ ID NO: 84 - protein, 19 aa, synthetic construct
  REGION 1..19: note = aTrop2 hVIB565 sdCDR3
AGDGGDYHCSGLVDYGMDY

SEQ ID NO: 85 - protein, 123 aa, synthetic construct
  REGION 1..123: note = aTrop2 hVIB575
QVQLLESGGGLVQPGGSLRLSCLASGRTVGRTAMGWFRQPPGKEREFVATISWAGGTTYYADFVKGRFTISRDNSKNTLYLQMNSLRAEDTAVYYCAASEPYSDYDPSGMVYWGKGTLVTVSS

SEQ ID NO: 86 - protein, 10 aa, synthetic construct
  REGION 1..10: note = aTrop2 hVIB575 sdCDR1
GRTVGRTAMG

SEQ ID NO: 87 - protein, 17 aa, synthetic construct
  REGION 1..17: note = aTrop2 hVIB575 sdCDR2
TISWAGGTTYYADFVKG

SEQ ID NO: 88 - protein, 14 aa, synthetic construct
  REGION 1..14: note = aTrop2 hVIB575 sdCDR3
SEPYSDYDPSGMVY

SEQ ID NO: 89 - protein, 123 aa, synthetic construct
  REGION 1..123: note = aTrop2 hVIB578
QVQLLESGGGLVQPGGSLRLSCAASGRTFGRAAMGWFRQPPGKEREFAATISWSGSNTYYADFVKGRFTISRDNSKNTVYLQMNSLRAEDTAVYYCAASEPYSDYDPSGMVYWGKGTLVTVSS

SEQ ID NO: 90 - protein, 10 aa, synthetic construct
  REGION 1..10: note = aTrop2 hVIB578 sdCDR1
GRTFGRAAMG

SEQ ID NO: 91 - protein, 17 aa, synthetic construct
  REGION 1..17: note = aTrop2 hVIB578 sdCDR2
TISWSGSNTYYADFVKG

SEQ ID NO: 92 - protein, 14 aa, synthetic construct
  REGION 1..14: note = aTrop2 hVIB578 sdCDR3
SEPYSDYDPSGMVY

SEQ ID NO: 93 - protein, 125 aa, synthetic construct
  REGION 1..125: note = aTrop2 hVIB609
QVQLLESGGGLVQPGGSLRLSCALSGLTFNTYPMAWFRQPPGQEREFVADMSWSGTNTYYADSVKGRFTISRDNSKNTLYLQMNSLRAEDTAVYYCAAGWPYSGTGRSTTDYTYWGQGTLVTVSS

SEQ ID NO: 94 - protein, 10 aa, synthetic construct
  REGION 1..10: note = aTrop2 hVIB609 sdCDR1
GLTFNTYPMA

SEQ ID NO: 95 - protein, 17 aa, synthetic construct
  REGION 1..17: note = aTrop2 hVIB609 sdCDR2
DMSWSGTNTYYADSVKG

SEQ ID NO: 96 - protein, 16 aa, synthetic construct
  REGION 1..16: note = aTrop2 hVIB609 sdCDR3
GWPYSGTGRSTTDYTY

SEQ ID NO: 97 - protein, 126 aa, synthetic construct
  REGION 1..126: note = aTrop2 hVIB619
QVQLLESGGGLVQPGGSLRLSCAASGRSFSRYGMGWLRQAPGKERELVASISWSGHSTYYADSVKGRFTISRDNSKNTLYLQMNSLRAEDTAVYYCAAESLPYESGSPRLTDFASWGQGTLVTVSS

SEQ ID NO: 98 - protein, 10 aa, synthetic construct
  REGION 1..10: note = aTrop2 hVIB619 sdCDR1
GRSFSRYGMG

SEQ ID NO: 99 - protein, 17 aa, synthetic construct
  REGION 1..17: note = aTrop2 hVIB619 sdCDR2
SISWSGHSTYYADSVKG

SEQ ID NO: 100 - protein, 17 aa, synthetic construct
  REGION 1..17: note = aTrop2 hVIB619 sdCDR3
ESLPYESGSPRLTDFAS

SEQ ID NO: 101 - protein, 119 aa, synthetic construct
  REGION 1..119: note = aCA9 hVIB456 sdAb
QVQLVESGGGLVQPGGSLRLSCAASGSALIINAMGWYRQAPGKQRELVATVTRSGRTNYADSVKGRFTISRDNSKNTLYLQMNSLRAEDTAVYYCNVALWIADGEYDYWGQGTLVTVSS

SEQ ID NO: 102 - protein, 10 aa, synthetic construct
  REGION 1..10: note = aCA9 hVIB456 sdAb sdCDR1
GSALIINAMG

SEQ ID NO: 103 - protein, 16 aa, synthetic construct
  REGION 1..16: note = aCA9 hVIB456 sdAb sdCDR2
TVTRSGRTNYADSVKG

SEQ ID NO: 104 - protein, 11 aa, synthetic construct
  REGION 1..11: note = aCA9 hVIB456 sdAb sdCDR3
ALWIADGEYDY

SEQ ID NO: 105 - protein, 116 aa, synthetic construct
  REGION 1..116: note = aCA9 hVIB476 sdAb
QVQLVESGGGLVQPGGSLRLSCAASGNIFIINVMGWYRQAPGKQRELVATITNGGRTHYADSVKGRFTISRDNSKNTLYLQMNSLRAEDTAVYYCNANHIELGDYWGQGTLVTVSS

SEQ ID NO: 106 - protein, 10 aa, synthetic construct
  REGION 1..10: note = aCA9 hVIB476 sdAb sdCDR1
GNIFIINVMG

SEQ ID NO: 107 - protein, 16 aa, synthetic construct
  REGION 1..16: note = aCA9 hVIB476 sdAb sdCDR2
TITNGGRTHYADSVKG

SEQ ID NO: 108 - protein, 8 aa, synthetic construct
  REGION 1..8: note = aCA9 hVIB476 sdAb sdCDR3
NHIELGDY

SEQ ID NO: 109 - protein, 115 aa, synthetic construct
  REGION 1..115: note = aCA9 hVIB407 sdAb
QVQLVESGGGLVQPGGSLRLSCTASGIIFSVYDMGWYRQTPGKQREFVARITAGGGTYLTDSVKGRFTISRDNSKNTLYLQMNSLRAEDTGVYYCNAAWIGDDYWGQGTLVTVSS

SEQ ID NO: 110 - protein, 10 aa, synthetic construct
  REGION 1..10: note = aCA9 hVIB407 sdAb sdCDR1
GIIFSVYDMG

SEQ ID NO: 111 - protein, 16 aa, synthetic construct
  REGION 1..16: note = aCA9 hVIB407 sdAb sdCDR2
RITAGGGTYLTDSVKG

SEQ ID NO: 112 - protein, 7 aa, synthetic construct
  REGION 1..7: note = aCA9 hVIB407 sdAb sdCDR3
AWIGDDY

SEQ ID NO: 113 - protein, 115 aa, synthetic construct
  REGION 1..115: note = aCA9 hVIB445 sdAb
QVQLVESGGGLVKPGGSLRLSCAASGITFNLHAMRWYRRAPGKQRELVAYISARDWTNYADSVKGRFTISRDNAKNSLYLQMNSLRAEDTAVYYCNTDLVGEDYWGRGTLVTVSS

SEQ ID NO: 114 - protein, 10 aa, synthetic construct
  REGION 1..10: note = aCA9 hVIB445 sdAb sdCDR1
GITFNLHAMR

SEQ ID NO: 115 - protein, 16 aa, synthetic construct
  REGION 1..16: note = aCA9 hVIB445 sdAb sdCDR2
YISARDWTNYADSVKG

SEQ ID NO: 116 - protein, 7 aa, synthetic construct
  REGION 1..7: note = aCA9 hVIB445 sdAb sdCDR3
DLVGEDY

SEQ ID NO: 117 - protein, 123 aa, synthetic construct
  REGION 1..123: note = alpha-LyPD3 h787 sdAb
QVQLLESGGGLVQPGGSLRLSCAASGFTLDYYAIGWFRQAPGKEHEAVSCISSSDGSTYYADSVKGRFTISRDNSKNTLYLQMNSLRAEDTAVYYCATVKSCYSDGYALWGSWGQGTLVTVSS

SEQ ID NO: 118 - protein, 10 aa, synthetic construct
  REGION 1..10: note = alpha-LyPD3 h787 sdAb sdCDR1
GFTLDYYAIG

SEQ ID NO: 119 - protein, 17 aa, synthetic construct
  REGION 1..17: note = alpha-LyPD3 h787 sdAb sdCDR2
CISSSDGSTYYADSVKG

SEQ ID NO: 120 - protein, 14 aa, synthetic construct
  REGION 1..14: note = alpha-LyPD3 h787 sdAb sdCDR3
VKSCYSDGYALWGS

SEQ ID NO: 121 - protein, 123 aa, synthetic construct
  REGION 1..123: note = alpha-LyPD3 h790 sdAb
QVQLLESGGGLVQPGGSLRLSCAASGFTLDYYSIGWFRQAPGKEREGVSCISSSDGSTYYADSVKGRFTISRDNSKNTLYLQMNSLRAEDTAVYYCATVKSCYVDGYILWGSWGQGTLVTVSS

SEQ ID NO: 122 - protein, 10 aa, synthetic construct
  REGION 1..10: note = alpha-LyPD3 h790 sdAb sdCDR1
GFTLDYYSIG

SEQ ID NO: 123 - protein, 17 aa, synthetic construct
  REGION 1..17: note = alpha-LyPD3 h790 sdAb sdCDR2
CISSSDGSTYYADSVKG

SEQ ID NO: 124 - protein, 14 aa, synthetic construct
  REGION 1..14: note = alpha-LyPD3 h790 sdAb sdCDR3
VKSCYVDGYILWGS

SEQ ID NO: 125 - protein, 121 aa, synthetic construct
  REGION 1..121: note = alpha-LyPD3 H804 sdAb
QVQLLESGGGLVQPGGSLRLSCAASGFTLDYYAIGWFRQAPGKEHEGVSCISSSDGSTYYADSVKGRFTISRDNSKNTLYLQMNSLRAEDTAVYYCATVRSCASPYELGSWGQGTLVTVSS

SEQ ID NO: 126 - protein, 10 aa, synthetic construct
  REGION 1..10: note = alpha-LyPD3 H804 sdAb sdCDR1
GFTLDYYAIG

SEQ ID NO: 127 - protein, 17 aa, synthetic construct
  REGION 1..17: note = alpha-LyPD3 H804 sdAb sdCDR2
CISSSDGSTYYADSVKG

SEQ ID NO: 128 - protein, 12 aa, synthetic construct
  REGION 1..12: note = alpha-LyPD3 H804 sdAb sdCDR3
VRSCASPYELGS

SEQ ID NO: 129 - protein, 124 aa, synthetic construct
  REGION 1..124: note = alpha-LyPD h773 sdAb (Pro929)
QVQLVESGGGLVQPGGSLRLSCAASGRSFSSYGMGWFRQAPGKEREIVAAISWGGSAVYYANSVKGRFTISRDNSKNTLYLQMNSLRAEDTAVYYCAANRQGRALVRKEFYDYWGQGTLVTVSS

SEQ ID NO: 130 - protein, 10 aa, synthetic construct
  REGION 1..10: note = alpha-LyPD h773 sdAb (Pro929) sdCDR1
GRSFSSYGMG

SEQ ID NO: 131 - protein, 17 aa, synthetic construct
  REGION 1..17: note = alpha-LyPD h773 sdAb (Pro929) sdCDR2
AISWGGSAVYYANSVKG

SEQ ID NO: 132 - protein, 15 aa, synthetic construct
  REGION 1..15: note = alpha-LyPD h773 sdAb (Pro929) sdCDR3
NRQGRALVRKEFYDY

SEQ ID NO: 133 - protein, 117 aa, synthetic construct
  REGION 1..117: note = alpha-LyPD3 h840 sdAb (Pro980)
QVQLLESGGGKVQPGGSLRLSCVVSGDTSSIYLMGWYRQTPGKQRELVAHIRGNGRTNYADSVKGRFTISRDNSKNTLYLQMNSLRAEDTAVYYCNVRNFWGSAEYWGQGTLVTVSS

SEQ ID NO: 134 - protein, 10 aa, synthetic construct
  REGION 1..10: note = alpha-LyPD3 h840 sdAb (Pro980) sdCDR1
GDTSSIYLMG

SEQ ID NO: 135 - protein, 16 aa, synthetic construct
  REGION 1..16: note = alpha-LyPD3 h840 sdAb (Pro980) sdCDR2
HIRGNGRTNYADSVKG

SEQ ID NO: 136 - protein, 9 aa, synthetic construct
  REGION 1..9: note = alpha-LyPD3 h840 sdAb (Pro980) sdCDR3
RNFWGSAEY

SEQ ID NO: 137 - protein, 124 aa, synthetic construct
  REGION 1..124: note = alpha-LyPD3 h885 sdAb (Pro981)
QVQLLESGGGLVQPGGSLRLSCVASRGTFSRYSMGWFRQAPGKEREFVGRISWSGDMTYYADSVKGRFTISRDNSKNTLYLQMNSLRAEDTAVYYCAASTLGYVWNHPNMYGYWGHGTLVTVSS

SEQ ID NO: 138 - protein, 10 aa, synthetic construct
  REGION 1..10: note = alpha-LyPD3 h885 sdAb (Pro981) sdCDR1
RGTFSRYSMG

SEQ ID NO: 139 - protein, 17 aa, synthetic construct
  REGION 1..17: note = alpha-LyPD3 h885 sdAb (Pro981) sdCDR2
RISWSGDMTYYADSVKG

SEQ ID NO: 140 - protein, 15 aa, synthetic construct
  REGION 1..15: note = alpha-LyPD3 h885 sdAb (Pro981) sdCDR3
STLGYVWNHPNMYGY

SEQ ID NO: 141 - protein, 118 aa, synthetic construct
  REGION 1..118: note = alpha-Her-2 1054 sdAb
QVQLQESGGGLVQAGGSLILSCAASGRTFSNYAMGWFRQAPGKEREFVAAINWSGTHTDYADSVKGRFTISRDNVKRTVYLQMNSLKPEDTATYFCSRGWSVFDPDYRGQGTQVTVSS

SEQ ID NO: 142 - protein, 10 aa, synthetic construct
  REGION 1..10: note = alpha-Her-2 1054 sdAb sdCDR1
GRTFSNYAMG

SEQ ID NO: 143 - protein, 17 aa, synthetic construct
  REGION 1..17: note = alpha-Her-2 1054 sdAb sdCDR2
AINWSGTHTDYADSVKG

SEQ ID NO: 144 - protein, 9 aa, synthetic construct
  REGION 1..9: note = alpha-Her-2 1054 sdAb sdCDR3
GWSVFDPDY

SEQ ID NO: 145 - protein, 118 aa, synthetic construct
  REGION 1..118: note = alpha-Her-2 1055 sdAb
QVQLQESGGGLVQPGGSLRLTCEVSGRAISSYAISWFRQAPGEEREFVAAINWSGTHTNYADSVKGRFTISRDNVKNTVYLQMNSLKPEDTATYFCSRGWSVFDPDYRGQGTQVTVSS

SEQ ID NO: 146 - protein, 10 aa, synthetic construct
  REGION 1..10: note = alpha-Her-2 1055 sdAb sdCDR1
GRAISSYAIS

SEQ ID NO: 147 - protein, 17 aa, synthetic construct
  REGION 1..17: note = alpha-Her-2 1055 sdAb sdCDR2
AINWSGTHTNYADSVKG

SEQ ID NO: 148 - protein, 10 aa, synthetic construct
  REGION 1..10: note = alpha-Her-2 1055 sdAb sdCDR3
GWSVFDPDYR

SEQ ID NO: 149 - protein, 118 aa, synthetic construct
  REGION 1..118: note = alpha-Her-2 1074 sdAb
QVQLQESGGGLVQPGGSLRLTCAASGRAISSYAISWFRQAPGEEREFVAAINWSGTHTNYADSVKGRFTISRDNVKNTVYLQMNSLKPEDTATYFCSRGWSVFDPDYRGQGTQVTVSS

SEQ ID NO: 150 - protein, 10 aa, synthetic construct
  REGION 1..10: note = alpha-Her-2 1074 sdAb sdCDR1
GRAISSYAIS

SEQ ID NO: 151 - protein, 17 aa, synthetic construct
  REGION 1..17: note = alpha-Her-2 1074 sdAb sdCDR2
AINWSGTHTNYADSVKG

SEQ ID NO: 152 - protein, 9 aa, synthetic construct
  REGION 1..9: note = alpha-Her-2 1074 sdAb sdCDR3
GWSVFDPDY

SEQ ID NO: 153 - protein, 118 aa, synthetic construct
  REGION 1..118: note = alpha-Her-2 1059 sdAb
QVQLQESGGGLVQPGGSLRLTCAASGRAISSYAISWFRQAPGEEREFVAAINWSGTHTDYADSVKGRFTISRDNAKNTVYLQMNSLKPEDTAVYFCSRGWSVFDPDYRGQGTQVTVSS

SEQ ID NO: 154 - protein, 10 aa, synthetic construct
  REGION 1..10: note = alpha-Her-2 1059 sdAb sdCDR1
GRAISSYAIS

SEQ ID NO: 155 - protein, 17 aa, synthetic construct
  REGION 1..17: note = alpha-Her-2 1059 sdAb sdCDR2
AINWSGTHTDYADSVKG

SEQ ID NO: 156 - protein, 9 aa, synthetic construct
  REGION 1..9: note = alpha-Her-2 1059 sdAb sdCDR3
GWSVFDPDY

SEQ ID NO: 157 - protein, 118 aa, synthetic construct
  REGION 1..118: note = alpha-Her-2 1065 sdAb
QVQLQESGGGLVQPGGSLRLTCEVSGRAISSYAISWFRQAPGEEREFVAAINWSGTHTNYADSVKGRFTISRDNVKRTVYLQMNSLKPEDTATYFCSRGWSVFDPDYRGQGTQVTVSS

SEQ ID NO: 158 - protein, 10 aa, synthetic construct
  REGION 1..10: note = alpha-Her-2 1065 sdAb sdCDR1
GRAISSYAIS

SEQ ID NO: 159 - protein, 17 aa, synthetic construct
  REGION 1..17: note = alpha-Her-2 1065 sdAb sdCDR2
AINWSGTHTNYADSVKG

SEQ ID NO: 160 - protein, 9 aa, synthetic construct
  REGION 1..9: note = alpha-Her-2 1065 sdAb sdCDR3
GWSVFDPDY

SEQ ID NO: 161 - protein, 118 aa, synthetic construct
  REGION 1..118: note = alpha-Her-2 1090 sdAb
QVQLQESGGGLVQTGGSLRLTCEVSGRAISSYAISWFRQAPGEEREFVAAINWSGTHTNYADSVKGRFTISRDNVKNTVYLQMNSLKPEDTATYFCSRGWSLFDPDYRGQGTQVTVSS

SEQ ID NO: 162 - protein, 10 aa, synthetic construct
  REGION 1..10: note = alpha-Her-2 1090 sdAb sdCDR1
GRAISSYAIS

SEQ ID NO: 163 - protein, 17 aa, synthetic construct
  REGION 1..17: note = alpha-Her-2 1090 sdAb sdCDR2
AINWSGTHTNYADSVKG

SEQ ID NO: 164 - protein, 9 aa, synthetic construct
  REGION 1..9: note = alpha-Her-2 1090 sdAb sdCDR3
GWSLFDPDY

SEQ ID NO: 165 - protein, 121 aa, synthetic construct
  REGION 1..121: note = alpha-Her-2 1091 sdAb
QVQLQESGGGLVQPGGSLRLSCAASGFTFDDYAMSWVRQAPGKGLEWVSSINWSGTHTDYADSVKGRFTISRDNAKNTLYLQMNSLKSEDSAVYYCVKGWRLTGGYYDSVQGQGTQVTVSS

SEQ ID NO: 166 - protein, 10 aa, synthetic construct
  REGION 1..10: note = alpha-Her-2 1091 sdAb sdCDR1
GFTFDDYAMS

SEQ ID NO: 167 - protein, 17 aa, synthetic construct
  REGION 1..17: note = alpha-Her-2 1091 sdAb sdCDR2
SINWSGTHTDYADSVKG

SEQ ID NO: 168 - protein, 12 aa, synthetic construct
  REGION 1..12: note = alpha-Her-2 1091 sdAb sdCDR3
GWRLTGGYYDSV

SEQ ID NO: 169 - protein, 121 aa, synthetic construct
  REGION 1..121: note = alpha-Her-2 1092 sdAb
QVQLQESGGGLVQAGASLRLSCAASGFTFDDYALSWVRQAPGKGLEWVSSINWSGTHTDYADSVKGRFTISRDNAKNTLYLQMNSLKSEDSAVYYCVKGWRLTGGYYDSVQGQGTQVTVSS

SEQ ID NO: 170 - protein, 10 aa, synthetic construct
  REGION 1..10: note = alpha-Her-2 1092 sdAb sdCDR1
GFTFDDYALS

SEQ ID NO: 171 - protein, 17 aa, synthetic construct
  REGION 1..17: note = alpha-Her-2 1092 sdAb sdCDR2
SINWSGTHTDYADSVKG

SEQ ID NO: 172 - protein, 12 aa, synthetic construct
  REGION 1..12: note = alpha-Her-2 1092 sdAb sdCDR3
GWRLTGGYYDSV

SEQ ID NO: 173 - protein, 121 aa, synthetic construct
  REGION 1..121: note = alpha-Her-2 1097 sdAb
QVQLQESGGGLVQPGGSLRLSCAASGFIFDDYAMTWVRQAPGKGLEWVSSINWSGTHTDYADSVKGRFTISRDNAKNTLYLQMNSLKSEDSAVYYCVKGWRHTDNYYAGVQGQGTQVTVSS

SEQ ID NO: 174 - protein, 10 aa, synthetic construct
  REGION 1..10: note = alpha-Her-2 1097 sdAb sdCDR1
GFIFDDYAMT

SEQ ID NO: 175 - protein, 17 aa, synthetic construct
  REGION 1..17: note = alpha-Her-2 1097 sdAb sdCDR2
SINWSGTHTDYADSVKG

SEQ ID NO: 176 - protein, 12 aa, synthetic construct
  REGION 1..12: note = alpha-Her-2 1097 sdAb sdCDR3
GWRHTDNYYAGV

SEQ ID NO: 177 - protein, 118 aa, synthetic construct
  REGION 1..118: note = alpha-Her-2 1118 sdAb
QVQLQESGGGLVQAGGSLRLSCAASGFTFDDYAMSWVRQTPGKGLEWVSSINWSGTHTNYADSVKGRFTISRDNAKKTLYLQMNSLKSEDTAVYYCARGWMVVPDSTRGQGTQVTVSS

SEQ ID NO: 178 - protein, 10 aa, synthetic construct
  REGION 1..10: note = alpha-Her-2 1118 sdAb sdCDR1
GFTFDDYAMS

SEQ ID NO: 179 - protein, 17 aa, synthetic construct
  REGION 1..17: note = alpha-Her-2 1118 sdAb sdCDR2
SINWSGTHTNYADSVKG

SEQ ID NO: 180 - protein, 9 aa, synthetic construct
  REGION 1..9: note = alpha-Her-2 1118 sdAb sdCDR3
GWMVVPDST

SEQ ID NO: 181 - protein, 118 aa, synthetic construct
  REGION 1..118: note = alpha-Her-2 1121 sdAb
QVQLQESGGGLVQAGGSLRLSCAASGFIFSDYAMTWVRQAPGKGLEWVSSINWSGTHTNYADSVKGRFTISRDNAKKTLYLQMNSLKSEDTAVYYCARGWMVVPDSTRGQGTQVTVSS

SEQ ID NO: 182 - protein, 10 aa, synthetic construct
  REGION 1..10: note = alpha-Her-2 1121 sdAb sdCDR1
GFIFSDYAMT

SEQ ID NO: 183 - protein, 17 aa, synthetic construct
  REGION 1..17: note = alpha-Her-2 1121 sdAb sdCDR2
SINWSGTHTNYADSVKG

SEQ ID NO: 184 - protein, 9 aa, synthetic construct
  REGION 1..9: note = alpha-Her-2 1121 sdAb sdCDR3
GWMVVPDST

SEQ ID NO: 185 - protein, 113 aa, synthetic construct
  REGION 1..113: note = alpha-Her-2 1134 sdAb
QVQLQESGGGLVQAGGSLRLSCAASGRIFSAAVMAWYRQPPGEQRELVASITHRGFTRYADSVKDRFTISRDNVNNTMYLQMNRLSPEDTAIYYCRLLNGDYWGQGTQVTVSS

SEQ ID NO: 186 - protein, 10 aa, synthetic construct
  REGION 1..10: note = alpha-Her-2 1134 sdAb sdCDR1
GRIFSAAVMA

SEQ ID NO: 187 - protein, 16 aa, synthetic construct
  REGION 1..16: note = alpha-Her-2 1134 sdAb sdCDR2
SITHRGFTRYADSVKD

SEQ ID NO: 188 - protein, 5 aa, synthetic construct
  REGION 1..5: note = alpha-Her-2 1134 sdAb sdCDR3
LNGDY

SEQ ID NO: 189 - protein, 118 aa, synthetic construct
  REGION 1..118: note = alpha-Her-2 1138 sdAb
QVQLQESGGGLVQPGGSLRLSCAASGFTFDDYAMSWVRQAPGKEMEWVSGINWSGTHTRYADSVKGRFTISRDNAKNTLDLQMNSLISEDTAVYYCARGFAADSRSTRGQGTQVTVSS

SEQ ID NO: 190 - protein, 10 aa, synthetic construct
  REGION 1..10: note = alpha-Her-2 1138 sdAb sdCDR1
GFTFDDYAMS

SEQ ID NO: 191 - protein, 17 aa, synthetic construct
  REGION 1..17: note = alpha-Her-2 1138 sdAb sdCDR2
GINWSGTHTRYADSVKG

SEQ ID NO: 192 - protein, 9 aa, synthetic construct
  REGION 1..9: note = alpha-Her-2 1138 sdAb sdCDR3
GFAADSRST

SEQ ID NO: 193 - protein, 118 aa, synthetic construct
  REGION 1..118: note = alpha-Her-2 1139 sdAb
QVQLQESGGGLVQPGGSLRLSCAASGFTFDDYAMSWIRQAPGKEMEWVSGINWSGTHTRYADSVKGRFTISRDNAKNTLDLQMNSLISEDTAVYYCARGFAADSRSTRGQGTQVTVSS

SEQ ID NO: 194 - protein, 10 aa, synthetic construct
  REGION 1..10: note = alpha-Her-2 1139 sdAb sdCDR1
GFTFDDYAMS

SEQ ID NO: 195 - protein, 17 aa, synthetic construct
  REGION 1..17: note = alpha-Her-2 1139 sdAb sdCDR2
GINWSGTHTRYADSVKG

SEQ ID NO: 196 - protein, 9 aa, synthetic construct
  REGION 1..9: note = alpha-Her-2 1139 sdAb sdCDR3
GFAADSRST

SEQ ID NO: 197 - protein, 118 aa, synthetic construct
  REGION 1..118: note = alpha-Her-2 1140 sdAb
QVQLQESGGGLVQPGGSLRLSCVASGFTFDDYAMSWVRQAPGKEMEWVSGINWSGTHTRYADSVKGRFTISRDNAKNTLDLQMNSLISEDTAVYYCARGFAADSRSTRGQGTQVTVSS

SEQ ID NO: 198 - protein, 10 aa, synthetic construct
  REGION 1..10: note = alpha-Her-2 1140 sdAb sdCDR1
GFTFDDYAMS

SEQ ID NO: 199 - protein, 17 aa, synthetic construct
  REGION 1..17: note = alpha-Her-2 1140 sdAb sdCDR2
GINWSGTHTRYADSVKG

SEQ ID NO: 200 - protein, 9 aa, synthetic construct
  REGION 1..9: note = alpha-Her-2 1140 sdAb sdCDR3
GFAADSRST

SEQ ID NO: 201 - protein, 125 aa, synthetic construct
  REGION 1..125: note = alpha-Her-2 1145 sdAb
QVQLQESGGGLVQAGGSLRLSCAASGRIFKNYPMGWFRQPPGKDREFVAAITSSGGTTHYRDSVKGRFTISRDNAKNTVYLQMNSLKPDDTAVYYCAGSSTPWDIPLGPNEVGYWGQGTQVTVSS

SEQ ID NO: 202 - protein, 10 aa, synthetic construct
  REGION 1..10: note = alpha-Her-2 1145 sdAb sdCDR1
GRIFKNYPMG

SEQ ID NO: 203 - protein, 17 aa, synthetic construct
  REGION 1..17: note = alpha-Her-2 1145 sdAb sdCDR2
AITSSGGTTHYRDSVKG

SEQ ID NO: 204 - protein, 16 aa, synthetic construct
  REGION 1..16: note = alpha-Her-2 1145 sdAb sdCDR3
SSTPWDIPLGPNEVGY

SEQ ID NO: 205 - protein, 125 aa, synthetic construct
  REGION 1..125: note = alpha-Her-2 1146 sdAb
QVQLQESGGGLVQAGGSLRLSCAASGRIFKNYPMGWFRQPPGKDREFVAAITSSGGTTPYRDSVKGRFTISRDNAKNTVYLQMNSLKPEDTAVYYCAGSSTPWDIPLGPNEVGFWGQGTQVTVSS

SEQ ID NO: 206 - protein, 10 aa, synthetic construct
  REGION 1..10: note = alpha-Her-2 1146 sdAb sdCDR1
GRIFKNYPMG

SEQ ID NO: 207 - protein, 17 aa, synthetic construct
  REGION 1..17: note = alpha-Her-2 1146 sdAb sdCDR2
AITSSGGTTPYRDSVKG

SEQ ID NO: 208 - protein, 16 aa, synthetic construct
  REGION 1..16: note = alpha-Her-2 1146 sdAb sdCDR3
SSTPWDIPLGPNEVGF

SEQ ID NO: 209 - protein, 115 aa, synthetic construct
  REGION 1..115: note = alpha-Her-2 1149 sdAb
QVQLQESGGGLVQAGGSLRLSCVASGNIFGFNVMGWYRQVPGKQRELVAISTGGRSTDYADSAKGRFTISRNNAKDTVYLQMNSLKPEDTAVYYCKMTMASSDYWGQGTQVTVSS

SEQ ID NO: 210 - protein, 10 aa, synthetic construct
  REGION 1..10: note = alpha-Her-2 1149 sdAb sdCDR1
GNIFGFNVMG

SEQ ID NO: 211 - protein, 16 aa, synthetic construct
  REGION 1..16: note = alpha-Her-2 1149 sdAb sdCDR2
ISTGGRSTDYADSAKG

SEQ ID NO: 212 - protein, 7 aa, synthetic construct
  REGION 1..7: note = alpha-Her-2 1149 sdAb sdCDR3
TMASSDY

SEQ ID NO: 213 - protein, 115 aa, synthetic construct
  REGION 1..115: note = alpha-Her-2 1150 sdAb
QVQLQESGGGLVQAGGSLRLSCVASGNIFGFNVMGWYRQVPGKQRELVAISTGGGSTNYADSAKGRFTVSRNNAKDTVYLQMNSLKPEDTAVYYCKMTMASVDYWGQGTQVTVSS

SEQ ID NO: 214 - protein, 10 aa, synthetic construct
  REGION 1..10: note = alpha-Her-2 1150 sdAb sdCDR1
GNIFGFNVMG

SEQ ID NO: 215 - protein, 16 aa, synthetic construct
  REGION 1..16: note = alpha-Her-2 1150 sdAb sdCDR2
ISTGGGSTNYADSAKG

SEQ ID NO: 216 - protein, 7 aa, synthetic construct
  REGION 1..7: note = alpha-Her-2 1150 sdAb sdCDR3
TMASVDY

SEQ ID NO: 217 - protein, 119 aa, synthetic construct
  REGION 1..119: note = alpha-Her-2 1156 sdAb
QVQLQESGGGLVQAGGSLKVSCAASRNIGSNYAVGWFRQAPGKEREPVAALRWTGSIIGYDDSLRGRFTISKDDAKNTVYLQMNSLKPEDTGVYYCAARILDRSSYDYWGQGTQVTVSS

SEQ ID NO: 218 - protein, 10 aa, synthetic construct
  REGION 1..10: note = alpha-Her-2 1156 sdAb sdCDR1
RNIGSNYAVG

SEQ ID NO: 219 - protein, 17 aa, synthetic construct
  REGION 1..17: note = alpha-Her-2 1156 sdAb sdCDR2
ALRWTGSIIGYDDSLRG

SEQ ID NO: 220 - protein, 10 aa, synthetic construct
  REGION 1..10: note = alpha-Her-2 1156 sdAb sdCDR3
RILDRSSYDY

SEQ ID NO: 221 - protein, 125 aa, synthetic construct
  REGION 1..125: note = alpha-Her-2 1158 sdAb
QVQLQESGGGLVQAGGSLRLSCTASGRTFSSYAMGWFRQAPEKEREFVAAITWSGGTTHYAESVKGRFTISRDNAKNTVYLQMNTLEPEDTAVYYCAARINYSTISSNEKMYHYWGQGTQVTVSS

SEQ ID NO: 222 - protein, 10 aa, synthetic construct
  REGION 1..10: note = alpha-Her-2 1158 sdAb sdCDR1
GRTFSSYAMG

SEQ ID NO: 223 - protein, 17 aa, synthetic construct
  REGION 1..17: note = alpha-Her-2 1158 sdAb sdCDR2
AITWSGGTTHYAESVKG

SEQ ID NO: 224 - protein, 16 aa, synthetic construct
  REGION 1..16: note = alpha-Her-2 1158 sdAb sdCDR3
RINYSTISSNEKMYHY

SEQ ID NO: 225 - protein, 123 aa, synthetic construct
  REGION 1..123: note = alpha-Her-2 1159 sdAb
QVQLQESGGGLVQAGGSLRLSCATSGRTFSSLAMGWFRQAPGKERELVAGISWSGSTTYYADSVKGRFAISRDNAKNTVYLQMNSLKPEDTAVYYCAARTSSIATIGREYDYWGQGTQVTVSS

SEQ ID NO: 226 - protein, 10 aa, synthetic construct
  REGION 1..10: note = alpha-Her-2 1159 sdAb sdCDR1
GRTFSSLAMG

SEQ ID NO: 227 - protein, 17 aa, synthetic construct
  REGION 1..17: note = alpha-Her-2 1159 sdAb sdCDR2
GISWSGSTTYYADSVKG

SEQ ID NO: 228 - protein, 14 aa, synthetic construct
  REGION 1..14: note = alpha-Her-2 1159 sdAb sdCDR3
RTSSIATIGREYDY

SEQ ID NO: 229 - protein, 119 aa, synthetic construct
  REGION 1..119: note = alpha-Her-2 1160 sdAb
QVQLQESGGGLVQAGGSLRLSCAASGRTFSNERLGWFRQAPGKERELVAAIRWSGVIIGYADSVRGRFTISRDNAKSTVYLQMNSLRPEDTAVYFCAADRGVYGTWDYWGQGTQVTVSS

SEQ ID NO: 230 - protein, 10 aa, synthetic construct
  REGION 1..10: note = alpha-Her-2 1160 sdAb sdCDR1
GRTFSNERLG

SEQ ID NO: 231 - protein, 17 aa, synthetic construct
  REGION 1..17: note = alpha-Her-2 1160 sdAb sdCDR2
AIRWSGVIIGYADSVRG

SEQ ID NO: 232 - protein, 10 aa, synthetic construct
  REGION 1..10: note = alpha-Her-2 1160 sdAb sdCDR3
DRGVYGTWDY

SEQ ID NO: 233 - protein, 113 aa, synthetic construct
  REGION 1..113: note = alpha-Her-2 1161 sdAb
QVQLQESGGGLVQPGKSLRLSCAVSGDIFGTYGMAWYRQAPGKQREPVASISIGGIINYADSVKGRFTISRDNVKNTVYLQMNSLKPEDTAVYYCKLLDWNYWGRGTQVTVSS

SEQ ID NO: 234 - protein, 10 aa, synthetic construct
  REGION 1..10: note = alpha-Her-2 1161 sdAb sdCDR1
GDIFGTYGMA

SEQ ID NO: 235 - protein, 16 aa, synthetic construct
  REGION 1..16: note = alpha-Her-2 1161 sdAb sdCDR2
SISIGGIINYADSVKG

SEQ ID NO: 236 - protein, 5 aa, synthetic construct
  REGION 1..5: note = alpha-Her-2 1161 sdAb sdCDR3
LDWNY

SEQ ID NO: 237 - protein, 127 aa, synthetic construct
  REGION 1..127: note = alpha-Her-2 1162 sdAb
QVQLQESGGGLVQAGGSLRLSCAASGRPFSGNAMGWFRQAPGKEREFVAAISWSGDSTYYADSVKGRFTISRDNDENTAYLQMNSLKPEDTAVYYCAATRSFYSRTYYTRPSDYNYWGPGTQVTVSS

SEQ ID NO: 238 - protein, 10 aa, synthetic construct
  REGION 1..10: note = alpha-Her-2 1162 sdAb sdCDR1
GRPFSGNAMG

SEQ ID NO: 239 - protein, 17 aa, synthetic construct
  REGION 1..17: note = alpha-Her-2 1162 sdAb sdCDR2
AISWSGDSTYYADSVKG

SEQ ID NO: 240 - protein, 18 aa, synthetic construct
  REGION 1..18: note = alpha-Her-2 1162 sdAb sdCDR3
TRSFYSRTYYTRPSDYNY

SEQ ID NO: 241 - protein, 125 aa, synthetic construct
  REGION 1..125: note = alpha-Her-2 1163 sdAb
QVQLQESGGGLVQAGGSLRLSCAASGRRFNNYFMGWFRQAPGKEREFVALLRESGDNTYYTNSVKGRFTISRDNARNTVYLQMNSLKPEDTAVYYCAARLDTGFVLYSANSYAYWGQGTQVTVSS

SEQ ID NO: 242 - protein, 10 aa, synthetic construct
  REGION 1..10: note = alpha-Her-2 1163 sdAb sdCDR1
GRRFNNYFMG

SEQ ID NO: 243 - protein, 17 aa, synthetic construct
  REGION 1..17: note = alpha-Her-2 1163 sdAb sdCDR2
LLRESGDNTYYTNSVKG

SEQ ID NO: 244 - protein, 16 aa, synthetic construct
  REGION 1..16: note = alpha-Her-2 1163 sdAb sdCDR3
RLDTGFVLYSANSYAY

SEQ ID NO: 245 - protein, 115 aa, synthetic construct
  REGION 1..115: note = alpha-HSA half-life extension domain (aHSA (10GE))
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 246 - protein, 10 aa, synthetic construct
  REGION 1..10: note = alpha-HSA half-life extension domain (aHSA (10GE)) sdCDR1
GFTFSKFGMS

SEQ ID NO: 247 - protein, 17 aa, synthetic construct
  REGION 1..17: note = alpha-HSA half-life extension domain (aHSA (10GE)) sdCDR2
SISGSGRDTLYAESVKG

SEQ ID NO: 248 - protein, 6 aa, synthetic construct
  REGION 1..6: note = alpha-HSA half-life extension domain (aHSA (10GE)) sdCDR3
GGSLSV

SEQ ID NO: 249 - protein, 121 aa, synthetic construct
  REGION 1..121: note = alpha-HSA half-life extension domain with His tag
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 250 - protein, 10 aa, synthetic construct
  REGION 1..10: note = alpha-HSA half-life extension domain with His tag sdCDR1
GFTFSKFGMS

SEQ ID NO: 251 - protein, 17 aa, synthetic construct
  REGION 1..17: note = alpha-HSA half-life extension domain with His tag sdCDR2
SISGSGRDTLYAESVKG

SEQ ID NO: 252 - protein, 6 aa, synthetic construct
  REGION 1..6: note = alpha-HSA half-life extension domain with His tag sdCDR3
GGSLSV

SEQ ID NO: 253 - skipped

SEQ ID NO: 254 - protein, 109 aa, synthetic construct
  REGION 1..109: note = alpha-CD3 VL
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVL

SEQ ID NO: 255 - protein, 14 aa, synthetic construct
  REGION 1..14: note = alpha-CD3 VL aVLCDR1
ASSTGAVTSGNYPN

SEQ ID NO: 256 - protein, 7 aa, synthetic construct
  REGION 1..7: note = alpha-CD3 VL aVL sdCDR2
GTKFLVP

SEQ ID NO: 257 - protein, 9 aa, synthetic construct
  REGION 1..9: note = alpha-CD3 VL aVL sdCDR3
TLWYSNRWV

SEQ ID NO: 258 - protein, 110 aa, synthetic construct
  REGION 1..110: note = alpha-CD3 VLi
QTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVL

SEQ ID NO: 259 - protein, 14 aa, synthetic construct
  REGION 1..14: note = alpha-CD3 VLi iVLCDR1
GSSTGAVTSGNYPN

SEQ ID NO: 260 - protein, 8 aa, synthetic construct
  REGION 1..8: note = alpha-CD3 VLi iVL sdCDR2
DYKDDDDK

SEQ ID NO: 261 - protein, 9 aa, synthetic construct
  REGION 1..9: note = alpha-CD3 VLi iVL sdCDR3
VLWYSNRWV

SEQ ID NO: 262 - protein, 109 aa, synthetic construct
  REGION 1..109: note = alpha-CD3 VLi2
QTVVTQEPSLTVSPGGTVTLTCGSSTGAVTSGNYPNWVQQKPGQAPRGLIGGTKDDAPGTPARFSGSLLGGKAALTLSGVQPEDEAEYYCVLWYSNRWVFGGGTKLTVL

SEQ ID NO: 263 - protein, 14 aa, synthetic construct
  REGION 1..14: note = alpha-CD3 VLi2 iVLCDR1
GSSTGAVTSGNYPN

SEQ ID NO: 264 - protein, 7 aa, synthetic construct
  REGION 1..7: note = alpha-CD3 VLi2 iVL sdCDR2
GTKDDAP

SEQ ID NO: 265 - protein, 9 aa, synthetic construct
  REGION 1..9: note = alpha-CD3 VLi2 iVL sdCDR3
VLWYSNRWV

SEQ ID NO: 266 - protein, 109 aa, synthetic construct
  REGION 1..109: note = alpha-CD3 VLiGL
QTVVTQEPSLTVSPGGTVTLTCGSSTGAVTSGHYPNWVQQKPGQAPRGLIGGTSNKHSWTPARFSGSLLGGKAALTLSGVQPEDEAEYYCVLWGSRRWVFGGGTKLTVL

SEQ ID NO: 267 - protein, 14 aa, synthetic construct
  REGION 1..14: note = alpha-CD3 VLiGL aVLiGLCDR1
GSSTGAVTSGHYPN

SEQ ID NO: 268 - protein, 7 aa, synthetic construct
  REGION 1..7: note = alpha-CD3 VLiGL aVLiGLCDR2
GTSNKHS

SEQ ID NO: 269 - protein, 9 aa, synthetic construct
  REGION 1..9: note = alpha-CD3 VLiGL aVLiGLCDR3
VLWGSRRWV

SEQ ID NO: 270 - protein, 125 aa, synthetic construct
  REGION 1..125: note = alpha-CD3 VH
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSS

SEQ ID NO: 271 - protein, 10 aa, synthetic construct
  REGION 1..10: note = alpha-CD3 VH aVHCDR1
GFTFNKYAIN

SEQ ID NO: 272 - protein, 19 aa, synthetic construct
  REGION 1..19: note = alpha-CD3 VH aVH sdCDR2
RIRSKYNNYATYYADQVKD

SEQ ID NO: 273 - protein, 14 aa, synthetic construct
  REGION 1..14: note = alpha-CD3 VH aVH sdCDR3
HANFGNSYISYWAY

SEQ ID NO: 274 - protein, 126 aa, synthetic construct
  REGION 1..126: note = alpha-CD3 VHi
EVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSS

SEQ ID NO: 275 - protein, 10 aa, synthetic construct
  REGION 1..10: note = alpha-CD3 VHi iVH sdCDR1
GFTFNKYAMN

SEQ ID NO: 276 - protein, 20 aa, synthetic construct
  REGION 1..20: note = alpha-CD3 VHi iVH sdCDR2
RIRSKYDYKDDDDKADSVKD

SEQ ID NO: 277 - protein, 14 aa, synthetic construct
  REGION 1..14: note = alpha-CD3 VHi2 iVH sdCDR3
HGNFGNSYISYWAY

SEQ ID NO: 278 - protein, 125 aa, synthetic construct
  REGION 1..125: note = alpha-CD3 VHi2
EVQLVESGGGLVQPGGSLKLSCAASGFTFNKHAMNWVRQAPGKGLEWVARIRSKYNNYATAYADSVKDRFTISRDDSKNTAYLQMNNLKTEDTAVYYCVRHGNFGNSYISYWAYWGQGTLVTVSS

SEQ ID NO: 279 - protein, 10 aa, synthetic construct
  REGION 1..10: note = alpha-CD3 VHi2 iVH sdCDR1
GFTFNKHAMN

SEQ ID NO: 280 - protein, 19 aa, synthetic construct
  REGION 1..19: note = alpha-CD3 VHi2 iVH sdCDR2
RIRSKYNNYATAYADSVKD

SEQ ID NO: 281 - protein, 14 aa, synthetic construct
  REGION 1..14: note = alpha-CD3 VHi2 iVH sdCDR3
HGNFGNSYISYWAY

SEQ ID NO: 282 - protein, 125 aa, synthetic construct
  REGION 1..125: note = alpha-CD3 VHiGL4
EVQLVESGGGLVQPGGSLKLSCAASGFTFSGYAMNWVRQAPGKGLEWVARIRSKANSYATEYAASVKDRFTISRDDSKNTAYLQMNNLKTEDTAVYYCVRHGNAGNSAISYWAYWGQGTLVTVSS

SEQ ID NO: 283 - protein, 10 aa, synthetic construct
  REGION 1..10: note = alpha-CD3 VHiGL4 aVHiGL4CDR1
GFTFSGYAMN

SEQ ID NO: 284 - protein, 19 aa, synthetic construct
  REGION 1..19: note = alpha-CD3 VHiGL4 aVHiGL4CDR2
RIRSKANSYATEYAASVKD

SEQ ID NO: 285 - protein, 14 aa, synthetic construct
  REGION 1..14: note = alpha-CD3 VHiGL4 aVHiGL4CDR3
HGNAGNSAISYWAY

SEQ ID NO: 286 - protein, 15 aa, synthetic construct
  REGION 1..15: note = Normal/Non-cleavable linker
GGGGSGGGGSGGGGS

SEQ ID NO: 287 - protein, 8 aa, synthetic construct
  REGION 1..8: note = Constrained linker
GGGSGGGS

SEQ ID NO: 288 - protein, 899 aa, synthetic construct
  REGION 1..899: note = Pro565
QVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 289 - protein, 899 aa, synthetic construct
  REGION 1..899: note = Pro566
QVQLLESGGGLVQPGGSLRLSCAASGRTFSDYDMGWFRQGPGKEREFVAAISWSGGHTNYADSVKGRFTISRDNSKNTLYLQMNSLRAEDTAVYYCAADLRFTGGDTTTPETYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRTFSDYDMGWFRQGPGKEREFVAAISWSGGHTNYADSVKGRFTISRDNSKNTLYLQMNSLRAEDTAVYYCAADLRFTGGDTTTPETYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 290 - protein, 899 aa, synthetic construct
  REGION 1..899: note = Pro567
QVQLVESGGGLVQPGRSLRLSCAASGRTLDNYDMGWFRQGPGKEREFVAAISWSGGSTDYAYSVTGRFTISRDNAKNSLYLQMNSLRAEDTALYYCAADLRFTGGDTMTPETYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVQPGRSLRLSCAASGRTLDNYDMGWFRQGPGKEREFVAAISWSGGSTDYAYSVTGRFTISRDNAKNSLYLQMNSLRAEDTALYYCAADLRFTGGDTMTPETYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 291 - protein, 899 aa, synthetic construct
  REGION 1..899: note = Pro568
QVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSSGGGGSGGGGSGGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 292 - protein, 899 aa, synthetic construct
  REGION 1..899: note = Pro727
QVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSSSGGGKKLADEPEGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 293 - protein, 901 aa, synthetic construct
  REGION 1..901: note = Pro728
QVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSSGSGGPGPAGMHGLPGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 294 - protein, 900 aa, synthetic construct
  REGION 1..900: note = Pro729
QVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSSGGGSARLQSAAPGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 295 - protein, 900 aa, synthetic construct
  REGION 1..900: note = Pro730
QVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSSGARLQSAAPAGLKGAGQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 296 - protein, 900 aa, synthetic construct
  REGION 1..900: note = Pro731
QVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSSGGGSLSGRSDNHGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 297 - protein, 903 aa, synthetic construct
  REGION 1..903: note = Pro676
QVQLLESGGGLVQPGGSLRLSCAASGFTFDYYAIGWFRQAPGKEREGVSCISSSHGSTYYADSVKGRFTISRDNSKNTVYLQMNSLRAEDTAVYYCATAGDGGDYHCSGLVDYGMDYWGKGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGFTFDYYAIGWFRQAPGKEREGVSCISSSHGSTYYADSVKGRFTISRDNSKNTVYLQMNSLRAEDTAVYYCATAGDGGDYHCSGLVDYGMDYWGKGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 298 - protein, 905 aa, synthetic construct
  REGION 1..905: note = Pro677
QVQLLESGGGLVQPGGSLRLSCAASGRTFSSQSMGWFRQAPGKEREFVSAISWTGANPTYADSVKGRFTISRDNSKNTLYLQMNSLRAEDTAVYYCAADTSGGSYYYERATAETSYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRTFSSQSMGWFRQAPGKEREFVSAISWTGANPTYADSVKGRFTISRDNSKNTLYLQMNSLRAEDTAVYYCAADTSGGSYYYERATAETSYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 299 - protein, 893 aa, synthetic construct
  REGION 1..893: note = Pro678
QVQLLESGGGLVQPGGSLRLSCLASGRTVGRTAMGWFRQPPGKEREFVATISWAGGTTYYADFVKGRFTISRDNSKNTLYLQMNSLRAEDTAVYYCAASEPYSDYDPSGMVYWGKGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCLASGRTVGRTAMGWFRQPPGKEREFVATISWAGGTTYYADFVKGRFTISRDNSKNTLYLQMNSLRAEDTAVYYCAASEPYSDYDPSGMVYWGKGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 300 - protein, 893 aa, synthetic construct
  REGION 1..893: note = Pro679
QVQLLESGGGLVQPGGSLRLSCAASGRTFGRAAMGWFRQPPGKEREFAATISWSGSNTYYADFVKGRFTISRDNSKNTVYLQMNSLRAEDTAVYYCAASEPYSDYDPSGMVYWGKGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRTFGRAAMGWFRQPPGKEREFAATISWSGSNTYYADFVKGRFTISRDNSKNTVYLQMNSLRAEDTAVYYCAASEPYSDYDPSGMVYWGKGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 301 - protein, 897 aa, synthetic construct
  REGION 1..897: note = Pro808
QVQLLESGGGLVQPGGSLRLSCALSGLTFNTYPMAWFRQPPGQEREFVADMSWSGTNTYYADSVKGRFTISRDNSKNTLYLQMNSLRAEDTAVYYCAAGWPYSGTGRSTTDYTYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCALSGLTFNTYPMAWFRQPPGQEREFVADMSWSGTNTYYADSVKGRFTISRDNSKNTLYLQMNSLRAEDTAVYYCAAGWPYSGTGRSTTDYTYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 302 - protein, 899 aa, synthetic construct
  REGION 1..899: note = Pro819
QVQLLESGGGLVQPGGSLRLSCAASGRSFSRYGMGWLRQAPGKERELVASISWSGHSTYYADSVKGRFTISRDNSKNTLYLQMNSLRAEDTAVYYCAAESLPYESGSPRLTDFASWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRSFSRYGMGWLRQAPGKERELVASISWSGHSTYYADSVKGRFTISRDNSKNTLYLQMNSLRAEDTAVYYCAAESLPYESGSPRLTDFASWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 303 - protein, 905 aa, synthetic construct
  REGION 1..905: note = Pro681
QVQLLESGGGLVQPGGSLRLSCAASGRTFSSQSMGWFRQAPGKEREFVSAISWTGANPTYADSVKGRFTISRDNSKNTLYLQMNSLRAEDTAVYYCAADTSGGSYYYERATAETSYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRTFSSQSMGWFRQAPGKEREFVSAISWTGANPTYADSVKGRFTISRDNSKNTLYLQMNSLRAEDTAVYYCAADTSGGSYYYERATAETSYDYWGQGTLVTVSSGGGGSGGGGSGGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 304 - protein, 896 aa, synthetic construct
  REGION 1..896: note = Pro621
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGGGSARLQSAAPGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 305 - protein, 892 aa, synthetic construct
  REGION 1..892: note = Pro622
QVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSSGGGSARLQSAAPGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 306 - protein, 897 aa, synthetic construct
  REGION 1..897: note = Pro640
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGSGGPGPAGMHGLPGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 307 - protein, 893 aa, synthetic construct
  REGION 1..893: note = Pro641
QVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSSGSGGPGPAGMHGLPGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 308 - protein, 895 aa, synthetic construct
  REGION 1..895: note = Pro642
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGSGGPGPAGMHGLPGGSQTVVTQEPSLTVSPGGTVTLTCGSSTGAVTSGNYPNWVQQKPGQAPRGLIGGTKDDAPGTPARFSGSLLGGKAALTLSGVQPEDEAEYYCVLWYSNRWVFGGGTKLTVLGGGSGGGSEVQLVESGGGLVQPGGSLKLSCAASGFTFNKHAMNWVRQAPGKGLEWVARIRSKYNNYATAYADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 309 - protein, 891 aa, synthetic construct
  REGION 1..891: note = Pro643
QVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSSGSGGPGPAGMHGLPGGSQTVVTQEPSLTVSPGGTVTLTCGSSTGAVTSGNYPNWVQQKPGQAPRGLIGGTKDDAPGTPARFSGSLLGGKAALTLSGVQPEDEAEYYCVLWYSNRWVFGGGTKLTVLGGGSGGGSEVQLVESGGGLVQPGGSLKLSCAASGFTFNKHAMNWVRQAPGKGLEWVARIRSKYNNYATAYADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 310 - protein, 895 aa, synthetic construct
  REGION 1..895: note = Pro744
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSSGGPGPAGMEGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 311 - protein, 895 aa, synthetic construct
  REGION 1..895: note = Pro746
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSSGGPGPAGPKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 312 - protein, 877 aa, synthetic construct
  REGION 1..877: note = Pro 108
QVQLVESGGGLVQPGGSLRLSCAASGFTVSNSVMAWYRQTPGNEREFVAIINSIGITNYADSVKGRFTISRDNSKNTLYLQMNSLRAEDTAVYVCNRNFDRIYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVQPGGSLRLSCAASGFTVSNSVMAWYRQTPGNEREFVAIINSIGITNYADSVKGRFTISRDNSKNTLYLQMNSLRAEDTAVYVCNRNFDRIYWGQGTLVTVSSGSGGPGPAGMHGLPGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 313 - protein, 875 aa, synthetic construct
  REGION 1..875: note = Pro 109
QVQLVESGGGLVQPGGSLRLSCAAPGNTFSISAMGWYRQAPGKQREWVAVTHSDYSTNYADSVKGRFTISRDNSKNTLYLQMNSLRAEDTAVYYCKHYGIDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVQPGGSLRLSCAAPGNTFSISAMGWYRQAPGKQREWVAVTHSDYSTNYADSVKGRFTISRDNSKNTLYLQMNSLRAEDTAVYYCKHYGIDYWGQGTLVTVSSGSGGPGPAGMHGLPGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 314 - protein, 897 aa, synthetic construct
  REGION 1..897: note = Pro396
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGSGGPGPAGMHGLPGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 315 - protein, 893 aa, synthetic construct
  REGION 1..893: note = Pro476
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGGPGPAGMKGLPGSQTVVTQEPSLTVSPGGTVTLTCGSSTGAVTSGNYPNWVQQKPGQAPRGLIGGTKDDAPGTPARFSGSLLGGKAALTLSGVQPEDEAEYYCVLWYSNRWVFGGGTKLTVLGGGSGGGSEVQLVESGGGLVQPGGSLKLSCAASGFTFNKHAMNWVRQAPGKGLEWVARIRSKYNNYATAYADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 316 - protein, 896 aa, synthetic construct
  REGION 1..896: note = Pro706
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSHTGRSAYFGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 317 - protein, 895 aa, synthetic construct
  REGION 1..895: note = Pro709
EVQLVESGGGVVRPGGSLRLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGVVRPGGSLRLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 318 - protein, 885 aa, synthetic construct
  REGION 1..885: note = Pro470
QVQLVESGGGLVQPGGSLRLSCAASGFTVSNSVMAWYRQTPGNEREFVAIINSIGITNYADSVKGRFTISRDNSKNTLYLQMNSLRAEDTAVYVCNRNFDRIYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 319 - protein, 885 aa, synthetic construct
  REGION 1..885: note = Pro471
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVQPGGSLRLSCAASGFTVSNSVMAWYRQTPGNEREFVAIINSIGITNYADSVKGRFTISRDNSKNTLYLQMNSLRAEDTAVYVCNRNFDRIYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 320 - protein, 884 aa, synthetic construct
  REGION 1..884: note = Pro551
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVQPGGSLRLSCAAPGNTFSISAMGWYRQAPGKQREWVAVTHSDYSTNYADSVKGRFTISRDNSKNTLYLQMNSLRAEDTAVYYCKHYGIDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 321 - protein, 884 aa, synthetic construct
  REGION 1..884: note = Pro552
QVQLVESGGGLVQPGGSLRLSCAAPGNTFSISAMGWYRQAPGKQREWVAVTHSDYSTNYADSVKGRFTISRDNSKNTLYLQMNSLRAEDTAVYYCKHYGIDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 322 - protein, 897 aa, synthetic construct
  REGION 1..897: note = Pro623
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 323 - protein, 897 aa, synthetic construct
  REGION 1..897: note = Pro624
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRTFSDYDMGWFRQGPGKEREFVAAISWSGGHTNYADSVKGRFTISRDNSKNTLYLQMNSLRAEDTAVYYCAADLRFTGGDTTTPETYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 324 - protein, 897 aa, synthetic construct
  REGION 1..897: note = Pro698
QVQLLESGGGLVQPGGSLRLSCAASGRTFSDYDMGWFRQGPGKEREFVAAISWSGGHTNYADSVKGRFTISRDNSKNTLYLQMNSLRAEDTAVYYCAADLRFTGGDTTTPETYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 325 - protein, 897 aa, synthetic construct
  REGION 1..897: note = Pro655
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 326 - protein, 897 aa, synthetic construct
  REGION 1..897: note = Pro656
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRTFSDYDMGWFRQGPGKEREFVAAISWSGGHTNYADSVKGRFTISRDNSKNTLYLQMNSLRAEDTAVYYCAADLRFTGGDTTTPETYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 327 - protein, 897 aa, synthetic construct
  REGION 1..897: note = Pro657
QVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 328 - protein, 897 aa, synthetic construct
  REGION 1..897: note = Pro658
QVQLLESGGGLVQPGGSLRLSCAASGRTFSDYDMGWFRQGPGKEREFVAAISWSGGHTNYADSVKGRFTISRDNSKNTLYLQMNSLRAEDTAVYYCAADLRFTGGDTTTPETYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 329 - protein, 885 aa, synthetic construct
  REGION 1..885: note = Pro516
QVQLVESGGGLVQPGGSLRLSCAASGSALIINAMGWYRQAPGKQRELVATVTRSGRTNYADSVKGRFTISRDNSKNTLYLQMNSLRAEDTAVYYCNVALWIADGEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVQPGGSLRLSCAASGSALIINAMGWYRQAPGKQRELVATVTRSGRTNYADSVKGRFTISRDNSKNTLYLQMNSLRAEDTAVYYCNVALWIADGEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 330 - protein, 879 aa, synthetic construct
  REGION 1..879: note = Pro517
QVQLVESGGGLVQPGGSLRLSCAASGNIFIINVMGWYRQAPGKQRELVATITNGGRTHYADSVKGRFTISRDNSKNTLYLQMNSLRAEDTAVYYCNANHIELGDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVQPGGSLRLSCAASGNIFIINVMGWYRQAPGKQRELVATITNGGRTHYADSVKGRFTISRDNSKNTLYLQMNSLRAEDTAVYYCNANHIELGDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 331 - protein, 877 aa, synthetic construct
  REGION 1..877: note = Pro518
QVQLVESGGGLVQPGGSLRLSCTASGIIFSVYDMGWYRQTPGKQREFVARITAGGGTYLTDSVKGRFTISRDNSKNTLYLQMNSLRAEDTGVYYCNAAWIGDDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVQPGGSLRLSCTASGIIFSVYDMGWYRQTPGKQREFVARITAGGGTYLTDSVKGRFTISRDNSKNTLYLQMNSLRAEDTGVYYCNAAWIGDDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 332 - protein, 877 aa, synthetic construct
  REGION 1..877: note = Pro519
QVQLVESGGGLVKPGGSLRLSCAASGITFNLHAMRWYRRAPGKQRELVAYISARDWTNYADSVKGRFTISRDNAKNSLYLQMNSLRAEDTAVYYCNTDLVGEDYWGRGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVKPGGSLRLSCAASGITFNLHAMRWYRRAPGKQRELVAYISARDWTNYADSVKGRFTISRDNAKNSLYLQMNSLRAEDTAVYYCNTDLVGEDYWGRGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 333 - protein, 880 aa, synthetic construct
  REGION 1..880: note = Pro513
QVQLVESGGGLVQPGGSLRLSCAASGNIFIINVMGWYRQAPGKQRELVATITNGGRTHYADSVKGRFTISRDNSKNTLYLQMNSLRAEDTAVYYCNANHIELGDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVQPGGSLRLSCAASGNIFIINVMGWYRQAPGKQRELVATITNGGRTHYADSVKGRFTISRDNSKNTLYLQMNSLRAEDTAVYYCNANHIELGDYWGQGTLVTVSSGGGSGGGSGGGSGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 334 - protein, 895 aa, synthetic construct
  REGION 1..895: note = Pro186
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 335 - protein, 895 aa, synthetic construct
  REGION 1..895: note = Pro214
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 336 - protein, 895 aa, synthetic construct
  REGION 1..895: note = Pro225
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 337 - protein, 895 aa, synthetic construct
  REGION 1..895: note = Pro295
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGGGGSGGGGSGGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 338 - protein, 1359 aa, synthetic construct
  REGION 1..1359: note = Pro 817
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 339 - protein, 8 aa, synthetic construct
  REGION 1..8: note = MMP 2/9
GPAGMKGL

SEQ ID NO: 340 - protein, 15 aa, synthetic construct
  REGION 1..15: note = MMP 2/9
SGGPGPAGMKGLPGS

SEQ ID NO: 341 - protein, 17 aa, synthetic construct
  REGION 1..17: note = MMP 2/9
SGGGPGPAGMKGLPGGS

SEQ ID NO: 342 - protein, 16 aa, synthetic construct
  REGION 1..16: note = Meprin A/B
GGGGKKLADEPEGGGS

SEQ ID NO: 343 - protein, 15 aa, synthetic construct
  REGION 1..15: note = Meprin A/B
SGGGKKLADEPEGGS

SEQ ID NO: 344 - protein, 8 aa, synthetic construct
  REGION 1..8: note = Meprin A/B
KKLADEPE

SEQ ID NO: 345 - protein, 13 aa, synthetic construct
  REGION 1..13: note = Meprin A/B (Variant, high efficiency)
GGGKFLADEPEGG

SEQ ID NO: 346 - protein, 15 aa, synthetic construct
  REGION 1..15: note = Cathepsin S, K, L
GGGARLQSAAPGGGS

SEQ ID NO: 347 - protein, 16 aa, synthetic construct
  REGION 1..16: note = Cathepsin S, K, L
SGGGARLQSAAPGGGS

SEQ ID NO: 348 - protein, 8 aa, synthetic construct
  REGION 1..8: note = Cathepsin S, K, L
ARLQSAAP

SEQ ID NO: 349 - protein, 18 aa, synthetic construct
  REGION 1..18: note = Meprin/Granzyme B
SGGGGVYADSLEDGGGGS

SEQ ID NO: 350 - protein, 10 aa, synthetic construct
  REGION 1..10: note = Meprin/Granzyme B
GVYADSLEDG

SEQ ID NO: 351 - protein, 16 aa, synthetic construct
  REGION 1..16: note = Matriptase/uPA (MS)
GGGSLSGRSDNHGGGS

SEQ ID NO: 352 - protein, 10 aa, synthetic construct
  REGION 1..10: note = Matriptase/uPA (MS)
GLSGRSDNHG

SEQ ID NO: 353 - protein, 17 aa, synthetic construct
  REGION 1..17: note = Matriptase (MV)
SGGGSFTRQARVVGGGS

SEQ ID NO: 354 - protein, 9 aa, synthetic construct
  REGION 1..9: note = Matriptase (MV)
SFTRQARVV

SEQ ID NO: 355 - protein, 14 aa, synthetic construct
  REGION 1..14: note = CathepsinS/MMP9/Meprin A
ARLQSAAPAGLKGA

SEQ ID NO: 356 - protein, 16 aa, synthetic construct
  REGION 1..16: note = CathepsinS/MMP9/Meprin A
GARLQSAAPAGLKGAG

SEQ ID NO: 357 - protein, 13 aa, synthetic construct
  REGION 1..13: note = MMP9 (Variant, high efficiency)
GGPGPAGMHGLPG

SEQ ID NO: 358 - protein, 17 aa, synthetic construct
  REGION 1..17: note = MMP9 (Variant, high efficiency)
GSGGPGPAGMHGLPGGS

SEQ ID NO: 359 - protein, 13 aa, synthetic construct
  REGION 1..13: note = MMP9 (Variant, low efficiency)
GGPGPAGMEGLPG

SEQ ID NO: 360 - protein, 15 aa, synthetic construct
  REGION 1..15: note = MMP9-15 (K>E)
SGGPGPAGMEGLPGS

SEQ ID NO: 361 - protein, 15 aa, synthetic construct
  REGION 1..15: note = MMP9-15 (M>P)
SGGPGPAGPKGLPGS

SEQ ID NO: 362 - protein, 16 aa, synthetic construct
  REGION 1..16: note = Thrombin 1
GGGGLVPRGSLGGGGS

SEQ ID NO: 363 - protein, 15 aa, synthetic construct
  REGION 1..15: note = Thrombin 2
SSGGGMPRSFRGGGS

SEQ ID NO: 364 - protein, 16 aa, synthetic construct
  REGION 1..16: note = Enterokinase/Flag
GGGGDYKDDDDKGGGS

SEQ ID NO: 365 - protein, 16 aa, synthetic construct
  REGION 1..16: note = KLK7-6
SGGGQNPYSAGRGGGS

SEQ ID NO: 366 - protein, 16 aa, synthetic construct
  REGION 1..16: note = KLK7-13
SGGGQNPYSAGGGSGG

SEQ ID NO: 367 - protein, 16 aa, synthetic construct
  REGION 1..16: note = KLK7-11
SGGGRNVYSAGGGSGG

SEQ ID NO: 368 - protein, 16 aa, synthetic construct
  REGION 1..16: note = KLK7-10
SGGGQNTWSAGKGGGS

SEQ ID NO: 369 - protein, 16 aa, synthetic construct
  REGION 1..16: note = uPA
GGGSHTGRSAYFGGGS

SEQ ID NO: 370 - protein, 15 aa, synthetic construct
  REGION 1..15: note = MMP9-2
SGGPGPAGLKGAPGS

SEQ ID NO: 371 - protein, 8 aa, synthetic construct
  REGION 1..8: note = MMP7
KRALGLPG

SEQ ID NO: 372 - protein, 40 aa, synthetic construct
  REGION 1..40: note = MMP7
DEDEDEDEDEDEDEDERPLALWRSDRDRDRDRDRDRDRDR

SEQ ID NO: 373 - skipped

SEQ ID NO: 374 - protein, 5 aa, synthetic construct
  REGION 1..5: note = MMP9
LEATA

SEQ ID NO: 375 - protein, 10 aa, synthetic construct
  REGION 1..10: note = MMP11
GGAANLVRGG

SEQ ID NO: 376 - protein, 10 aa, synthetic construct
  REGION 1..10: note = MMP14
SGRIGFLRTA

SEQ ID NO: 377 - protein, 6 aa, synthetic construct
  REGION 1..6: note = MMP
RLQLKL

SEQ ID NO: 378 - protein, 7 aa, synthetic construct
  REGION 1..7: note = MMP
RLQLKAC

SEQ ID NO: 379 - protein, 7 aa, synthetic construct
  REGION 1..7: note = MMP2, MMP9, MMP14
  MOD_RES 3: note = Cit
  MOD_RES 5: note = Hof
EPXGXYL

SEQ ID NO: 380 - protein, 5 aa, synthetic construct
  REGION 1..5: note = Urokinase plasminogen activator (upa)
SGRSA

SEQ ID NO: 381 - protein, 4 aa, synthetic construct
  REGION 1..4: note = Urokinase plasminogen activator (upa)
DAFK

SEQ ID NO: 382 - protein, 5 aa, synthetic construct
  REGION 1..5: note = Urokinase plasminogen activator (upa)
GGGRR

SEQ ID NO: 383 - protein, 4 aa, synthetic construct
  REGION 1..4: note = Lysosomal enzyme
GFLG

SEQ ID NO: 384 - protein, 4 aa, synthetic construct
  REGION 1..4: note = Lysosomal enzyme
ALAL

SEQ ID NO: 385-386 - skipped (2 entries)

SEQ ID NO: 387 - protein, 5 aa, synthetic construct
  REGION 1..5: note = Cathepsin D
  MOD_RES 3: note = Cys(Et)
PICFF

SEQ ID NO: 388 - protein, 8 aa, synthetic construct
  REGION 1..8: note = Cathepsin K
GGPRGLPG

SEQ ID NO: 389 - protein, 6 aa, synthetic construct
  REGION 1..6: note = Prostate specific antigen
HSSKLQ

SEQ ID NO: 390 - protein, 7 aa, synthetic construct
  REGION 1..7: note = Prostate specific antigen
HSSKLQL

SEQ ID NO: 391 - protein, 9 aa, synthetic construct
  REGION 1..9: note = Prostate specific antigen
HSSKLQEDA

SEQ ID NO: 392 - protein, 10 aa, synthetic construct
  REGION 1..10: note = Herpes simplex virus protease
LVLASSSFGY

SEQ ID NO: 393 - protein, 10 aa, synthetic construct
  REGION 1..10: note = HIV protease
GVSQNYPIVG

SEQ ID NO: 394 - protein, 10 aa, synthetic construct
  REGION 1..10: note = CMV protease
GVVQASCRLA

SEQ ID NO: 395 - skipped

SEQ ID NO: 396 - protein, 6 aa, synthetic construct
  REGION 1..6: note = Thrombin
DPRSFL

SEQ ID NO: 397 - protein, 6 aa, synthetic construct
  REGION 1..6: note = Thrombin
PPRSFL

SEQ ID NO: 398 - protein, 4 aa, synthetic construct
  REGION 1..4: note = Caspase-3
DEVD

SEQ ID NO: 399 - protein, 5 aa, synthetic construct
  REGION 1..5: note = Caspase-3
DEVDP

SEQ ID NO: 400 - protein, 8 aa, synthetic construct
  REGION 1..8: note = Caspase-3
KGSGDVEG

SEQ ID NO: 401 - protein, 6 aa, synthetic construct
  REGION 1..6: note = Interleukin 1Beta converting enzyme
GWEHDG

SEQ ID NO: 402 - protein, 7 aa, synthetic construct
  REGION 1..7: note = Enterokinase
EDDDDKA

SEQ ID NO: 403 - protein, 9 aa, synthetic construct
  REGION 1..9: note = Fap
KQEQNPGST

SEQ ID NO: 404 - protein, 6 aa, synthetic construct
  REGION 1..6: note = Kallikrein 2
GKAFRR

SEQ ID NO: 405 - protein, 4 aa, synthetic construct
  REGION 1..4: note = Plasmin
DAFK

SEQ ID NO: 406 - protein, 4 aa, synthetic construct
  REGION 1..4: note = Plasmin
DVLK

SEQ ID NO: 407 - protein, 4 aa, synthetic construct
  REGION 1..4: note = Plasmin
DAFK

SEQ ID NO: 408 - protein, 7 aa, synthetic construct
  REGION 1..7: note = Top
ALLLALL

SEQ ID NO: 409 - protein, 889 aa, synthetic construct
  REGION 1..889: note = Pro140 (Format 1)
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGPAGMKGLQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 410 - protein, 890 aa, synthetic construct
  REGION 1..890: note = Pro140b Format 1
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSKKLADEPEQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSKKLADEPEGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 411 - protein, 889 aa, synthetic construct
  REGION 1..889: note = Pro186 Format 2
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 412 - protein, 890 aa, synthetic construct
  REGION 1..890: note = Pro187 Format 2
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSKKLADEPEGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 413 - protein, 895 aa, synthetic construct; identical to SEQ ID NO: 336
  REGION 1..895: note = Pro225 (FL aB7H3 hF7 MMP9 linker) Format 2

SEQ ID NO: 414 - protein, 891 aa, synthetic construct
  REGION 1..891: note = Pro226 (FL aB7H3 hF12 MMP9 linker) Format 2
QVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 415 - protein, 895 aa, synthetic construct
  REGION 1..895: note = Pro233 (humanized Pro186) Format 2
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 416 - protein, 875 aa, synthetic construct
  REGION 1..875: note = Pro311 (FL aFOLR1 h77.2 MMP9 linker) format 2
QVQLVESGGGLVQPGGSLRLSCAASGFTVSNSVMAWYRQTPGNEREFVAIINSIGITNYADSVKGRFTISRDNSKNTLYLQMNSLRAEDTAVYVCNRNFDRIYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVQPGGSLRLSCAASGFTVSNSVMAWYRQTPGNEREFVAIINSIGITNYADSVKGRFTISRDNSKNTLYLQMNSLRAEDTAVYVCNRNFDRIYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 417 - protein, 873 aa, synthetic construct
  REGION 1..873: note = Pro312 (FL aFOLR1 h59.3 MMP9 linker)
QVQLVESGGGLVQPGGSLRLSCAAPGNTFSISAMGWYRQAPGKQREWVAVTHSDYSTNYADSVKGRFTISRDNSKNTLYLQMNSLRAEDTAVYYCKHYGIDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVQPGGSLRLSCAAPGNTFSISAMGWYRQAPGKQREWVAVTHSDYSTNYADSVKGRFTISRDNSKNTLYLQMNSLRAEDTAVYYCKHYGIDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 418 - protein, 881 aa, synthetic construct
  REGION 1..881: note = Pro313 (FL aFOLR1 h22.4 MMP9 linker) format 2
QVQLVESGGGLVQPGGSLRLSCEASGTTFSRDVMGWYRQAPGKQRELVAIISRGGSTNYADSVKGRFTISRDNSKNTLYLQMNSLRAEDTAVYYCNANTATWGRVFWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVQPGGSLRLSCEASGTTFSRDVMGWYRQAPGKQRELVAIISRGGSTNYADSVKGRFTISRDNSKNTLYLQMNSLRAEDTAVYYCNANTATWGRVFWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 419 - protein, 898 aa, synthetic construct
  REGION 1..898: note = Pro246 (FL haEGFR1/haEGFR2 heterologous COBRA with MMP9 linker)
EVQLVESGGGLVQPGGSLRLSCAASGRTFSSYAMGWFRQAPGKEREFVVAINWSSGSTYYADSVKGRFTISRDNSKNTLYLQMNSLRAEDTAVYYCAAGYQINSGNYNFKDYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 420 - protein, 893 aa, synthetic construct
  REGION 1..893: note = Pro256 (FL haEpCAM VIB13/haEGFR1 heterologous COBRA MMP9 linker)
QVQLVESGGGLVQPGGSLTLSCAASGTGSIFSINLMGWYRQAPGKQRELVARITSGDSTVYADSVKGRFTISRDNSKNTLYLQMNSLRPEDTAVYYCNLLLRSSPGATTPYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 421 - protein, 885 aa, synthetic construct; identical to SEQ ID NO: 318
  REGION 1..885: note = Pro420 (FL aFOLR1 h77.2/haEGFR1 heterologous COBRA MMP9 linker)

SEQ ID NO: 422 - protein, 885 aa, synthetic construct; identical to SEQ ID NO: 319
  REGION 1..885: note = Pro421 (FL haEGFR1/aFOLR1 h77.2 heterologous COBRA MMP9 linker)

SEQ ID NO: 423 - protein, 896 aa, synthetic construct
  REGION 1..896: note = Pro393 (Pro186 S9 linker)
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGARLQSAAPAGLKGAGQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 424 - protein, 896 aa, synthetic construct
  REGION 1..896: note = Pro394 (Pro186 ST14(MV) linker)
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSFTRQARVVGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 425 - protein, 896 aa, synthetic construct
  REGION 1..896: note = Pro395 (Pro186 CathS linker)
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSARLQSAAPGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 426 - protein, 897 aa, synthetic construct; identical to SEQ ID NO: 314
  REGION 1..897: note = Pro396 (Pro186 MMP9v linker)

SEQ ID NO: 427 - protein, 896 aa, synthetic construct
  REGION 1..896: note = Pro429 (Pro186 Meprin/GranzymeB linker)
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGGGVYADSLEDGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 428 - protein, 895 aa, synthetic construct
  REGION 1..895: note = Pro430 (Pro186 MMP9-2 linker)
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGGPGPAGLKGAPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 429 - protein, 896 aa, synthetic construct
  REGION 1..896: note = Pro431 (Pro186 ST14(MS) linker)
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSLSGRSDNHGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 430 - protein, 763 aa, synthetic construct
  REGION 1..763: note = Pro258 (Pro186 with a single aEGFR domain and a central aHSA domain)
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSHHHHHH

SEQ ID NO: 431 - protein, 896 aa, synthetic construct
  REGION 1..896: note = Pro221
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGGGQNPYSAGRGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 432 - protein, 896 aa, synthetic construct
  REGION 1..896: note = Pro222
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGGGQNPYSAGGGSGGQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 433 - protein, 896 aa, synthetic construct
  REGION 1..896: note = Pro223
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGGGRNVYSAGGGSGGQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 434 - protein, 896 aa, synthetic construct
  REGION 1..896: note = Pro224
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGGGQNTWSAGKGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 435 - protein, 898 aa, synthetic construct
  REGION 1..898: note = Pro254
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLRLSCAASGRTFSSYAMGWFRQAPGKEREFVVAINWSSGSTYYADSVKGRFTISRDNSKNTLYLQMNSLRAEDTAVYYCAAGYQINSGNYNFKDYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 436 - protein, 893 aa, synthetic construct
  REGION 1..893: note = Pro255
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVQPGGSLTLSCAASGTGSIFSINLMGWYRQAPGKQRELVARITSGDSTVYADSVKGRFTISRDNSKNTLYLQMNSLRPEDTAVYYCNLLLRSSPGATTPYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 437 - protein, 902 aa, synthetic construct
  REGION 1..902: note = Pro262
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 438 - protein, 766 aa, synthetic construct
  REGION 1..766: note = Pro356
EVQLVESGGGLVQPGGSLRLSCAASGRTFSSYAMGWFRQAPGKEREFVVAINWSSGSTYYADSVKGRFTISRDNSKNTLYLQMNSLRAEDTAVYYCAAGYQINSGNYNFKDYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSHHHHHH

SEQ ID NO: 439 - protein, 763 aa, synthetic construct
  REGION 1..763: note = Pro359
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSHHHHHH

SEQ ID NO: 440 - protein, 752 aa, synthetic construct
  REGION 1..752: note = Pro364
QVQLVESGGGLVQPGGSLRLSCAAPGNTFSISAMGWYRQAPGKQREWVAVTHSDYSTNYADSVKGRFTISRDNSKNTLYLQMNSLRAEDTAVYYCKHYGIDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSHHHHHH

SEQ ID NO: 441 - protein, 753 aa, synthetic construct
  REGION 1..753: note = Pro388
QVQLVESGGGLVQPGGSLRLSCAASGFTVSNSVMAWYRQTPGNEREFVAIINSIGITNYADSVKGRFTISRDNSKNTLYLQMNSLRAEDTAVYVCNRNFDRIYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSGGGGSGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSHHHHHH

SEQ ID NO: 442 - protein, 896 aa, synthetic construct; identical to SEQ ID NO: 427
  REGION 1..896: note = Pro429

SEQ ID NO: 443 - protein, 895 aa, synthetic construct; identical to SEQ ID NO: 428
  REGION 1..895: note = Pro430

SEQ ID NO: 444 - protein, 896 aa, synthetic construct; identical to SEQ ID NO: 429
  REGION 1..896: note = Pro431

SEQ ID NO: 445 - protein, 896 aa, synthetic construct
  REGION 1..896: note = Pro432
QVQLVESGGGLVQPGGSLTLSCAASGTGSIFSINLMGWYRQAPGKQRELVARITSGDSTVYADSVKGRFTISRDNSKNTLYLQMNSLRPEDTAVYYCNLLLRSSPGATTPYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSEVQLVESGGGLVQPGGSLRLSCAASGRTFSSYAMGWFRQAPGKEREFVVAINWSSGSTYYADSVKGRFTISRDNSKNTLYLQMNSLRAEDTAVYYCAAGYQINSGNYNFKDYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 446 - protein, 636 aa, synthetic construct
  REGION 1..636: note = Pro448
QVQLVESGGGLVQPGGSLRLSCAASGFTVSNSVMAWYRQTPGNEREFVAIINSIGITNYADSVKGRFTISRDNSKNTLYLQMNSLRAEDTAVYVCNRNFDRIYWGQGTLVTVSSGGGGSGGGSQVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 447 - protein, 636 aa, synthetic construct
  REGION 1..636: note = Pro449
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGGSGGGSQVQLVESGGGLVQPGGSLRLSCAASGFTVSNSVMAWYRQTPGNEREFVAIINSIGITNYADSVKGRFTISRDNSKNTLYLQMNSLRAEDTAVYVCNRNFDRIYWGQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 448 - protein, 636 aa, synthetic construct
  REGION 1..636: note = Pro450
QVQLVESGGGLVQPGGSLRLSCAASGFTVSNSVMAWYRQTPGNEREFVAIINSIGITNYADSVKGRFTISRDNSKNTLYLQMNSLRAEDTAVYVCNRNFDRIYWGQGTLVTVSSGGGGSGGGSQVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 449 - protein, 636 aa, synthetic construct
  REGION 1..636: note = Pro451
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGGSGGGSQVQLVESGGGLVQPGGSLRLSCAASGFTVSNSVMAWYRQTPGNEREFVAIINSIGITNYADSVKGRFTISRDNSKNTLYLQMNSLRAEDTAVYVCNRNFDRIYWGQGTLVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 450 - protein, 893 aa, synthetic construct
  REGION 1..893: note = Pro479
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 451 - protein, 893 aa, synthetic construct
  REGION 1..893: note = Pro480
QVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 452 - protein, 893 aa, synthetic construct
  REGION 1..893: note = Pro495
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSSGGPGPAGMKGLPGSQTVVTQEPSLTVSPGGTVTLTCGSSTGAVTSGNYPNWVQQKPGQAPRGLIGGTKDDAPGTPARFSGSLLGGKAALTLSGVQPEDEAEYYCVLWYSNRWVFGGGTKLTVLGGGSGGGSEVQLVESGGGLVQPGGSLKLSCAASGFTFNKHAMNWVRQAPGKGLEWVARIRSKYNNYATAYADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 453 - protein, 893 aa, synthetic construct
  REGION 1..893: note = alpha-LyPD3 h787 COBRA
QVQLLESGGGLVQPGGSLRLSCAASGFTLDYYAIGWFRQAPGKEHEAVSCISSSDGSTYYADSVKGRFTISRDNSKNTLYLQMNSLRAEDTAVYYCATVKSCYSDGYALWGSWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGFTLDYYAIGWFRQAPGKEHEAVSCISSSDGSTYYADSVKGRFTISRDNSKNTLYLQMNSLRAEDTAVYYCATVKSCYSDGYALWGSWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 454 - protein, 893 aa, synthetic construct
  REGION 1..893: note = alpha-LyPD3 h790 COBRA
QVQLLESGGGLVQPGGSLRLSCAASGFTLDYYSIGWFRQAPGKEREGVSCISSSDGSTYYADSVKGRFTISRDNSKNTLYLQMNSLRAEDTAVYYCATVKSCYVDGYILWGSWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGFTLDYYSIGWFRQAPGKEREGVSCISSSDGSTYYADSVKGRFTISRDNSKNTLYLQMNSLRAEDTAVYYCATVKSCYVDGYILWGSWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 455 - protein, 889 aa, synthetic construct
  REGION 1..889: note = alpha-LyPD3 H804 COBRA
QVQLLESGGGLVQPGGSLRLSCAASGFTLDYYAIGWFRQAPGKEHEGVSCISSSDGSTYYADSVKGRFTISRDNSKNTLYLQMNSLRAEDTAVYYCATVRSCASPYELGSWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGFTLDYYAIGWFRQAPGKEHEGVSCISSSDGSTYYADSVKGRFTISRDNSKNTLYLQMNSLRAEDTAVYYCATVRSCASPYELGSWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 456 - protein, 895 aa, synthetic construct
  REGION 1..895: note = alpha-LyPD3 h773 COBRA
QVQLVESGGGLVQPGGSLRLSCAASGRSFSSYGMGWFRQAPGKEREIVAAISWGGSAVYYANSVKGRFTISRDNSKNTLYLQMNSLRAEDTAVYYCAANRQGRALVRKEFYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGLVQPGGSLRLSCAASGRSFSSYGMGWFRQAPGKEREIVAAISWGGSAVYYANSVKGRFTISRDNSKNTLYLQMNSLRAEDTAVYYCAANRQGRALVRKEFYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 457 - protein, 881 aa, synthetic construct
  REGION 1..881: note = alpha-LyPD3 h840 COBRA
QVQLLESGGGKVQPGGSLRLSCVVSGDTSSIYLMGWYRQTPGKQRELVAHIRGNGRTNYADSVKGRFTISRDNSKNTLYLQMNSLRAEDTAVYYCNVRNFWGSAEYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGKVQPGGSLRLSCVVSGDTSSIYLMGWYRQTPGKQRELVAHIRGNGRTNYADSVKGRFTISRDNSKNTLYLQMNSLRAEDTAVYYCNVRNFWGSAEYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 458 - protein, 895 aa, synthetic construct
  REGION 1..895: note = alpha-LyPD3 h885 COBRA
QVQLLESGGGLVQPGGSLRLSCVASRGTFSRYSMGWFRQAPGKEREFVGRISWSGDMTYYADSVKGRFTISRDNSKNTLYLQMNSLRAEDTAVYYCAASTLGYVWNHPNMYGYWGHGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCVASRGTFSRYSMGWFRQAPGKEREFVGRISWSGDMTYYADSVKGRFTISRDNSKNTLYLQMNSLRAEDTAVYYCAASTLGYVWNHPNMYGYWGHGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 459 - protein, 883 aa, synthetic construct
  REGION 1..883: note = alpha-HER2 1054 COBRA
QVQLQESGGGLVQAGGSLILSCAASGRTFSNYAMGWFRQAPGKEREFVAAINWSGTHTDYADSVKGRFTISRDNVKRTVYLQMNSLKPEDTATYFCSRGWSVFDPDYRGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQAGGSLILSCAASGRTFSNYAMGWFRQAPGKEREFVAAINWSGTHTDYADSVKGRFTISRDNVKRTVYLQMNSLKPEDTATYFCSRGWSVFDPDYRGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 460 - protein, 883 aa, synthetic construct
  REGION 1..883: note = alpha-HER21055 COBRA
QVQLQESGGGLVQPGGSLRLTCEVSGRAISSYAISWFRQAPGEEREFVAAINWSGTHTNYADSVKGRFTISRDNVKNTVYLQMNSLKPEDTATYFCSRGWSVFDPDYRGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLTCEVSGRAISSYAISWFRQAPGEEREFVAAINWSGTHTNYADSVKGRFTISRDNVKNTVYLQMNSLKPEDTATYFCSRGWSVFDPDYRGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 461 - protein, 883 aa, synthetic construct
  REGION 1..883: note = alpha-HER2 1058 COBRA
QVQLQESGGGLVQPGGSLRLTCAASGRAISSYAISWFRQAPGEEREFVAAINWSGTHTNYADSVKGRFTISRDNVKNTVYLQMNSLKPEDTATYFCSRGWSVFDPDYRGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLTCAASGRAISSYAISWFRQAPGEEREFVAAINWSGTHTNYADSVKGRFTISRDNVKNTVYLQMNSLKPEDTATYFCSRGWSVFDPDYRGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 462 - protein, 883 aa, synthetic construct
  REGION 1..883: note = alpha-HER2 1059 COBRA
QVQLQESGGGLVQPGGSLRLTCAASGRAISSYAISWFRQAPGEEREFVAAINWSGTHTDYADSVKGRFTISRDNAKNTVYLQMNSLKPEDTAVYFCSRGWSVFDPDYRGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLTCAASGRAISSYAISWFRQAPGEEREFVAAINWSGTHTDYADSVKGRFTISRDNAKNTVYLQMNSLKPEDTAVYFCSRGWSVFDPDYRGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 463 - protein, 883 aa, synthetic construct
  REGION 1..883: note = alpha-HER2 1065 COBRA
QVQLQESGGGLVQPGGSLRLTCEVSGRAISSYAISWFRQAPGEEREFVAAINWSGTHTNYADSVKGRFTISRDNVKRTVYLQMNSLKPEDTATYFCSRGWSVFDPDYRGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLTCEVSGRAISSYAISWFRQAPGEEREFVAAINWSGTHTNYADSVKGRFTISRDNVKRTVYLQMNSLKPEDTATYFCSRGWSVFDPDYRGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 464 - protein, 883 aa, synthetic construct
  REGION 1..883: note = alpha-HER2 1090 COBRA
QVQLQESGGGLVQTGGSLRLTCEVSGRAISSYAISWFRQAPGEEREFVAAINWSGTHTNYADSVKGRFTISRDNVKNTVYLQMNSLKPEDTATYFCSRGWSLFDPDYRGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQTGGSLRLTCEVSGRAISSYAISWFRQAPGEEREFVAAINWSGTHTNYADSVKGRFTISRDNVKNTVYLQMNSLKPEDTATYFCSRGWSLFDPDYRGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 465 - protein, 889 aa, synthetic construct
  REGION 1..889: note = alpha-HER2 1091 COBRA
QVQLQESGGGLVQPGGSLRLSCAASGFTFDDYAMSWVRQAPGKGLEWVSSINWSGTHTDYADSVKGRFTISRDNAKNTLYLQMNSLKSEDSAVYYCVKGWRLTGGYYDSVQGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCAASGFTFDDYAMSWVRQAPGKGLEWVSSINWSGTHTDYADSVKGRFTISRDNAKNTLYLQMNSLKSEDSAVYYCVKGWRLTGGYYDSVQGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 466 - protein, 889 aa, synthetic construct
  REGION 1..889: note = alpha-HER2 1092 COBRA
QVQLQESGGGLVQAGASLRLSCAASGFTFDDYALSWVRQAPGKGLEWVSSINWSGTHTDYADSVKGRFTISRDNAKNTLYLQMNSLKSEDSAVYYCVKGWRLTGGYYDSVQGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQAGASLRLSCAASGFTFDDYALSWVRQAPGKGLEWVSSINWSGTHTDYADSVKGRFTISRDNAKNTLYLQMNSLKSEDSAVYYCVKGWRLTGGYYDSVQGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 467 - protein, 889 aa, synthetic construct
  REGION 1..889: note = alpha-HER2 1097 COBRA
QVQLQESGGGLVQPGGSLRLSCAASGFIFDDYAMTWVRQAPGKGLEWVSSINWSGTHTDYADSVKGRFTISRDNAKNTLYLQMNSLKSEDSAVYYCVKGWRHTDNYYAGVQGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCAASGFIFDDYAMTWVRQAPGKGLEWVSSINWSGTHTDYADSVKGRFTISRDNAKNTLYLQMNSLKSEDSAVYYCVKGWRHTDNYYAGVQGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 468 - protein, 883 aa, synthetic construct
  REGION 1..883: note = alpha-HER2 1118 COBRA
QVQLQESGGGLVQAGGSLRLSCAASGFTFDDYAMSWVRQTPGKGLEWVSSINWSGTHTNYADSVKGRFTISRDNAKKTLYLQMNSLKSEDTAVYYCARGWMVVPDSTRGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQAGGSLRLSCAASGFTFDDYAMSWVRQTPGKGLEWVSSINWSGTHTNYADSVKGRFTISRDNAKKTLYLQMNSLKSEDTAVYYCARGWMVVPDSTRGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 469 - protein, 883 aa, synthetic construct
  REGION 1..883: note = alpha-HER2 1121 COBRA
QVQLQESGGGLVQAGGSLRLSCAASGFIFSDYAMTWVRQAPGKGLEWVSSINWSGTHTNYADSVKGRFTISRDNAKKTLYLQMNSLKSEDTAVYYCARGWMVVPDSTRGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQAGGSLRLSCAASGFIFSDYAMTWVRQAPGKGLEWVSSINWSGTHTNYADSVKGRFTISRDNAKKTLYLQMNSLKSEDTAVYYCARGWMVVPDSTRGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 470 - protein, 873 aa, synthetic construct
  REGION 1..873: note = alpha-HER2 1134 COBRA
QVQLQESGGGLVQAGGSLRLSCAASGRIFSAAVMAWYRQPPGEQRELVASITHRGFTRYADSVKDRFTISRDNVNNTMYLQMNRLSPEDTAIYYCRLLNGDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQAGGSLRLSCAASGRIFSAAVMAWYRQPPGEQRELVASITHRGFTRYADSVKDRFTISRDNVNNTMYLQMNRLSPEDTAIYYCRLLNGDYWGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 471 - protein, 883 aa, synthetic construct
  REGION 1..883: note = alpha-HER2 1138 COBRA
QVQLQESGGGLVQPGGSLRLSCAASGFTFDDYAMSWVRQAPGKEMEWVSGINWSGTHTRYADSVKGRFTISRDNAKNTLDLQMNSLISEDTAVYYCARGFAADSRSTRGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCAASGFTFDDYAMSWVRQAPGKEMEWVSGINWSGTHTRYADSVKGRFTISRDNAKNTLDLQMNSLISEDTAVYYCARGFAADSRSTRGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 472 - protein, 883 aa, synthetic construct
  REGION 1..883: note = alpha-HER2 1139 COBRA
QVQLQESGGGLVQPGGSLRLSCAASGFTFDDYAMSWIRQAPGKEMEWVSGINWSGTHTRYADSVKGRFTISRDNAKNTLDLQMNSLISEDTAVYYCARGFAADSRSTRGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCAASGFTFDDYAMSWIRQAPGKEMEWVSGINWSGTHTRYADSVKGRFTISRDNAKNTLDLQMNSLISEDTAVYYCARGFAADSRSTRGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 473 - protein, 883 aa, synthetic construct
  REGION 1..883: note = alpha-HER2 1140 COBRA
QVQLQESGGGLVQPGGSLRLSCVASGFTFDDYAMSWVRQAPGKEMEWVSGINWSGTHTRYADSVKGRFTISRDNAKNTLDLQMNSLISEDTAVYYCARGFAADSRSTRGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCVASGFTFDDYAMSWVRQAPGKEMEWVSGINWSGTHTRYADSVKGRFTISRDNAKNTLDLQMNSLISEDTAVYYCARGFAADSRSTRGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 474 - protein, 897 aa, synthetic construct
  REGION 1..897: note = alpha-HER2 1145 COBRA
QVQLQESGGGLVQAGGSLRLSCAASGRIFKNYPMGWFRQPPGKDREFVAAITSSGGTTHYRDSVKGRFTISRDNAKNTVYLQMNSLKPDDTAVYYCAGSSTPWDIPLGPNEVGYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQAGGSLRLSCAASGRIFKNYPMGWFRQPPGKDREFVAAITSSGGTTHYRDSVKGRFTISRDNAKNTVYLQMNSLKPDDTAVYYCAGSSTPWDIPLGPNEVGYWGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 475 - protein, 897 aa, synthetic construct
  REGION 1..897: note = alpha-HER2 1146 COBRA
QVQLQESGGGLVQAGGSLRLSCAASGRIFKNYPMGWFRQPPGKDREFVAAITSSGGTTPYRDSVKGRFTISRDNAKNTVYLQMNSLKPEDTAVYYCAGSSTPWDIPLGPNEVGFWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQAGGSLRLSCAASGRIFKNYPMGWFRQPPGKDREFVAAITSSGGTTPYRDSVKGRFTISRDNAKNTVYLQMNSLKPEDTAVYYCAGSSTPWDIPLGPNEVGFWGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 476 - protein, 877 aa, synthetic construct
  REGION 1..877: note = alpha-HER2 1149 COBRA
QVQLQESGGGLVQAGGSLRLSCVASGNIFGFNVMGWYRQVPGKQRELVAISTGGRSTDYADSAKGRFTISRNNAKDTVYLQMNSLKPEDTAVYYCKMTMASSDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQAGGSLRLSCVASGNIFGFNVMGWYRQVPGKQRELVAISTGGRSTDYADSAKGRFTISRNNAKDTVYLQMNSLKPEDTAVYYCKMTMASSDYWGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 477 - protein, 877 aa, synthetic construct
  REGION 1..877: note = alpha-HER2 1150 COBRA
QVQLQESGGGLVQAGGSLRLSCVASGNIFGFNVMGWYRQVPGKQRELVAISTGGGSTNYADSAKGRFTVSRNNAKDTVYLQMNSLKPEDTAVYYCKMTMASVDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQAGGSLRLSCVASGNIFGFNVMGWYRQVPGKQRELVAISTGGGSTNYADSAKGRFTVSRNNAKDTVYLQMNSLKPEDTAVYYCKMTMASVDYWGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 478 - protein, 885 aa, synthetic construct
  REGION 1..885: note = alpha-HER2 1156 COBRA
QVQLQESGGGLVQAGGSLKVSCAASRNIGSNYAVGWFRQAPGKEREPVAALRWTGSIIGYDDSLRGRFTISKDDAKNTVYLQMNSLKPEDTGVYYCAARILDRSSYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQAGGSLKVSCAASRNIGSNYAVGWFRQAPGKEREPVAALRWTGSIIGYDDSLRGRFTISKDDAKNTVYLQMNSLKPEDTGVYYCAARILDRSSYDYWGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 479 - protein, 897 aa, synthetic construct
  REGION 1..897: note = alpha-HER2 1158 COBRA
QVQLQESGGGLVQAGGSLRLSCTASGRTFSSYAMGWFRQAPEKEREFVAAITWSGGTTHYAESVKGRFTISRDNAKNTVYLQMNTLEPEDTAVYYCAARINYSTISSNEKMYHYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQAGGSLRLSCTASGRTFSSYAMGWFRQAPEKEREFVAAITWSGGTTHYAESVKGRFTISRDNAKNTVYLQMNTLEPEDTAVYYCAARINYSTISSNEKMYHYWGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 480 - protein, 893 aa, synthetic construct
  REGION 1..893: note = alpha-HER2 1159 COBRA
QVQLQESGGGLVQAGGSLRLSCATSGRTFSSLAMGWFRQAPGKERELVAGISWSGSTTYYADSVKGRFAISRDNAKNTVYLQMNSLKPEDTAVYYCAARTSSIATIGREYDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQAGGSLRLSCATSGRTFSSLAMGWFRQAPGKERELVAGISWSGSTTYYADSVKGRFAISRDNAKNTVYLQMNSLKPEDTAVYYCAARTSSIATIGREYDYWGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 481 - protein, 885 aa, synthetic construct
  REGION 1..885: note = alpha-HER2 1160 COBRA
QVQLQESGGGLVQAGGSLRLSCAASGRTFSNERLGWFRQAPGKERELVAAIRWSGVIIGYADSVRGRFTISRDNAKSTVYLQMNSLRPEDTAVYFCAADRGVYGTWDYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQAGGSLRLSCAASGRTFSNERLGWFRQAPGKERELVAAIRWSGVIIGYADSVRGRFTISRDNAKSTVYLQMNSLRPEDTAVYFCAADRGVYGTWDYWGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 482 - protein, 873 aa, synthetic construct
  REGION 1..873: note = alpha-HER2 1161 COBRA
QVQLQESGGGLVQPGKSLRLSCAVSGDIFGTYGMAWYRQAPGKQREPVASISIGGIINYADSVKGRFTISRDNVKNTVYLQMNSLKPEDTAVYYCKLLDWNYWGRGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGKSLRLSCAVSGDIFGTYGMAWYRQAPGKQREPVASISIGGIINYADSVKGRFTISRDNVKNTVYLQMNSLKPEDTAVYYCKLLDWNYWGRGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 483 - protein, 901 aa, synthetic construct
  REGION 1..901: note = alpha-HER2 1162 COBRA
QVQLQESGGGLVQAGGSLRLSCAASGRPFSGNAMGWFRQAPGKEREFVAAISWSGDSTYYADSVKGRFTISRDNDENTAYLQMNSLKPEDTAVYYCAATRSFYSRTYYTRPSDYNYWGPGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQAGGSLRLSCAASGRPFSGNAMGWFRQAPGKEREFVAAISWSGDSTYYADSVKGRFTISRDNDENTAYLQMNSLKPEDTAVYYCAATRSFYSRTYYTRPSDYNYWGPGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 484 - protein, 897 aa, synthetic construct
  REGION 1..897: note = alpha-HER2 1163 COBRA
QVQLQESGGGLVQAGGSLRLSCAASGRRFNNYFMGWFRQAPGKEREFVALLRESGDNTYYTNSVKGRFTISRDNARNTVYLQMNSLKPEDTAVYYCAARLDTGFVLYSANSYAYWGQGTQVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQAGGSLRLSCAASGRRFNNYFMGWFRQAPGKEREFVALLRESGDNTYYTNSVKGRFTISRDNARNTVYLQMNSLKPEDTAVYYCAARLDTGFVLYSANSYAYWGQGTQVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 485 - protein, 902 aa, synthetic construct
  REGION 1..902: note = Pro824 (aEpCAM h665/aTrop-2 h557)
QVQLLESGGGLVQPGGSLRLSCAASGRTFSDYDMGWFRQGPGKEREFVAAISWSGGHTNYADSVKGRFTISRDNSKNTLYLQMNSLRAEDTAVYYCAADLRFTGGDTTTPETYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRTFSSQSMGWFRQAPGKEREFVSAISWTGANPTYADSVKGRFTISRDNSKNTLYLQMNSLRAEDTAVYYCAADTSGGSYYYERATAETSYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 486 - protein, 902 aa, synthetic construct
  REGION 1..902: note = Pro826 (aTrop-2 h557/aEpCAM h665)
QVQLLESGGGLVQPGGSLRLSCAASGRTFSSQSMGWFRQAPGKEREFVSAISWTGANPTYADSVKGRFTISRDNSKNTLYLQMNSLRAEDTAVYYCAADTSGGSYYYERATAETSYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRTFSDYDMGWFRQGPGKEREFVAAISWSGGHTNYADSVKGRFTISRDNSKNTLYLQMNSLRAEDTAVYYCAADLRFTGGDTTTPETYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 487 - protein, 895 aa, synthetic construct
  REGION 1..895: note = Pro751
QVQLQESGGGLVQPGGSLRLSCEASPRTFSTYSMAWFRQAPGKERSFVAAINWSGGNTYYADSVKGRFTISRDNSKNTLYLQMNSLRAEDTAVYYCAAGGVLAHHNYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRTFSDYDMGWFRQGPGKEREFVAAISWSGGHTNYADSVKGRFTISRDNSKNTLYLQMNSLRAEDTAVYYCAADLRFTGGDTTTPETYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 488 - protein, 895 aa, synthetic construct
  REGION 1..895: note = Pro752
QVQLLESGGGLVQPGGSLRLSCAASGRTFSDYDMGWFRQGPGKEREFVAAISWSGGHTNYADSVKGRFTISRDNSKNTLYLQMNSLRAEDTAVYYCAADLRFTGGDTTTPETYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCEASPRTFSTYSMAWFRQAPGKERSFVAAINWSGGNTYYADSVKGRFTISRDNSKNTLYLQMNSLRAEDTAVYYCAAGGVLAHHNYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 489 - protein, 902 aa, synthetic construct; identical to SEQ ID NO: 485
  REGION 1..902: note = Pro824

SEQ ID NO: 490 - protein, 902 aa, synthetic construct; identical to SEQ ID NO: 486
  REGION 1..902: note = Pro826

SEQ ID NO: 491 - protein, 893 aa, synthetic construct
  REGION 1..893: note = Pro1109
QVQLLESGGGLVQPGGSLRLSCATSGRTFSSLAMGWFRQAPGKERELVAGISWSGSTTYYADSVKGRFAISRDNSKNTLYLQMNSLRAEDTAVYYCAARTSSIATIGREYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCATSGRTFSSLAMGWFRQAPGKERELVAGISWSGSTTYYADSVKGRFAISRDNSKNTLYLQMNSLRAEDTAVYYCAARTSSIATIGREYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 492 - protein, 901 aa, synthetic construct
  REGION 1..901: note = Pro1111
QVQLLESGGGLVQPGGSLRLSCAASGRPFSGNAMGWFRQAPGKEREFVAAISWSGDSTYYADSVKGRFTISRDNSKNTLYLQMNSLRAEDTAVYYCAATRSFYSRTYYTRPSDYNYWGPGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASGRPFSGNAMGWFRQAPGKEREFVAAISWSGDSTYYADSVKGRFTISRDNSKNTLYLQMNSLRAEDTAVYYCAATRSFYSRTYYTRPSDYNYWGPGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 493 - protein, 883 aa, synthetic construct
  REGION 1..883: note = Pro1117
QVQLVESGGGVVRPGGSLRLSCAASGFTFDDYAMSWIRQAPGKEMEWVSGINWSGTHTRYADSVKGRFTISRDNAKNSLYLQMNSLRAEDTALYYCARGFAADSRSTRGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLVESGGGVVRPGGSLRLSCAASGFTFDDYAMSWIRQAPGKEMEWVSGINWSGTHTRYADSVKGRFTISRDNAKNSLYLQMNSLRAEDTALYYCARGFAADSRSTRGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 494 - protein, 885 aa, synthetic construct
  REGION 1..885: note = Pro1118
QVQLLESGGGLVQPGGSLRLSCAASRNIGSNYAVGWFRQAPGKEREPVAALRWTGSIIGYDDSLRGRFTISKDNSKNTLYLQMNSLKPEDTAVYYCAARILDRSSYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLLESGGGLVQPGGSLRLSCAASRNIGSNYAVGWFRQAPGKEREPVAALRWTGSIIGYDDSLRGRFTISKDNSKNTLYLQMNSLKPEDTAVYYCAARILDRSSYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 495 - protein, 126 aa, synthetic construct
  REGION 1..126: note = h664
QVQLLESGGGLVQPGGSLRLSCAASGRTLDNYDMGWFRQGPGKEREFVAAISWSGGSTDYAYSVKGRFTISRDNSKNTLYLQMNSLRAEDTAVYYCAADLRFTGGDTMTPETYDYWGQGTLVTVSS

SEQ ID NO: 496 - protein, 10 aa, synthetic construct
  REGION 1..10: note = h664 sdCDR1
GRTLDNYDMG

SEQ ID NO: 497 - protein, 17 aa, synthetic construct
  REGION 1..17: note = h664 sdCDR2
AISWSGGSTDYAYSVKG

SEQ ID NO: 498 - protein, 17 aa, synthetic construct
  REGION 1..17: note = h664 sdCDR3
DLRFTGGDTMTPETYDY

SEQ ID NO: 499 - protein, 118 aa, synthetic construct
  REGION 1..118: note = h1139
QVQLVESGGGVVRPGGSLRLSCAASGFTFDDYAMSWIRQAPGKEMEWVSGINWSGTHTRYADSVKGRFTISRDNAKNSLYLQMNSLRAEDTALYYCARGFAADSRSTRGQGTLVTVSS

SEQ ID NO: 500 - protein, 10 aa, synthetic construct
  REGION 1..10: note = h1139 sdCDR1
GFTFDDYAMS

SEQ ID NO: 501 - protein, 17 aa, synthetic construct
  REGION 1..17: note = h1139 sdCDR2
GINWSGTHTRYADSVKG

SEQ ID NO: 502 - protein, 9 aa, synthetic construct
  REGION 1..9: note = h1139 sdCDR3
GFAADSRST

SEQ ID NO: 503 - protein, 119 aa, synthetic construct
  REGION 1..119: note = h1156
QVQLLESGGGLVQPGGSLRLSCAASRNIGSNYAVGWFRQAPGKEREPVAALRWTGSIIGYDDSLRGRFTISKDNSKNTLYLQMNSLKPEDTAVYYCAARILDRSSYDYWGQGTLVTVSS

SEQ ID NO: 504 - protein, 10 aa, synthetic construct
  REGION 1..10: note = h1156 sdCDR1
RNIGSNYAVG

SEQ ID NO: 505 - protein, 17 aa, synthetic construct
  REGION 1..17: note = h1156 sdCDR2
ALRWTGSIIGYDDSLRG

SEQ ID NO: 506 - protein, 10 aa, synthetic construct
  REGION 1..10: note = h1156 sdCDR3
RILDRSSYDY

SEQ ID NO: 507 - protein, 123 aa, synthetic construct
  REGION 1..123: note = h1159
QVQLLESGGGLVQPGGSLRLSCATSGRTFSSLAMGWFRQAPGKERELVAGISWSGSTTYYADSVKGRFAISRDNSKNTLYLQMNSLRAEDTAVYYCAARTSSIATIGREYDYWGQGTLVTVSS

SEQ ID NO: 508 - protein, 10 aa, synthetic construct
  REGION 1..10: note = h1159 sdCDR1
GRTFSSLAMG

SEQ ID NO: 509 - protein, 17 aa, synthetic construct
  REGION 1..17: note = h1159 sdCDR2
GISWSGSTTYYADSVKG

SEQ ID NO: 510 - protein, 14 aa, synthetic construct
  REGION 1..14: note = h1159 sdCDR3
RTSSIATIGREYDY

SEQ ID NO: 511 - protein, 127 aa, synthetic construct
  REGION 1..127: note = h1162
QVQLLESGGGLVQPGGSLRLSCAASGRPFSGNAMGWFRQAPGKEREFVAAISWSGDSTYYADSVKGRFTISRDNSKNTLYLQMNSLRAEDTAVYYCAATRSFYSRTYYTRPSDYNYWGPGTLVTVSS

SEQ ID NO: 512 - protein, 10 aa, synthetic construct
  REGION 1..10: note = h1162 sdCDR1
GRPFSGNAMG

SEQ ID NO: 513 - protein, 17 aa, synthetic construct
  REGION 1..17: note = h1162 sdCDR2
AISWSGDSTYYADSVKG

SEQ ID NO: 514 - protein, 18 aa, synthetic construct
  REGION 1..18: note = h1162 sdCDR3
TRSFYSRTYYTRPSDYNY

SEQ ID NO: 515 - protein, 5 aa, synthetic construct
  REGION 1..5: note = AA157-161
LALTL

SEQ ID NO: 516 - protein, 5 aa, synthetic construct
  REGION 1..5: note = AA194-198
TRTVC

SEQ ID NO: 517 - protein, 12 aa, synthetic construct
  REGION 1..12: note = AA462-472
QLTFRNPHQALL

SEQ ID NO: 518 - protein, 40 aa, synthetic construct
  SITE 1..40: note = This sequence may encompass 1-10 "Gly Gly Gly Ser" repeating units
GGGSGGGSGGGSGGGSGGGSGGGSGGGSGGGSGGGSGGGS

SEQ ID NO: 519 - protein, 40 aa, synthetic construct
  SITE 1..40: note = This sequence may encompass 1-10 "Gly Gly Ser Gly" repeating units
GGSGGGSGGGSGGGSGGGSGGGSGGGSGGGSGGGSGGGSG

SEQ ID NO: 520 - protein, 50 aa, synthetic construct
  SITE 1..50: note = This sequence may encompass 1-10 "Gly Gly Ser Gly Gly" repeating units
GGSGGGGSGGGGSGGGGSGGGGSGGGGSGGGGSGGGGSGGGGSGGGGSGG

SEQ ID NO: 521 - protein, 50 aa, synthetic construct
  SITE 1..50: note = This sequence may encompass 1-10 "Gly Gly Gly Gly Ser" repeating units
GGGGSGGGGSGGGGSGGGGSGGGGSGGGGSGGGGSGGGGSGGGGSGGGGS

SEQ ID NO: 522 - protein, 893 aa, synthetic construct; identical to SEQ ID NO: 452
  REGION 1..893: note = Pro601

SEQ ID NO: 523 - protein, 889 aa, synthetic construct
  REGION 1..889: note = Pro602
QVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSSSGGPGPAGMKGLPGSQTVVTQEPSLTVSPGGTVTLTCGSSTGAVTSGNYPNWVQQKPGQAPRGLIGGTKDDAPGTPARFSGSLLGGKAALTLSGVQPEDEAEYYCVLWYSNRWVFGGGTKLTVLGGGSGGGSEVQLVESGGGLVQPGGSLKLSCAASGFTFNKHAMNWVRQAPGKGLEWVARIRSKYNNYATAYADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 524 - protein, 891 aa, synthetic construct
  REGION 1..891: note = V3
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSSSGGPGPAGMKGLPGSQTVVTQEPSLTVSPGGTVTLTCGSSTGAVTSGNYPNWVQQKPGQAPRGLIGGTKDDAPGTPARFSGSLLGGKAALTLSGVQPEDEAEYYCVLWYSNRWVFGGGTKLTVLGGGSGGGSEVQLVESGGGLVQPGGSLKLSCAASGFTFNKHAMNWVRQAPGKGLEWVARIRSKYNNYATAYADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 525 - protein, 891 aa, synthetic construct
  REGION 1..891: note = V4
QVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSSSGGPGPAGMKGLPGSQTVVTQEPSLTVSPGGTVTLTCGSSTGAVTSGNYPNWVQQKPGQAPRGLIGGTKDDAPGTPARFSGSLLGGKAALTLSGVQPEDEAEYYCVLWYSNRWVFGGGTKLTVLGGGSGGGSEVQLVESGGGLVQPGGSLKLSCAASGFTFNKHAMNWVRQAPGKGLEWVARIRSKYNNYATAYADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 526 - protein, 891 aa, synthetic construct
  REGION 1..891: note = Pro664
QVQLQESGGGLVQPGGSLRLSCEASPRTFSTYSMAWFRQAPGKERSFVAAINWSGGNTYYADSVKGRFTISRDNSKNTLYLQMNSLRAEDTAVYYCAAGGVLAHHNYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCEASPRTFSTYSMAWFRQAPGKERSFVAAINWSGGNTYYADSVKGRFTISRDNSKNTLYLQMNSLRAEDTAVYYCAAGGVLAHHNYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 527 - protein, 891 aa, synthetic construct
  REGION 1..891: note = Pro665
QVQLQESGGGLVQPGGSLRLSCEASPRTFSTYSMAWFRQAPGKERSFVAAINWSGGQTSYADSVKGRFTISRDNSKNTLYLQMNSLRAEDTAVYYCAAGGVLAHHNYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCEASPRTFSTYSMAWFRQAPGKERSFVAAINWSGGQTSYADSVKGRFTISRDNSKNTLYLQMNSLRAEDTAVYYCAAGGVLAHHNYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 528 - protein, 891 aa, synthetic construct
  REGION 1..891: note = Pro667
QVQLQESGGGLVQPGGSLRLSCEASPRTFSTYSMAWFRQAPGKERSFVAAINWSGGETSYADSVKGRFTISRDNSKNTLYLQMNSLRAEDTAVYYCAAGGVLAHHNYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCEASPRTFSTYSMAWFRQAPGKERSFVAAINWSGGETSYADSVKGRFTISRDNSKNTLYLQMNSLRAEDTAVYYCAAGGVLAHHNYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 529 - protein, 891 aa, synthetic construct
  REGION 1..891: note = Pro694
QVQLQESGGGLVQPGGSLRLSCEASPRTFSTYSMAWFRQAPGKERSFVAAINWSGGNTAYADSVKGRFTISRDNSKNTLYLQMNSLRAEDTAVYYCAAGGVLAHHNYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCEASPRTFSTYSMAWFRQAPGKERSFVAAINWSGGNTAYADSVKGRFTISRDNSKNTLYLQMNSLRAEDTAVYYCAAGGVLAHHNYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 530 - protein, 891 aa, synthetic construct
  REGION 1..891: note = Pro695
QVQLQESGGGLVQPGGSLRLSCEASPRTFSTYSMAWFRQAPGKERSFVAAINWSGGDTSYADSVKGRFTISRDNSKNTLYLQMNSLRAEDTAVYYCAAGGVLAHHNYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCEASPRTFSTYSMAWFRQAPGKERSFVAAINWSGGDTSYADSVKGRFTISRDNSKNTLYLQMNSLRAEDTAVYYCAAGGVLAHHNYEYDYWGQGTLVTVSSSGGPGPAGMKGLPGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 531 - protein, 891 aa, synthetic construct
  REGION 1..891: note = Pro766
QVQLQESGGGLVQPGGSLRLSCEASPRTFSTYSMAWFRQAPGKERSFVAAINWSGGNTYYADSVKGRFTISRDNSKNTLYLQMNSLRAEDTAVYYCAAGGVLAHHNYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGSGGGSQVQLQESGGGLVQPGGSLRLSCEASPRTFSTYSMAWFRQAPGKERSFVAAINWSGGNTYYADSVKGRFTISRDNSKNTLYLQMNSLRAEDTAVYYCAAGGVLAHHNYEYDYWGQGTLVTVSSGGGGSGGGGSGGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGSGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 532 - protein, 6 aa, synthetic construct
  REGION 1..6: note = MMP
PLGLAG

SEQ ID NO: 533 - protein, 6 aa, synthetic construct
  REGION 1..6: note = MMP
PLGLAX

SEQ ID NO: 534 - protein, 6 aa, synthetic construct
  REGION 1..6: note = MMP
  MOD_RES 4: note = Cys(Me)
PLGCAG

SEQ ID NO: 535 - protein, 8 aa, synthetic construct
  REGION 1..8: note = MMP
ESPAYYTA

SEQ ID NO: 536 - protein, 118 aa, synthetic construct
  REGION 1..118: note = Description of Artificial Sequence: Synthetic peptide - wildtype VIB1058
QVQLQESGGGLVQAGGSLRLTCAASGRAISSYAISWFRQAPGEEREFVAAINWSGTHTDYADSVKGRFTISRDNVKRTVYLQMNSLKPEDTATYFCSRGWSVFDPDYRGQGTQVTVSS

SEQ ID NO: 537 - protein, 118 aa, synthetic construct
  REGION 1..118: note = Description of Artificial Sequence: Synthetic peptide - humanized huVIB1058
QVQLLESGGGLVQAGGSLRLSCAASGRAISSYAISWFRQAPGEEREFVAAINWSGTHTDYADSVKGRFTISRDNSKRTVYLQMNSLRAEDTAVYFCSRGWSVFDPDYRGQGTLVTVSS

(19) United States
(12) Patent Application Publication
(10) Pub. No.: US 2023/0340159 A1
(43) Pub. Date: Oct. 26, 2023

(54) CONSTRAINED CONDITIONALLY ACTIVATED BINDING PROTEINS

(71) Applicant: TAKEDA PHARMACEUTICAL COMPAMY LIMITED, OSAKA (JP)

(72) Inventors: Robert B. DuBridge, Belmont, CA (US); Tseng-hui Timothy Chen, Burlingame, CA (US); Patricia A. Culp, Oakland, CA (US); Chad Michael May, San Carlos, CA (US); Danielle Dettling, San Francisco, CA (US); Jeremiah Daniel Degenhardt, El Granada, CA (US)

(73) Assignee: TAKEDA PHARMACEUTICAL COMPANY LIMITED, OSAKA (JP)

(21) Appl. No.: 18/172,433

(22) Filed: Feb. 22, 2023

Related U.S. Application Data
(60) Provisional application No. 63/313,213, filed on Feb. 23, 2022.

(51) Int. Cl. C07K 16/46; C07K 16/32; C07K 16/18; C07K 16/28; A61P 35/00

(52) U.S. Cl. CPC C07K 16/468; A61P 35/00; C07K 16/18; C07K 16/2809; C07K 16/32; A61K 2039/505

(57) ABSTRACT

The invention relates to COnditional Bispecific Redirected Activation constructs, or COBRAs, that are administered in an active pro-drug format. Upon exposure to tumor proteases, the constructs are cleaved and activated, such that they can bind one or more tumor target antigens (TTAs) as well as CD3, thus recruiting T cells expressing CD3 to the tumor, resulting in treatment. In some embodiments, the tumor target antigen includes B7H3, CA9 (CAIX), EGFR, EpCAM, FOLR1, HER2, LyPD3, and/or Trop2.

### RELATED APPLICATION

This application claims priority under 35 U.S.C. § 119(e) to U.S. Provisional Pat. Application No. 63/313,213, filed Feb. 23, 2022, entitled “CONSTRAINED CONDITIONALLY ACTIVATED BINDING PROTEINS,” which is hereby incorporated by reference in its entirety.

### REFERENCE TO AN ELECTRONIC SEQUENCE LISTING

The contents of the electronic sequence listing (T083370042US01-SEQ-ZJG.xml; size: 805,875 bytes; and date of creation: May 4, 2023) is herein incorporated by reference in its entirety.

### BACKGROUND OF THE INVENTION

The selective destruction of an individual cell or a specific cell type is often desirable in a variety of clinical settings. For example, it is a primary goal of cancer therapy to specifically destroy tumor cells, while leaving healthy cells and tissues as intact and undamaged as possible. One such method is by inducing an immune response against the tumor, to make immune effector cells such as natural killer (NK) cells or cytotoxic T lymphocytes (CTLs) attack and destroy tumor cells.

The use of intact monoclonal antibodies (mAb), which provide superior binding specificity and affinity for a tumor-associated antigen, have been successfully applied in the area of cancer treatment and diagnosis. However, the large size of intact mAbs, their poor biodistribution, low potency and long persistence in the blood pool have limited their clinical applications. For example, intact antibodies can exhibit specific accumulation within the tumor area. In biodistribution studies, an inhomogeneous antibody distribution with primary accumulation in the peripheral regions is noted when precisely investigating the tumor. Due to tumor necrosis, inhomogeneous antigen distribution and increased interstitial tissue pressure, it is not possible to reach central portions of the tumor with intact antibody constructs. In contrast, smaller antibody fragments show rapid tumor localization, penetrate deeper into the tumor, and also, are removed relatively rapidly from the bloodstream. However, many antibodies, including scFvs and other constructs, show “on target/off tumor” effects, wherein the molecule is active on non-tumor cells, causing side effects, some of which can be toxic. The present invention is related to novel constructs that are selectively activated in the presence of tumor proteases.

### SUMMARY OF THE INVENTION

In one aspect, provided is a fusion protein comprising, from N- to C-terminal: (a) a first sdABD that binds HER2 (sdABD-HER2); (b) a first domain linker; (c) a constrained Fv domain comprising: (i) a first variable heavy domain comprising a vhCDR1, vhCDR2 and vhCDR3; (ii) a constrained non-cleavable linker (CNCL); and (iii) a first variable light domain comprising vlCDR1, vlCDR2 and vlCDR3; (d) a second domain linker; (e) a second sdABD-HER2; (f) a cleavable linker (CL); (g) a constrained pseudo Fv domain comprising: (i) a first pseudo variable light domain; (ii) a non-cleavable linker (NCL); and (iii) a first pseudo variable heavy domain; (h) a third domain linker; and (i) a third sdABD that binds to human serum albumin (sdABD-HSA); wherein the first variable heavy domain and the first variable light domain of the constrained Fv domain are capable of binding human CD3 but the constrained pseudo Fv domain does not bind CD3; the first variable heavy domain and the first pseudo variable light domain intramolecularly associate to form an inactive Fv; and the first variable light domain and the first pseudo variable heavy domain intramolecularly associate to form an inactive Fv.

In some embodiments, the first and/or second sdABD-HER2 has an amino acid sequence comprising a set of CDRs selected from the group consisting of: (a) a sdCDR1 of SEQ ID NO:194 a sdCDR2 of SEQ ID NO:195 and a sdCDR3 of SEQ ID NO:196; (b) a sdCDR1 of SEQ ID NO:218, a sdCDR2 of SEQ ID NO:219 and a sdCDR3 of SEQ ID NO:220; (c) a sdCDR1 of SEQ ID NO:226, a sdCDR2 of SEQ ID NO:227 and a sdCDR3 of SEQ ID NO:228; (d) a sdCDR1 of SEQ ID NO:238, a sdCDR2 of SEQ ID NO:239 and a sdCDR3 of SEQ ID NO:240; (e) a sdCDR1 of SEQ ID NO:142, a sdCDR2 of SEQ ID NO:143 and a sdCDR3 of SEQ ID NO:144; (f) a sdCDR1 of SEQ ID NO146, a sdCDR2 of SEQ ID NO:147 and a sdCDR3 of SEQ ID NO:148; (g) a sdCDR1 of SEQ ID NO:150, a sdCDR2 of SEQ ID NO:151 and a sdCDR3 of SEQ ID NO:152; (h) a sdCDR1 of SEQ ID NO:154, a sdCDR2 of SEQ ID NO:155, and a sdCDR3 of SEQ ID NO:156; (i) a sdCDR1 of SEQ ID NO:158, a sdCDR2 of SEQ ID NO:159, and a sdCDR3 of SEQ ID NO:160; (j) a sdCDR1 of SEQ ID NO:162, a sdCDR2 of SEQ ID NO:163, and a sdCDR3 of SEQ ID NO:164; (k) a sdCDR1 of SEQ ID NO:166, a sdCDR2 of SEQ ID NO:167, and a sdCDR3 of SEQ ID NO:168; (1) a sdCDR1 of SEQ ID NO:170, a sdCDR2 of SEQ ID NO:171, and a sdCDR3 of SEQ ID NO:172; (m) a sdCDR1 of SEQ ID NO:174, a sdCDR2 of SEQ ID NO:175, and a sdCDR3 of SEQ ID NO:176; (n) a sdCDR1 of SEQ ID NO:178, a sdCDR2 of SEQ ID NO:179, and a sdCDR3 of SEQ ID NO:180; (o) a sdCDR1 of SEQ ID NO:182, a sdCDR2 of SEQ ID NO:183, and a sdCDR3 of SEQ ID NO:184; (p) a sdCDR1 of SEQ ID NO:186, a sdCDR2 of SEQ ID NO:187, and a sdCDR3 of SEQ ID NO:188; (q) a sdCDR1 of SEQ ID NO:190, a sdCDR2 of SEQ ID NO:191, and a sdCDR3 of SEQ ID NO:192;(r) a sdCDR1 of SEQ ID NO:194, a sdCDR2 of SEQ ID NO:195, and a sdCDR3 of SEQ ID NO:196; (s) a sdCDR1 of SEQ ID NO:198, a sdCDR2 of SEQ ID NO:199, and a sdCDR3 of SEQ ID NO:200; (t) a sdCDR1 of SEQ ID NO:202, a sdCDR2 of SEQ ID NO:203, and a sdCDR3 of SEQ ID NO:204; (u) a sdCDR1 of SEQ ID NO:206, a sdCDR2 of SEQ ID NO:207, and a sdCDR3 of SEQ ID NO:203; (v) a sdCDR1 of SEQ ID NO:210, a sdCDR2 of SEQ ID NO:211, and a sdCDR3 of SEQ ID NO:212; (w) a sdCDR1 of SEQ ID NO:214, a sdCDR2 of SEQ ID NO:215, and a sdCDR3 of SEQ ID NO:216; (x) a sdCDR1 of SEQ ID NO:218, a sdCDR2 of SEQ ID NO:219, and a sdCDR3 of SEQ ID NO:220; (y) a sdCDR1 of SEQ ID NO:222, a sdCDR2 of SEQ ID NO:223, and a sdCDR3 of SEQ ID NO:224; (z) a sdCDR1 of SEQ ID NO:226, a sdCDR2 of SEQ ID NO:227, and a sdCDR3 of SEQ ID NO:228; (aa) a sdCDR1 of SEQ ID NO:230, a sdCDR2 of SEQ ID NO:231, and a sdCDR3 of SEQ ID NO:232; (ab) a sdCDR1 of SEQ ID NO:234, a sdCDR2 of SEQ ID NO:235, and a sdCDR3 of SEQ ID NO:236; (ac) a sdCDR1 of SEQ ID NO:238, a sdCDR2 of SEQ ID NO:239, and a sdCDR3 of SEQ ID NO:240; (ad) a sdCDR1 of SEQ ID NO:242, a sdCDR2 of SEQ ID NO:243, and a sdCDR3 with SEQ ID NO:244; and (ae) a sdCDR1 of SEQ ID NO:500, a sdCDR2 of SEQ ID NO:501, and a sdCDR3 with SEQ ID NO:502; (af) a sdCDR1 of SEQ ID NO:504, a sdCDR2 of SEQ ID NO:505, and a sdCDR3 with SEQ ID NO:506; (ag) a sdCDR1 of SEQ ID NO:508, a sdCDR2 of SEQ ID NO:509, and a sdCDR3 with SEQ ID NO:510; and (ah) a sdCDR1 of SEQ ID NO:512, a sdCDR2 of SEQ ID NO:513, and a sdCDR3 with SEQ ID NO:5.

In some embodiments, the first and/or second sdABD-HER2 comprises an amino acid sequence selected from the group consisting of SEQ ID NO:193, SEQ ID NO:217, SEQ ID NO:225, SEQ ID NO:237, SEQ ID NO:141, SEQ ID NO:145, SEQ ID NO:149, SEQ ID NO:153, SEQ ID NO:157, SEQ ID NO:161, SEQ ID NO:165, SEQ ID NO:169, SEQ ID NO:173, SEQ ID NO:177, SEQ ID NO:181, SEQ ID NO:185, SEQ ID NO:189,, SEQ ID NO:197, SEQ ID NO:201, SEQ ID NO:205, SEQ ID NO:209, SEQ ID NO:213, SEQ ID NO:221, SEQ ID NO:229, SEQ ID NO:233, SEQ ID NO:241, SEQ ID NO:499, SEQ ID NO:503, SEQ ID NO:507, and SEQ ID NO:511.

In some embodiments, the first sdABD-HER2 and the second sdABD-HER2 are the same.

In some embodiments, the first sdABD-HER2 and the second sdABD-HER2 are different.

In some embodiments, the first variable heavy domain is N-terminal to the first variable light domain and the pseudo variable light domain is N-terminal to the pseudo variable heavy domain.

In some embodiments, the first variable heavy domain is N-terminal to the first variable light domain and the pseudo variable heavy domain is N-terminal to the pseudo variable light domain.

In some embodiments, the first variable light domain is N-terminal to the first variable heavy domain and the pseudo variable light domain is N-terminal to the pseudo variable heavy domain.

In some embodiments, the first variable light domain is N-terminal to the first variable heavy domain and the pseudo variable heavy domain is N-terminal to the pseudo variable light domain.

In some embodiments, the third sdABD that binds to HSA (sdABD-HSA) has an amino acid sequence comprising: (a) a set of CDRs selected from the group consisting of (i) a sdCDR1 of SEQ ID NO:246, a sdCDR2 of SEQ ID NO:247, and a sdCDR3 of SEQ ID NO:248, and (ii) a sdCDR1 of SEQ ID NO:250, a sdCDR2 of SEQ ID NO:251, and a sdCDR3 of SEQ ID NO:252; or (b) an amino acid sequence selected from the group consisting of SEQ ID NO:245 and SEQ ID NO:249.

In some embodiments, the cleavable linker comprises a cleavage domain sequence selected from the group consisting of SEQ ID NOS:339-408 and 532-535.

In some embodiments, the cleavable linker is cleaved by a human protease selected from the group consisting of MMP2, MMP9, meprin A, meprin B, cathepsin S, capthepsin K, capthesin L, granzyme B, uPA, kallekriein7, matriptase, and thrombin.

In some embodiments, the fusion protein has an amino acid sequence selected from group consisting of SEQ ID NOS:459-484 and 491-494.

Provided herein is a nucleic acid encoding any of the fusion proteins described.

Provided herein is an expression vector comprising any of the nucleic acids described.

Provided herein is a host cell comprising any of the expression vectors described

In some aspects, provided is a method of making a fusion protein of the present disclosure comprising: (i) culturing the host cell described under conditions wherein the fusion protein is expressed and (ii) recovering the fusion protein.

In some aspects, provided is a method of treating cancer in a subject comprising administering any of the fusion proteins described to the subject.

In some aspects, provided is a single domain antigen binding domain (sdABD) that binds human HER2 (sdABD-HER2) comprising (i) an amino acid sequence selected from the group consisting of SEQ ID NO:141, SEQ ID NO:145, SEQ ID NO:149, SEQ ID NO:153, SEQ ID NO:157, SEQ ID NO:161, SEQ ID NO:165, SEQ ID NO:169, SEQ ID NO:173, SEQ ID NO:177, SEQ ID NO:181, SEQ ID NO:185, SEQ ID NO:189, SEQ ID NO:193, SEQ ID NO:197, SEQ ID NO:201, SEQ ID NO:205, SEQ ID NO:209, SEQ ID NO:213, SEQ ID NO:217, SEQ ID NO:221, SEQ ID NO:225, SEQ ID NO:229, SEQ ID NO:233, SEQ ID NO:237, SEQ ID NO:241, SEQ ID NO:499, SEQ ID NO:503, SEQ ID NO:507, and SEQ ID NO:511; or (ii) an amino acid sequence comprising a set of CDRs selected from the group consisting of: (a) a sdCDR1 of SEQ ID NO:194 a sdCDR2 of SEQ ID NO:195 and a sdCDR3 of SEQ ID NO:196; (b) a sdCDR1 of SEQ ID NO:218, a sdCDR2 of SEQ ID NO:219 and a sdCDR3 of SEQ ID NO:220; (c) a sdCDR1 of SEQ ID NO:226, a sdCDR2 of SEQ ID NO:227 and a sdCDR3 of SEQ ID NO:228; (d) a sdCDR1 of SEQ ID NO:238, a sdCDR2 of SEQ ID NO:239 and a sdCDR3 of SEQ ID NO:240; (e) a sdCDR1 of SEQ ID NO:142, a sdCDR2 of SEQ ID NO:143 and a sdCDR3 of SEQ ID NO:144; (f) a sdCDR1 of SEQ ID NO146, a sdCDR2 of SEQ ID NO:147 and a sdCDR3 of SEQ ID NO:148; (g) a sdCDR1 of SEQ ID NO:150, a sdCDR2 of SEQ ID NO:151 and a sdCDR3 of SEQ ID NO:152; (h) a sdCDR1 of SEQ ID NO:154, a sdCDR2 of SEQ ID NO:155, and a sdCDR3 of SEQ ID NO:156; (i) a sdCDR1 of SEQ ID NO:158, a sdCDR2 of SEQ ID NO:159, and a sdCDR3 of SEQ ID NO:160; (j) a sdCDR1 of SEQ ID NO:162, a sdCDR2 of SEQ ID NO:163, and a sdCDR3 of SEQ ID NO:164; k) a sdCDR1 of SEQ ID NO:166, a sdCDR2 of SEQ ID NO:167, and a sdCDR3 of SEQ ID NO:168; (1) a sdCDR1 of SEQ ID NO:170, a sdCDR2 of SEQ ID NO:171, and a sdCDR3 of SEQ ID NO:172; (m) a sdCDR1 of SEQ ID NO:174, a sdCDR2 of SEQ ID NO:175, and a sdCDR3 of SEQ ID NO:176; (n) a sdCDR1 of SEQ ID NO:178, a sdCDR2 of SEQ ID NO:179, and a sdCDR3 of SEQ ID NO:180; (o) a sdCDR1 of SEQ ID NO:182, a sdCDR2 of SEQ ID NO:183, and a sdCDR3 of SEQ ID NO:184; (p) a sdCDR1 of SEQ ID NO:186, a sdCDR2 of SEQ ID NO:187, and a sdCDR3 of SEQ ID NO:188; (q) a sdCDR1 of SEQ ID NO:190, a sdCDR2 of SEQ ID NO:191, and a sdCDR3 of SEQ ID NO:192; (r) a sdCDR1 of SEQ ID NO:194, a sdCDR2 of SEQ ID NO:195, and a sdCDR3 of SEQ ID NO:196; (s) a sdCDR1 of SEQ ID NO:198, a sdCDR2 of SEQ ID NO:199, and a sdCDR3 of SEQ ID NO:200; (t) a sdCDR1 of SEQ ID NO:202, a sdCDR2 of SEQ ID NO:203, and a sdCDR3 of SEQ ID NO:204; (u) a sdCDR1 of SEQ ID NO:206, a sdCDR2 of SEQ ID NO:207, and a sdCDR3 of SEQ ID NO:203; (v) a sdCDR1 of SEQ ID NO:210, a sdCDR2 of SEQ ID NO:211, and a sdCDR3 of SEQ ID NO:212; (w) a sdCDR1 of SEQ ID NO:214, a sdCDR2 of SEQ ID NO:215, and a sdCDR3 of SEQ ID NO:216; (x) a sdCDR1 of SEQ ID NO:218, a sdCDR2 of SEQ ID NO:219, and a sdCDR3 of SEQ ID NO:220; (y) a sdCDR1 of SEQ ID NO:222, a sdCDR2 of SEQ ID NO:223, and a sdCDR3 of SEQ ID NO:224; (z) a sdCDR1 of SEQ ID NO:226, a sdCDR2 of SEQ ID NO:227, and a sdCDR3 of SEQ ID NO:228; aa) a sdCDR1 of SEQ ID NO:230, a sdCDR2 of SEQ ID NO:231, and a sdCDR3 of SEQ ID NO:232; ab) a sdCDR1 of SEQ ID NO:234, a sdCDR2 of SEQ ID NO:235, and a sdCDR3 of SEQ ID NO:236; ac) a sdCDR1 of SEQ ID NO:238, a sdCDR2 of SEQ ID NO:239, and a sdCDR3 of SEQ ID NO:240; ad) a sdCDR1 of SEQ ID NO:242, a sdCDR2 of SEQ ID NO:243, and a sdCDR3 with SEQ ID NO:244; ae) a sdCDR1 of SEQ ID NO:500, a sdCDR2 of SEQ ID NO:501, and a sdCDR3 with SEQ ID NO:502; af) a sdCDR1 of SEQ ID NO:504, a sdCDR2 of SEQ ID NO:505, and a sdCDR3 with SEQ ID NO:506; ag) a sdCDR1 of SEQ ID NO:508, a sdCDR2 of SEQ ID NO:509, and a sdCDR3 with SEQ ID NO:510; and ah) a sdCDR1 of SEQ ID NO:512, a sdCDR2 of SEQ ID NO:513, and a sdCDR3 with SEQ ID NO:514.

In some aspects, provided is a fusion protein comprising, from N- to C-terminal: (a) a first sdABD that binds a tumor target antigen (sdABD-TTA); (b) a first domain linker; (c) a constrained Fv domain comprising: (i) a first variable heavy domain comprising a vhCDR1, vhCDR2 and vhCDR3; (ii) a constrained non-cleavable linker (CNCL); and (iii) a first variable light domain comprising vlCDR1, vlCDR2 and vlCDR3; d) a second domain linker; e) a second sdABD-TTA; f) a cleavable linker (CL); (g) a constrained pseudo Fv domain comprising: (i) a first pseudo variable light domain; (ii) a non-cleavable linker (NCL); and (iii) a first pseudo variable heavy domain; (h) a third domain linker; and (i) a third sdABD that binds to human serum albumin (sdABD-HSA); wherein the first variable heavy domain and the first variable light domain of the constrained Fv domain are capable of binding human CD3 but the constrained pseudo Fv domain does not bind CD3; the first variable heavy domain and the first pseudo variable light domain intramolecularly associate to form an inactive Fv; the first variable light domain and the first pseudo variable heavy domain intramolecularly associate to form an inactive Fv and wherein either (1) the first sdABD-TTA is a sdABD-HER2 or a sdABD-LyPD3, and the second sdABD-TTA is selected from the group consisting of a sdABD-B7H3, a sdABD-CA9, a sdABD-EGFR, a sdABD-EpCAM, a sdABD-FOLR1, a sdABD-HER2, a sdABD-LyPD3 and a sdABD-Trop2; or (2) the first sdABD-TTA is selected from the group consisting of a sdABD-B7H3, a sdABD-CA9, a sdABD-EGFR, a sdABD-EpCAM, a sdABD-FOLR1, a sdABD-HER2, a sdABD-LyPD3 and a sdABD-Trop2, and the second sdABD-TTA is a sdABD-HER2 or a sdABD-LyPD3.

In some embodiments, the first and second sdABD-TTA are each a sdABD-LyPD3. In some embodiments, the first and second sdABD-LPYD3 are the same. In some embodiments, the first and second sdABD-LPYD3 are different.

In some embodiments of the fusion protein, (a) the first sdABD-TTA is a sdABD-HER2 and the second sdABD-TTA is selected from the group consisting of a sdABD-B7H3, a sdABD-CA9, a sdABD-EGFR, a sdABD-EpCAM, a sdABD-FOLR1, a sdABD-LyPD3, and a sdABD-Trop2; (b) the first sdABD-TTA is a sdABD-LyPD3 and the second sdABD-TTA is selected from the group consisting a sdABD-B7H3, a sdABD-CA9, a sdABD-EGFR, a sdABD-EpCAM, a sdABD-FOLR1, a sdABD-HER2, and a sdABD-Trop2; (c) the first sdABD-TTA is selected from the group consisting a sdABD-B7H3, a sdABD-CA9, a sdABD-EGFR, a sdABD-EpCAM, a sdABD-FOLR1, a sdABD-LyPD3, and a sdABD-Trop2 and the second TTA is a sdABD-HER2; or (d) the first sdABD-TTA is selected from the group consisting a sdABD-B7H3, a sdABD-CA9, a sdABD-EGFR, a sdABD-EpCAM, a sdABD-FOLR1, a sdABD-LyPD3, and a sdABD-Trop2 and the second TTA is a sdABD-LyPD3.

In some embodiments, the sdABD-HER2 comprises an amino acid sequence selected from the group consisting of: (a) a set of CDRs comprising a sdCDR1 of SEQ ID NO:194 a sdCDR2 of SEQ ID NO:195 and a sdCDR3 of SEQ ID NO:196; (b) a set of CDRs comprising a sdCDR1 of SEQ ID NO:218, a sdCDR2 of SEQ ID NO:219 and a sdCDR3 of SEQ ID NO:220; (c) a set of CDRs comprising a sdCDR1 of SEQ ID NO:226, a sdCDR2 of SEQ ID NO:227 and a sdCDR3 of SEQ ID NO:228; (d) a set of CDRs comprising a sdCDR1 of SEQ ID NO:238, a sdCDR2 of SEQ ID NO:239 and a sdCDR3 of SEQ ID NO:240; (e) a set of CDRs comprising a sdCDR1 of SEQ ID NO:142, a sdCDR2 of SEQ ID NO:143 and a sdCDR3 of SEQ ID NO:144; (f) a set of CDRs comprising a sdCDR1 of SEQ ID NO:146, a sdCDR2 of SEQ ID NO:147 and a sdCDR3 of SEQ ID NO:148; (g) a set of CDRs comprising a sdCDR1 of SEQ ID NO:150, a sdCDR2 of SEQ ID NO:151 and a sdCDR3 of SEQ ID NO:152; (h) a set of CDRs comprising a sdCDR1 of SEQ ID NO:154, a sdCDR2 of SEQ ID NO:155, and a sdCDR3 of SEQ ID NO:156; (i) a set of CDRs comprising a sdCDR1 of SEQ ID NO:158, a sdCDR2 of SEQ ID NO:159, and a sdCDR3 of SEQ ID NO:160; (j) a set of CDRs comprising a sdCDR1 of SEQ ID NO:162, a sdCDR2 of SEQ ID NO:163, and a sdCDR3 of SEQ ID NO:164; (k) a set of CDRs comprising a sdCDR1 of SEQ ID NO:166, a sdCDR2 of SEQ ID NO:167, and a sdCDR3 of SEQ ID NO:168; (1) a set of CDRs comprising a sdCDR1 of SEQ ID NO:170, a sdCDR2 of SEQ ID NO:171, and a sdCDR3 of SEQ ID NO:172; (m) a set of CDRs comprising a sdCDR1 of SEQ ID NO:174, a sdCDR2 of SEQ ID NO:175, and a sdCDR3 of SEQ ID NO:176; (n) a set of CDRs comprising a sdCDR1 of SEQ ID NO:178, a sdCDR2 of SEQ ID NO:179, and a sdCDR3 of SEQ ID NO:180; (o) a set of CDRs comprising a sdCDR1 of SEQ ID NO:182, a sdCDR2 of SEQ ID NO:183, and a sdCDR3 of SEQ ID NO:184; (p) a set of CDRs comprising a sdCDR1 of SEQ ID NO:186, a sdCDR2 of SEQ ID NO:187, and a sdCDR3 of SEQ ID NO:188; (q) a set of CDRs comprising a sdCDR1 of SEQ ID NO:190, a sdCDR2 of SEQ ID NO:191, and a sdCDR3 of SEQ ID NO:192; (r) a set of CDRs comprising a sdCDR1 of SEQ ID NO:194, a sdCDR2 of SEQ ID NO:195, and a sdCDR3 of SEQ ID NO:196; (s) a set of CDRs comprising a sdCDR1 of SEQ ID NO:198, a sdCDR2 of SEQ ID NO:199, and a sdCDR3 of SEQ ID NO:200; (t) a set of CDRs comprising a sdCDR1 of SEQ ID NO:202, a sdCDR2 of SEQ ID NO:203, and a sdCDR3 of SEQ ID NO:204; (u) a set of CDRs comprising a sdCDR1 of SEQ ID NO:206, a sdCDR2 of SEQ ID NO:207, and a sdCDR3 of SEQ ID NO:203; (v) a set of CDRs comprising a sdCDR1 of SEQ ID NO:210, a sdCDR2 of SEQ ID NO:211, and a sdCDR3 of SEQ ID NO:212; (w) a set of CDRs comprising a sdCDR1 of SEQ ID NO:214, a sdCDR2 of SEQ ID NO:215, and a sdCDR3 of SEQ ID NO:216; (x) a set of CDRs comprising a sdCDR1 of SEQ ID NO:218, a sdCDR2 of SEQ ID NO:219, and a sdCDR3 of SEQ ID NO:220; (y) a set of CDRs comprising a sdCDR1 of SEQ ID NO:222, a sdCDR2 of SEQ ID NO:223, and a sdCDR3 of SEQ ID NO:224; (z) a set of CDRs comprising a sdCDR1 of SEQ ID NO:226, a sdCDR2 of SEQ ID NO:227, and a sdCDR3 of SEQ ID NO:228; (aa) a set of CDRs comprising a sdCDR1 of SEQ ID NO:230, a sdCDR2 of SEQ ID NO:231, and a sdCDR3 of SEQ ID NO:232; (ab) a set of CDRs comprising a sdCDR1 of SEQ ID NO:234, a sdCDR2 of SEQ ID NO:235, and a sdCDR3 of SEQ ID NO:236; (ac) a set of CDRs comprising a sdCDR1 of SEQ ID NO:238, a sdCDR2 of SEQ ID NO:239, and a sdCDR3 of SEQ ID NO:240; and (ad) a set of CDRs comprising a sdCDR1 of SEQ ID NO:242, a sdCDR2 of SEQ ID NO:243, and a sdCDR3 with SEQ ID NO:244; (ae) SEQ ID NO:141; (af) SEQ ID NO:145; (ag) SEQ ID NO:149; (ah) SEQ ID NO:153; (ai) SEQ ID NO:157; (aj) SEQ ID NO:161; (ak) SEQ ID NO:165; (al) SEQ ID NO:169; (am) SEQ ID NO:173; (an) SEQ ID NO:177; (ao) SEQ ID NO:181; (ap) SEQ ID NO:185; (aq) SEQ ID NO:189; (ar) SEQ ID NO:193; (as) SEQ ID NO:197; (at) SEQ ID NO:201; (au) SEQ ID NO:205; (av) SEQ ID NO:209; (aw) SEQ ID NO:213; (ax) SEQ ID NO:217; (ay) SEQ ID NO:221; (az) SEQ ID NO:225; (ba) SEQ ID NO:229; (bb) SEQ ID NO:233; (bc) SEQ ID NO:237; and (bd) SEQ ID NO:241.

The fusion protein of any one of claims [00459]-[0027], wherein the sdABD-LyPD3 comprises an amino acid sequence selected from the group consisting of: (a) a set of CDRs comprising a sdCDR1 of SEQ ID NO:118, a sdCDR2 of SEQ ID NO:119 and a sdCDR3 of SEQ ID NO:120; (b) a set of CDRs comprising a sdCDR1 of SEQ ID NO:122, a sdCDR2 of SEQ ID NO:123 and a sdCDR3 of SEQ ID NO:124; (c) a set of CDRs comprising a sdCDR1 of SEQ ID NO:126, a sdCDR2 of SEQ ID NO:127 and a sdCDR3 of SEQ ID NO:128; (d) a set of CDRs comprising a sdCDR1 of SEQ ID NO:130, a sdCDR2 of SEQ ID NO:131, and a sdCDR3 of SEQ ID NO:132; (e) a set of CDRs comprising a sdCDR1 of SEQ ID NO:134, a sdCDR2 of SEQ ID NO:135, and a sdCDR3 of SEQ ID NO:136; (f) a set of CDRs comprising a sdCDR1 of SEQ ID NO:138, a sdCDR2 of SEQ ID NO:139, and a sdCDR3 of SEQ ID NO:140; (g) SEQ ID NO:117; (h) SEQ ID NO:121; (i) SEQ ID NO:125; (j) SEQ ID NO:129; (k) SEQ ID NO:133; and (1) SEQ ID NO:137.

In some embodiments, the sdABD-B7H3 comprises an amino acid sequence selected from the group consisting of: (i) a set of CDRs comprising a sdCDR1 of SEQ ID NO:34 a sdCDR2 of SEQ ID NO:35 and a sdCDR3 of SEQ ID NO:36; (ii) a set of CDRs comprising a sdCDR1 of SEQ ID NO:38, a sdCDR2 of SEQ ID NO:39 and a sdCDR3 of SEQ ID NO:40; (iii) a set of CDRs comprising a sdCDR1 of SEQ ID NO:42, a sdCDR2 of SEQ ID NO:43 and a sdCDR3 of SEQ ID NO:44; (iv) a set of CDRs comprising a sdCDR1 of SEQ ID NO:46, a sdCDR2 of SEQ ID NO:47 and a sdCDR3 of SEQ ID NO:48; (v) a set of CDRs comprising a sdCDR1 of SEQ ID NO:50, a sdCDR2 of SEQ ID NO:51 and a sdCDR3 of SEQ ID NO:52; (vi) a set of CDRs comprising a sdCDR1 of SEQ ID NO:54, a sdCDR2 of SEQ ID NO:55 and a sdCDR3 of SEQ ID NO:56; (vii) a set of CDRs comprising a sdCDR1 of SEQ ID NO:58, a sdCDR2 of SEQ ID NO:59 and a sdCDR3 of SEQ ID NO:60; (ix) SEQ ID NO:33; (x) SEQ ID NO:37; (xi) SEQ ID NO:41; (xii) SEQ ID NO:45; (xiii) SEQ ID NO:49; (xiv) SEQ ID NO:53; and (xv) SEQ ID NO:57.

In some embodiments, the sdABD-CA9 comprises an amino acid sequence selected from the group consisting of: (i) a set of CDRs comprising a sdCDR1 of SEQ ID NO:102, a sdCDR2 of SEQ ID NO:103 and a sdCDR3 of SEQ ID NO:104; (ii) a set of CDRs comprising a sdCDR1 of SEQ ID NO:106, a sdCDR2 of SEQ ID NO:107 and a sdCDR3 of SEQ ID NO:108; (iii) a set of CDRs comprising a sdCDR1 of SEQ ID NO:110, a sdCDR2 of SEQ ID NO:111 and a sdCDR3 of SEQ ID NO:112; (iv) a set of CDRs comprising a sdCDR1 of SEQ ID NO:114, a sdCDR2 of SEQ ID NO:115 and a sdCDR3 of SEQ ID NO:116; (v) SEQ ID NO:101; (vi) SEQ ID NO:105; (vii) SEQ ID NO:109; and (viiii) SEQ ID NO:113.

In some embodiments, the sdABD-EGFR comprises an amino acid sequence selected from the group consisting of: (i) a set of CDRs comprising a sdCDR1 of SEQ ID NO:2 a sdCDR2 of SEQ ID NO:3 and a sdCDR3 of SEQ ID NO:4; (ii) a set of CDRs comprising a sdCDR1 of SEQ ID NO:6, a sdCDR2 of SEQ ID NO:7 and a sdCDR3 of SEQ ID NO:8; (iii) a set of CDRs comprising a sdCDR1 of SEQ ID NO:10, a sdCDR2 of SEQ ID NO:11 and a sdCDR3 of SEQ ID NO:12; (iv) a set of CDRs comprising a sdCDR1 of SEQ ID NO:14, a sdCDR2 of SEQ ID NO:15 and a sdCDR3 of SEQ ID NO:16; (v) a set of CDRs comprising a sdCDR1 of SEQ ID NO:18, a sdCDR2 of SEQ ID NO:19 and a sdCDR3 of SEQ ID NO:20; (vi) SEQ ID NO:1; (vii) SEQ ID NO:5; (viii) SEQ ID NO:9; (ix) SEQ ID NO:13; and (x) SEQ ID NO:17.

In some embodiments, the sdABD-EpCAM comprises an amino acid sequence selected from the group consisting of: (i) a set of CDRs comprising a sdCDR1 of SEQ ID NO:62 a sdCDR2 of SEQ ID NO:63 and a sdCDR3 of SEQ ID NO:64; (ii) a set of CDRs comprising a sdCDR1 of SEQ ID NO:66, a sdCDR2 of SEQ ID NO:67 and a sdCDR3 of SEQ ID NO:68; (iii) a set of CDRs comprising a sdCDR1 of SEQ ID NO:70, a sdCDR2 of SEQ ID NO:71 and a sdCDR3 of SEQ ID NO:72; (iv) a set of CDRs comprising a sdCDR1 of SEQ ID NO:74, a sdCDR2 of SEQ ID NO:75 and a sdCDR3 of SEQ ID NO:76; (v) a set of CDRs comprising a sdCDR1 of SEQ ID NO:496, a sdCDR2 of SEQ ID NO:497 and a sdCDR3 of SEQ ID NO:498; (vi) SEQ ID NO:61; (vii) SEQ ID NO:65; (viii) SEQ ID NO:69; (ix) SEQ ID NO:73; and (x) SEQ ID NO:495.

In some embodiments, the sdABD-FOLR1 comprises an amino acid sequence selected from the group consisting of: (i) a set of CDRs comprising a sdCDR1 of SEQ ID NO:22 a sdCDR2 of SEQ ID NO:23 and a sdCDR3 of SEQ ID NO:24; (ii) a set of CDRs comprising a sdCDR1 of SEQ ID NO:26, a sdCDR2 of SEQ ID NO:27 and a sdCDR3 of SEQ ID NO:28; (iii) a set of CDRs comprising a sdCDR1 of SEQ ID NO:30, a sdCDR2 of SEQ ID NO:31 and a sdCDR3 of SEQ ID NO:32; (iv) SEQ ID NO:21; (v) SEQ ID NO:25; and (vi) SEQ ID NO:29.

In some embodiments, the sdABD-Trop2 comprises an amino acid sequence selected from the group consisting of: (i) a set of CDRs comprising a sdCDR1 of SEQ ID NO:78, a sdCDR2 of SEQ ID NO:79 and a sdCDR3 of SEQ ID NO:80; (ii) a set of CDRs comprising a sdCDR1 of SEQ ID NO:82, a sdCDR2 of SEQ ID NO:83 and a sdCDR3 of SEQ ID NO:84; (iii) a set of CDRs comprising a sdCDR1 of SEQ ID NO:86, a sdCDR2 of SEQ ID NO:87 and a sdCDR3 of SEQ ID NO:88; (iv) a set of CDRs comprising a sdCDR1 of SEQ ID NO:90, a sdCDR2 of SEQ ID NO:91, and a sdCDR3 of SEQ ID NO:92; (v) a set of CDRs comprising a sdCDR1 of SEQ ID NO:94, a sdCDR2 of SEQ ID NO:95, and a sdCDR3 of SEQ ID NO:96; (vi) a set of CDRs comprising a sdCDR1 of SEQ ID NO:98, a sdCDR2 of SEQ ID NO:99, and a sdCDR3 of SEQ ID NO:100; (vii) SEQ ID NO:77; (viii) SEQ ID NO:81; (ix) SEQ ID NO:85; (x) SEQ ID NO:89; (xi) SEQ ID NO:93; and (xii) SEQ ID NO:97.

In some embodiments, the first variable heavy domain is N-terminal to the first variable light domain and the pseudo variable light domain is N-terminal to the pseudo variable heavy domain.

In some embodiments, the first variable heavy domain is N-terminal to the first variable light domain and the pseudo variable heavy domain is N-terminal to the pseudo variable light domain.

In some embodiments, the first variable light domain is N-terminal to the first variable heavy domain and the pseudo variable light domain is N-terminal to the pseudo variable heavy domain.

In some embodiments, the first variable light domain is N-terminal to the first variable heavy domain and the pseudo variable heavy domain is N-terminal to the pseudo variable light domain.

In some embodiments, the third sdABD that bind to HSA has an amino acid sequence comprising: (a) set of CDRs selected from the group consisting of (i) a sdCDR1 of SEQ ID NO:246, a sdCDR2 of SEQ ID NO:247, and a sdCDR3 of SEQ ID NO:248, and (ii) a sdCDR1 of SEQ ID NO:250, a sdCDR2 of SEQ ID NO:251, and a sdCDR3 of SEQ ID NO:252; or (b) an amino acid sequence selected from the group consisting of SEQ ID NO:245 and SEQ ID NO:249.

In some embodiments, the cleavable linker comprises a cleavage domain sequence selected from the group consisting of SEQ ID NOS:339-408 and 532-535.

In some embodiments, the cleavable linker is cleaved by a human protease selected from the group consisting of MMP2, MMP9, meprin A, meprin B, cathepsin S, capthepsin K, capthesin L, granzyme B, uPA, kallekriein7, matriptase, and thrombin.

In some embodiments, the fusion proteins comprises an amino acid sequence selected from the group consisting of SEQ ID NO:453, SEQ ID NO:454, SEQ ID NO:455, SEQ ID NO:456, SEQ ID NO:457, and SEQ ID NO:458.

Provided herein is a nucleic acid encoding any of the fusion proteins described. Provided herein is an expression vector comprising any of the nucleic acids described. Provided herein is a host cell comprising any of the expression vectors described

In some aspects, provided is a method of making a fusion protein of the present disclosure comprising: (i) culturing the host cell described under conditions wherein the fusion protein is expressed and (ii) recovering the fusion protein.

In some aspects, provided is a single domain antigen binding domain that binds human LyPD3 (sdABD-LyPD3) comprising (i) an amino acid sequence selected from the group consisting of SEQ ID NO:117, SEQ ID NO:121, SEQ ID NO:125, SEQ ID NO:129, SEQ ID NO:133 and, SEQ ID NO:137 or (ii) an amino acid sequence comprising a set of CDRs selected from the group consisting of: (a) a sdCDR1 of SEQ ID NO:118, a sdCDR2 of SEQ ID NO:119 and a sdCDR3 of SEQ ID NO:120; (b) a sdCDR1 of SEQ ID NO:122, a sdCDR2 of SEQ ID NO:123 and a sdCDR3 of SEQ ID NO:124; (c) a sdCDR1 of SEQ ID NO:126, a sdCDR2 of SEQ ID NO:127 and a sdCDR3 of SEQ ID NO:128; (d) a sdCDR1 of SEQ ID NO:130, a sdCDR2 of SEQ ID NO:131, and a sdCDR3 of SEQ ID NO:132; (e) a sdCDR1 of SEQ ID NO:134, a sdCDR2 of SEQ ID NO:135, and a sdCDR3 of SEQ ID NO:136; and (f) a sdCDR1 of SEQ ID NO:138, a sdCDR2 of SEQ ID NO:139, and a sdCDR3 of SEQ ID NO:140.

Also provided is a nucleic acid encoding any of the single domain antigen binding domains (sdABDs) described. Also provided is an expression vector comprising any of the nucleic acids. Provided is a host cell comprising any of the expression vectors described.

In some aspects, provided is a method of making a single domain antigen binding domain (sdABD) comprising (a) culturing any of the host cells described herein under conditions wherein the sdABD is expressed and (b) recovering the sdABD.

Also provided is a pharmaceutical composition comprising any of the fusion proteins described or any of the single domain antigen binding domains (sdABDs) described.

In some embodiments, the pharmaceutical composition further comprises a pharmaceutically acceptable carrier or excipient.

In some aspects, provided is a method of treating cancer in a subject comprising administering any of the fusion proteins described, any of the single domain antigen binding domains (sdABDs) described, or any of the pharmaceutical compositions of the present disclosure.

### BRIEF DESCRIPTION OF THE DRAWINGS

FIG. 1 depicts the “format 1” type of protease activation of the present invention, referred to herein as “constrained, cleavable constructs” or “cc constructs”. In this embodiment, a representative construct includes ABDs for two TTA (as depicted in FIG. 1, these are both the same, although as described herein they can be different). Upon cleavage, the prodrug construct splits into three components, one containing an α-TTA domain linked via a domain linker to an active VH of αCD3, the second containing an α-TTA domain linked via a domain linker to an active VL of αCD3, and a “leftover” piece comprising the half-life extension domain linked to the inactive VH and VL. The two active variable domains then are free to associate to form a functional anti-CD3 binding domain. It should be noted that in “format 1” embodiments, the resulting active component is trivalent: there is monovalent binding to CD3 and bivalent binding to the TTA, rendering a bispecific binding protein, although in some cases this trivalency could be trispecifics, with monovalent binding to CD3, monovalent binding to a first TTA and monovalent binding to a second TTA. FIG. 1 also shows an anti-human serum albumin (HSA) domain as a half-life extension domain, in many embodiments a sdABD as defined herein, although as discussed herein, this is optional and/or can be replaced by other half-life extension domains; additionally, the half-life extension domain can also be N-terminal to the construct or internal as well. FIG. 1 also has the VH and VL of the Fv and iVH and iVL of the pseudo Fv in a specific order, e.g. from N- to C-terminal, VH-linker-VL (and iVL-linker-iVH) although as will be appreciated by those in the art, these can be reversed (VL-linker-VH and iVH-linker-iVL). Alternatively, one of these Fvs can be in one orientation and the other in the other orientation, although the expression of protein in the orientation as shown here was surprisingly higher than the other orientations.

FIG. 2 depicts the “format 2” type of protease activation of the present invention, referred to herein as “constrained, non-cleavable constructs”, or “CNCL constructs”, also sometimes referred to herein as “dimerization constructs” as discussed herein. These constructs do not isomerize as discussed herein. Upon cleavage, two prodrug construct splits into four components, two half-life extension domains (in this case, sdABDs to HSA) linked to two pseudo domains (which may or may not be able to self-associate, depending on the length of the linkers and the inactivating mutations), and two active moieties that self-assemble into a dimeric active moiety that contains four anti-TTA domains (which can be all the same or two are the same and the other two are different). It should be noted that in “format 2” embodiments, the resulting active component is hexavalent: there is bivalent binding to CD3 and quadrivalent binding to the TTA, rendering a bispecific binding protein, although in some cases this hexavalency could be trispecifics, with bivalent binding to CD3, bivalent binding to a first TTA and bivalent binding to a second TTA. FIG. 2 also shows an anti-human serum albumin (HSA) domain as a half-life extension domain, in many embodiments a sdABD as defined herein, although as discussed herein, this is optional and/or can be replaced by other half-life extension domains; additionally, the half-life extension domain can also be N-terminal to the construct or internal as well. FIG. 2 also has the VH and VL of the Fv and iVH and iVL of the pseudo Fv in a specific order, e.g. from N- to C-terminal, VH-linker-VL (and iVL-linker-iVH) although as will be appreciated by those in the art, these can be reversed (VL-linker-VH and iVH-linker-iVL). Alternatively, one of these Fvs can be in one orientation and the other in the other orientation, although the expression of protein in the orientation as shown here was surprisingly higher than the other orientations.

FIG. 3A - FIG. 3B depict “format 3” type of constructs, also sometimes referred to as “hemi-constructs” or “hemi-COBRA™” as outlined herein, as these are two different polypeptide chains that together make up an MCE therapeutic as is further discussed herein. In this embodiment, the constructs are delivered in pairs, with the pre-cleavage intramolecular self-assembly resulting in inactive anti-CD3 Fv domains. Upon cleavage, the inert variable domains are released, and the two active variable domains then intermolecularly assemble, to form an active anti-CD3 binding domain. The two sdABD-TTAs bind to the corresponding receptor on the tumor cell surface, and the cleavage is done by a protease. This allows the intermolecular assembly, since the molecules are physically held in place, favoring the assembly of the active anti-CD3 domain. As above for formats 1 and 2, in this embodiment, the N- to C-terminal order of the variable domains can be reversed, or mixed as well. Furthermore, the sdABD(HSA) can be either at the N- or C-terminus of each hemi-construct. Pro16 has the sdABD(HSA) at the C terminus and Pro17 has it at the N-terminus. Pro19, has the sdABD(HSA) at the C-terminus. FIG. 3A shows Format 3 constructs with a single sdABD-TTA domain per hemi-construct, and FIG. 3B shows Format 3 constructs with two sdABD-TTAs per hemi-construct, in a “dual targeting” or “hetero-targeting” format. Note that FIG. 3B uses FOLR1 and EGFR as the two TTAs, but other combinations as outlined herein can also be used.

FIG. 4 depicts “format 4” type of constructs that are similar to “format 2” constructs but have only a single sdABD-TTA. The figure shows the sdABD-TTA to EGFR, but as will be appreciated by those in the art, other TTA can be used as well. Upon cleavage, the prodrug construct splits into two components, a half-life extension domain (in this case, sdABDs to HSA) linked to a pseudo Fv and an active moiety, that in the presence of a second active moiety from a different cleaved molecule, self-assembles into a dimeric active moiety that contains two anti-TTA domains. It should be noted that in “format 4” embodiments, the resulting active component is quadrivalent: there is bivalent binding to CD3 and bivalent binding to the TTA, rendering a bispecific binding protein. FIG. 4 also shows an anti-human serum albumin (HSA) domain as a half-life extension domain, in many embodiments a sdABD(½) as defined herein, although as discussed herein, this is optional and/or can be replaced by other half-life extension domains; additionally, the half-life extension domain can also be N-terminal to the construct or internal as well. FIG. 4 also has the VH and VL of the Fv and iVH and iVL of the pseudo Fv in a specific order, e.g. from N- to C-terminal, VH-linker-VL (and iVL-linker-iVH) although as will be appreciated by those in the art, these can be reversed (VL-linker-VH and iVH-linker-iVL). Alternatively, one of these Fvs can be in one orientation and the other in the other orientation, although the expression of protein in the orientation as shown here was surprisingly higher than the other orientations.

FIG. 5A - FIG. 5N depict a number of single domain tumor target antigen binding domain (sdTTA-ABDs) sequences of the invention, with the CDRs underlined. As is more fully outlined herein, these domains can be assembled in a wide variety of configurations in the present invention, including “format 1”, “format 2”, “format 3” and “format 4” orientations.

FIG. 6 depict a number of half-life extension domains.

FIGS. 7A and 7B depict a number of αCD3 variable heavy and variable light domains, including the active (e.g. “V_{L}” or “V_{H}”, sometimes also referred to as “aVL” or “aVH”) and inactive (e.g. “V_{Li}” or “V_{Hi}”, sometimes also referred to as “iVL” or “iVH”) domains. The CDRs are underlined.

FIGS. 8A, 8B and 8C, 8D depict a number of suitable protease cleavage sites. As will be appreciated by those in the art, these cleavage sites can be used as cleavable linkers. In some embodiments, for example when more flexible cleavable linkers are required, there can be additional amino acids (generally glycines and serines) that are either or both N- and C-terminal to these cleavage sites.

FIG. 9A - FIG. 9V shows a number of sequences of the invention, although many additional sequences are also found in the sequence listing. CDRs are underlined and bolded, linkers are double underlined (with cleavable linkers being italicized and double underlined) and domain separations are indicated by “/”. All His6 tags are optional, as they can be used to reduce immunogenicity in humans as well as be purification tags.

FIG. 10A to 10EE depict amino acid sequences of exemplary Format 2 constructs comprising a number of sdABD-B7H3 and a pseudo Fv domain (e.g., Vli2/Vhi2 domains).

FIG. 11 illustrates the COBRA design and the predicted folding mechanism, with the predicted structure of the uncleaved molecule on the top, which still binds tumor antigen (EGFR, in the case of the MVC-101), has impaired CD3 binding and binds human serum albumin. The middle shows the predicted cleavage products and the left shows the active dimer.

FIG. 12A-FIG. 12Q depicts additional sequences of some COBRAs of the present invention.

FIG. 13 shows that the format 2 constructs of the invention, once cleaved and dimerized, clear quickly from injected mice.

FIG. 14 shows the binding kinetics of Pro225.

FIGS. 15A and 15B shows that format 2 constructs, in this case Pro225, regresses established solid tumors in mice.

FIGS. 16A and 16B shows that the format 2 constructs of the invention, in this case Pro225, shows increased tolerability relative to inherently active T cell engagers. FIGS. 16C and 16D show that treatment with Pro225 results in lower cytokine release in mice, compared to an inherently active bispecific. Pro 225 does not induce IL2, TNFa, and IL10 in NHP and mouse IL6 in mice in comparison to inherently active T cell engagers

FIG. 17 shows the efficacy of a number of format 2 constructs of the invention in a T cell Dependent Cellular Cytotoxicity (TDCC) assay as outlined in Example 2. Pro233 is an aEGFR construct with an MMP9 cleavage site; Pro565 is an aEpCAM (h664) construct with an MMP9 cleavage site; Pro566 is an aEpCAM (h665) construct with an MMP9 cleavage site; Pro623 is a heteroCOBRA of aEGFR and aEpCAM (h664) and an MMP9 site; and Pro624 is a heteroCOBRA of aEGFR and aEpCAM (h665) and an MMP9 site.

FIG. 18 shows the efficacy of a number of format 2 constructs of the invention in a TDCC assay as outlined in Example 2. Pro233 is an aEGFR construct with an MMP9 cleavage site; Pro311 is an aFOLR1 construct with an MMP9 cleavage site; and Pro421 is a heteroCOBRA of aEGFR and aFOLR1 and an MMP9 site.

FIG. 19 shows the efficacy of a number of format 2 constructs of the invention in a TDCC assay as outlined in Example 2. Pro225 is an aB7H3 construct with an MMP9 cleavage site; Pro566 is an aEpCAM construct with an MMP9 cleavage site; Pro656 is a heteroCOBRA of aB7H3 and aEpCAM and an MMP9 site; and Pro658 is a heteroCOBRA of aEpCAM and aB7H3 and an MMP9 site.

FIG. 20 shows the efficacy of a number of format 2 constructs of the invention in a TDCC assay as outlined in Example 2 on two different cell lines. Pro225 is an aB7H3 construct with an MMP9 cleavage site; Pro566 is an aEpCAM construct with an MMP9 cleavage site; and Pro656 is a heteroCOBRA of aB7H3 and aEpCAM and an MMP9 site. HT29 is an epithelial cell line that, unlike Raji cell lines, make good xenografts in mice. HT29 expresses both target genes, (B7H3 and EpCAM), and in this case, the B7H3 expression was knocked out using CRISPR. Thus, the heteroCOBRA and the EpCAM single targeting COBRA killed both, while the B7H3 single targeting COBRA did not.

FIG. 21 shows the efficacy of a number of format 2 constructs of the invention in a TDCC assay as outlined in Example 2 on the HT29 cell line that has high EpCAM expression and low Trop2 expression. Pro824 is an aEpCAM X aTrop2 (with an MMP9 linker) heteroCOBRA. Pro825 is an aEpCAM X aTrop2 heteroCOBRA with a NCL (non-cleavable control). Pro826 is an aTrop2 X aEpCAM HeteroCOBRA with an MMP9 linker. Pro827 is an aTrop2 X aEpCAM HeteroCOBRA with a NCL (non-cleavable control). Pro677 is an aTrop2/MMP9 COBRA and Pro566 is an aEpCAM/MMP9 COBRA. As the levels of the two antigens vary, the heteroCOBRAs maintain good killing while the killing with the monospecific COBRAs varies. The monospecific COBRAs don’t kill as well when the the expression level of their specific antigen drops (in this case Trop2); the same is true for FIGS. 22 and 23.

FIG. 22 shows the efficacy of a number of format 2 constructs of the invention in a TDCC assay as outlined in Example 2 on the HT116 cell line that has high EpCAM expression and very low Trop2 expression. Pro824 is an aEpCAM X aTrop2 (with an MMP9 linker) heteroCOBRA. Pro825 is an aEpCAM X aTrop2 heteroCOBRA with a NCL (non-cleavable control). Pro826 is an aTrop2 X aEpCAM HeteroCOBRA with an MMP9 linker. Pro827 is an aTrop2 X aEpCAM HeteroCOBRA with a NCL (non-cleavable control). Pro677 is an aTrop2/MMP9 COBRA and Pro566 is aEpCAM/MMP9 COBRA.

FIG. 23 shows the efficacy of a number of format 2 constructs of the invention in a TDCC assay as outlined in Example 2 on the BXPC3 cell line that has medium EpCAM expression and high Trop2 expression. Pro824 is an aEpCAM X aTrop2 (with an MMP9 linker) heteroCOBRA. Pro825 is an aEpCAM X aTrop2 heteroCOBRA with a NCL (non-cleavable control). Pro826 is an aTrop2 X aEpCAM HeteroCOBRA with an MMP9 linker. Pro827 is an aTrop2 X aEpCAM HeteroCOBRA with a NCL (non-cleavable control). Pro677 is an aTrop2/MMP9 COBRA and Pro566 is an aEpCAM/MMP9 COBRA.

FIG. 24 shows the in vivo efficacy of an aEpCAM COBRA with an MMP9 cleavage site using Protocol 2 of Example 3. Pro566 showed efficacy on LoVo tumors, as well as HT29, BxPC3 and SW403 tumor xenografts.

FIG. 25 shows the in vivo efficacy of an aTrop2 COBRA with an MMP9 cleavage site using Protocol 2 of Example 3. Pro677 showed efficacy on BxPC3 tumors, as well as HCC827 tumor xenografts.

FIG. 26 shows the in vivo efficacy of an aB7H3 COBRA with an MMP9 cleavage site using Protocol 3 of Example 3. Pro225 showed efficacy on A549 tumors.

FIGS. 27A-27E are a series of graphs demonstrating that a mono-specific COBRA containing two sdABD-HER2 (aHer2 hVIB1139) killed human or cyno HER2 expressing tumor cell lines conditionally in T-cell dependent cellular cytotoxicity (TDCC) assays. FIG. 27A: Human HER2-Raji cells were tested with various fusion proteins. FIG. 27B: Cyno Her2-Raji cells were tested with various fusion proteins. FIG. 27C: Raji cells tested with various fusion proteins. FIG. 27D: SKOV3 cells, with high expression of HER2, were tested with various fusion proteins. FIG. 27E: HT29 cells, with low expression of HER2, were tested with various fusion proteins. The tested fusion proteins were: Pro1123 NCL (non-cleavable control), Pro1117 MMP9 (uncleaved MMP9-containing COBRA) or, Pro1117 MMP9cl (cleaved MMP9-containing COBRA), or Pro1060 Pro51 format (positive control similar in format to anti-EGFR x CD3 positive control Pro51 as described in US2020/0347132 and WO2020/181140) and Pro1069 AD (active domain only). The amino acid sequence of Pro1117 is provided in FIG. 74 and SEQ ID NO:493.

FIGS. 28A-28E are a series of graphs demonstrating that a mono-specific COBRA containing two sdABD-HER2 (aHer2 h1159) killed human or cyno HER2 expressing tumor cell lines conditionally in TDCC assays. FIG. 28A: Human HER2-Raji cells were tested with various fusion proteins. FIG. 28B: Cyno HER2-Raji cells were tested with various fusion proteins. FIG. 28C: Raji cells tested with various fusion proteins. FIG. 28D: SKOV3 cells, with high expression of HER2, were tested with various fusion proteins. FIG. 28E: HT29 cells, with low expression of HER2, tested with various fusion proteins. The tested fusion proteins were: Pro1110 NCL, Pro1109 MMP9, Pro1109 MMP9cl, Pro 1062 Pro51 (positive control similar in format to anti-EGFR x CD3 positive control Pro51 as described in US2020/0347132 and WO2020/181140) and Pro1071 AD. The amino acid sequence of Pro1109 is provided in FIG. 73 and SEQ ID NO:491.

FIGS. 29A-29E are a series of graphs demonstrating that a mono-specific COBRA containing two sdABD-HER2 (aHER2 h1162) killed human or cyno HER2 expressing tumor cell lines conditionally in TDCC assays. FIG. 29A: Human HER2-Raji cells were tested with various fusion proteins FIG. 29B: Cyno HER2-Raji cells were tested with various fusion proteins. FIG. 29C: Raji cells tested with various fusion proteins. FIG. 29D: SKOV3 cells, with high expression of HER2, were tested with various fusion proteins. FIG. 29E: HT29 cells, with low expression of HER2, were tested with various fusion proteins. The tested fusion proteins were: Pro1112 NCL, Pro1111 MMP9, Pro 1111 MMP9cl, Pro1064 Pro51 and Pro1073 AD. The amino acid sequence of Pro1111 is provided in FIG. 73 and SEQ ID NO:492.

FIGS. 30A-30E are a series of graphs demonstrating that a mono-specific COBRA containing two sdABD-HER2 (aHer2 h1156) killed human or cyno HER2 expressing tumor cell lines conditionally in TDCC assays conditionally. FIG. 30A: Human HER2-Raji cells were tested with various fusion proteins FIG. 30B: Cyno HER2-Raji cells tested with various fusion proteins. FIG. 30C: Raji cells tested with various fusion proteins. FIG. 30D: SKOV3 cells, with high expression of HER2, were tested with various fusion proteins. FIG. 30E: HT29 cells, with low expression of HER2, were tested with various fusion proteins. The tested fusion proteins were: Pro1124 NCL, Pro1118 MMP9, Pro 1118 MMP9cl and Pro106 Pro51. The amino acid sequence of Pro1118 is provided in FIG. 74 and SEQ ID NO:494.

FIGS. 31A-31C are series of graphs depicting the results of aHER2 Pro51 fusion proteins leads Pro1043 VIB1139, Pro 1044 VIB1156, Pro1045 VIB1159 and Pro1047 VIB1162 which were selected for demonstrating good activity and cynomolgus cross-reactivity in TDCC assays, whereas Pro1036 VIB1055 and Pro1038 VIB1059 showed poor activity.

FIG. 32 is a graph demonstrating that HER2/MMP9 COBRA regresses established N87 Xenografts. Pro1118 was used in this assay at a dose of 100 ug/kg.

FIG. 33 is a graph demonstrating that HER2/MMP9 COBRA PK was consistent with murine HER2 Binding. Pro1111 was used in this assay at a dose of 30 ug/kg.

FIG. 34 is a table depicting the epitope binning of various HER2 sdAbs. Competing antibodies at 100 nM were tested with saturating antibodies at 333 nM. The tested aHER2 antibodies were: VIB1121, VIB1139, VIB1058, VIB1097, trastuzumab, VIB1156, VIB1160, VIB1159, and VIB1162. “B” refers to binding of competing Ab and “NB” refers to no binding of a competing Ab.

FIG. 35 is a table depicting the epitope binning of various HER2 sdAbs. Competing antibodies at 100 nM were tested with saturating antibodies at 333 nM. The tested antibodies were: Pro1118, Pro1111, Trastuzumab, and Pertuzumab. “B” refers to binding of competing Ab and “NB” refers to no binding of a competing Ab.

FIG. 36 are listing the amino acids locations and sequences for the epitope mapping of HER2 sdAb h1156 (SEQ ID NO:503) and HER2 sdAb h1162 (SEQ ID NO:511).

FIG. 37 is a table depicting the affinities of HER2 sdAbs in a Pro51 format. Various sdAb and fusion proteins combinations were assessed in human, cyno and mouse. The combinations were the following: 1055 and Pro1036; 1058 and Pro1037; 1059 and Pro1038; 1091 and Pro1039; 1092 and Pro1040; 1097 and Pro1041; 1121 and Pro1042; 1139 and Pro1043; 1156 and Pro1044; 1159 and Pro1045; 1160 and Pro1046; 1162 and Pro1047; h1074 and Pro1056; h1092 and Pro1057; h1097 and Pro1058; h1121 and Pro1059; h1139 and Pro1060; h1156 and Pro1061; h1159 and Pro1062; h1160 and Pro1063; and h1162 and Pro1064.

FIGS. 38A-38C are series of graphs demonstrating that a mono-specific COBRA containing two sdABD-CA9 (aCA9 h407) killed human or cyno CA9 expressing tumor cell lines conditionally in TDCC assays. FIG. 38A: Human CA9-Raji cells were tested with various fusion proteins. FIG. 38B: Cyno CA9-Raji cells were tested with various fusion proteins. FIG. 38C: HT29 cells were tested with various fusion proteins. The tested fusion proteins were: Pro514 NCL, Pro518 MMP9, Pro518 MMP9cl, Pro511 Pro51, and Pro521 AD. The amino acid sequence of Pro518 is provided in FIG. 10AA and SEQ ID NO:331.

FIGS. 39A-39C are series of graphs demonstrating that a mono-specific COBRA containing two sdABD-CA9 (aCA9 h445) killed human or cyno CA9 expressing tumor cell lines conditionally. FIG. 39A: Human CA9-Raji cells were tested with various fusion proteins. FIG. 39B: Cyno CA9-Raji cells were tested with various fusion proteins. FIG. 39C: HT29 cells were tested with various fusion proteins. The tested fusion proteins were: Pro515 NCL, Pro519 MMP9, Pro519 MMP9cl, and Pro512 Pro51. The amino acid sequence of Pro519 is provided in FIG. 10BB and SEQ ID NO:332.

FIGS. 40A-40C are a series of graphs demonstrating that a mono-specific COBRA containing two sdABD-CA9 (aCA9 h456) killed human or cyno CA9 expressing tumor cell lines conditionally in TDCC assays. FIG. 40A: Human CA9-Raji cells were tested with various fusion proteins. FIG. 40B: Cyno CA9-Raji cells were tested with various fusion proteins. FIG. 40C: HT29 cells tested with various fusion proteins. The tested fusion proteins were: Pro1095 NCL, Pro516 MMP9, Pro516 MMP9cl, and Pro509 Pro51. The amino acid sequence of Pro516 is provided in FIG. 10Z and SEQ ID NO:329.

FIGS. 41A-41C are a series of graphs demonstrating that a mono-specific COBRA containing two sdABD-CA9 (aCA9 h476) killed human or cyno CA9 expressing tumor cell lines conditionally in TDCC assays. FIG. 41A: Human CA9-Raji cells were tested with various fusion proteins FIG. 41B: Cyno CA9-Raji cells were tested with various fusion proteins. FIG. 41C: HT29 cells were tested with various fusion proteins. The tested fusion proteins were: Pro513 NCL, Pro517 MMP9, Pro517 MMP9cl, Pro520 AD and Pro510 Pro51. The amino acid sequence of Pro517 is provided in FIG. 10AA and SEQ ID NO:330.

FIG. 42 is a table depicting the affinities of CA9 sdAbs in Pro51 format. Various sdAbs, combinations of sdAbs, and fusion proteins were assessed in human, cyno and mouse. The sdAbs were the following: h407, h445, h456, h472 and h476 and the combinations were the following: h445 and Pro512; h456 and Pro509; and h476 and Pro510.

FIGS. 43A-43B are series of graphs demonstrating that CA9/MMP9 hetero-COBRAs regressed established tumor xenografts. Tumor SNU-16 in presence of Pro513, non cleavable control, Pro517 and Pro518, all at a dose of 300 ug/kg. Tumor 786-O in presence of Pro513 and Pro517, all at a dose of 100 ug/kg.

FIG. 44 is a graph showing that CA9/MMP9 hetero-COBRAs. The PK for Pro516 is consistent with it binding to the mouse CA9 protein. Pro517 and Pro 516 were used at dose of 100 ug/kg.

FIGS. 45A-45D are series of graphs demonstrating that EGFR/EpCAM heteroCOBRAs induced TDCC of Raji cells expressing one or both antigens. Raji-parental cells (FIG. 45A), Raji-EGFR cells (FIG. 45B), Raji-EpCAM cells (FIG. 45C), and Raji-EGFR/EpCAM cells (FIG. 45D) were tested with mono-specific COBRAs (Pro233 EGFR/EGFR mono-specific COBRAs) and Pro566 (EpCAM/EpCAM mono-specific COBRAs) and with hetero-COBRAs (Pro624 EGFR/EpCAM hetero-COBRAs) and Pro698 (EpCAM/EGFR hetero-COBRAs). All COBRAs were pre-cleaved.

FIGS. 46A-46C are series of graphs demonstrating that EGFR/EpCAM heteroCOBRAs comprising EGFR sdABD hD12 and EpCAM sdABD h665 induced TDCC on HT29 cells expressing both antigens. FIG. 46A: the EGFR/EpCAM hetero-COBRA were tested with Pro623 MMP9, Pro623 cleaved, Pro625 NCL, and with a buffer as a control. FIG. 46B: the EGFR/EpCAM hetero-COBRA (EpCAM sdABD h665/EGFR sdABD hD12) were tested with Pro698 MMP9, Pro698 MMP9cl, Pro699 NCL, and with a buffer as a control. FIG. 46C: the EGFR/EpCAM hetero-COBRA (hD12/h665) were tested with Pro624 MMP9, Pro624 MMP9cl, Pro626 NCL, and with a buffer as a control. The amino acid sequence of Pro624 are provided in FIG. 10W and SEQ ID NO:323. The amino acid sequence of Pro623 are provided in FIG. 10X and SEQ ID NO:322. The amino acid sequence of Pro698 are provided in FIG. 10X and SEQ ID NO:324.

FIGS. 47A-47C are series of graphs demonstrating that EGFR/FOLR1 heteroCOBRA induces TDCC on Raji cells expressing one or both antigens. Raji-EGFR cells (FIG. 47A), Raji-FOLR1 cells (FIG. 47B), Raji-EGFR/FOLR1 cells (FIG. 47C) were tested with mono-specific COBRAs: Pro233 (EGFR/EGFR) and Pro311 (FOLR1/ FOLR1) and with hetero-COBRAs: Pro421 (EGFR/FOLR1) and Pro420 (FOLR1/EGFR). All COBRAs were pre-cleaved. The amino acid sequence of Pro420 are provided in FIG. 9G and SEQ ID NO:421. The amino acid sequence of Pro421 are provided in FIG. 9G and SEQ ID NO:422. The amino acid sequence of Pro233 are provided in FIG. 9D and SEQ ID NO:415. The amino acid sequence of Pro311 are provided in FIG. 9D and SEQ ID NO:416.

FIGS. 48A-48C are a series of graphs demonstrating that aFOLR1/aEGFR heteroCOBRA comprising EGFR D12 and FOLR1 h59-3 killed tumor cell lines expressing both FOLR1 and EGFR conditionally. FIG. 48A: H292 cells were tested with the mono-specific COBRAs: Pro214 NCL (EGFR D12), Pro186 MMP9 (EGFR D12), and Pro186 MMP9cl (EGFR D12). FIG. 48B: H292 cells were tested with the mono-specific COBRAs: Pro303 NCL (FOLR1 h59-3), Pro312 MMP9 (FOLR1 h59-3), and Pro312 MMP9cl (FOLR1 h59-3). FIG. 48C: H292 cells were tested with the hetero-COBRAs: Pro550 NCL (EGFR D12/FOLR1 h59-3), Pro551 MMP9 (EGFR D12/FOLR1 h59-3), and Pro551 (MMP9cl EGFR D12/FOLR1 h59-3). The amino acid sequence of Pro551 are provided in FIG. 10V and SEQ ID NO:320.

FIGS. 49A-49D are series of graphs demonstrating that aFOLR1(h77.2)/aEGFR (hD12) killed tumor cell lines expressing both FOLR1 and EGFR conditionally. FIG. 49A: H292 cells were tested with the mono-specific COBRAs: Pro600 NCL (EGFR/EGFR), Pro233 MMP9 EGFR/EGFR, and Pro233 MMP9cl (EGFR/EGFR). FIG. 49B: H292 cells were tested with the mono-specific COBRAs: Pro299 NCL FOLR1/FOLR1, Pro311 MMP9 (FOLR1/FOLR1), and Pro311 MMP9cl (FOLR1/FOLR1). FIG. 49C: H292 cells were tested with the hetero-COBRAs: Pro420 MMP9 (FOLR1/EGFR), and Pro420 MMP9cl (FOLR1/EGFR). FIG. 49D: H292 cells were tested with the hetero-COBRAs: Pro421 MMP9 (EGFR/FOLR1), and Pro421 MMP9cl (EGFR/FOLR1). The amino acid sequence of Pro420 are provided in FIG. 9G and SEQ ID NO:421. The amino acid sequence of Pro421 are provided in FIG. 9G and SEQ ID NO:422.

FIG. 50 is a table listing the affinities of EGFR/FOLR1 HeteroCOBRA vs Pro51 format molecules.

FIGS. 51A-51D are series of graphs demonstrating that Pro566 aEpCAM (h664) killed EpCAM Raji transfectants and tumor cell lines expressing EpCAM conditionally. Trop2-Raji cells (FIG. 51A), EpCAM-Raji cells (FIG. 51B), SKOV3 cells (FIG. 51C), and HT29 cells (FIG. 51D) were all tested with Pro566 and cleaved Pro566 (Pro566cl).

FIGS. 52A-52D are series of graphs demonstrating that Pro677 aTrop2 (h557) kills Trop2 Raji transfectants and tumor cell lines expressing Trop2 conditionally. Trop2-Raji cells (FIG. 52A), EpCAM-Raji cells (FIG. 52B), SKOV3 cells (FIG. 52C), and HT29 cells (FIG. 52D) were all tested with Pro677 and cleaved Pro677 (Pro677cl.)

FIGS. 53A-53D are series of graphs demonstrating that Pro824 aEpCAM (h664)/aTROP2 (h557) kills Raji transfectants and tumor cell lines expressing both TROP2 and EpCAM conditionally. Trop2-Raji cells (FIG. 53A), EpCAM-Raji cells (FIG. 53B), SKOV3 cells (FIG. 53C), and HT29 cells (FIG. 53D) were all tested with Pro824 and cleaved Pro824 (Pro824cl.)

FIGS. 54A-54D are series of graphs demonstrating that Pro826 aTROP2 (h557)/aEpCAM (h664) kills Raji transfectants and tumor cell lines expressing both TROP2 and EpCAM conditionally. Trop2-Raji cells (FIG. 54A), EpCAM-Raji cells (FIG. 54B), SKOV3 cells (FIG. 54C), and HT29 cells (FIG. 54D) were all tested with Pro826 and cleaved Pro826 (Pro826cl).

FIGS. 55A-55D are series of graphs demonstrating that EpCAM and Trop2 COBRAs and HeteroCOBRAs all work well on BXPC3. FIG. 55A: BXPC3 cells were tested with Pro569 NCL, Pro566 MMP9 and Pro566 MMP9cl. FIG. 55B: BXPC3 cells were tested with Pro681 NCL, Pro677 MMP9 and Pro677 MMP9cl. FIG. 55C: BXPC3 cells were tested with Pro825 NCL, Pro824 MMP9 and Pro824 MMP9cl. FIG. 55D: BXPC3 cells were tested with Pro827 NCL, Pro826 MMP9 and Pro826 MMP9cl.

FIGS. 56A-56D are series of graphs demonstrating that EpCAM and Trop2 COBRAS and Hetero-COBRAs all work well on HCT116. FIG. 56A: HCT116 cells (human colon cancer cell line) were tested with Pro569 NCL, Pro566 MMP9, and Pro566 MMP9cl. FIG. 56B: HCT116 cells were tested with Pro681 NCL, Pro677 MMP9, and Pro677MMP9cl. FIG. 56C: HCT116 cells were tested with Pro825 NCL, Pro824 MMP9, and Pro824 MMP9cl. FIG. 56D: HCT116 cells were tested with Pro827 NCL, Pro826 MMP9 and Pro846 MMP9cl.

FIGS. 57A-57D are series of graphs demonstrating that EpCAM and Trop2 COBRAS and Hetero-COBRAs all work well on SCC25. FIG. 57A: SCC25 cells were tested with Pro569 NCL, Pro566 MMP9, and Pro566 MMP9cl. The amino acid sequence of Pro566 is provided in FIG. 10F and SEQ ID NO:289. FIG. 57B: SCC25 cells were tested with Pro681 NCL, Pro677 MMP9, and Pro677 MMP9cl. The amino acid sequence of Pro677 is provided in FIG. 10K and SEQ ID NO:298. FIG. 57C: SCC25 cells were tested with Pro825 NCL, Pro824 MMP9, and Pro824 MMP9cl. The amino acid sequence of Pro824 is provided in FIG. 12Q and SEQ ID NO:485. FIG. 57D: SCC25 cells were tested with Pro827 NCL, Pro826 MMP9, and Pro826 MMP9cl. The amino acid sequence of Pro826 is provided in FIG. 12Q and SEQ ID NO:486.

FIGS. 58A-58D are series of graphs demonstrating that B7H3/EpCAM HeteroCOBRAs induce TDCC on cells expressing one or both antigens. Raji-parental cells (FIG. 58A), Raji-B7H3 cells (FIG. 58B), Raji-EpCAM cells (FIG. 58C), and Raji-B7H3/EpCAM cells (FIG. 58D) were tested with mono-specifc COBRAs: Pro225 (B7H3/B7H3) and Pro566 (EpCAM/EpCAM) and with hetero-COBRAs: Pro656 (B7H3/EpCAM) and Pro658 (EpCAM/B7H3). All COBRAs were pre-cleaved. The amino acid sequence of Pro225 is provided in FIG. 10DD and SEQ ID NO:336. The amino acid sequence of Pro566 is provided in FIG. 10F and SEQ ID NO:289. The amino acid sequence of Pro656 is provided in FIG. 10Y and SEQ ID NO:326. The amino acid sequence of Pro658 is provided in FIG. 10Z and SEQ ID NO:328.

FIGS. 59A-59D are series of graphs depicting the results for CRISPR knockout lines. HT29 cells (FIG. 59A), HT29-B7H3 KO cells (FIG. 59B), HT29-EpCAM KO cells (FIG. 59C), and HT29-B7H3/EpCAM KO cells (FIG. 59D) were all tested with mono-specific COBRAs: Pro225 (B7H3/B7H3) and Pro566 (EpCAM/EpCAM) and with a hetero-COBRA: Pro656 (B7H3/EpCAM). All COBRAs were pre-cleaved. The amino acid sequence of Pro225 is provided in FIG. 10DD and SEQ ID NO:336. The amino acid sequence of Pro566 is provided in FIG. 10F and SEQ ID NO:289. The amino acid sequence of Pro656 is provided in FIG. 10Y and SEQ ID NO:326.

FIGS. 60A-60D are series of graphs demonstrating that EpCAM/B7H3 heteroCOBRAs comprising aEpCAM sdABD (h664) and aB7H3 sdABD (hF7) killed tumor cell lines expressing both EpCAM and B7H3 conditionally. FIG. 60A: IGROV cells were tested with Pro295 NCL (B7H3), Pro225 MMP9 (B7H3) and Pro225 MMP9cl (B7H3). FIG. 60B: IGROV cells were tested with Pro568 NCL (EpCAM), Pro565 MMP9 (EpCAM), and Pro565 MMP9cl (EpCAM). FIG. 60C: IGROV cells were tested with Pro659 NCL (B7H3/EpCAM), Pro655 MMP9 (B7H3/EpCAM) and Pro655 MMP9cl (B7H3/EpCAM). FIG. 60D: IGROV cells were tested with Pro661 NCL (EpCAM/B7H3), Pro657 MMP9 (EpCAM/B7H3) and Pro657 MMP9cl (EpCAM/B7H3). The amino acid sequence of Pro655 is provided in FIG. 10X and SEQ ID NO:325. The amino acid sequence of Pro657 is provided in FIG. 10Y and SEQ ID NO:327.

FIGS. 61A-61D are a series of graphs demonstrating that aEpCAM/aB7H3 hetero-COBRAs comprising an aEpCAM sdABD (h665) and an aB7H3 sdABD (hF7) sdABD killed tumor cell lines expressing both EpCAM and B7H3 conditionally. FIG. 61A: IGROV cells were tested with Pro295 NCL (B7H3), Pro225 MMP9 (B7H3) and Pro225 MMP9cl (B7H3). FIG. 61B: IGROV cells were tested with Pro569 NCL (EpCAM), Pro566 MMP9 (EpCAM), and Pro566 MMP9cl (EpCAM). FIG. 61C: IGROV cells were tested with Pro660 NCL (B7H3/EpCAM), Pro656 MMP9 (B7H3/EpCAM) and Pro656 MMP9cl (B7H3/EpCAM). FIG. 61D: IGROV cells were tested with Pro662 NCL (EpCAM/B7H3), Pro658 MMP9 (EpCAM/B7H3) and Pro658 MMP9cl (EpCAM/B7H3). The amino acid sequence of Pro656 is provided in FIG. 10Y and SEQ ID NO:326. The amino acid sequence of Pro658 is provided in FIG. 10Z and SEQ ID NO:328.

FIGS. 62A-62D are a series of graphs demonstrating that aEpCAM/aB7H3 hetero-COBRAs comprising an aEpCAM sdABD (h664) and an aB7H3 sdABD (hF7) killed tumor cell lines expressing both EpCAM and B7H3 conditionally. FIG. 62A: H292 cells were tested with Pro295 NCL (B7H3), Pro225 MMP9 (B7H3) and Pro225 MMP9cl (B7H3). FIG. 62B: H292 cells were tested with Pro568 NCL (EpCAM), Pro565 MMP9 (EpCAM), and Pro565 MMP9cl (EpCAM). FIG. 62C: H292 cells were tested with Pro659 NCL (B7H3/EpCAM), Pro655 MMP9 (B7H3/EpCAM) and Pro655 MMP9cl (B7H3/EpCAM). FIG. 62D: H292 cells were tested with Pro661 NCL (EpCAM/B7H3), Pro657 MMP9 (EpCAM/B7H3) and Pro657 MMP9cl (EpCAM/B7H3).

FIGS. 63A-63D are series of graphs demonstrating that aEpCAM/aB7H3 hetero-COBRAs comprising an aEpCAM sdABD (h665) and an aB7H3 sdABD (hF7) killed tumor cell lines expressing both EpCAM and B7H3 conditionally. FIG. 63A: H292 cells were tested with Pro295 NCL (B7H3), Pro225 MMP9 (B7H3) and Pro225 MMP9cl (B7H3). FIG. 63B: H292 cells were tested with Pro569 NCL (EpCAM), Pro566 MMP9 (EpCAM), and Pro566 MMP9cl (EpCAM). FIG. 63C: H292 cells were tested with Pro660 NCL (B7H3/EpCAM), Pro656 MMP9 (B7H3/EpCAM) and Pro656 MMP9cl (B7H3/EpCAM). FIG. 63D: H292 cells were tested with Pro662 NCL (EpCAM/B7H3), Pro658 MMP9 (EpCAM/B7H3) and Pro658 MMP9cl (EpCAM/B7H3).

FIGS. 64A-64D are a series of graphs depicting the effect of T cell Dependent Cellular Cytotoxicity (TDCC) on tumor cell lines. HT29 cells (FIG. 64A), U87-MG (EpCAM-negative) cells (FIG. 64B), Capan2 cells (FIG. 64C), and VCAP cells (FIG. 64D) were all tested with mono-specific COBRAs: Pro225 (B7H3/B7H3) and Pro566 (EpCAM/EpCAM) and with hetero-COBRAs: Pro656 (B7H3/EpCAM) and Pro658 (EpCAM/B7H3). All COBRAs were pre-cleaved.

FIG. 65 is a graph depicting Jurkat luciferase T cell activation on HT29 Cells. The HT29 cells were tested with mono-specific COBRAs: Pro225 (B7H3/B7H3) and Pro566 (EpCAM/EpCAM) and with hetero-COBRAs: Pro656 (B7H3/EpCAM) and Pro658 (EpCAM/B7H3). All COBRAs were pre-cleaved.

FIGS. 66A-66D are series of graphs showing that the activity of heteroCOBRAs on HT29 cells is less sensitive to inhibition by soluble antigen compared to monospecific COBRAs. The cells were assayed with soluble EpCAM, soluble B7H3 4Ig and with no antigen (control) together with mono-specific COBRAs: Pro225 (B7H3/B7H3) in FIG. 66A and Pro566 (EpCAM/EpCAM) in FIG. 66B and with hetero-COBRAs: Pro656 (B7H3/EpCAM) in FIG. 66C and Pro658 (EpCAM/B7H3) in FIG. 66D. A stronger inhibition was detected with the mono-specific COBRAs. All COBRAs were pre-cleaved.

FIG. 67 is a table depicting the binding affinities of B7H3/EpCAM heteroCOBRAs. Antigens including huB7H3-4Ig alone, huEpCAM and huB7H3-4Ig, and huEpCAM alone were assayed with hetero-COBRAs Pro656 (B7H3/EpCAM) and Pro658 (EpCAM/B7H3).

FIG. 68 is a graph depicting the pharmacokinetics of various B7H3/EpCAM heteroCOBRAs.

FIG. 69 is a graph showing that B7H3/EpCAM hetero-COBRAs are active in the HT29 cell line derived xenograft model in mice. Dosages of hetero-COBRAa such as Pro660 NCL (B7H3/EpCAM; 0.3 mg/kg), Pro656 MMP9 (B7H3/EpCAM; 0.01 mg/kg), Pro656 MMP9 (B7H3/EpCAM; 0.03 mg/kg) and Pro656 MMP9 (0 B7H3/EpCAM; 1 mg/kg) were administered at various time intervals.

FIG. 70 is a graph showing that B7H3/EpCAM heteroCOBRAs are active in the HT29 cell line derived xenograft model in mice. Dosages of heteroCOBRAs such as Pro662 NCL (EpCAM/B7H3; 0.1 mg/kg) and Pro658 MMP9 (EpCAM/B7H3; 0.1 mg/kg) were administered at various time intervals.

FIGS. 71-72 provide additional sequences of exemplary heteroCOBRAs (dual targeting COBRAs) described herein.

FIGS. 73-74 provided additional sequences of exemplary monospecific HER2 COBRAs (single targeting COBRAs).

FIG. 75 provides sequences of a humanized anti-EpCAM sdAb h664 and humanized anti-HER2 sdAbs h1139, h1156, h1159 and h1162 described herein.

### DETAILED DESCRIPTION OF THE INVENTION

### Introduction

The present invention is directed to methods of reducing the toxicity and side effects of bispecific antibodies (including antibody-like functional proteins) that bind to important physiological targets such as CD3 and tumor antigens. Many antigen binding proteins, such as antibodies, can have significant side effects by targeting normal tissues, and thus there is a need to only activate the binding capabilities of a therapeutic molecule in the vicinity of the disease tissue, to avoid normal tissue interactions. Accordingly, the present invention is directed to multivalent conditionally effective (“MCE”) proteins that have a number of functional protein domains. In general, one of these domains is an antigen binding domain (ABD) that will bind a target tumor antigen (TTA), and another is an ABD that will bind a T-cell antigen such as CD3 under certain conditions. Additionally, the MCE proteins also include one or more protease cleavage sites. That is, the therapeutic molecules are made in a “pro-drug” like format, wherein the CD3 binding domain is inactive until exposed to a tumor environment. The tumor environment contains proteases, such that upon exposure to the protease, the prodrug is cleaved and becomes active.

This is generally accomplished herein by using proteins that include a “pseudo” variable heavy domain and a “pseudo” variable light domain directed to the T-cell antigen such as CD3, that restrain the CD3 Fvs of the MCE into an inactive format as is discussed herein. As the TTA targets the MCE into the proximity of the tumor, the MCE is thus exposed to the protease. Upon cleavage, the active variable heavy domain and active light domain are now able to pair to form one or more active ABDs to CD3 and thus recruit T cells to the tumor, resulting in treatment.

In general, the CD3 binding domain (“Fv”) is in a constrained format, wherein the linker between the active variable heavy domain and the active variable light domain that traditionally form an Fv is too short to allow the two active variable domains to bind each other; this is referred to as “constrained linker”; these can be constrained and cleavable (CCL, as used in Format 1) or constrained and not cleavable (CNCL, as used in Format 2). Rather, in the prodrug (e.g., uncleaved) format, the prodrug polypeptide also comprises a “pseudo Fv domain”. The pseudo Fv domain comprises a variable heavy and light domain, with standard framework regions, but “inert” or “inactive” CDRs. The pseudo Fv domain also has a constrained linker between the inactive variable heavy and inactive variable light domains. Since neither Fv nor pseudo Fv domains can self-assemble due to the steric constraints, there is an intramolecular assembly that pairs the aVL with the iVH and the aVH with the iVL, due to the affinity of the framework regions of each. However, due to the “inert” CDRs of the pseudo domain, the resulting ABDs will not bind CD3, thus preventing toxicities outside the diseased tissue, such as a tumor. However, in the presence of proteases that are in or near the tumor, the prodrug construct is cleaved such that the pseudo-Fv domain is released from the surface and thus allows the “real” variable heavy and variable light domains to associate intermolecularly (e.g. two cleaved constructs come together), thus triggering active CD3 binding and the resulting tumor efficacy. These constructs are generally referred to herein as COnditional Bispecific Redirected Activation constructs, or “COBRAs™”. The stability of the intramolecular assembly is shown by the conditionality experiments herein, whereby in the absence of protease, the uncleaved constructs have no activity (e.g. no active CD3 binding domain is formed).

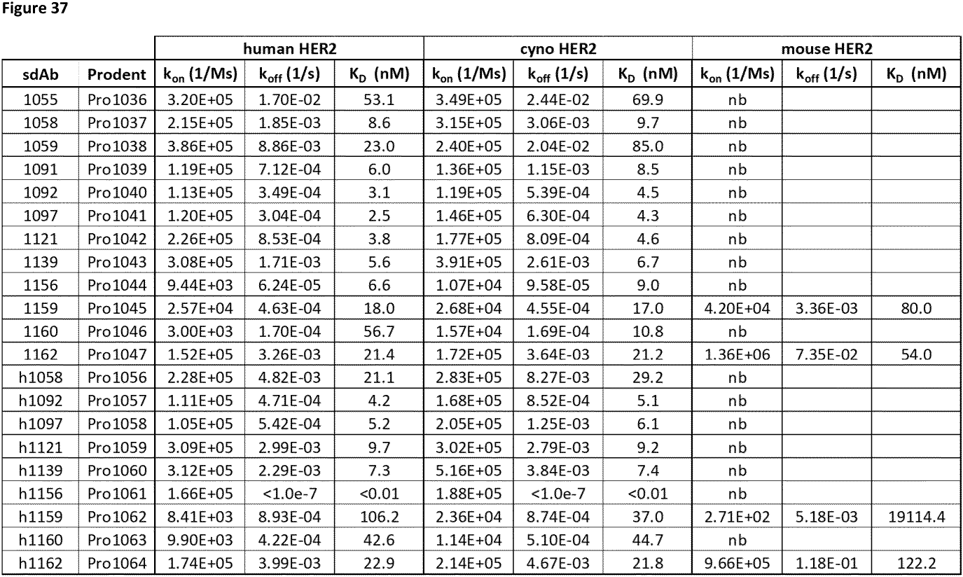

Interestingly, for ease of description, while these constructs are all referred to herein as “constrained”, additional work shows that the intramolecular assembly is favored even if one of the Fv domains is not constrained, e.g. one of the domains can have a longer, flexible linker. That is, as shown in the FIGS. 37-39, intramolecular assembly still occurs (e.g. the uncleaved constructs are inactive in the absence of protease cleavage) if only one of the Fv domains, either the one with an active VL and VH, or the pseudo Fv domain, is constrained. However, in the current systems, when both linkers are constrained, the protein has better expression. However, as will be appreciated by those of skill in the art, any of the Format 1, Format 2 or Format 4 constructs herein can have one of these Fv domains with an “unconstrained” or “flexible” linker. For ease of reference, the constructs are shown with both Fv domains in a constrained format.

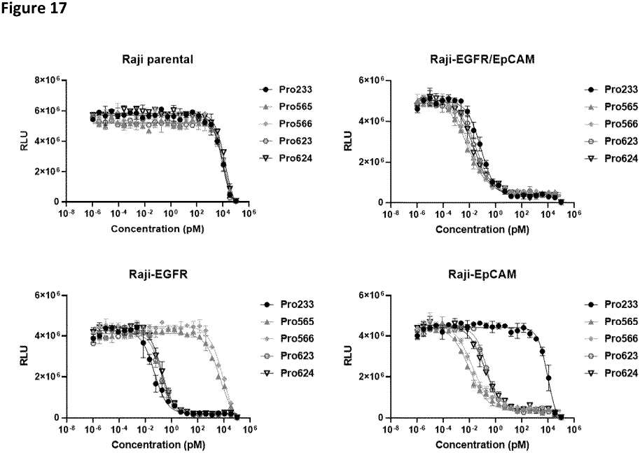

The constructs and formats of the invention are variations over embodiments described in WO2017/156178, WO2019/051102, WO2020/181140, US2019/0076524, and US2020/0347132, hereby expressly incorporated by reference in their entireties. As shown in FIGS. 17-21 of WO2017/156178, the Figures of WO2019/051102, and the Figures of WO2020/181140, previous constructs have the ability to isomerize due to the presence of two sets of VH and VL domains in a single polypeptide, forming both a bivalent scFv and a single chain diabody. Even after purification of each isoform, the bivalent construct can still reach equilibrium with the diabody isoform. As the single chain diabody has the ability to bind to CD3 in the absence of protease cleavage, the utility of the construct is diminished.

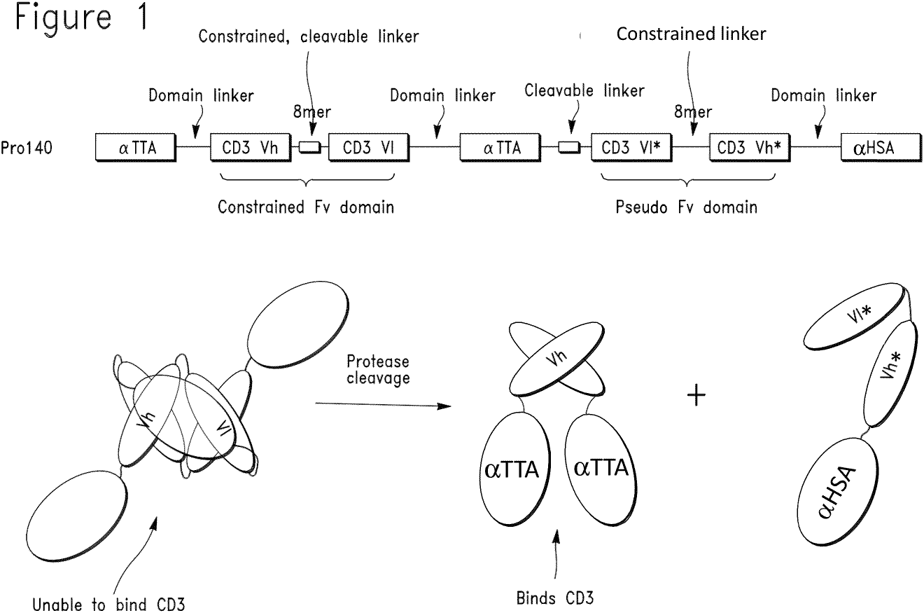

To solve this issue, the present invention provides for four separate types of constructs to accomplish this conditional activation. The prodrug activation can happen in one of four general ways, as is generally shown in the Figures. In FIG. 1, a “format 1” mechanism is shown. In this embodiment, the prodrug construct has two cleavage sites: one between the VH and vl domains of the constrained Fv, thus freeing the two variable domains to associate, and a second at a site that releases the pseudo Fv domain from the prodrug construct, leaving two molecules that associate due to the innate self-assembly of the variable heavy and variable light domains, each having an antigen binding domain to a tumor antigen as well, thus allowing the recruitment of T cells to the tumor site.

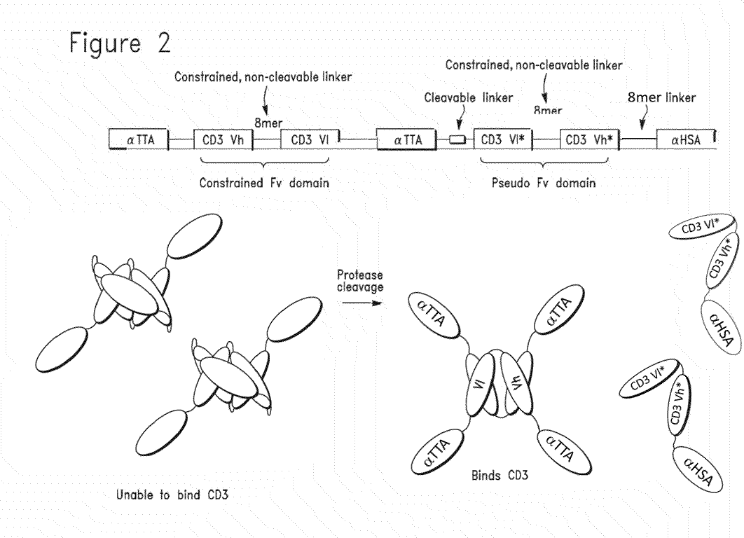

In an alternate embodiment, the prodrug construct is shown in FIG. 2, a “format 2” mechanism. In this embodiment, the domain linker between the active variable heavy and active light chains is a constrained but not cleavable linker (“CNCL”). In the prodrug format, the inactive VH and VL of the constrained pseudo Fv domain associate with the VH and VL of the constrained Fv domain, such that there is no CD3 binding. However, once cleavage in the tumor environment happens, two different activated proteins, each comprising an active variable heavy and light domain, associate to form two anti-CD3 binding domains. This format 2 has two target tumor antigen binding domains (“TTA-ABDs”) which as more fully described below, can either be identical (e.g. “homo-COBRAs”), or different (e.g. “hetero-COBRAs”). If different, they can each be directed to a different tumor antigen, or they can be directed to the same tumor antigen, but different epitopes, as is more fully described below.

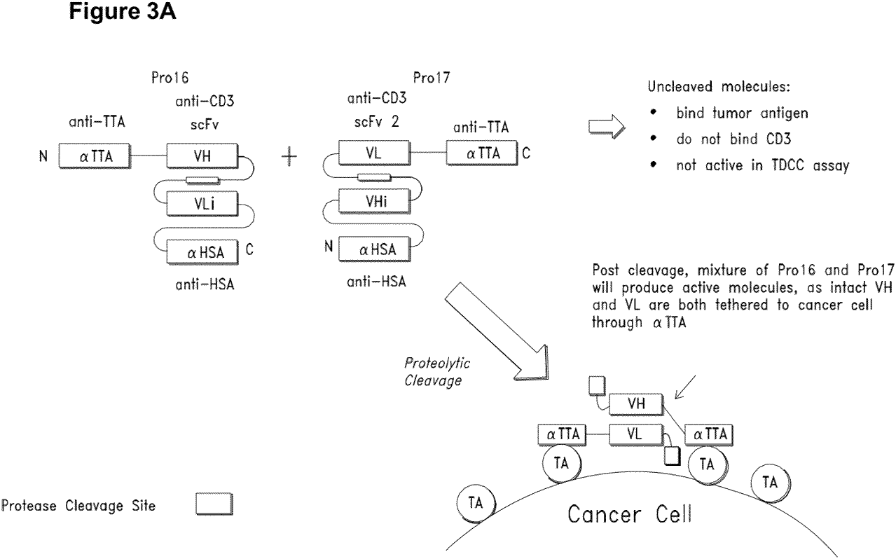

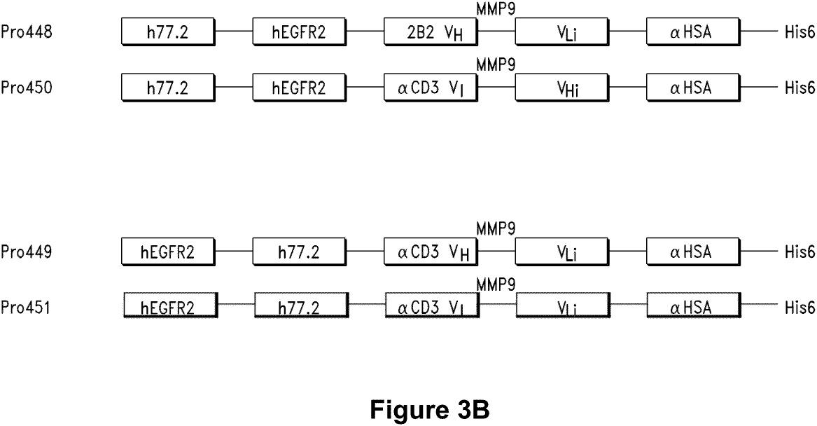

In addition to the “single chain protein” COBRA formats discussed above, where all of the components are contained on a single amino acid sequence, there are also constructs that rely on two proteins “hemi-COBRAs”, which act in pairs, as shown in FIG. 3. In this embodiment, each protein has one active and one inert variable domain separated by a protease cleavage site. Each molecule contains a TTA binding domain, such that when the molecules are bound to the TTA and exposed to tumor protease, the inert domains are cleaved off and the two active variable domains self-assemble to form an anti-CD3 binding domain.

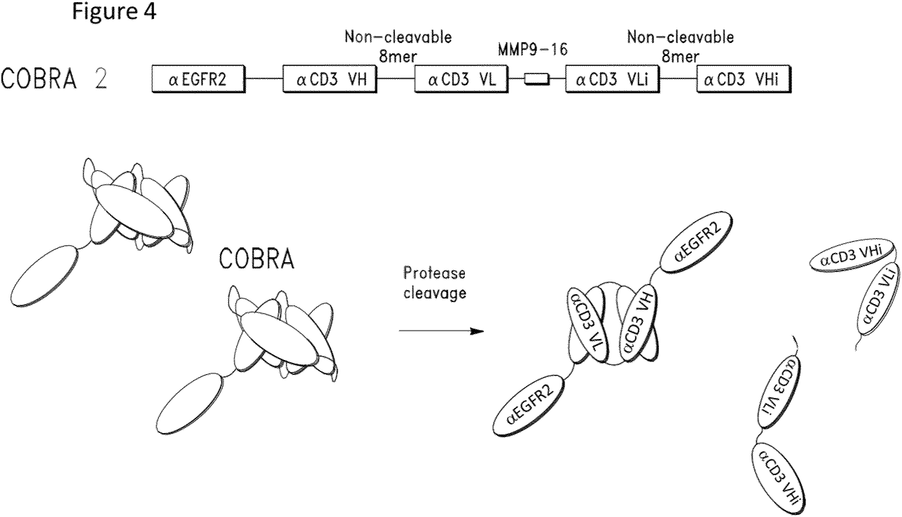

Furthermore, the invention provides “format 4” constructs as well, as depicted in FIG. 4. These are similar to the “format 2” designs, except that a single ABD to a TTA is used, such that upon cleavage, two of the pro-drug molecules now form a tetravalent, bispecific construct containing two active anti-CD3 domains, as is further described below.

Accordingly, the formats and constructs of the invention find use in the treatment of disease.

### Definitions

In order that the application may be more completely understood, several definitions are set forth below. Such definitions are meant to encompass grammatical equivalents.

By “amino acid” and “amino acid identity” as used herein is meant one of the 20 naturally occurring amino acids or any non-natural analogues that may be present at a specific, defined position. In many embodiments, “amino acid” means one of the 20 naturally occurring amino acids. By “protein” herein is meant at least two covalently attached amino acids, which includes proteins, polypeptides, oligopeptides and peptides.

By “amino acid modification” herein is meant an amino acid substitution, insertion, and/or deletion in a polypeptide sequence or an alteration to a moiety chemically linked to a protein. For example, a modification may be an altered carbohydrate or PEG structure attached to a protein. For clarity, unless otherwise noted, the amino acid modification is always to an amino acid coded for by DNA, e.g. the 20 amino acids that have codons in DNA and RNA. The preferred amino acid modification herein is a substitution.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence.

By “amino acid deletion” or “deletion” as used herein is meant the removal of an amino acid sequence at a particular position in a parent polypeptide sequence.

The polypeptides of the invention specifically bind to CD3 and target tumor antigens (TTAs) such as target cell receptors, as outlined herein. “Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore assay or Octet as is known in the art.

By “parent polypeptide” or “precursor polypeptide” (including Fc parent or precursors) as used herein is meant a polypeptide that is subsequently modified to generate a variant. Said parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent Fc polypeptide” as used herein is meant an unmodified Fc polypeptide that is modified to generate a variant, and by “parent antibody” as used herein is meant an unmodified antibody that is modified to generate a variant antibody.

By “position” as used herein is meant a location in the sequence of a protein. Positions may be numbered sequentially, or according to an established format, for example the EU index for antibody numbering.

By “target antigen” as used herein is meant the molecule that is bound specifically by the variable region of a given antibody. A target antigen may be a protein, carbohydrate, lipid, or other chemical compound. A range of suitable exemplary target antigens are described herein.

By “target cell” as used herein is meant a cell that expresses a target antigen. Generally, for the purposes of the invention, target cells are either tumor cells that express TTAs or T cells that express the CD3 antigen.

By “Fv” or “Fv domain” or “Fv region” as used herein is meant a polypeptide that comprises the VL and VH domains of an antigen binding domain, generally from an antibody. Fv domains usually form an “antigen binding domain” or “ABD” as discussed herein, if they contain active VH and VL domains (although in some cases, an Fv containing a constrained linker is used, such that an active ABD isn’t formed prior to cleavage). As discussed below, Fv domains can be organized in a number of ways in the present invention, and can be “active” or “inactive”, such as in a scFv format, a constrained Fv format, a pseudo Fv format, etc. It should be understood that in the present invention, in some cases an Fv domain is made up of a VH and VL domain on a single polypeptide chain, such as shown in FIG. 1 and FIG. 2 but with a constrained linker such that an intramolecular ABD cannot be formed. In these embodiments, it is after cleavage that two active ABDs are formed. In some cases, an Fv domain is made up of a VH and a VL domain, one of which is inert, such that only after cleavage is an intermolecular ABD formed. As discussed below, Fv domains can be organized in a number of ways in the present invention, and can be “active” or “inactive”, such as in a scFv format, a constrained Fv format, a pseudo Fv format, etc. In addition, as discussed herein, Fv domains containing VH and VL can be/form ABDs, and other ABDs that do not contain VH and VL domains can be formed using sdABDs.

By “variable domain” herein is meant the region of an immunoglobulin that comprises one or more Ig domains substantially encoded by any of the Vκ, Vλ, and/or VH genes that make up the kappa, lambda, and heavy chain immunoglobulin genetic loci respectively. In some cases, a single variable domain, such as a sdFv (also referred to herein as sdABD) can be used.

In embodiments utilizing both variable heavy (VH) and variable light (VL) domains, each VH and VL is composed of three hypervariable regions (“complementary determining regions,” “CDRs”) and four “framework regions”, or “FRs”, arranged from amino-terminus to carboxy-terminus in the following order: FR1-CDR1-FR2-CDR2-FR3-CDR3-FR4. Thus, the VH domain has the structure vhFR1-vhCDR1-vhFR2-vhCDR2-vhFR3-vhCDR3-vhFR4 and the VL domain has the structure v1FR1-v1CDR1-v1FR2-vC1DR2-v1FR3-vlCDR3-vlFR4. As is more fully described herein, the vhFR regions and the v1FR regions self-assemble to form Fv domains. In general, in the prodrug formats of the invention, there are “constrained Fv domains” wherein the VH and VL domains cannot self associate, and “pseudo Fv domains” for which the CDRs do not form antigen binding domains when self associated.

The hypervariable regions confer antigen binding specificity and generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the v1CDRs (e.g. vlCDR1, vlCDR2 and vlCDR3).

A useful comparison of CDR numbering is as below, see Lafranc et al., Dev. Comp. Immunol. 27(1):55-77 (2003):

TABLE 1

| | Kabat+ Chothia | IMGT | Kabat | AbM | Chothia | Contact |
|---|---|---|---|---|---|---|
| vhCDR1 | 26-35 | 27-38 | 31-35 | 26-35 | 26-32 | 30-35 |
| vhCDR2 | 50-65 | 56-65 | 50-65 | 50-58 | 52-56 | 47-58 |
| vhCDR3 | 95-102 | 105-117 | 95-102 | 95-102 | 95-102 | 93-101 |
| v1CDR1 | 24-34 | 27-38 | 24-34 | 24-34 | 24-34 | 30-36 |
| vlCDR2 | 50-56 | 56-65 | 50-56 | 50-56 | 50-56 | 46-55 |
| v1CDR3 | 89-97 | 105-117 | 89-97 | 89-97 | 89-97 | 89-96 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g, Kabat et al., supra (1991)).

The present invention provides a large number of different CDR sets. In this case, a “full CDR set” in the context of the anti-CD3 component comprises the three variable light and three variable heavy CDRs, e.g. a vlCDR1, vlCDR2, vlCDR3, vhCDR1, vhCDR2 and vhCDR3. As will be appreciated by those in the art, each set of CDRs, the VH and VL CDRs, can bind to antigens, both individually and as a set. For example, in constrained Fv domains, the vhCDRs can bind, for example to CD3 and the vlCDRs can bind to CD3, but in the constrained format they cannot bind to CD3.

In the context of a single domain ABD (“sdABD”) such as are generally used herein to bind to target tumor antigens (TTA), a CDR set is only three CDRs; these are sometimes referred to in the art as “VHH” domains as well.

These CDRs can be part of a larger variable light or variable heavy domain, respectfully. In addition, as more fully outlined herein, the variable heavy and variable light domains can be on separate polypeptide chains or on a single polypeptide chain in the case of scFv sequences, depending on the format and configuration of the moieties herein.

The CDRs contribute to the formation of the antigen-binding, or more specifically, epitope binding sites. “Epitope” refers to a determinant that interacts with a specific antigen binding site in the variable regions known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specific antigen binding peptide; in other words, the amino acid residue is within the footprint of the specific antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

The variable heavy and variable light domains of the invention can be “active” or “inactive”.

As used herein, “inactive VH” (“iVH”) and “inactive VL” (“iVL”) refer to components of a pseudo Fv domain, which, when paired with their cognate VL or VH partners, respectively, form a resulting VH/VL pair that does not specifically bind to the antigen to which the “active” VH or “active” VL would bind were it bound to an analogous VL or VH, which was not “inactive”. Exemplary “inactive VH” and “inactive VL” domains are formed by mutation of a wild type VH or VL sequence as more fully outlined below. Exemplary mutations are within CDR1, CDR2 or CDR3 of VH or VL. An exemplary mutation includes placing a domain linker within CDR2, thereby forming an “inactive VH” or “inactive VL” domain. In contrast, an “active VH” or “active VL” is one that, upon pairing with its “active” cognate partner, i.e., VL or VH, respectively, is capable of specifically binding to its target antigen. Thus, it should be understood that a pseudo Fv can be a VH/iVL pair, a iVH/VL pair, or a iVH/iVL pair.

In contrast, as used herein, the term “active” refers to a CD3 binding domain that is capable of specifically binding to CD3. This term is used in two contexts: (a) when referring to a single member of an Fv binding pair (i.e., VH or VL), which is of a sequence capable of pairing with its cognate partner and specifically binding to CD3; and (b) the pair of cognates (i.e., VH and VL) of a sequence capable of specifically binding to CD-. An exemplary “active” VH, VL or VH/VL pair is a wild type or parent sequence.

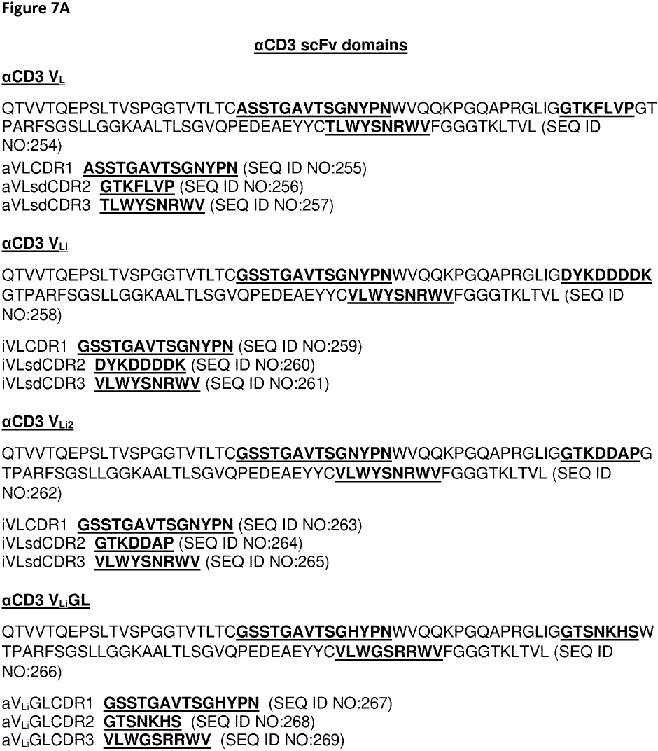

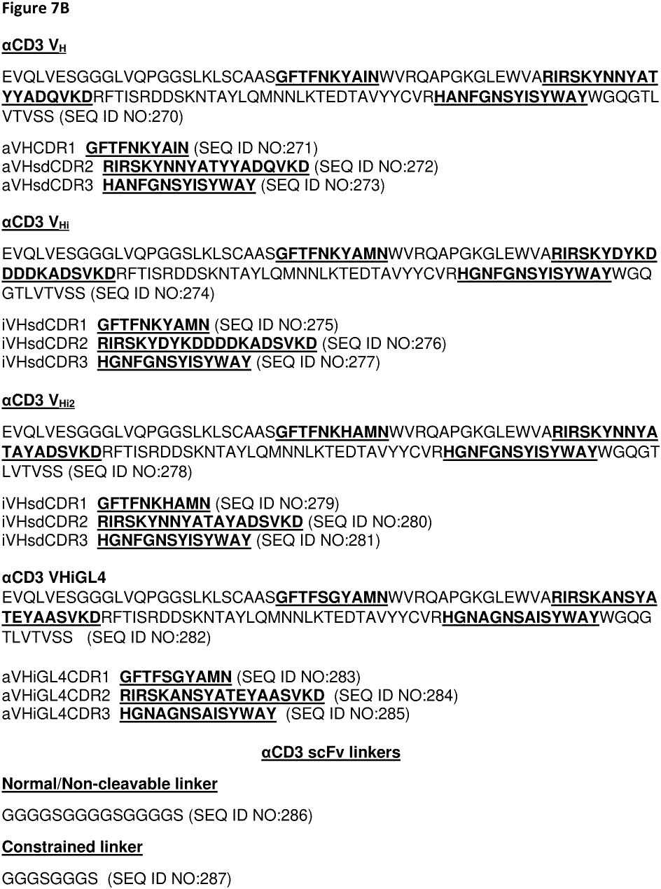

“CD-x” refers to a cluster of differentiation (CD) protein. In exemplary embodiments, CD-x is selected from those CD proteins having a role in the recruitment or activation of T-cells in a subject to whom a polypeptide construct of the invention has been administered. In an exemplary embodiment, CD-x is CD3, the sequence of which is shown in FIG. 7.

The term “binding domain” characterizes, in connection with the present invention, a domain which (specifically) binds to/interacts with/recognizes a given target epitope or a given target site on the target molecules (antigens), for example: EGFR and CD3, respectively. The structure and function of the target antigen binding domain (recognizing EGFR), and preferably also the structure and/or function of the CD3 binding domain (recognizing CD3), is/are based on the structure and/or function of an antibody, e.g. of a fulllength or whole immunoglobulin molecule, including sdABDs. According to the invention, the target antigen binding domain is generally characterized by the presence of three CDRs that bind the target tumor antigen (generally referred to in the art as variable heavy domains, although no corresponding light chain CDRs are present). Alternatively, ABDs to TTAs can include three light chain CDRs (i.e. CDR1, CDR2 and CDR3 of the VL region) and/or three heavy chain CDRs (i.e. CDR1, CDR2 and CDR3 of the VH region). The CD3 binding domain preferably also comprises at least the minimum structural requirements of an antibody which allow for the target binding. More preferably, the CD3 binding domain comprises at least three light chain CDRs (i.e. CDR1, CDR2 and CDR3 of the VL region) and/or three heavy chain CDRs (i.e. CDR1, CDR2 and CDR3 of the VH region). It is envisaged that in exemplary embodiments the target antigen and/or CD3 binding domain is produced by or obtainable by phage-display or library screening methods.

By “domain” as used herein is meant a protein sequence with a function, as outlined herein. Domains of the invention include tumor target antigen binding domains (TTA domains), variable heavy domains, variable light domains, scFv domains, linker domains, and half life extension domains.

By “domain linker” herein is meant an amino acid sequence that joins two domains as outlined herein. Domain linkers can be cleavable linkers, constrained cleavable linkers, non-cleavable linkers, constrained non-cleavable linkers, scFv linkers, etc.

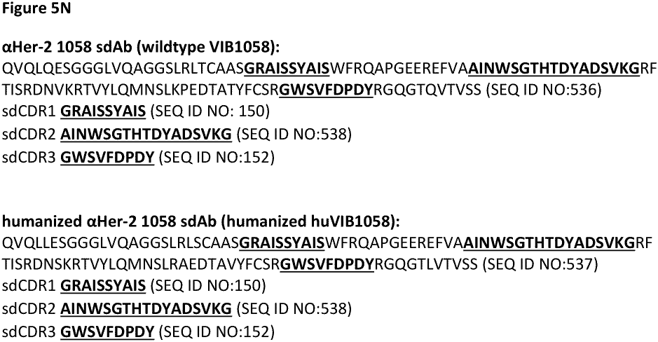

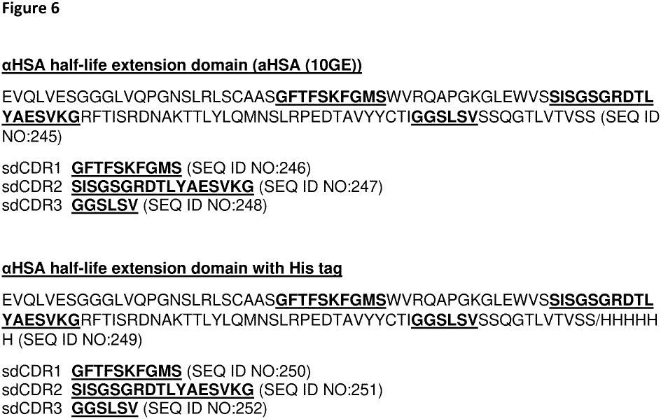

By “cleavable linker” (“CL”) herein is meant an amino acid sequence that can be cleaved by a protease, preferably a human protease in a disease tissue as outlined herein. Cleavable linkers generally are at least 3 amino acids in length, with from 4, 5, 6, 7, 8, 9, 10, 11, 12, 13, 14, 15 or more amino acids finding use in the invention, depending on the required flexibility. A number of cleavable linker sequences are found in FIG. 6 and FIG. 7.

By “non cleavable linker” (“NCL”) herein is meant an amino acid sequence that cannot be cleaved by a human protease under normal physiological conditions.

By “constrained cleavable linker” (“CCL”) herein is meant a short polypeptide that contains a protease cleavage site (as defined herein) that joins two domains as outlined herein in such a manner that the two domains cannot significantly interact with each other until after they reside on different polypeptide chains, e.g. after cleavage. When the CCL joins a VH and a VL domain as defined herein, the VH and VL cannot self- assemble to form a functional Fv prior to cleavage due to steric constraints in an intramolecular way (although they may assemble into pseudo Fv domains in an intermolecular way). Upon cleavage by the relevant protease, the VH and VL can assemble to form an active antigen binding domain in an intermolecular way. In general, CCLs are less than 10 amino acids in length, with 9, 8, 7, 6, 5 and 4 amino acids finding use in the invention. In general, protease cleavage sites generally are at least 4+ amino acids in length to confer sufficient specificity, as is shown in FIG. 6.

By “constrained non-cleavable linker” (“CNCL”) herein is meant a short polypeptide that that joins two domains as outlined herein in such a manner that the two domains cannot significantly interact with each other, and that is not significantly cleaved by human proteases under physiological conditions.

By “constrained Fv domain” herein is meant an Fv domain that comprises an active variable heavy domain and an active variable light domain, linked covalently with a constrained linker as outlined herein, in such a way that the active heavy and light variable domains cannot intramolecularly interact to form an active Fv that will bind an antigen such as CD3. Thus, a constrained Fv domain is one that is similar to an scFv but is not able to bind an antigen due to the presence of a constrained linker (although they may assemble intermolecularly with inert variable domains to form pseudo Fv domains).

By “pseudo Fv domain” herein is meant a domain that comprises a pseudo or inactive variable heavy domain or a pseudo or inactive variable light domain, or both, linked using a domain linker (which can be cleavable, constrained, non-cleavable, non-constrained, etc.). The iVH and iVL domains of a pseudo Fv domain do not bind to a human antigen when either associated with each other (iVH/iVL) or when associated with an active VH or VL; thus iVH/iVL, iVH/VL and iVL/VH Fv domains do not appreciably bind to a human protein, such that these domains are inert in the human body.

By “single chain Fv” or “scFv” herein is meant a variable heavy (VH) domain covalently attached to a variable light (VL) domain, generally using a domain linker as discussed herein, to form a scFv or scFv domain. A scFv domain can be in either orientation from N- to C-terminus (VH -linker- VL or VL-linker-VH).

By “single domain Fv”, “sdFv” or “sdABD” herein is meant an antigen binding domain that only has three CDRs, generally based on camelid antibody technology. See: Protein Engineering 9(7):1129-35 (1994); Rev Mol Biotech 74:277-302 (2001); Ann Rev Biochem 82:775-97 (2013). As outlined herein, there are two general types of sdABDs used herein: sdABDs that bind to TTAs, and are annotated as such (sdABD-TTA for the generic term, or sdABD-EGFR for one that binds to EGFR, sdABD-FOLR1 for one that binds to FOLR1, etc.) and sdABDs that bind to HSA (“sdABD-HSA” or “sdABD(½)”.

By “protease cleavage site” refers to the amino acid sequence recognized and cleaved by a protease. Suitable protease cleavage sites are outlined below and shown in FIG. 7 and FIG. 6.

As used herein, “protease cleavage domain” refers to the peptide sequence incorporating the “protease cleavage site” and any linkers between individual protease cleavage sites and between the protease cleavage site(s) and the other functional components of the constructs of the invention (e.g., V_{H}, V_{L}, iVH, iVL, target antigen binding domain(s), half-life extension domain, etc.). As outlined herein, a protease cleavage domain may also include additional amino acids if necessary, for example to confer flexibility.

The term “COBRA™” and “conditional bispecific redirected activation” refers to a bispecific conditionally effective protein that has a number of functional protein domains. In some embodiments, one of the functional domains is an antigen binding domain (ABD) that binds a target tumor antigen (TTA). In certain embodiments, another domain is an ABD that binds to a T cell antigen under certain conditions. The T cell antigen includes but is not limited to CD3. The term “hemi-COBRA™” refers to a conditionally effective protein that can bind a T cell antigen when a variable heavy chain of a hemi-COBRA can associate to a variable light chain of another hemi-COBRA™ (a complementary hemi-COBRA™) due to innate self-assembly when concentrated on the surface of a target expressing cell.

### Detailed Description of the Embodiments

### I. Fusion Proteins of the Invention

The fusion proteins of the invention have a number of different components, generally referred to herein as domains that are linked together in a variety of ways. Some of the domains are binding domains, that each bind to a target antigen (e.g. a TTA or CD3, for example). As they bind to more than one antigen, they are referred to herein as “multispecific”; for example, a prodrug construct of the invention may bind to a TTA and CD3, and thus are “bispecific”. A protein can also have higher specificities; for example, if the first αTTA binds to EGFR, the second to EpCAM and there is an anti-CD3 binding domain, this would be a “trispecific” molecule. Similarly, the addition of an anti-HSA binding domain to this construct would be “tetraspecific”, as shown in FIG. 3B.

As will be appreciated by those in the art, the proteins of the invention can have different valencies as well as be multispecific. That is, proteins of the invention can bind a target with more than one binding site; for example, Pro186 is bivalent for EGFR.

The proteins of the invention can include CD3 antigen binding domains arranged in a variety of ways as outlined herein, tumor target antigen binding domains, half-life extension domains, linkers, etc.

### A. CD3 Antigen Binding Domains

The specificity of the response of T cells is mediated by the recognition of antigen (displayed in context of a major histocompatibility complex, MHC) by the T cell receptor complex. As part of the T cell receptor complex, CD3 is a protein complex that includes a CD3γ (gamma) chain, a CD3δ (delta) chain, two CD3e (epsilon) chains and two CD3ζ (zeta) chains, which are present at the cell surface. CD3 molecules associate with the α (alpha) and β (beta) chains of the T cell receptor (TCR) to comprise the TCR complex. Clustering of CD3 on T cells, such as by Fv domains that bind to CD3 leads to T cell activation similar to the engagement of the T cell receptor but independent of its clonal-typical specificity.

However, as is known in the art, CD3 activation can cause a number of toxic side effects, and accordingly the present invention is directed to providing active CD3 binding of the polypeptides of the invention only in the presence of tumor cells, where specific proteases are found, that then cleave the prodrug polypeptides of the invention to provide an active CD3 binding domain. Thus, in the present invention, binding of an anti-CD3 Fv domain to CD3 is regulated by a protease cleavage domain which restricts binding of the CD3 Fv domain to CD3 only in the microenvironment of a diseased cell or tissue with elevated levels of proteases, for example in a tumor microenvironment as is described herein.

Accordingly, the present invention provides two sets of VH and VL domains, an active set (VH and VL) and an inactive set (inactive VH and inactive VL; also referred to as “iVH” and “iVL”, respectively) with all four being present in the prodrug construct. The construct is formatted such that the VH and VL set cannot self-associate, but rather associates with an inactive partner, e.g. iVH and VL and iVL and VH as is shown herein.

### 1. Active anti-CD3 Variable Heavy and Variable Light Domains

There are a number of suitable active CDR sets, and/or VH and VL domains, that are known in the art that find use in the present invention. For example, the CDRs and/or VH and VL domains are derived from known anti-CD3 antibodies, such as, for example, muromonab-CD3 (OKT3), otelixizumab (TRX4), teplizumab (MGA031), visilizumab (Nuvion), SP34 or I2C, TR-66 or X35-3, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1 and WT-31.

In one embodiment, the VH and VL sequences that form an active Fv domain that binds to human CD3 are shown in FIGS. 7A-7B. As is shown herein, these active VH (“aVH”) and active VL (“aVL”) domains can be used in different configurations and Formats 1, 2, 3 and 4.

### 2. Inactive anti-CD3 Variable Heavy and Variable Light Domains

The inactive iVH and iVL domains contain “regular” framework regions (FRs) that allow association, such that an inactive variable domain will associate with an active variable domain, rendering the pair inactive, e.g. unable to bind CD3.

As will be appreciated by those in the art, there are a number of “inactive” variable domains that find use in the invention. Basically, any variable domain with human framework regions that allows self-assembly with another variable domain, no matter what amino acids are in the CDR location in the variable region, can be used. For clarity, the inactive domains are said to include CDRs, although technically the inactive variable domains do not confer binding capabilities.

As will be appreciated in the art, it is generally straightforward to generate inactive VH or VL domains, and can be done in a variety of ways. In some embodiments, the generation of inactive variable domains is generally done by altering one or more of the CDRs of an active Fv, including making changes in one or more of the three CDRs of an active variable domain. This can be done by making one or more amino acid substitutions at functionally important residues in one or more CDRs, replacing some or all CDR residues with random sequences, replacing one or more CDRs with tag or flag sequences, and/or swapping CDRs and/or variable regions with those from an irrelevant antibody (one directed to a different organism’s protein for example.

In some cases, only one of the CDRs in a variable region can be altered to render it inactive, although other embodiments include alterations in one, two, three, four, five or six CDRs.

In some cases, the inactive domains can be engineered to promote selective binding in the prodrug format, to encourage formation of intramolecular iVH-VL and VH-iVL domains prior to cleavage (over, for example, intermolecular pair formation). See for example Igawa et al., Protein Eng. Des. Selection 23(8):667-677 (2010), hereby expressly incorporated by reference in its entirety and specifically for the interface residue amino acid substitutions.

In certain embodiments, the CD3 binding domain of the polypeptide constructs described herein exhibit not only potent CD3 binding affinities with human CD3, but show also excellent cross reactivity with the respective cynomolgus monkey CD3 proteins. In some instances, the CD3 binding domain of the polypeptide constructs is cross-reactive with CD3 from cynomolgus monkey. In certain instances, human:cynomolgous KD ratios for CD3 are between 5 and 0.2.

In some embodiments, the CD3 binding domain of the antigen binding protein can be any domain that binds to CD3 including but not limited to domains from a monoclonal antibody, a polyclonal antibody, a recombinant antibody, a human antibody, a humanized antibody. In some instances, it is beneficial for the CD3 binding domain to be derived from the same species in which the antigen binding protein will ultimately be used in. For example, for use in humans, it may be beneficial for the CD3 binding domain of the antigen binding protein to comprise human or humanized residues from the antigen binding domain of an antibody or antibody fragment.

Thus, in one aspect, the antigen-binding domain comprises a humanized or human binding domain. In one embodiment, the humanized or human anti-CD3 binding domain comprises one or more (e.g., all three) light chain complementary determining region 1 (LC CDR1), light chain complementary determining region 2 (LC CDR2), and light chain complementary determining region 3 (LC CDR3) of a humanized or human anti-CD3 binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a humanized or human anti-CD3 binding domain described herein, e.g., a humanized or human anti-CD3 binding domain comprising one or more, e.g., all three, LC CDRs and one or more, e.g., all three, HC CDRs.

In some embodiments, the humanized or human anti-CD3 binding domain comprises a humanized or human light chain variable region specific to CD3 where the light chain variable region specific to CD3 comprises human or non-human light chain CDRs in a human light chain framework region. In certain instances, the light chain framework region is a λ (lambda) light chain framework. In other instances, the light chain framework region is a κ (kappa) light chain framework.

In some embodiments, one or more CD3 binding domains are humanized or fully human. In some embodiments, one or more activated CD3 binding domains have a KD binding of 1000 nM or less to CD3 on CD3 expressing cells. In some embodiments, one or more activated CD3 binding domains have a KD binding of 100 nM or less to CD3 on CD3 expressing cells. In some embodiments, one or more activated CD3 binding domains have a KD binding of 10 nM or less to CD3 on CD3 expressing cells. In some embodiments, one or more CD3 binding domains have crossreactivity with cynomolgus CD3. In some embodiments, one or more CD3 binding domains comprise an amino acid sequence provided herein.

In some embodiments, the humanized or human anti-CD3 binding domain comprises a humanized or human heavy chain variable region specific to CD3 where the heavy chain variable region specific to CD3 comprises human or non-human heavy chain CDRs in a human heavy chain framework region.

In one embodiment, the anti-CD3 binding domain is an Fv comprising a light chain and a heavy chain of an amino acid sequence provided herein. In an embodiment, the anti-CD3 binding domain comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 95-99% identity to an amino acid sequence provided herein. In one embodiment, the humanized or human anti-CD3 binding domain is a scFv, and a light chain variable region comprising an amino acid sequence described herein, is attached to a heavy chain variable region comprising an amino acid sequence described herein, via a scFv linker. The light chain variable region and heavy chain variable region of a scFv can be, e.g., in any of the following orientations: light chain variable region- scFv linker-heavy chain variable region or heavy chain variable region- scFv linker-light chain variable region.

In some embodiments, CD3 binding domain of an antigen binding protein has an affinity to CD3 on CD3 expressing cells with a KD of 1000 nM or less, 100 nM or less, 50 nM or less, 20 nM or less, 10 nM or less, 5 nM or less, 1 nM or less, or 0.5 nM or less. In some embodiments, the CD3 binding domain of an antigen binding protein has an affinity to CD3ε with a KD of 1000 nM or less, 100 nM or less, 50 nM or less, 20 nM or less, 10 nM or less, 5 nM or less, 1 nM or less, or 0.5 nM or less. In further embodiments, CD3 binding domain of an antigen binding protein has low affinity to CD3, i.e., about 100 nM or greater.

The affinity to bind to CD3 can be determined, for example, by the ability of the antigen binding protein itself or its CD3 binding domain to bind to CD3 coated on an assay plate; displayed on a microbial cell surface; in solution; etc., as is known in the art, generally using Biacore or Octet assays. The binding activity of the antigen binding protein itself or its CD3 binding domain of the present disclosure to CD3 can be assayed by immobilizing the ligand (e.g., CD3) or the antigen binding protein itself or its CD3 binding domain, to a bead, substrate, cell, etc. Agents can be added in an appropriate buffer and the binding partners incubated for a period of time at a given temperature. After washes to remove unbound material, the bound protein can be released with, for example, SDS, buffers with a high pH, and the like and analyzed, for example, by Surface Plasmon Resonance (SPR).

In many embodiments, preferred active and inert binding domains are those shown in FIG. 7. FIG. 7 depicts one active VH and VL and three inactive VHi and three inactive VLis that have been inactivated in different ways.

As shown in FIG. 7, a particularly useful pair of active anti-CD3 VL and VH domains has a VL with a v1CDR1 with SEQ ID NO:255, a vlCDR2 with SEQ ID NO:256 and a vlCDR3 with SEQ ID NO:257 and a VH with a vhCDR1 with SEQ ID NO:271, a vhCDR2 with SEQ ID NO:272 and a vhCDR3 with SEQ ID NO:273.

As shown in FIG. 7, a particularly useful pair of active anti-CD3 VL and VH domains has a VL with SEQ ID NO:254 and a VH with SEQ ID NO:270.

### B. Antigen Binding Domains to Tumor Target Antigens

In addition to the described CD3 and half-life extension domains, the polypeptide constructs described herein also comprise target domains that bind to one or more target antigens or one or more regions on a single target antigen. It is contemplated herein that a polypeptide construct of the invention is cleaved, for example, in a disease-specific microenvironment or in the blood of a subject at the protease cleavage domain and that each target antigen binding domain will bind to a target antigen on a target cell, thereby activating the CD3 binding domain to bind a T cell. In general, the TTA binding domains can bind to their targets before protease cleavage, so they can “wait” on the target cell to be activated as T-cell engagers. At least one target antigen is involved in and/or associated with a disease, disorder or condition. Exemplary target antigens include those associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In some embodiments, a target antigen is a tumor antigen expressed on a tumor cell. Alternatively, in some embodiments, a target antigen is associated with a pathogen such as a virus or bacterium. At least one target antigen may also be directed against healthy tissue.

In some embodiments, a target antigen is a cell surface molecule such as a protein, lipid or polysaccharide. In some embodiments, a target antigen is a on a tumor cell, virally infected cell, bacterially infected cell, damaged red blood cell, arterial plaque cell, or fibrotic tissue cell.

Preferred embodiments of the invention utilize sdABDs as the targeting domains. These are preferred over scFv ABDs, since the addition of other VH and VL domains into a construct of the invention may complicate the formation of pseudo Fv domains.

In some embodiments, the pro-drug constructs of the invention utilize a single TTA binding domain, such as generally depicted in FIG. 3A, as pairs of sdABD-TTAs, and FIG. 4, as a “format 4” configuration. FIG. 4 shows the use of a single anti-EGFR ABD, although other TTA binding domains can be used.

In some embodiments, particularly in the Format 1 and Format 2 constructs, the pro-drug constructs of the invention utilize two TTA ABDs, again preferably in the sdABD-TTA format. When dual targeting domains are used, they can bind to the same epitope of the same TTA. For example, as discussed herein, many of the constructs herein utilize two identical targeting domains. In some embodiments, two targeting domains can be used that bind to different epitopes of the same TTA, for example as shown in FIG. 7, the two EGFR sdABDs bind to different epitopes on human EGFR. In some embodiments, the two targeting domains bind to different TTAs as more fully described below.

Polypeptide constructs contemplated herein include at least one antigen binding domain, wherein the antigen binding domain binds to at least one target antigen. In some embodiments, the target antigen binding domains specifically bind to a cell surface molecule. In some embodiments, the target antigen binding domains specifically bind to a tumor antigen. In some embodiments, the target antigen binding domains specifically and independently bind to a tumor target antigen (“TTA”) selected from at least one of EpCAM, EGFR, HER-2, LyPD3, B7H3, CA9, Trop2 and FOLR1. As discussed below, these can be combined in a variety of ways.

### (A) EGFR sdABDs

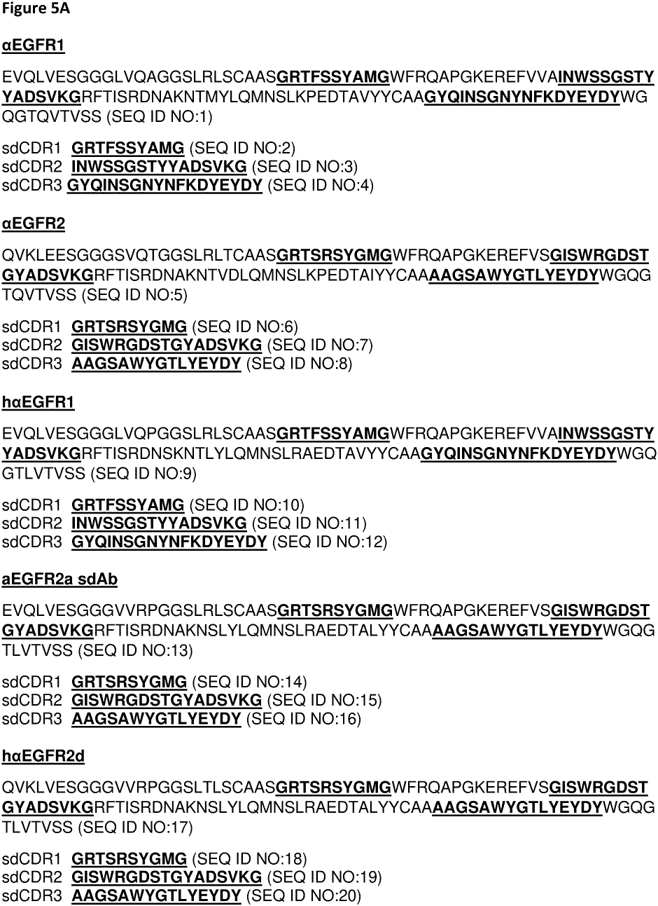

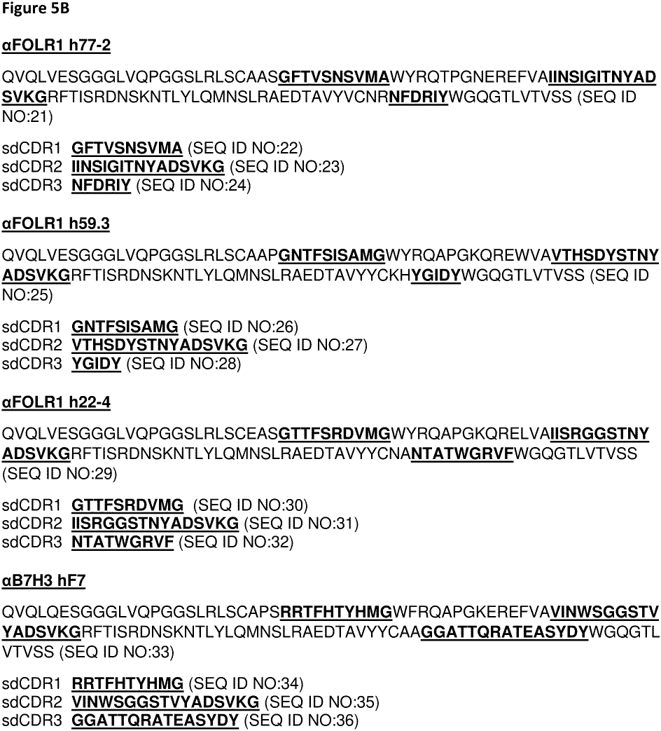

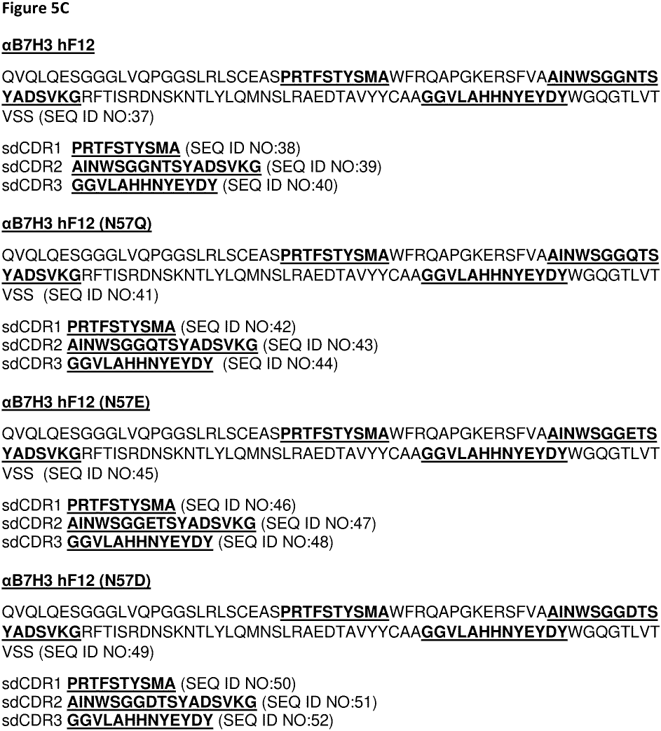

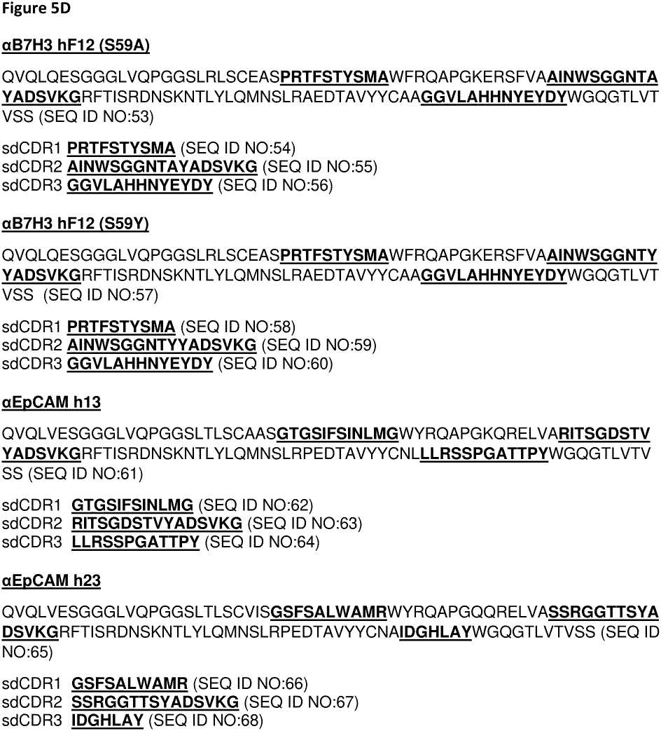

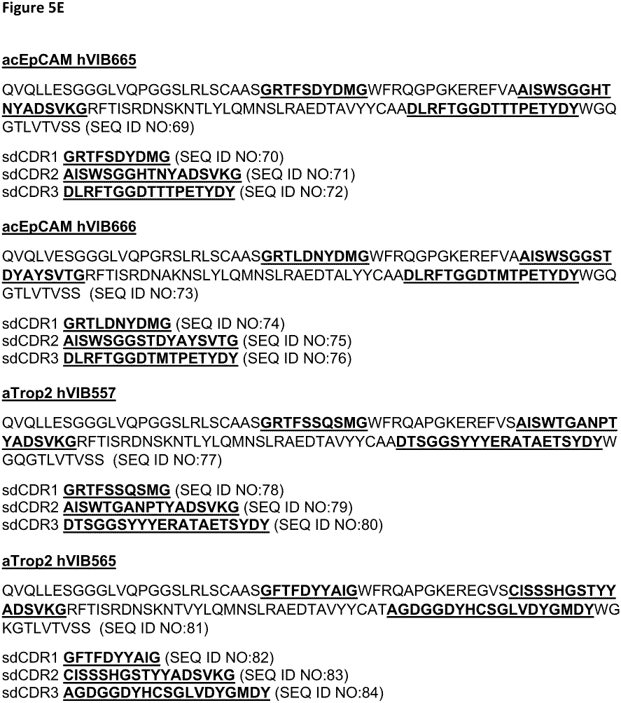

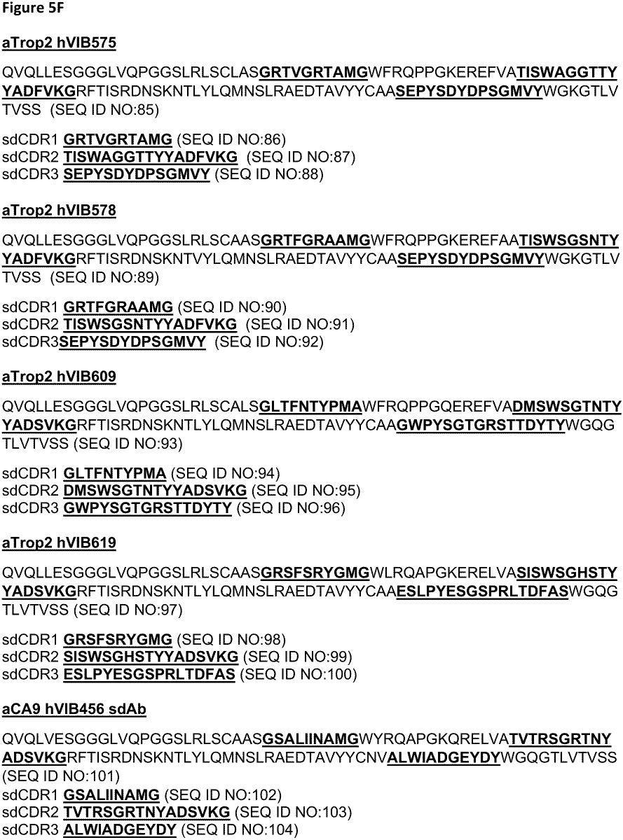

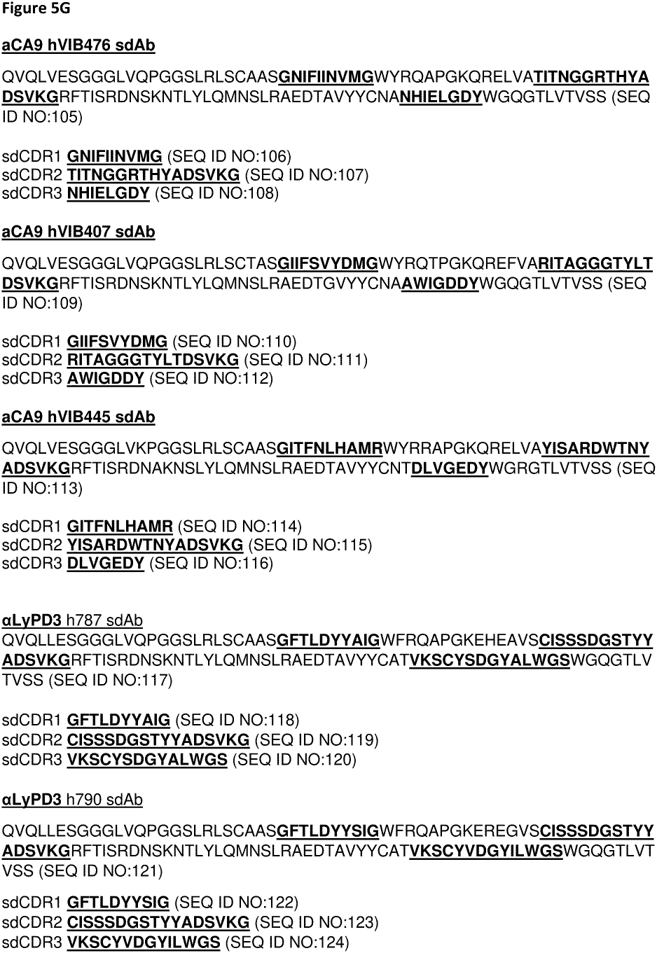

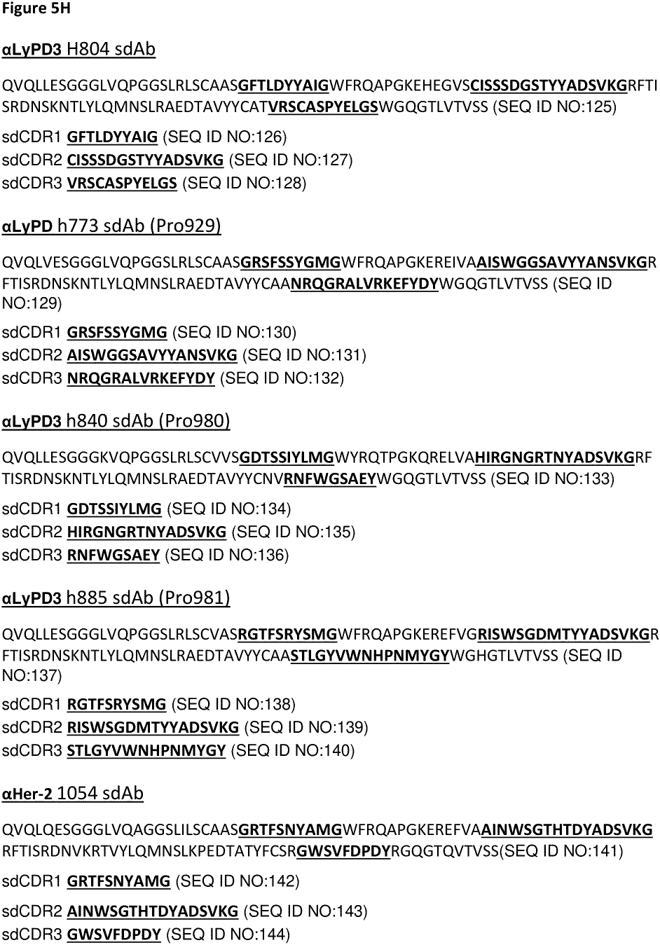

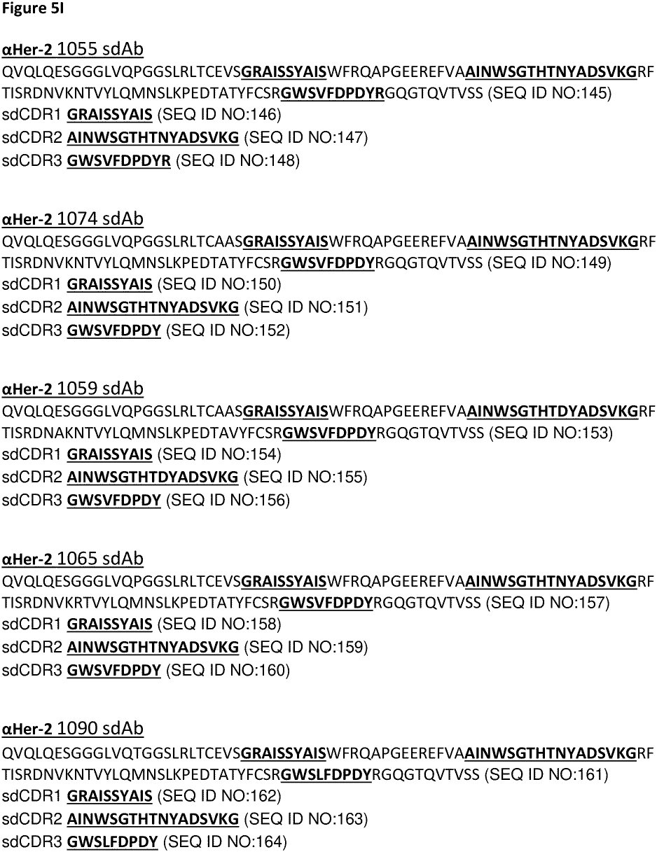

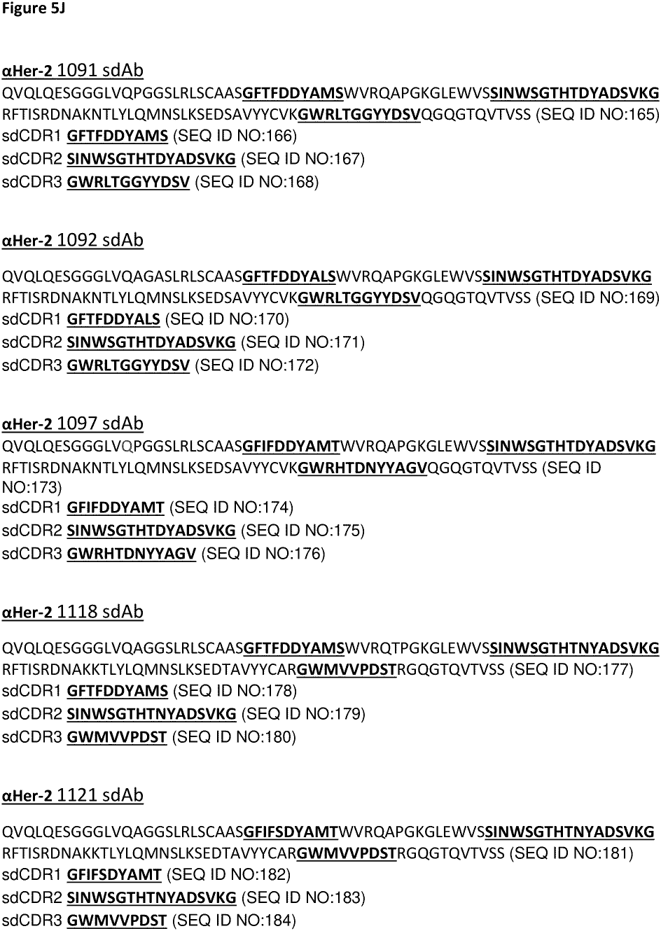

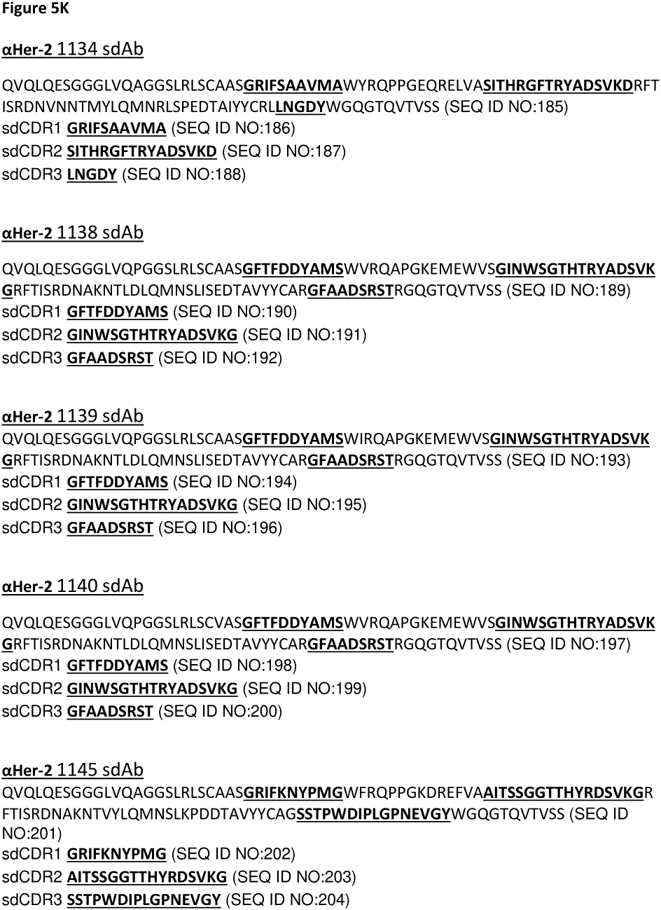

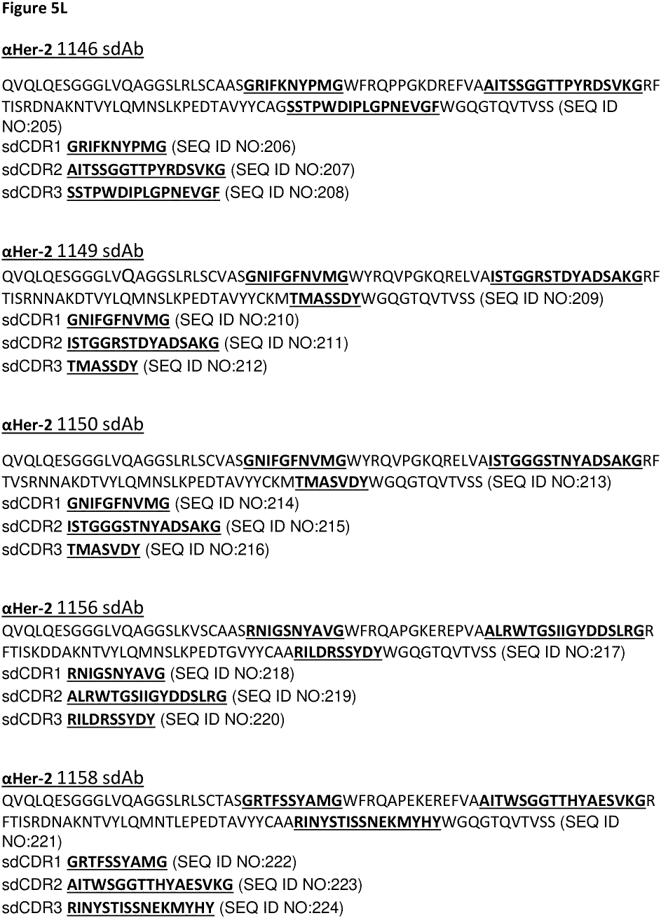

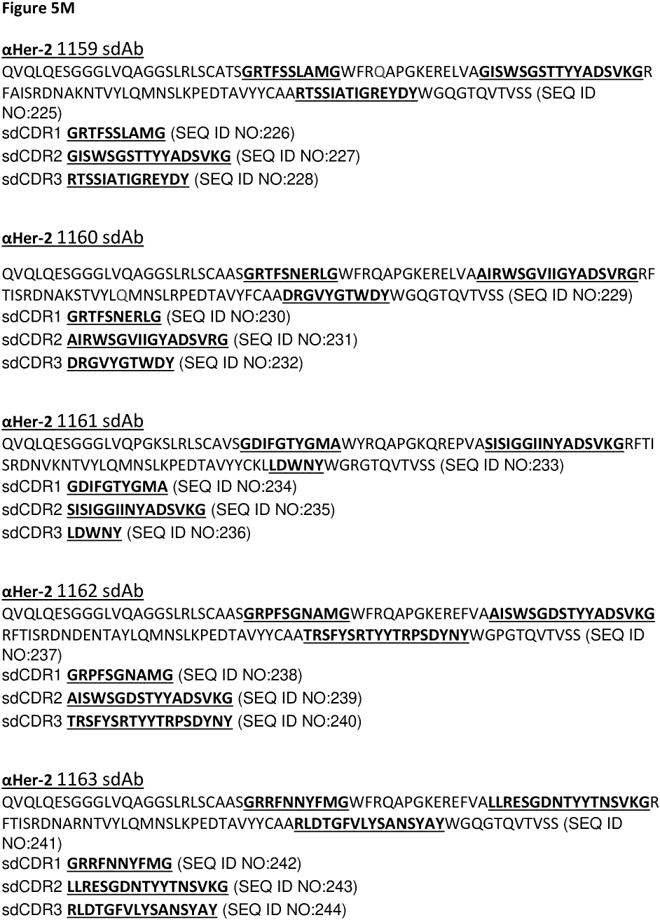

As shown in FIG. 5A, there are a number of particularly useful sdABDs that binding to human EGFR, referred to herein as “αEGFR”, “aEGFR”, “sdABD-EGFR”, “EGFR sdABDs”, “EGFR sdAbs”, “EGFR ABDs” or “EGFRABDs”.

In some embodiments, the sdABD-EGFR (e.g., sdABD-αEGFR1) has a sdCDR1 with SEQ ID NO:2 a sdCDR2 with SEQ ID NO:3 and a sdCDR3 with SEQ ID NO:4. In some cases, the sdABD-EGFR has the amino acid sequence of SEQ ID NO:1, as provided in FIG. 5A.

In some embodiments, the sdABD-EGFR (e.g., sdABD-αEGFR2) has a sdCDR1 with SEQ ID NO:6, a sdCDR2 with SEQ ID NO:7 and a sdCDR3 with SEQ ID NO:8. In some cases, the sdABD-EGFR has the amino acid sequence of SEQ ID NO:5, as provided in FIG. 5A.

In some embodiments, the sdABD-EGFR (e.g., sdABD-hαEGFR1) has a sdCDR1 with SEQ ID NO:10, a sdCDR2 with SEQ ID NO:11 and a sdCDR3 with SEQ ID NO:12. In some cases, the sdABD-EGFR has the amino acid sequence of SEQ ID NO:9, as provided in FIG. 5A.

In some embodiments, the sdABD-EGFR (e.g., sdABD-aEGFR2a) has a sdCDR1 with SEQ ID NO:14, a sdCDR2 with SEQ ID NO:15 and a sdCDR3 with SEQ ID NO:16. In some cases, the sdABD-EGFR has the amino acid sequence of SEQ ID NO:13, as provided in FIG. 5A.

In some embodiments, the sdABD-EGFR (e.g., sdABD-hαEGFR2d) has a sdCDR1 with SEQ ID NO:18, a sdCDR2 with SEQ ID NO:19 and a sdCDR3 with SEQ ID NO:20. In some cases, the sdABD-EGFR has the amino acid sequence of SEQ ID NO:17, as provided in FIG. 5A.

### (B) EpCAM sdABDs

As shown in FIGS. 5D-5E, there are a number of particularly useful sdABDs that binding to human EpCAM, referred to herein as “αEpCAM”, “aEpCAM”, “sdABD-EpCAM”, EpCAM sdABDs″, “EpCAM sdAbs”, “EpCAM ABDs” or “EpCAMABDs”.

In some embodiments, the sdABD-EpCAM (e.g., sdABD-EpCAM h13) has a sdCDR1 with SEQ ID NO:62, a sdCDR2 with SEQ ID NO:63, a sdCDR3 with SEQ ID NO:64. In some cases, the sdABD-EpCAM has the amino acid sequence of SEQ ID NO:61, as provided in FIG. 5D.

In some embodiments, the sdABD-EpCAM (e.g., sdABD-EpCAM h23) has a sdCDR1 with SEQ ID NO:66, a sdCDR2 with SEQ ID NO:67, a sdCDR3 with SEQ ID NO:68. In some cases, the sdABD-EpCAM has the amino acid sequence of SEQ ID NO:65, as provided in FIG. 5D.

In some embodiments, the sdABD-EpCAM (e.g., sdABD-EpCAM hVIB665) has a sdCDR1 with SEQ ID NO:70, a sdCDR2 with SEQ ID NO:71, a sdCDR3 with SEQ ID NO:72. In some cases, the sdABD-EpCAM has the amino acid sequence of SEQ ID NO:69, as provided in FIG. 5E. It should be noted that in contrast to the h13 and h23 EpCAM sdABDs, hVIB665 (also referred to as “acEpCAM hVIB665”) binds to both the cleaved and uncleaved form of EpCAM (which is known to undergo a cleavage in vivo).

In some embodiments, the sdABD-EpCAM (e.g., sdABD-EpCAM hVIB666) has a sdCDR1 with SEQ ID NO:74, a sdCDR2 with SEQ ID NO:75, a sdCDR3 with SEQ ID NO:76. In some cases, the sdABD-EpCAM has the amino acid sequence of SEQ ID NO:73, as provided in FIG. 5E. It should be noted that in contrast to the h13 and h23 EpCAM sdABDs, hVIB666 (also referred to as “acEpCAM hVIB666”) binds to both the cleaved and uncleaved form of EpCAM (which is known to undergo a cleavage in vivo).

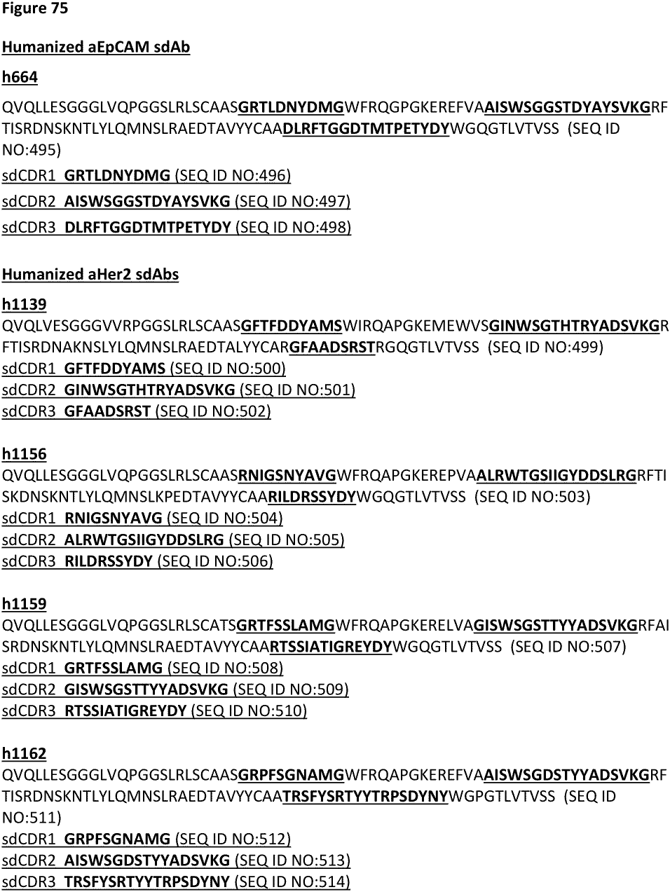

In some embodiments, the sdABD-EpCAM (e.g., humanized a EpCAM sdAb) has a sdCDR1 with SEQ ID NO:496, a sdCDR2 with SEQ ID NO:497, a sdCDR3 with SEQ ID NO:498. In some cases, the sdABD-EpCAM has the amino acid sequence of SEQ ID NO:495, as provided in FIG. 75.

### (C) B7H3 sdABDs

As shown in FIGS. 5B-5D, there are a number of particularly useful sdABDs that binding to human B7H3, referred to herein as “αB7H3”, “aB7H3”, “sdABD-B7H3”, “B7H3 sdAbs”, “B7H3 ABDs”, “B7H3ABDs” or “B7H3-ABDs”.

In one useful embodiment, the sdABD-B7H3 (e.g., sdABD-B7H3 hF7) has a sdCDR1 with SEQ ID NO:34, a sdCDR2 with SEQ ID NO:35, a sdCDR3 with SEQ ID NO:36. In some cases, the sdABD-B7H3 has the amino acid sequence of SEQ ID NO:33, as provided as FIG. 5B.

In one useful embodiment, the sdABD-B7H3 (e.g., sdABD-B7H3 hF12) has a sdCDR1 with SEQ ID NO:38, a sdCDR2 with SEQ ID NO:39, a sdCDR3 with SEQ ID NO:40. In some cases, the sdABD-B7H3 has the amino acid sequence of SEQ ID NO:37, as provided as FIG. 5C.

In one useful embodiment, the sdABD-B7H3 (e.g., sdABD-B7H3 hF12 (N57Q)) has a sdCDR1 with SEQ ID NO:42, a sdCDR2 with SEQ ID NO:43, a sdCDR3 with SEQ ID NO:44. IN some cases, the sdABD-B7H3 has the amino acid sequence of SEQ ID NO:41, as provided as FIG. 5C. In contrast to the hF7 and hF12 B7H3 sdABDs, the amino acid substitution N57Q removes a glycosylation site.

In one useful embodiment, the sdABD-B7H3 (e.g., sdABD-B7H3 HF12 (N57E)) has a sdCDR1 with SEQ ID NO:46, a sdCDR2 with SEQ ID NO:47, and a sdCDR3 with SEQ ID NO:48. In some cases, the sdABD-B7H3 has the amino acid sequence of SEQ ID NO:45, as provided as FIG. 5C. In contrast to the hF7 and hF12 B7H3 sdABDs, the amino acid substitution N57E removes a glycosylation site.

In one useful embodiment, the sdABD-B7H3 (e.g., sdABD-B7H3 hF12 (N57D)) has a sdCDR1 with SEQ ID NO:50, a sdCDR2 with SEQ ID NO:51, a sdCDR3 with SEQ ID NO:52. In some cases, the sdABD-B7H3 has the amino acid sequence of SEQ ID NO:49, as provided as FIG. 5B. In contrast to the hF7 and hF12 B7H3 sdABDs, the amino acid substitution N57D removes a glycosylation site.

In one useful embodiment, the sdABD-B7H3 (e.g., sdABD-B7H3 hF12(S59A)) has a sdCDR1 with SEQ ID NO:54, a sdCDR2 with SEQ ID NO:55, a sdCDR3 with SEQ ID NO:56. IN some cases, the sdABD-B7H3 has the amino acid sequence of SEQ ID NO:53, as provided as FIG. 5D. In contrast to the hF7 and hF12 B7H3 sdABDs, the amino acid substitution S59A removes a glycosylation site.

In one useful embodiment, the sdABD-B7H3 (e.g., sdABD-B7H3 hF12 (S59Y)) has a sdCDR1 with SEQ ID NO:58, a sdCDR2 with SEQ ID NO:59, a sdCDR3 with SEQ ID NO:60. IN some cases, the sdABD-B7H3 has the amino acid sequence of SEQ ID NO:57, as provided as FIG. 5D. In contrast to the hF7 and hF12 B7H3 sdABDs, the amino acid substitution NS59Y removes a glycosylation site.

### (D) FOLR1 sdABDs

As shown in FIG. 5B, there are a number of particularly useful sdABDs that binding to human FOLR1, referred to herein as “αFOLR1”, “aFOLR1”, “sdABD-FOLR1”, “FOLR1 sdAbs”, “sdABDs FOLR1”,“FOLR1 ABDs”, “FOLR1ABDs” or “FOLR1-ABDs”.

In one useful embodiment, the sdABD-FOLR1 (e.g., sdABD-FOLR1 h77-2) has a sdCDR1 with SEQ ID NO:22, a sdCDR2 with SEQ ID NO:23, a sdCDR3 with SEQ ID NO:24. In some cases, the sdABD-FOLR1 has the amino acid sequence of SEQ ID NO:21, as provided in FIG. 5B.

In one useful embodiment, the sdABD-FOLR1 (e.g., sdABD-FOLR1 h59.3) has a sdCDR1 with SEQ ID NO:26, a sdCDR2 with SEQ ID NO:27, a sdCDR3 with SEQ ID NO:28. In some cases, the sdABD-FOLR1 has the amino acid sequence of SEQ ID NO:25, as provided in FIG. 5B.

In one useful embodiment, the sdABD-FOLR1 (e.g., sdABD-FOLR1 h22-4) has a sdCDR1 with SEQ ID NO:30, a sdCDR2 with SEQ ID NO:31, a sdCDR3 with SEQ ID NO:32. In some cases, the sdABD-FOLR1 has the amino acid sequence of SEQ ID NO:29, as provided in FIG. 5B.

### (E) Trop2 sdABDs

As shown in FIG. 5E, there are a number of particularly useful sdABDs that binding to human Trop2, referred to herein as “αTrop2”, “aTrop2”, “sdABD-Trop2”, “sdABDs Trop2”, “Trop2 sdAbs”, “Trop2ABDs” or “Trop2-ABDs”.

In one useful embodiment, the sdABD-Trop2 (e.g., sdABD-Trop2 hVIB557) has a sdCDR1 with SEQ ID NO:78, a sdCDR2 with SEQ ID NO:79, a sdCDR3 with SEQ ID NO:80. In some cases, the sdABD-Trop2 has the amino acid sequence of SEQ ID NO:77, as provided in FIG. 5E.

In some embodiments, the sdABD-Trop2 (e.g., sdABD-Trop2 hVIB565) has a sdCDR1 with SEQ ID NO:82, a sdCDR2 with SEQ ID NO:83, a sdCDR3 with SEQ ID NO:84. In some cases, the sdABD-Trop2 has the amino acid sequence of SEQ ID NO:81, as provided in FIG. 5E.

In some embodiments, the sdABD-Trop2 (e.g., sdABD-Trop2 hVIB575) has a sdCDR1 with SEQ ID NO:86, a sdCDR2 with SEQ ID NO:87, a sdCDR3 with SEQ ID NO:88. In some cases, the sdABD-Trop2 has the amino acid sequence of SEQ ID NO:85, as provided in FIG. 5F.

In some embodiments, the sdABD-Trop2 (e.g., sdABD-Trop2 hVIB578) has a sdCDR1 with SEQ ID NO:90, a sdCDR2 with SEQ ID NO:91, a sdCDR3 with SEQ ID NO:92. In some cases, the sdABD-Trop2 has the amino acid sequence of SEQ ID NO:89, as provided in FIG. 5F.

In some embodiments, the sdABD-Trop2 (e.g., sdABD-Trop2 hVIB609) has a sdCDR1 with SEQ ID NO:94, a sdCDR2 with SEQ ID NO:95, a sdCDR3 with SEQ ID NO:96. In some cases, the sdABD-Trop2 has the amino acid sequence of SEQ ID NO:93, as provided in FIG. 5F.

In some embodiments, the sdABD-Trop2 (e.g., sdABD-Trop2 hVIB619) has a sdCDR1 with SEQ ID NO:98, a sdCDR2 with SEQ ID NO:99, a sdCDR3 with SEQ ID NO:100. In some cases, the sdABD-Trop2 has the amino acid sequence of SEQ ID NO:97, as provided in FIG. 5F.

### (F) CA9 sdABDs

As shown in FIGS. 5F-5G, there are a number of particularly useful sdABDs that binding to human CA9, referred to herein as “αCA9”, “aCA9”, “sdABD-CA9”, “sdABDs CA9”, “CA9 sdAbs”, “CA9 ABDs” or “CA9-ABDs”.

In some embodiments, the sdABD-CA9 (e.g., sdABD-CA9 hVIB456) has a sdCDR1 with SEQ ID NO:102, a sdCDR2 with SEQ ID NO:103, a sdCDR3 with SEQ ID NO:104. In some cases, the sdABD-Trop2 has the amino acid sequence of SEQ ID NO:101, as provided in FIG. 5F.

In some embodiments, the sdABD-CA9 (e.g., sdABD-CA9 hVIB476) has a sdCDR1 with SEQ ID NO:106, a sdCDR2 with SEQ ID NO:107, a sdCDR3 with SEQ ID NO:108. In some cases, the sdABD-Trop2 has the amino acid sequence of SEQ ID NO:105, as provided in FIG. 5G.

In some embodiments, the sdABD-CA9 (e.g., sdABD-CA9 hVIB407) has a sdCDR1 with SEQ ID NO:110, a sdCDR2 with SEQ ID NO:111, a sdCDR3 with SEQ ID NO:112. IN some cases, the sdABD-Trop2 has the amino acid sequence of SEQ ID NO:109, as provided in FIG. 5G.

In some embodiments, the sdABD-CA9 (e.g., sdABD-CA9 hVIB445) has a sdCDR1 with SEQ ID NO:114, a sdCDR2 with SEQ ID NO:115, a sdCDR3 with SEQ ID NO:116. In some cases, the sdABD-Trop2 has the amino acid sequence of SEQ ID NO:113, as provided in FIG. 5G.

### (G) LyPD3 sdABDs

As shown in FIGS. 5G-5H, there are a number of particularly useful sdABDs that binding to human LyPD3, referred to herein as “αLyPD3”, “sdABD-LyPD3”, “sdABDs LyPD3”, “LyPD3 sdAbs”, “LyPD3 ABDs”, “LyPD3ABDs” or “LyPD3-ABDs”.

In one useful embodiment, the sdABD-LyPD3 (e.g., sdABD-LyPD3 h787) has a sdCDR1 with SEQ ID NO:118, a sdCDR2 with SEQ ID NO:119, a sdCDR3 with SEQ ID NO:120. In some cases, the sdABD-LyPD3 has the amino acid sequence of SEQ ID NO:117, as provided in FIG. 5G.

In one useful embodiment, the sdABD-LyPD3 (e.g., sdABD-LyPD3 h790) has a sdCDR1 with SEQ ID NO:122, a sdCDR2 with SEQ ID NO:123, a sdCDR3 with SEQ ID NO:124. In some cases, the sdABD-LyPD3 has the amino acid sequence of SEQ ID NO:121, as provided in FIG. 5G.

In one useful embodiment, the sdABD-LyPD3 (e.g., sdABD-LyPD3 H804) has a sdCDR1 with SEQ ID NO:126, a sdCDR2 with SEQ ID NO:127, a sdCDR3 with SEQ ID NO:128. In some cases, the sdABD-LyPD3 has the amino acid sequence of SEQ ID NO:125, as provided in FIG. 5H.

In one useful embodiment, the sdABD-LyPD3 (e.g., sdABD-LyPD3 h773) has a sdCDR1 with SEQ ID NO:130, a sdCDR2 with SEQ ID NO:131, a sdCDR3 with SEQ ID NO:132. In some cases, the sdABD-LyPD3 has the amino acid sequence of SEQ ID NO:129, as provided in FIG. 5H.

In one useful embodiment, the sdABD-LyPD3 (e.g., sdABD-LyPD3 h840) has a sdCDR1 with SEQ ID NO:134, a sdCDR2 with SEQ ID NO:135, a sdCDR3 with SEQ ID NO:136. In some cases, the sdABD-LyPD3 has the amino acid sequence of SEQ ID NO:133, as provided in FIG. 5H.

In one useful embodiment, the sdABD-LyPD3 (e.g., sdABD-LyPD3 h885) has a sdCDR1 with SEQ ID NO:138, a sdCDR2 with SEQ ID NO:139, a sdCDR3 with SEQ ID NO:140. In some cases, the sdABD-LyPD3 has the amino acid sequence of SEQ ID NO:137, as provided in FIG. 5H.

### (H) HER2 sdABDs

As shown in FIGS. 5H-5N, there are a number of particularly useful sdABDs that binding to human HER2, referred to herein as “αHER2”, “aHER2”, “sdABD-HER2”, “sdABDs HER2”, “HER2 sdAbs”, “HER2 ABDs”, “HER2ABDs” or “HER2-ABDs”.

In some embodiments, the sdABD-HER2 (e.g., sdABD-HER2 1054) has a sdCDR1 with SEQ ID NO:142, a sdCDR2 with SEQ ID NO:143, a sdCDR3 with SEQ ID NO:144. In some cases, the sdABD-HER2 has the amino acid sequence of SEQ ID NO:141, as provided in FIG. 5H.

In some embodiments, the sdABD-HER2 (e.g., sdABD-HER2 1055) has a sdCDR1 with SEQ ID NO:146, a sdCDR2 with SEQ ID NO:147, a sdCDR3 with SEQ ID NO:148. In some cases, the sdABD-HER2 has the amino acid sequence of SEQ ID NO:145, as provided in FIG. 5I.

In some embodiments, the sdABD-HER2 (e.g., sdABD-HER2 1074) has a sdCDR1 with SEQ ID NO:150, a sdCDR2 with SEQ ID NO:151, a sdCDR3 with SEQ ID NO:152. In some cases, the sdABD-HER2 has the amino acid sequence of SEQ ID NO:149, as provided in FIG. 5I.

In some embodiments, the sdABD-HER2 (e.g., sdABD-HER2 1059) has a sdCDR1 with SEQ ID NO:154, a sdCDR2 with SEQ ID NO:155, a sdCDR3 with SEQ ID NO:156. In some cases, the sdABD-HER2 has the amino acid sequence of SEQ ID NO:153, as provided in FIG. 5I.

some embodiments, the sdABD-HER2 (e.g., sdABD-HER2 1065) has a sdCDR1 with SEQ ID NO:158, a sdCDR2 with SEQ ID NO:159, a sdCDR3 with SEQ ID NO:160. In some cases, the sdABD-HER2 has the amino acid sequence of SEQ ID NO:157, as provided in FIG. 5I.

In some embodiments, the sdABD-HER2 (e.g., sdABD-HER2 1090) has a sdCDR1 with SEQ ID NO:162, a sdCDR2 with SEQ ID NO:163, a sdCDR3 with SEQ ID NO:164. In some cases, the sdABD-HER2 has the amino acid sequence of SEQ ID NO:161, as provided in FIG. 5I.

In some embodiments, the sdABD-HER2 (e.g., sdABD-HER2 1191) has a sdCDR1 with SEQ ID NO:166, a sdCDR2 with SEQ ID NO:167, a sdCDR3 with SEQ ID NO:168. In some cases, the sdABD-HER2 has the amino acid sequence of SEQ ID NO:165, as provided in FIG. 5J.

In some embodiments, the sdABD-HER2 (e.g., sdABD-HER2 1092) has a sdCDR1 with SEQ ID NO:170, a sdCDR2 with SEQ ID NO:171, a sdCDR3 with SEQ ID NO:172. In some cases, the sdABD-HER2 has the amino acid sequence of SEQ ID NO:169, as provided in FIG. 5J.

In some embodiments, the sdABD-HER2 (e.g., sdABD-HER2 1097) has a sdCDR1 with SEQ ID NO:174, a sdCDR2 with SEQ ID NO:175, a sdCDR3 with SEQ ID NO:176. In some cases, the sdABD-HER2 has the amino acid sequence of SEQ ID NO:173, as provided in FIG. 5J.

In some embodiments, the sdABD-HER2 (e.g., sdABD-HER2 1118) has a sdCDR1 with SEQ ID NO:178, a sdCDR2 with SEQ ID NO:179, a sdCDR3 with SEQ ID NO:180. In some cases, the sdABD-HER2 has the amino acid sequence of SEQ ID NO:177, as provided in FIG. 5J.

In some embodiments, the sdABD-HER2 (e.g., sdABD-HER2 1121) has a sdCDR1 with SEQ ID NO:182, a sdCDR2 with SEQ ID NO:183, a sdCDR3 with SEQ ID NO:184. In some cases, the sdABD-HER2 has the amino acid sequence of SEQ ID NO:181, as provided in FIG. 5J.

In some embodiments, the sdABD-HER2 (e.g., sdABD-HER2 1134) has a sdCDR1 with SEQ ID NO:186, a sdCDR2 with SEQ ID NO:187, a sdCDR3 with SEQ ID NO:188. In some cases, the sdABD-HER2 has the amino acid sequence of SEQ ID NO:185, as provided in FIG. 5K.

In some embodiments, the sdABD-HER2 (e.g., sdABD-HER2 1138) has a sdCDR1 with SEQ ID NO:190, a sdCDR2 with SEQ ID NO:191, a sdCDR3 with SEQ ID NO:192. In some cases, the sdABD-HER2 has the amino acid sequence of SEQ ID NO:189, as provided in FIG. 5K.

In some embodiments, the sdABD-HER2 (e.g., sdABD-HER2 1139) has a sdCDR1 with SEQ ID NO:194, a sdCDR2 with SEQ ID NO:195, a sdCDR3 with SEQ ID NO:196. In some cases, the sdABD-HER2 has the amino acid sequence of SEQ ID NO:193, as provided in FIG. 5K.

In some embodiments, the sdABD-HER2 (e.g., sdABD-HER2 1140) has a sdCDR1 with SEQ ID NO:198, a sdCDR2 with SEQ ID NO:199, a sdCDR3 with SEQ ID NO:200. In some cases, the sdABD-HER2 has the amino acid sequence of SEQ ID NO:197, as provided in FIG. 5K.

In some embodiments, the sdABD-HER2 (e.g., sdABD-HER2 1145) has a sdCDR1 with SEQ ID NO:202, a sdCDR2 with SEQ ID NO:203, a sdCDR3 with SEQ ID NO:204. In some cases, the sdABD-HER2 has the amino acid sequence of SEQ ID NO:201, as provided in FIG. 5K.

some embodiments, the sdABD-HER2 (e.g., sdABD-HER2 1146) has a sdCDR1 with SEQ ID NO:206, a sdCDR2 with SEQ ID NO:207, a sdCDR3 with SEQ ID NO:203. In some cases, the sdABD-HER2 has the amino acid sequence of SEQ ID NO:205, as provided in FIG. 5L.

In some embodiments, the sdABD-HER2 (e.g., sdABD-HER2 1149) has a sdCDR1 with SEQ ID NO:210, a sdCDR2 with SEQ ID NO:211, a sdCDR3 with SEQ ID NO:212. In some cases, the sdABD-HER2 has the amino acid sequence of SEQ ID NO:209, as provided in FIG. 5L.

In some embodiments, the sdABD-HER2 (e.g., sdABD-HER2 1150) has a sdCDR1 with SEQ ID NO:214, a sdCDR2 with SEQ ID NO:215, a sdCDR3 with SEQ ID NO:216. In some cases, the sdABD-HER2 has the amino acid sequence of SEQ ID NO:213, as provided in FIG. 5L.

In some embodiments, the sdABD-HER2 (e.g., sdABD-HER2 1156) has a sdCDR1 with SEQ ID NO:218, a sdCDR2 with SEQ ID NO:219, a sdCDR3 with SEQ ID NO:220. In some cases, the sdABD-HER2 has the amino acid sequence of SEQ ID NO:217, as provided in FIG. 5L.

In some embodiments, the sdABD-HER2 (e.g., sdABD-HER2 1158) has a sdCDR1 with SEQ ID NO:222, a sdCDR2 with SEQ ID NO:223, a sdCDR3 with SEQ ID NO:224. In some cases, the sdABD-HER2 has the amino acid sequence of SEQ ID NO:221, as provided in FIG. 5L.

In some embodiments, the sdABD-HER2 (e.g., sdABD-HER2 1159) has a sdCDR1 with SEQ ID NO:226, a sdCDR2 with SEQ ID NO:227, a sdCDR3 with SEQ ID NO:228. In some cases, the sdABD-HER2 has the amino acid sequence of SEQ ID NO:225, as provided in FIG. 5M.

In some embodiments, the sdABD-HER2 (e.g., sdABD-HER2 1160) has a sdCDR1 with SEQ ID NO:230, a sdCDR2 with SEQ ID NO:231, a sdCDR3 with SEQ ID NO:232. In some cases, the sdABD-HER2 has the amino acid sequence of SEQ ID NO:229, as provided in FIG. 5M.

In some embodiments, the sdABD-HER2 (e.g., sdABD-HER2 1161) has a sdCDR1 with SEQ ID NO:234, a sdCDR2 with SEQ ID NO:235, a sdCDR3 with SEQ ID NO:236. In some cases, the sdABD-HER2 has the amino acid sequence of SEQ ID NO:233, as provided in FIG. 5M.

In some embodiments, the sdABD-HER2 (e.g., sdABD-HER2 1162) has a sdCDR1 with SEQ ID NO:238, a sdCDR2 with SEQ ID NO:239, a sdCDR3 with SEQ ID NO:240. In some cases, the sdABD-HER2 has the amino acid sequence of SEQ ID NO:237, as provided in FIG. 5M.

In some embodiments, the sdABD-HER2 (e.g., sdABD-HER2 1163) has a sdCDR1 with SEQ ID NO:242, a sdCDR2 with SEQ ID NO:243, a sdCDR3 with SEQ ID NO:244. In some cases, the sdABD-HER2 has the amino acid sequence of SEQ ID NO:241, as provided in FIG. 5M.

In some embodiments, the sdABD-HER2 (e.g., sdABD-HER2 1058) has a sdCDR1 with SEQ ID NO:150, a sdCDR2 with SEQ ID NO:538, a sdCDR3 with SEQ ID NO:152. In some cases, the sdABD-HER2 has the amino acid sequence of SEQ ID NO:536, as provided in FIG. 5N.

In some embodiments, the humanized sdABD-HER2 (e.g., sdABD-HER2 h1058) has a sdCDR1 with SEQ ID NO:150, a sdCDR2 with SEQ ID NO:538, a sdCDR3 with SEQ ID NO:152. In some cases, the sdABD-HER2 has the amino acid sequence of SEQ ID NO:537, as provided in FIG. 5N.

In some embodiments, the sdABD-HER2 (e.g., humanized aHER2 sdAb h1130) has a sdCDR1 with SEQ ID NO:500, a sdCDR2 with SEQ ID NO:501, a sdCDR3 with SEQ ID NO:502. In some cases, the sdABD-HER2 has the amino acid sequence of SEQ ID NO:499, as provided in FIG. 75.

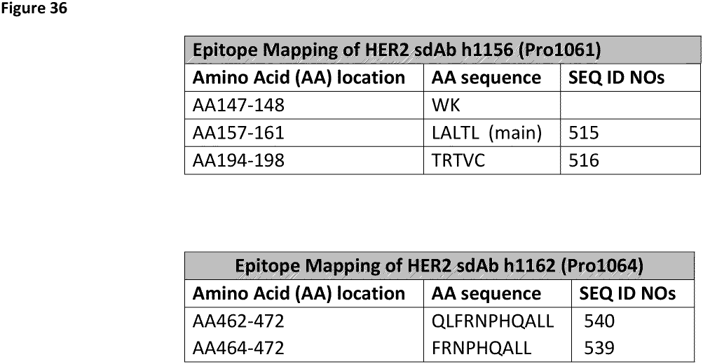

In some embodiments, the sdABD-HER2 (e.g., humanized aHER2 sdAb h1156) has a sdCDR1 with SEQ ID NO:504, a sdCDR2 with SEQ ID NO:505, a sdCDR3 with SEQ ID NO:506. In some cases, the sdABD-HER2 has the amino acid sequence of SEQ ID NO:503, as provided in FIG. 75. Epitope mapping revealed that the humanized aHER2 sdAb h1156 binds to the amino acid sequence WK at amino acid positions 147-148, the amino acid sequence LALTL (SEQ ID NO:515) at amino acid positions 157-161, and the amino acid sequence TRTVC (SEQ ID NO:516) at amino acid positions 194-198 of the HER2 protein (FIG. 36).

In some embodiments, the sdABD-HER2 (e.g., humanized aHER2 sdAb h1159) has a sdCDR1 with SEQ ID NO:508, a sdCDR2 with SEQ ID NO:509, a sdCDR3 with SEQ ID NO:510. In some cases, the sdABD-HER2 has the amino acid sequence of SEQ ID NO:507, as provided in FIG. 75.

In some embodiments, the sdABD-HER2 (e.g., humanized aHER2 sdAb h1162) has a sdCDR1 with SEQ ID NO:512, a sdCDR2 with SEQ ID NO:513, a sdCDR3 with SEQ ID NO:514. In some cases, the sdABD-HER2 has the amino acid sequence of SEQ ID NO:511, as provided in FIG. 75. Epitope mapping revealed that the humanized aHER2 sdAb h1162 binds to the amino acid sequence QLTFRNPHQALL at amino acid positions 462-472 the HER2 protein (FIG. 36).

In some embodiments, the protein prior to cleavage of the protease cleavage domain is less than about 100 kDa. In some embodiments, the protein after cleavage of the protease cleavage domain is about 25 to about 75 kDa. In some embodiments, the protein prior to protease cleavage has a size that is above the renal threshold for first-pass clearance. In some embodiments, the protein prior to protease cleavage has an elimination half-time of at least about 50 hours. In some embodiments, the protein prior to protease cleavage has an elimination half-time of at least about 100 hours. In some embodiments, the protein has increased tissue penetration as compared to an IgG to the same target antigen. In some embodiments, the protein has increased tissue distribution as compared to an IgG to the same target antigen.

### C. Half Life Extension Domains

The MCE proteins of the invention (again, also referred to herein as “COBRA™” proteins or constructs) optionally include half-life extension domains. Such domains are contemplated to include but are not limited to HSA binding domains, Fc domains, small molecules, and other half-life extension domains known in the art.

Human serum albumin (HSA) (molecular mass ~67 kDa) is the most abundant protein in plasma, present at about 50 mg/ml (600 µM), and has a half-life of around 20 days in humans. HSA serves to maintain plasma pH, contributes to colloidal blood pressure, functions as carrier of many metabolites and fatty acids, and serves as a major drug transport protein in plasma.

Noncovalent association with albumin extends the elimination half-time of short lived proteins. For example, a recombinant fusion of an albumin binding domain to a Fab fragment resulted in a reduced in vivo clearance of 25- and 58-fold and a half-life extension of 26- and 37-fold when administered intravenously to mice and rabbits respectively as compared to the administration of the Fab fragment alone. In another example, when insulin is acylated with fatty acids to promote association with albumin, a protracted effect was observed when injected subcutaneously in rabbits or pigs. Together, these studies demonstrate a linkage between albumin binding and prolonged action.

In many embodiments, the half-life extension domain is a single domain antigen binding domain from a single domain antibody that binds to HSA. This domain is generally referred to herein as “sdABD” to human HSA (sdABD-HSA), or alternatively “sdABD(½)”, to distinguish these binding domains from the sdABDs to TTAs. A particularly useful sdABD(½) is shown in FIG. 6.

In some embodiments, the sdABD-HSA (e.g., sdABD-HSA (10GE)) has a sdCDR1 with SEQ ID NO:246, a sdCDR2 with SEQ ID NO:247, a sdCDR3 with SEQ ID NO:248. In some embodiments, the sdABD-HSA has the amino acid sequences of SEQ ID NO:245. In certain embodiments, the sdABD-HSA (e.g., sdABD-HSA with a histidine (His) tag) has a sdCDR1 with SEQ ID NO:250, a sdCDR2 with SEQ ID NO:251, a sdCDR3 with SEQ ID NO:252. In some embodiments, the sdABD-HSA has the amino acid sequences of SEQ ID NO:249.

The half-life extension domain of an antigen binding protein provides for altered pharmacodynamics and pharmacokinetics of the antigen binding protein itself. As above, the half-life extension domain extends the elimination half-time. The half-life extension domain also alters pharmacodynamic properties including alteration of tissue distribution, penetration, and diffusion of the antigen-binding protein. In some embodiments, the half-life extension domain provides for improved tissue (including tumor) targeting, tissue penetration, tissue distribution, diffusion within the tissue, and enhanced efficacy as compared with a protein without a half-life extension binding domain. In one embodiment, therapeutic methods effectively and efficiently utilize a reduced amount of the antigen-binding protein, resulting in reduced side effects, such as reduced non-tumor cell cytotoxicity.

Further, characteristics of the half-life extension domain, for example a HSA binding domain, include the binding affinity of the HSA binding domain for HSA. Affinity of the HSA binding domain can be selected so as to target a specific elimination half-time in a particular polypeptide construct. Thus, in some embodiments, the HSA binding domain has a high binding affinity. In other embodiments, the HSA binding domain has a medium binding affinity. In yet other embodiments, the HSA binding domain has a low or marginal binding affinity. Exemplary binding affinities include KD concentrations at 10 nM or less (high), between 10 nM and 100 nM (medium), and greater than 100 nM (low). As above, binding affinities to HSA are determined by known methods such as Surface Plasmon Resonance (SPR).

### D. Protease Cleavage Sites

The protein compositions of the invention, and particularly the prodrug constructs, include one or more protease cleavage sites, generally resident in cleavable linkers, as outlined herein.

As described herein, the prodrug constructs of the invention include at least one protease cleavage site comprising an amino acid sequence that is cleaved by at least one protease. In some cases, the MCE proteins described herein comprise 1, 2, 3, 4, 5, 6, 7, 8, 9, 10, 11, 12, 13, 14, 15, 16, 17, 18, 19, 20, or more protease cleavage sites that are cleaved by at least one protease. As is more fully discussed herein, when more than one protease cleavage site is used in a prodrug construction, they can be the same (e.g. multiple sites that are cleaved by a single protease) or different (two or more cleavage sites are cleaved by at least two different proteases). As will be appreciated by those in the art, constructs containing three or more protease cleavage sites can utilize one, two, three, etc.; e.g. some constructs can utilize three sites for two different proteases, etc.

The amino acid sequence of the protease cleavage site will depend on the protease that is targeted. As is known in the art, there are a number of human proteases that are found in the body and can be associated with disease states.

Proteases are known to be secreted by some diseased cells and tissues, for example tumor or cancer cells, creating a microenvironment that is rich in proteases or a protease-rich microenvironment. In some cases, the blood of a subject is rich in proteases. In some cases, cells surrounding the tumor secrete proteases into the tumor microenvironment. Cells surrounding the tumor secreting proteases include but are not limited to the tumor stromal cells, myofibroblasts, blood cells, mast cells, B cells, NK cells, regulatory T cells, macrophages, cytotoxic T lymphocytes, dendritic cells, mesenchymal stem cells, polymorphonuclear cells, and other cells. In some cases, proteases are present in the blood of a subject, for example proteases that target amino acid sequences found in microbial peptides. This feature allows for targeted therapeutics such as antigen-binding proteins to have additional specificity because T cells will not be bound by the antigen binding protein except in the protease rich microenvironment of the targeted cells or tissue.

Proteases are proteins that cleave proteins, in some cases, in a sequence-specific manner. Proteases include but are not limited to serine proteases, cysteine proteases, aspartate proteases, threonine proteases, glutamic acid proteases, metalloproteases, asparagine peptide lyases, serum proteases, cathepsins (e.g. cathepsin B, cathepsin C, cathepsin D, cathepsin E, cathepsin K, cathepsin L, cathepsinS, etc.), kallikreins, hK1, hK10, hK15, KLK7, granzymeB, plasmin, collagenase, Type IV collagenase, stromelysin, factor XA, chymotrypsin-like protease, trypsin-like protease, elastase-like protease, subtilisin-like protease, actinidain, bromelain, calpain, caspases (e.g. caspase-3), Mir1-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP8, MMP9, MMP13, MMP11, MMP14, meprin, urokinase plasminogen activator (uPA), enterokinase, prostate-specific antigen (PSA, hK3), interleukin-1β converting enzyme, thrombin, FAP (FAP-α), dipeptidyl peptidase, and dipeptidyl peptidase IV (DPPIV/CD26).

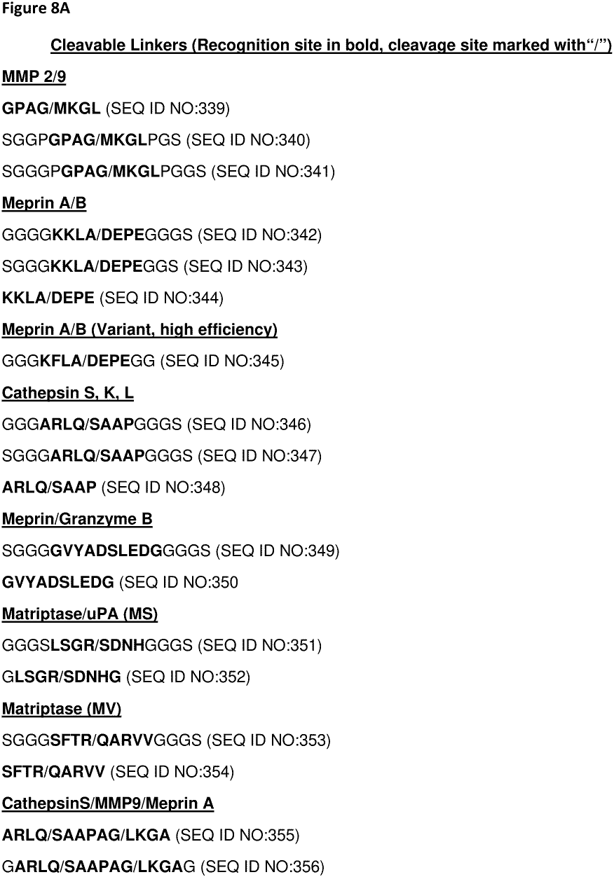

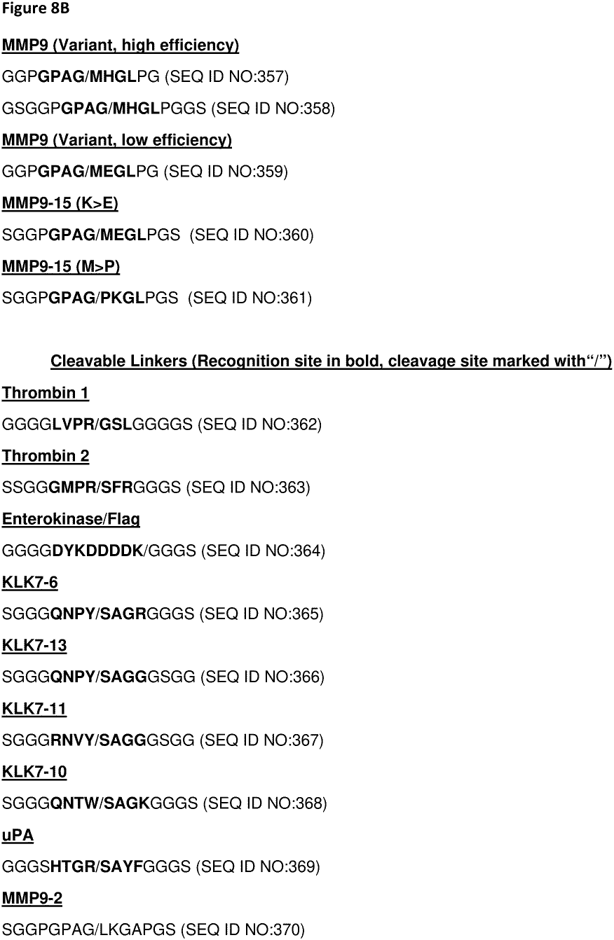

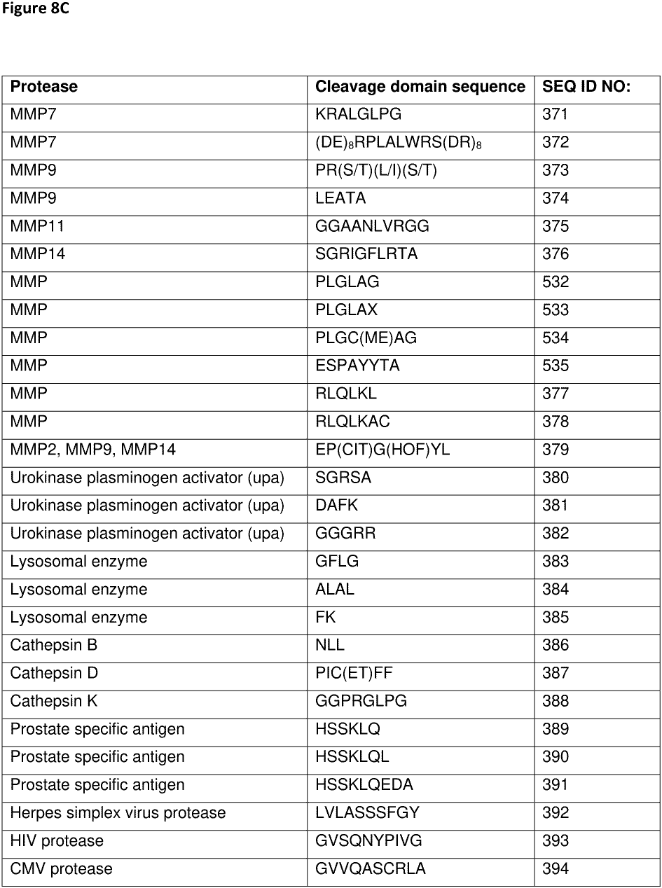

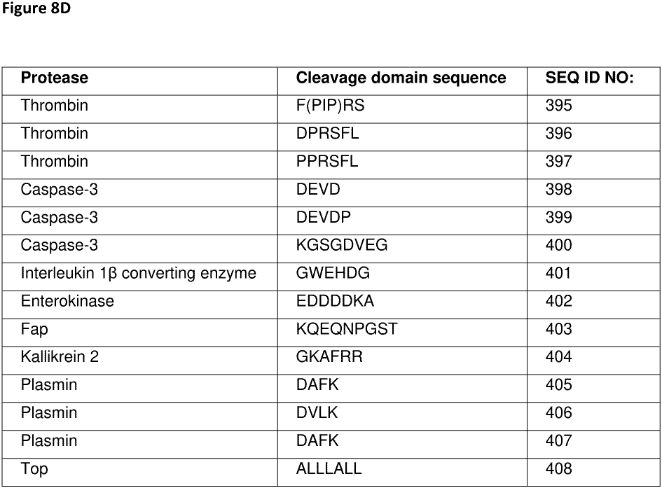

Some suitable proteases and protease cleavage sequences are shown in FIGS. 8A-8D. In some embodiments, any one of fusion proteins described herein comprise a cleavable linker comprising a protease cleavage domain sequence set forth in any one of SEQ ID NOS:339-408 and 532-535.

### E. Linkers

As is discussed herein, the different domains of the invention are generally linked together using amino acid linkers, which can confer functionality as well, including flexibility or inflexibility (e.g. steric constraint) as well as the ability to be cleaved using an in situ protease. These linkers can be classified in a number of ways.

The invention provides “domain linkers”, which are used to join two or more domains (e.g. a VH and a VL, a target tumor antigen binding domain (TTABD, sometimes also referred to herein as “αTTA” (for “anti-TTA”) to a VH or VL, a half life extension domain to another component, etc.). Domain linkers can be non-cleavable (“NCL”), cleavable (“CL”), constrained and cleavable (“CCL”) and constrained and non-cleavable “(CNCL”), for example.

### 1. Non-cleavable Linkers

In some embodiments, the domain linker is non-cleavable. Generally, these can be one of two types: non-cleavable and flexible, allowing for the components “upstream” and “downstream” of the linker in the constructs to intramolecularly self-assemble in certain ways; or non-cleavable and constrained, where the two components separated by the linker are not able to intramolecularly self-assemble. It should be noted, however, that in the latter case, while the two component domains that are separated by the non-cleavable constrained linker do not intramolecularly self-assemble, other intramolecular components will self-assemble to form the pseudo Fv domains.

### (A) Non-cleavable but Flexible Linkers

In this embodiment, the linker is used to join domains to preserve the functionality of the domains, generally through longer, flexible domains that are not cleaved by in situ proteases in a patient. Examples of internal, non-cleavable linkers suitable for linking the domains in the polypeptides of the invention include but are not limited to (GS)n, (GGS)n, (GGGS)n [SEQ ID NO:518], (GGSG)n [SEQ ID NO:519], (GGSGG)n [SEQ ID NO:520], or (GGGGS)n [SEQ ID NO:521], wherein n is 1, 2, 3, 4, 5, 6, 7, 8, 9, or 10. In some embodiments the length of the linker can be about 15 amino acids.

### (B) Non-Cleavable and Constrained Linkers

In some cases, the linkers do not contain a cleavage site and are also too short to allow the protein domains separated by the linker to intramolecularly self-assemble, and are “constrained non-cleavable linkers” or “CNCLs”. For example, in Pro186, an active VH and an active VL are separated by 8 amino acids (an “8-mer” or “8mer”) that does not allow the VH and VL to self-assemble into an active antigen binding domain. In some embodiments, the linker is still flexible; for example, (GGGS)n where n = 2. In other embodiments, although generally less preferred, more rigid linkers can be used, such as those that include proline or bulky amino acids.

### 2. Cleavable Linkers

All of the prodrug constructs herein include at least one cleavable linker. Thus, in one embodiment, the domain linker is cleavable (CL), sometimes referred to herein as a “protease cleavage domain” (“PCD”). In this embodiment, the CL contains a protease cleavage site, as outlined herein and as depicted in FIGS. 8A-8D. In some cases, the CL contains just the protease cleavage site. Optionally, depending on the length of the cleavage recognition site, there can be an extra few linking amino acids at either or both of the N- or C-terminal end of the CL; for example, there may be from 1, 2, 3, 4 or 5, or more amino acids on either or both of the N- and C-termini of the cleavage site. Thus, cleavable linkers can also be constrained (e.g. 8mers) or flexible.

Of particular interest in the present invention are MMP9 cleavable linkers and meprin cleavable linkers, particularly MMP9 constrained cleavable linkers and meprin constrained cleavable linkers.

### II. Domains of the Invention

The present invention provides a number of different formats for the prodrug polypeptides of the invention. The present invention provides constrained Fv domains and constrained pseudo Fv domains. Additionally, the present invention provides multivalent conditionally effective (“MCE”) proteins which contain two Fv domains but are non-isomerizing constructs. As outlined herein, these can be non-isomerizing cleavable formats or non-isomerizing non-cleavable formats, although every construct contains at least one protease cleavage domain.

Importantly, while both of these domains (Fv domains and pseudo Fv domains) are referred to herein as “constrained”, meaning that as discussed above and shown in FIGS. 1-5, only one of these needs to be constrained, although generally, when both linkers are constrained, the protein has better expression.

Those of skill in the art will appreciate that for Formats 1, 2 and 4, there are four possibilities for the N- to C-terminal order of the constrained and pseudo Fv domains of the invention (not showing the linkers): aVH-aVL and iVL-iVH, aVH-aVL and iVH-iVL, aVL-aVH and iVL-iVH, aVL-aVH and iVH-iVL. All four have been tested and all four have activity, although the first order, aVH-aVL and iVL-iVH, shows better expression than the other three. Thus while the description herein is generally shown in this aVH-aVL and iVL-iVH format, all disclosure herein includes the other orders for these domains as well.

Note that generally, the N to C-terminal order for the full length constructs of the invention is based on the aVH-aVL and iVL-iVH orientation.

Additionally, it is known in the art that there can be immunogenicity in humans originating from the C-terminal sequences of certain ABDs. Accordingly, in general, particularly when the C-terminus of the constructs terminates in a sdABD (for example, the sdABD-HSA domains of many of the constructs, a histidine tag (either His6 or His10) can be used. Many or most of the sequences herein were generated using His6 C-terminal tags for purification reasons, but these sequences can also be used to reduce immunogenicity in humans, as is shown by Holland et al., DOI 10.1007/s10875-013-9915-0 and WO2013/024059.

### A. Constrained Fv Domains

The present invention provides constrained Fv domains, that comprise an active VH and an active VL domain that are covalently attached using a constrained linker (which, as outlined herein, can be cleavable (Format 1) or non-cleavable (Formats 2 and 4)). The constrained linker prevents intramolecular association between the aVH and aVL in the absence of cleavage. Thus, a constrained Fv domain general comprises a set of six CDRs contained within variable domains, wherein the vhCDR1, vhCDR2 and vhCDR3 of the VH bind human CD3 and the vlCDR1, vCDR2 and vlCDR3 of the VL bind human CD3, but in the prodrug format (e.g. uncleaved), the VH and VL are unable to sterically associate to form an active binding domain, preferring instead to pair intramolecularly with the pseudo Fv.

The constrained Fv domains can comprise active VH and active VL (aVH and aVL) or inactive VH and VL (iVH and iVL, in which case it is a constrained pseudo Fv domain) or combinations thereof as described herein.

As will be appreciated by those in the art, the order of the VH and VL in a constrained Fv domain can be either (N- to C-terminal) VH-linker-VL or VL-linker-VH.

As outlined herein, for Format 1 constructs, the constrained Fv domains can comprise a VH and a VL linked using a cleavable linker, in cases such as those shown in FIG. 1. In this embodiment, the constrained Fv domain has the structure (N- to C-terminus) vhFR1-vhCDR1-vhFR2-vhCDR2-vhFR3-vhCDR3-vhFR4-CCL-vlFR1-vlCDR1-vlFR2-vlCDR2-vlFR3-vlCDR3-vlFR4In general, the constrained Fv domain contains active VH and VL domains (e.g., able to bind CD3 when associated) and thus has the structure (N- to C-terminus) vhFR1-avhCDR1-vhFR2-avhCDR2-vhFR3-avhCDR3-vhFR4-CCL-vlFR1-avlCDR1-vlFR2-avlCDR2-vlFR3-avlCDR3-vlFR4.

As outlined herein, for Format 2 constructs, the constrained Fv domains can comprise a VH and a VL linked using a non-cleavable linker. In this embodiment, the constrained Fv domain has the structure (N- to C-terminus) vhFR1-vhCDR1-vhFR2-vhCDR2-vhFR3-vhCDR3-vhFR4-CNCL-vlFR1-vlCDR1-vlFR2-vlCDR2-vlFR3-vlCDR3-vlFR4. In general, the constrained Fv domain contains active VH and VL domains (e.g. able to bind CD3 when associated) and thus has the structure (N- to C-terminus) vhFR1-avhCDR1-vhFR2-avhCDR2-vhFR3-avhCDR3-vhFR4-CNCL-vlFR1-avlCDR1-vlFR2-avlCDR2-vlFR3-avlCDR3-vlFR4.

Of particular use in the present invention are constrained non-cleavable Fv domains having an aVH having SEQ ID NO:270, an aVL having SEQ ID NO:254, and a domain linker having SEQ ID NO:287.

### B. Constrained Pseudo Fv Domains

The present invention provides constrained pseudo Fv domains, comprising inactive or pseudo iVH and iVL domains that are covalently attached using a constrained linker (which, as outlined herein, can be cleavable or non-cleavable). The constrained linker prevents intramolecular association between the iVH and iVL in the absence of cleavage. Thus, a constrained pseudo Fv domain general comprises an iVH and an iVL with framework regions that allow association (when in a non-constrained format) of the iVH and iVL, although the resulting pseudo Fv domain does not bind to a human protein. iVH domains can assemble with aVL domains, and iVL domains can assemble with aVH domains, although the resulting structures do not bind to CD3.

The constrained pseudo Fv domains comprise inactive VH and VL (iVH and iVL).

As will be appreciated by those in the art, the order of the VH and VL in a constrained pseudo Fv domain can be either (N- to C-terminal) VH-linker-VL or VL-linker-VH.

As outlined herein, the constrained pseudo Fv domains can comprise a iVH and an iVL linked using a non-cleavable linker, as shown in Formats 1, 2 and 4, or with cleavable linkers, as shown in Format 3.

In general, the constrained Fv domain contains inert VH and VL domains (e.g. able to bind CD3 when associated) and thus has the structure (N- to C-terminus) vhFR1-ivlCDR1-vhFR2-ivlCDR2-vhFR3-ivlCDR3-vhFR4-CNCL-vlFR1-ivhCDR1-vlFR2-ivhCDR2-vlFR3-ivhCDR3-vlFR4.

Of particular use in the present invention are constrained non-cleavable pseudo Fv domains having (i) an iVH having SEQ ID NO:274 (αCD3 VHi), SEQ ID NO:278 (αCD3 VHi2) or SEQ ID NO:282 (αCD3 VHiGL4), (ii) an iVL having SEQ ID NO:258 (αCD3 VLi), SEQ ID NO:262 (αCD3 VLi2) or SEQ ID NO:266 (αCD3 VLiGL), and (iii) a domain linker having SEQ ID NO:287. In some embodiments, a constrained non-cleavable pseudo Fv domain comprises (i) an iVH having an amino acid sequence of SEQ ID NO:274 (αCD3 VHi), (ii) an iVL having an amino acid sequence of SEQ ID NO:258 (αCD3 VLi), and (iii) a domain linker having an amino acid sequence of SEQ ID NO:287. In some embodiments, a constrained non-cleavable pseudo Fv domain comprises (i) an iVH having an amino acid sequence of SEQ ID NO:278 (αCD3 VHi2), (ii) an iVL having an amino acid sequence of SEQ ID NO:262 (αCD3 VLi2), and (iii) a domain linker having an amino acid sequence of SEQ ID NO:287. In some embodiments, a constrained non-cleavable pseudo Fv domain comprises (i) an iVH having an amino acid sequence of SEQ ID NO:282 (αCD3 VHi2GL4), (ii) an iVL having an amino acid sequence of SEQ ID NO:266 (αCD3 VLi2GL), and (iii) a domain linker having an amino acid sequence of SEQ ID NO:287

### III. Formats of the Invention

As discussed herein, the pro-drug constructs of the invention can take on a number of different formats, including cleavable formats with dual TTA binding domains, non-cleavable formats with dual TTA binding domains (either of which can have the same TTA binding domains or different binding domains), and non-cleavable formats with a single targeting domain.

### A. “Format 2” Constructs

As shown in FIG. 2, the invention provides non-isomerizing non-cleavable formats. In this embodiment, it is understood that the “non-cleavable” applies only to the linkage of the constrained Fv domain, as there is the activating cleavage site in the prodrug construct. In this embodiment, the constrained Fv domain comprise VH and VL domains that are linked using constrained non-cleavable linkers and the constrained pseudo Fv domain uses constrained non-cleavable linkers.

As will be appreciated by those in the art, the order of the VH and VL in either a constrained Fv domain or a constrained pseudo Fv domain can be either (N- to C-terminal) VH-linker-VL or VL-linker-VH.

The invention provides prodrug proteins, comprising, from N- to C-terminal: (sdABD-TTA1)-domain linker-constrained Fv domain-domain linker-(sdABD-TTA2)-cleavable linker-constrained pseudo Fv domain-domain linker-(sdABD-HSA).

As will be appreciated by those in the art, the order of the VH and VL in either a constrained Fv domain or a constrained pseudo Fv domain can be either (N- to C-terminal) VH-linker-VL or VL-linker-VH.

Thus, in one embodiment, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA).

Thus, in one embodiment, the prodrug protein comprises, from N- to C-terminal: (sdABD-TTA1)-domain linker-aVH-CNCL-aVL-domain linker-(sdABD-TTA2)-CL-iVH-CNCL-iVL-domain linker-(sdABD-HSA).

Thus, in one embodiment, the prodrug protein comprises, from N- to C-terminal: (sdABD-TTA1)-domain linker-aVL-CNCL-aVH-domain linker-(sdABD-TTA2)-CL-iVL-CNCL-iVH-domain linker-(sdABD-HSA).

Thus, in one embodiment, the prodrug protein comprises, from N- to C-terminal: (sdABD-TTA1)-domain linker-aVL-CNCL-aVH-domain linker-(sdABD-TTA2)-CL-iVH-CNCL-iVL-domain linker-(sdABD-HSA).

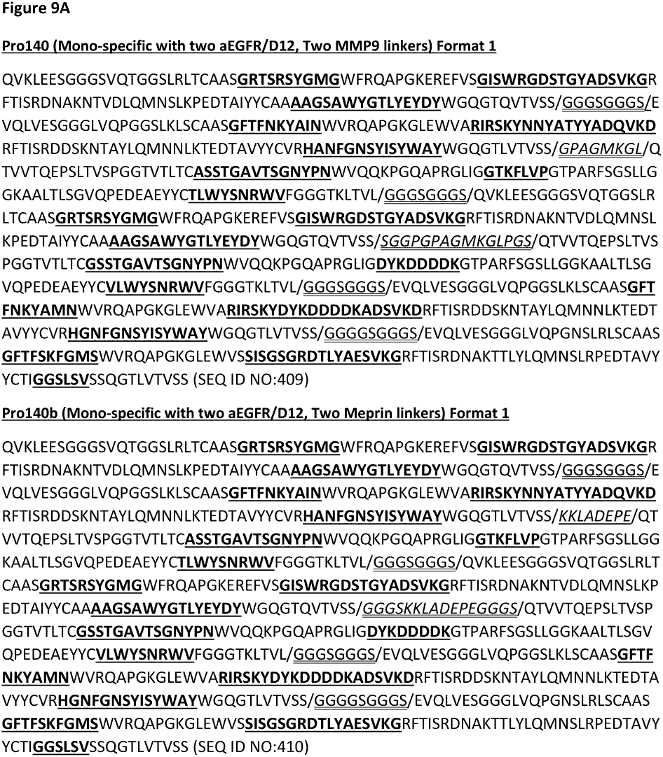

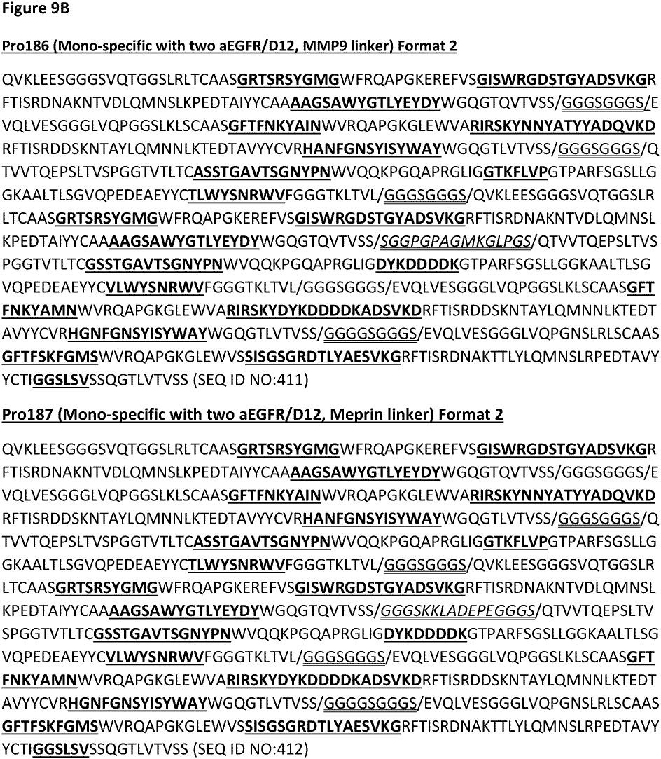

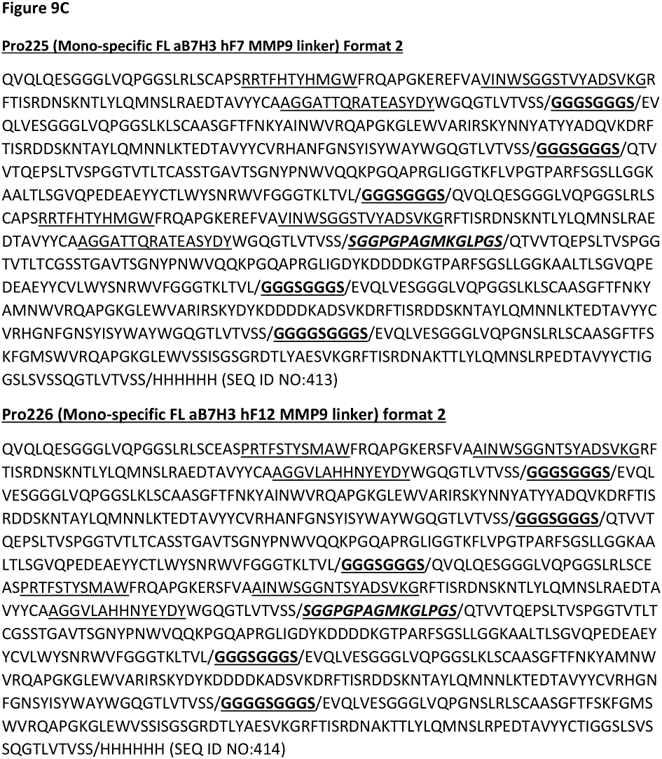

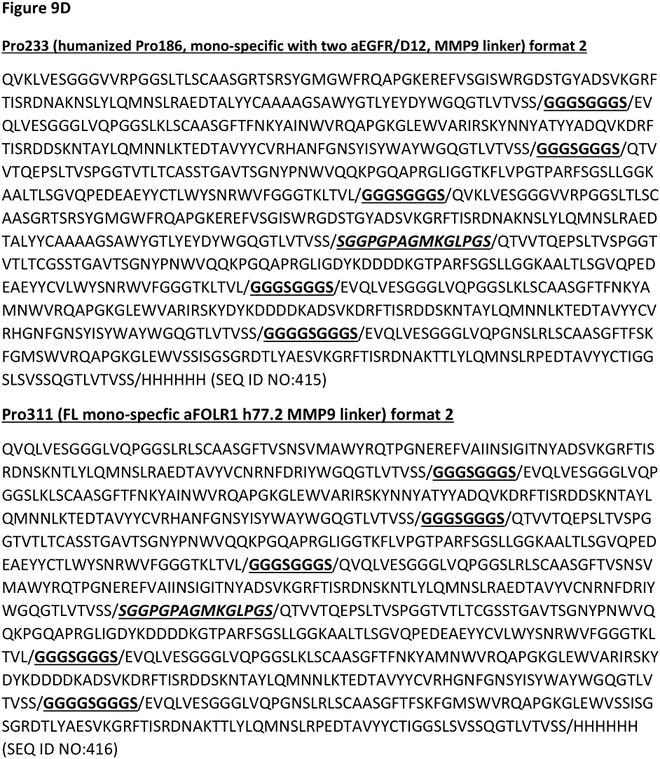

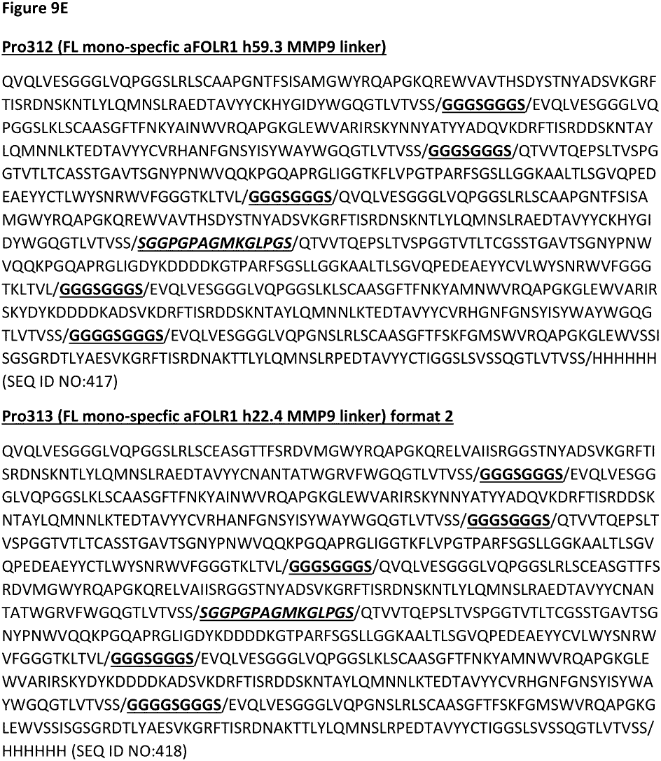

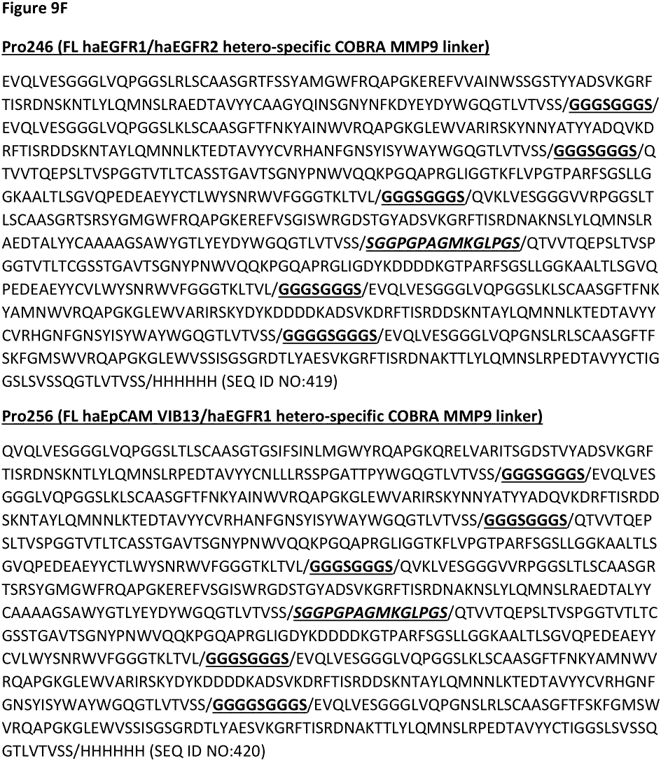

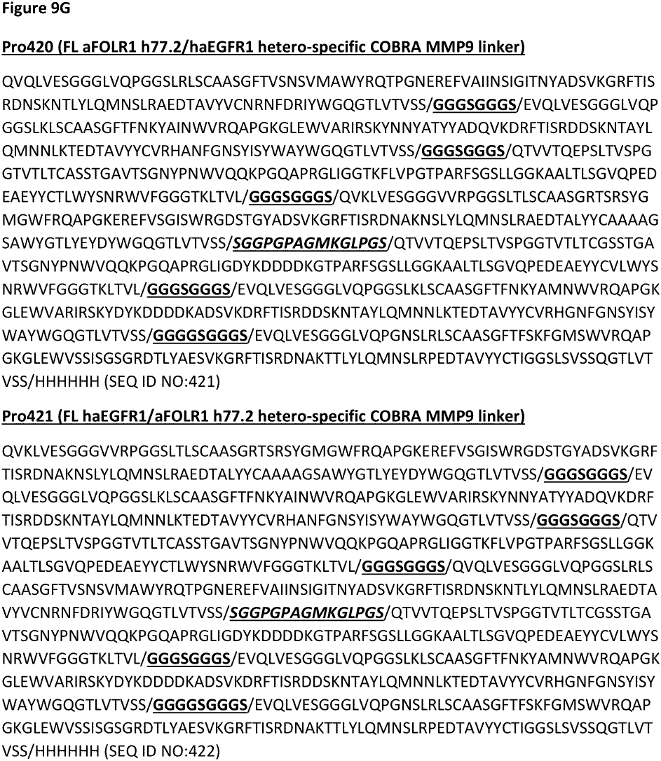

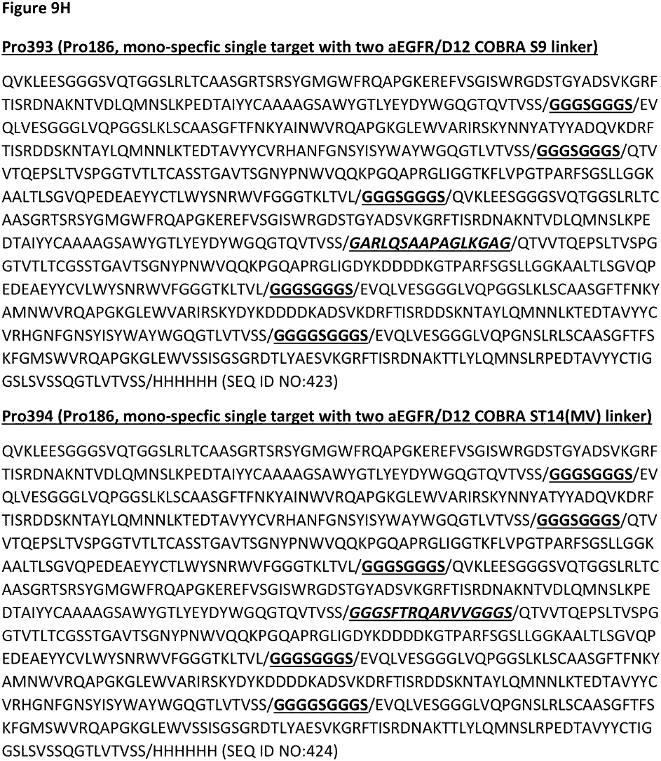

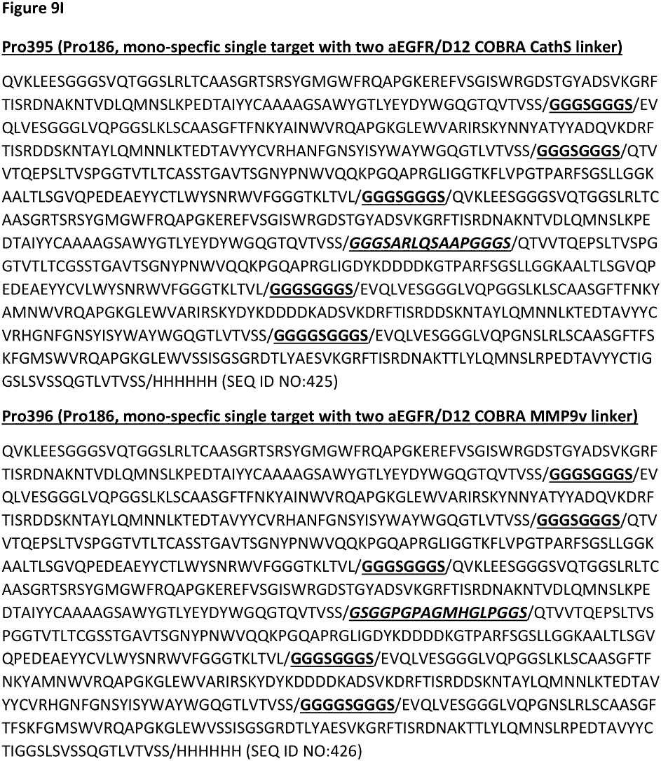

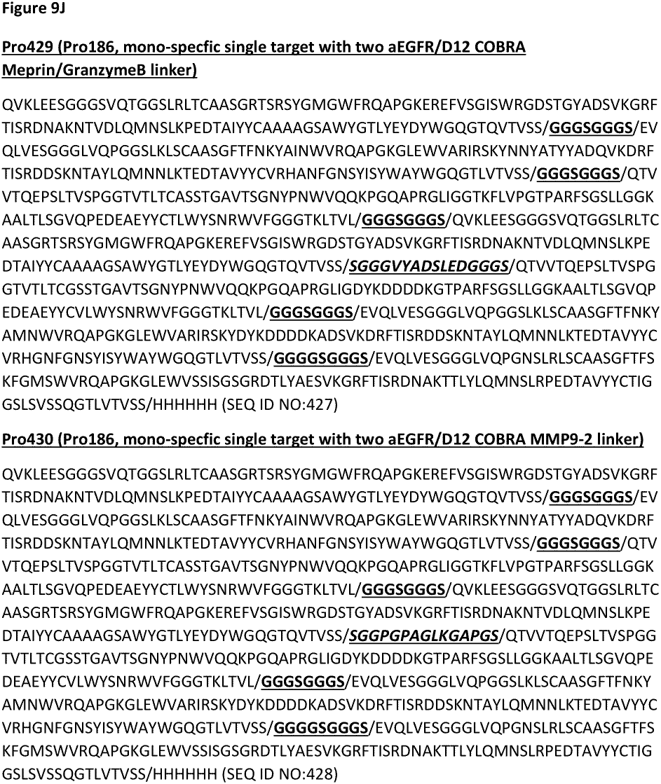

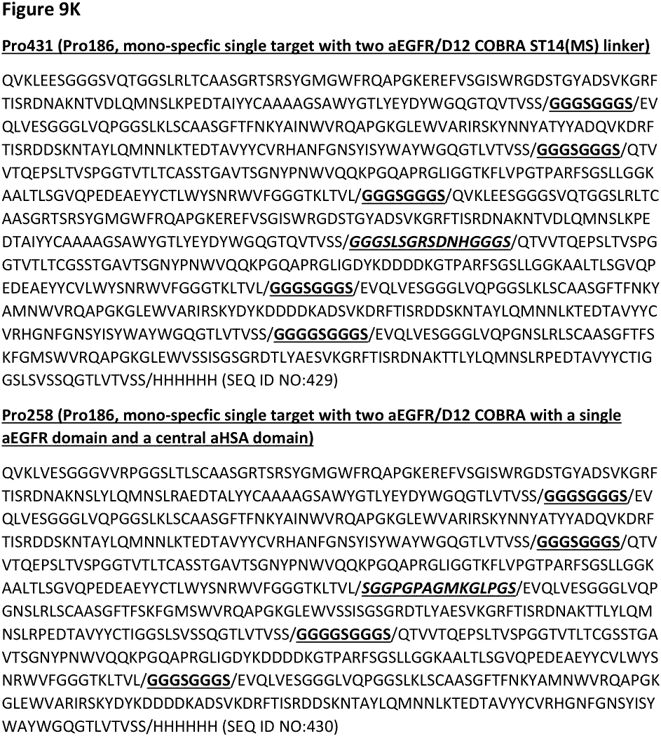

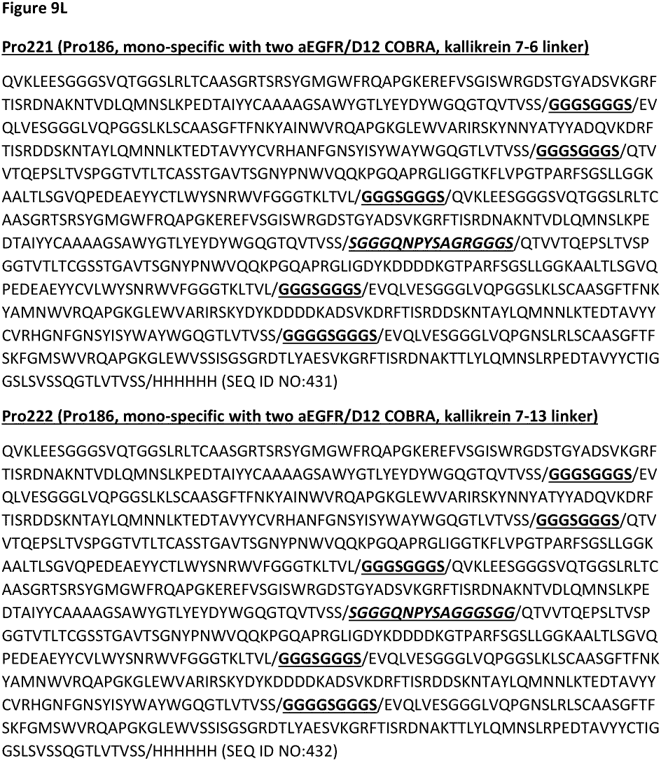

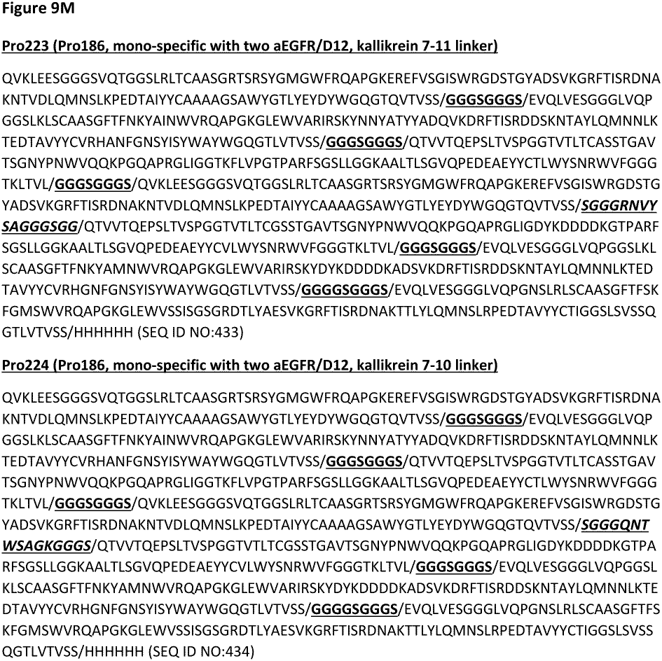

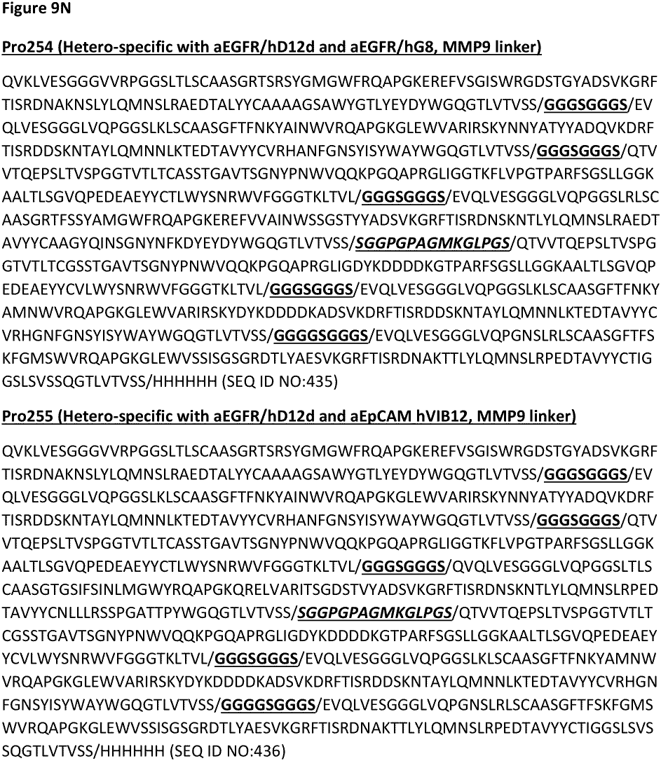

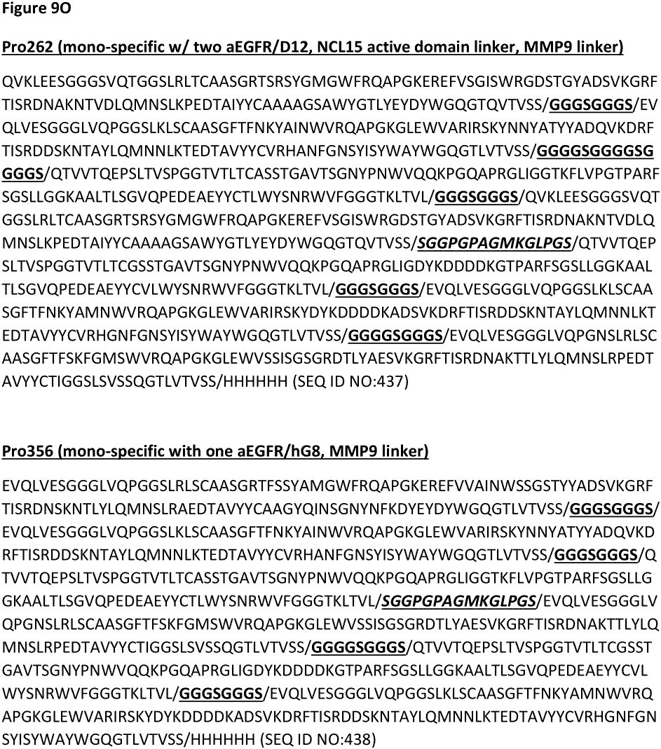

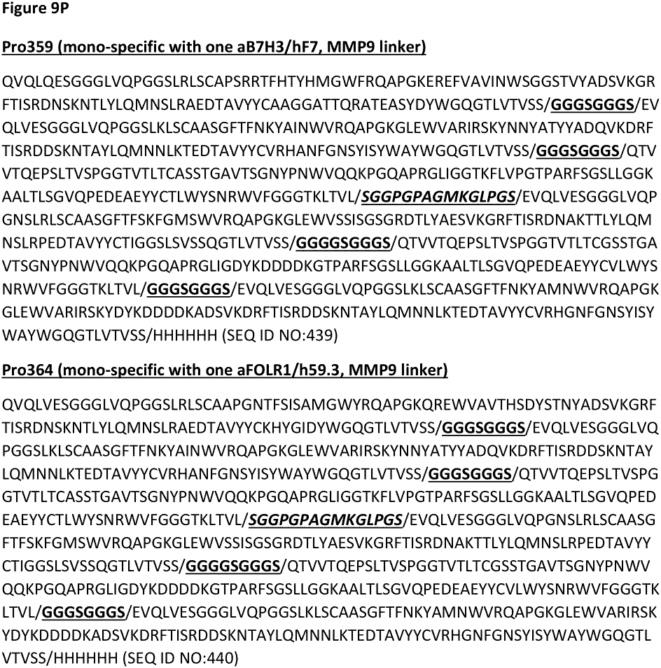

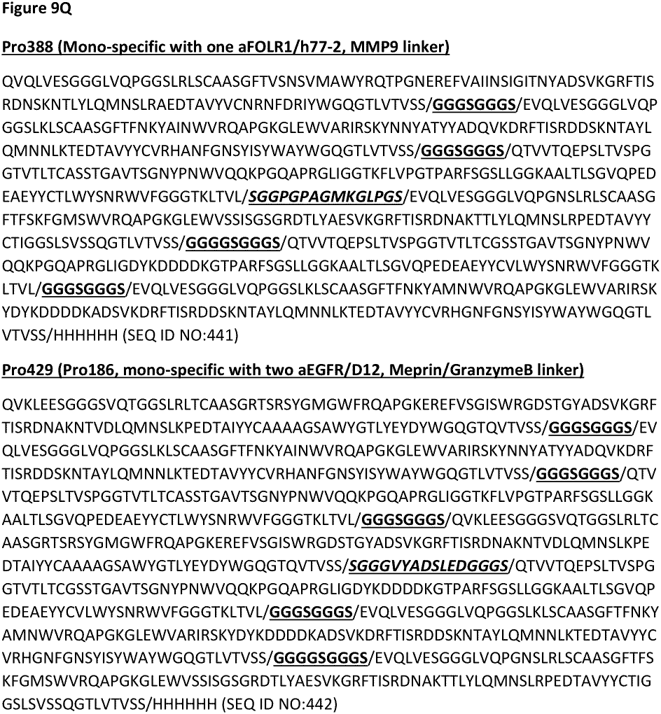

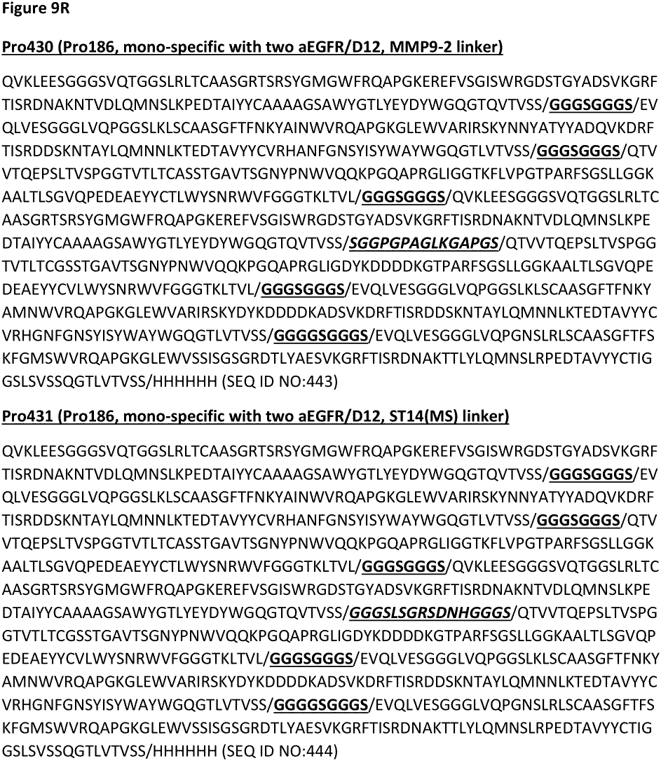

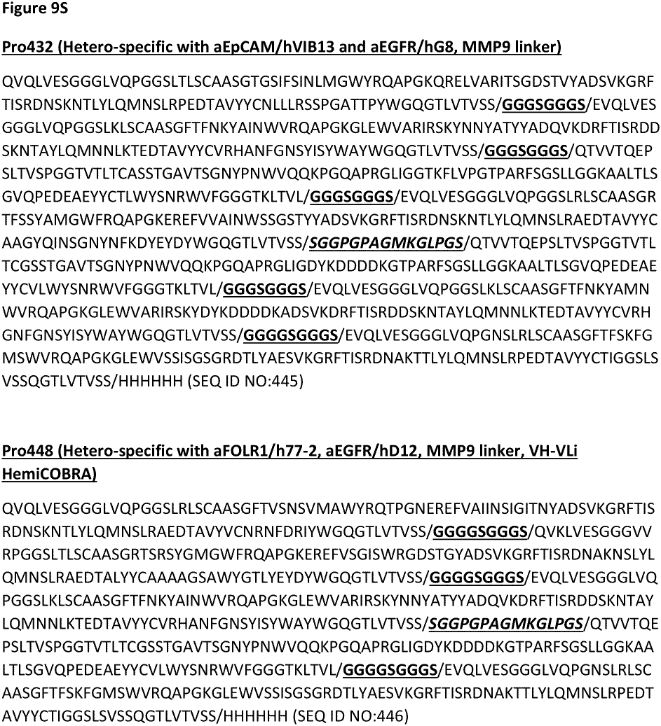

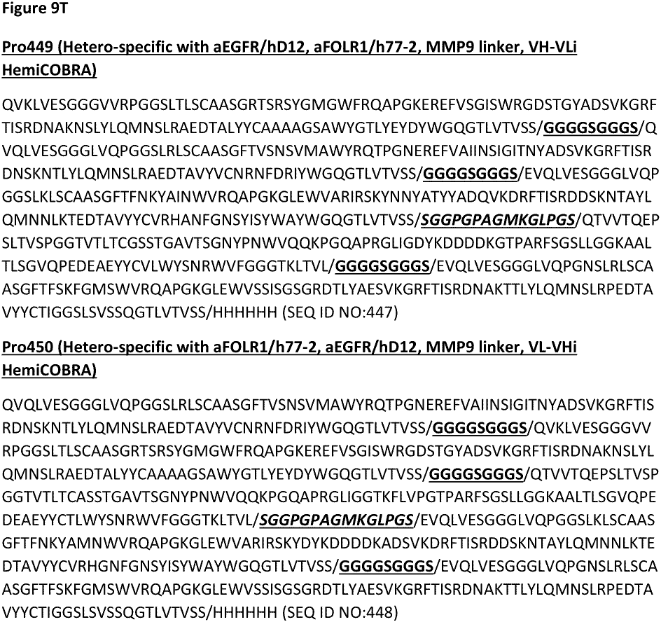

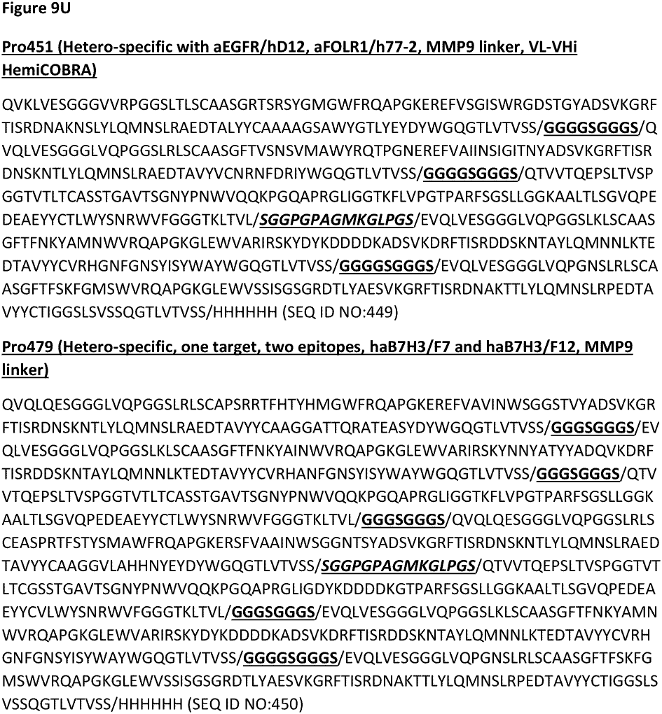

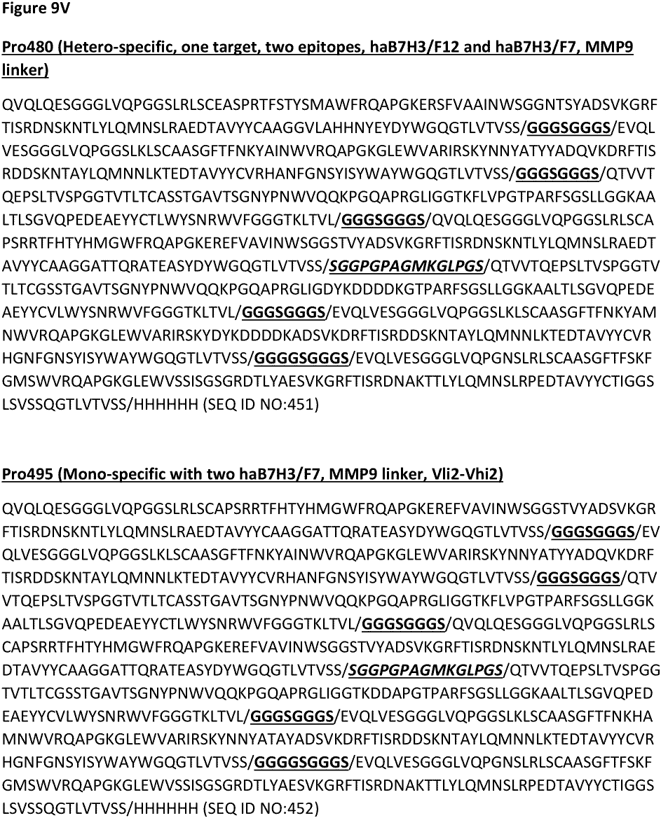

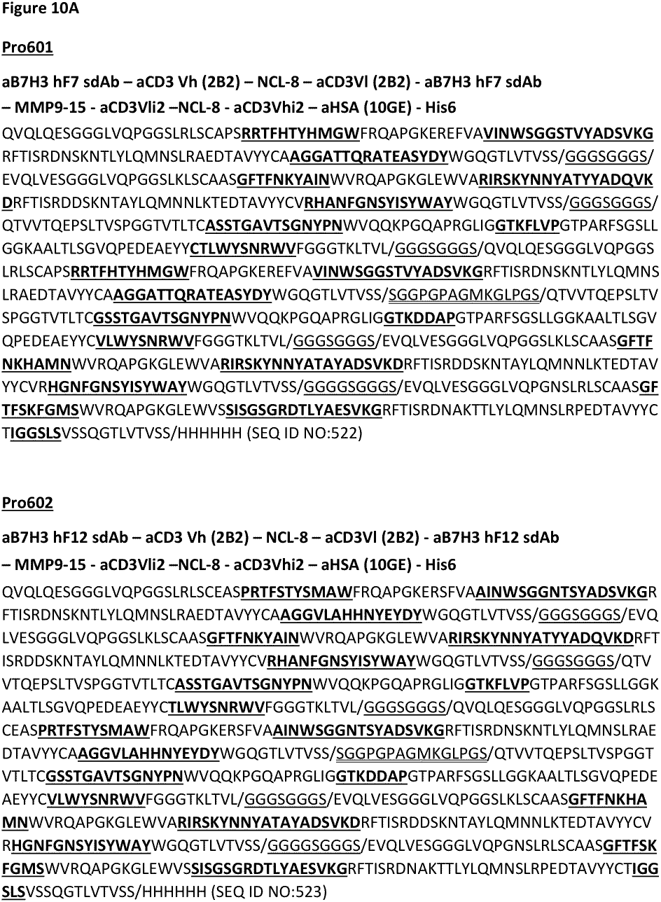

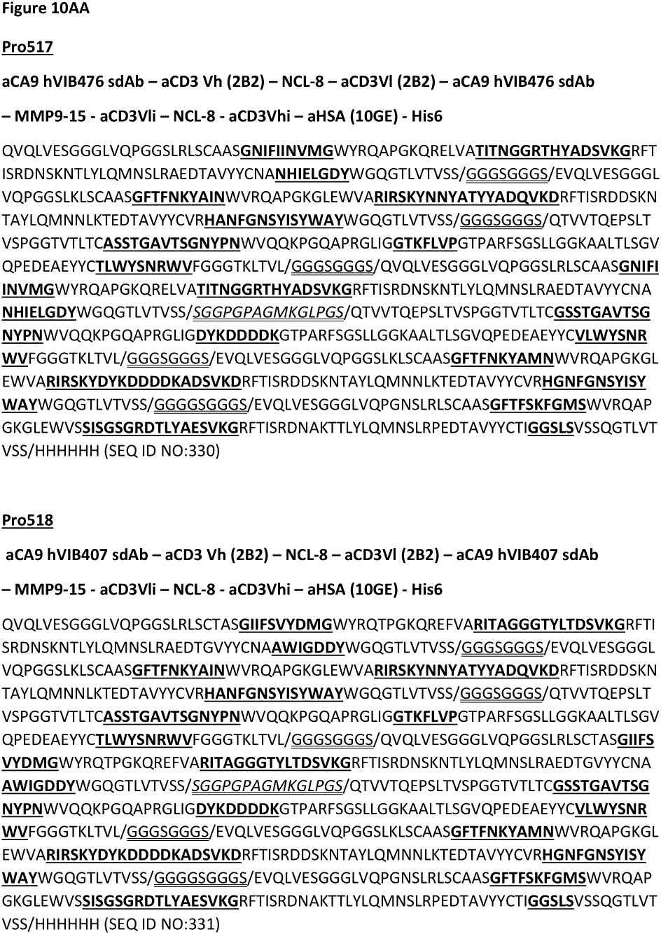

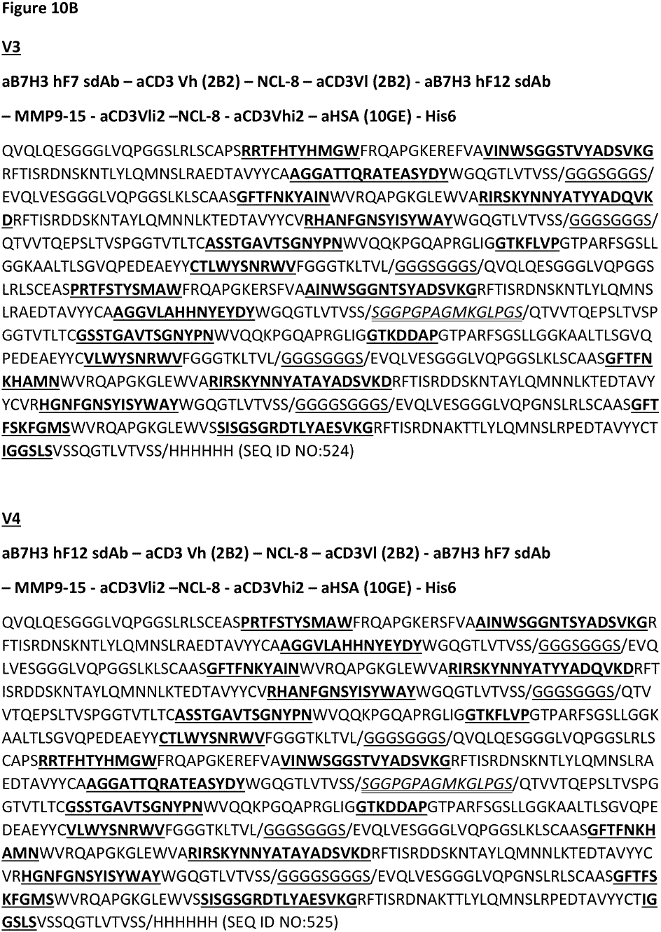

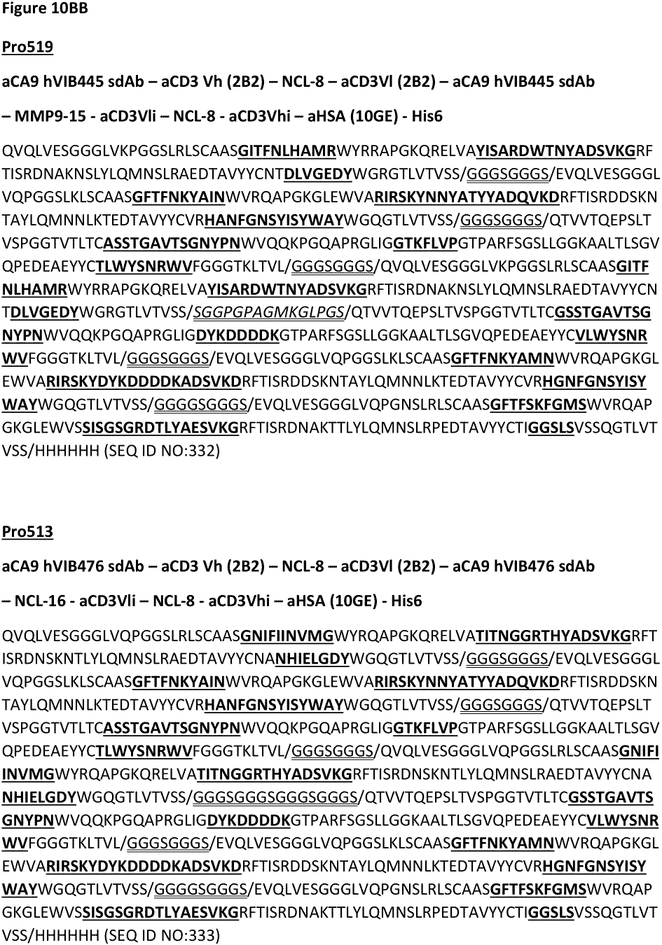

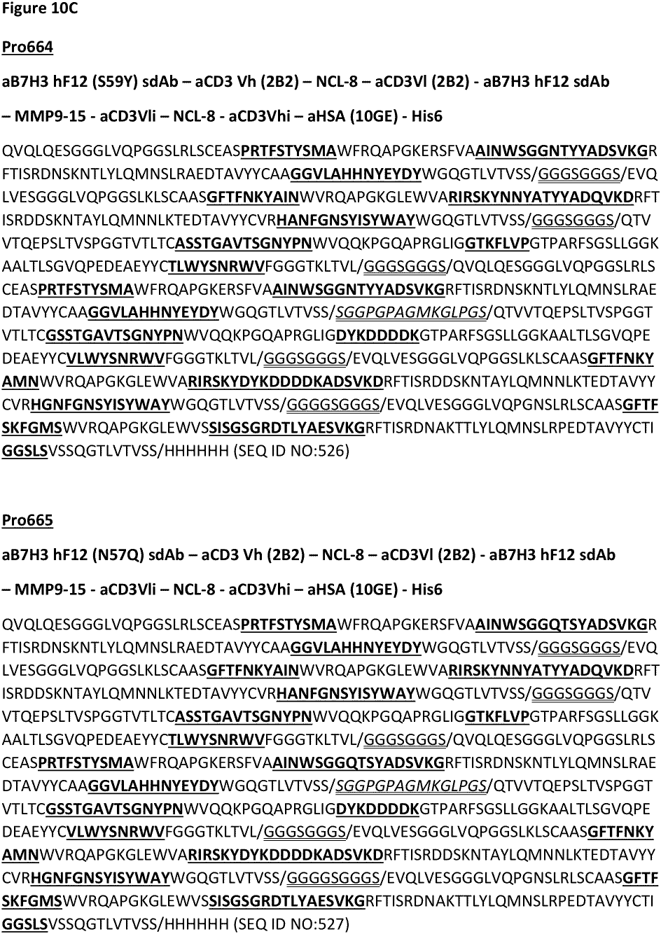

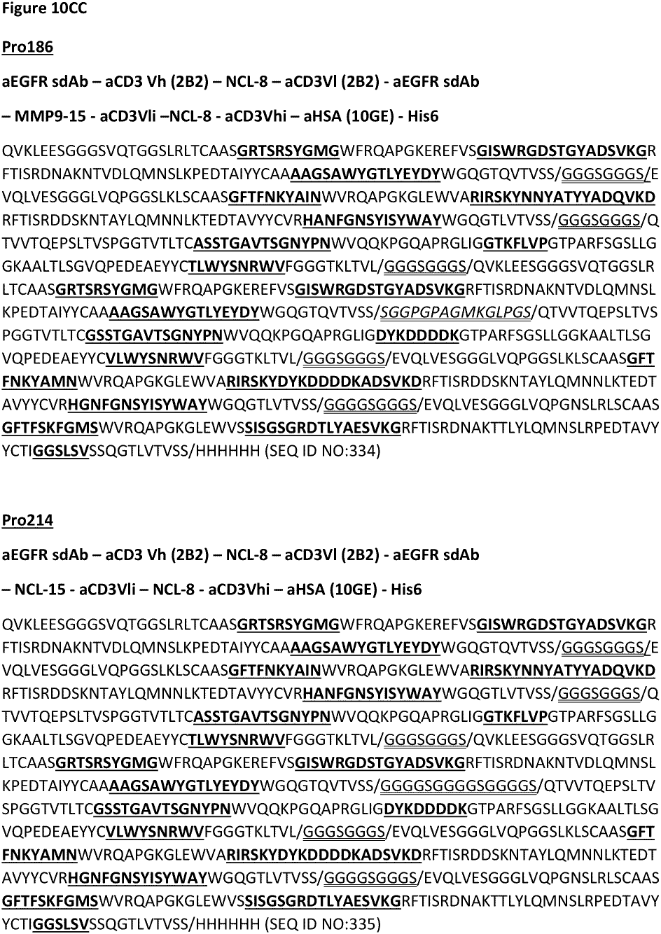

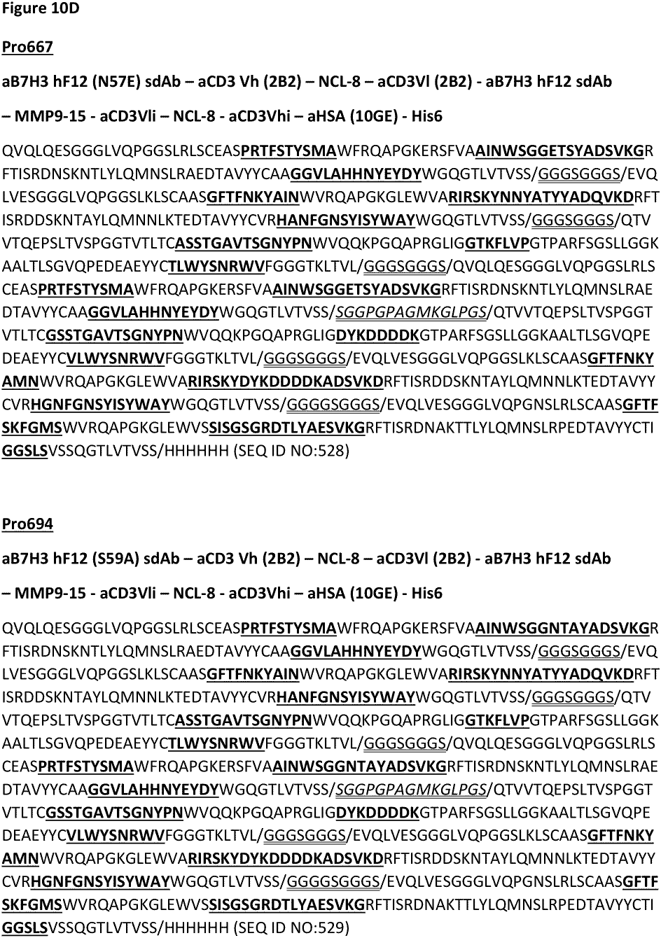

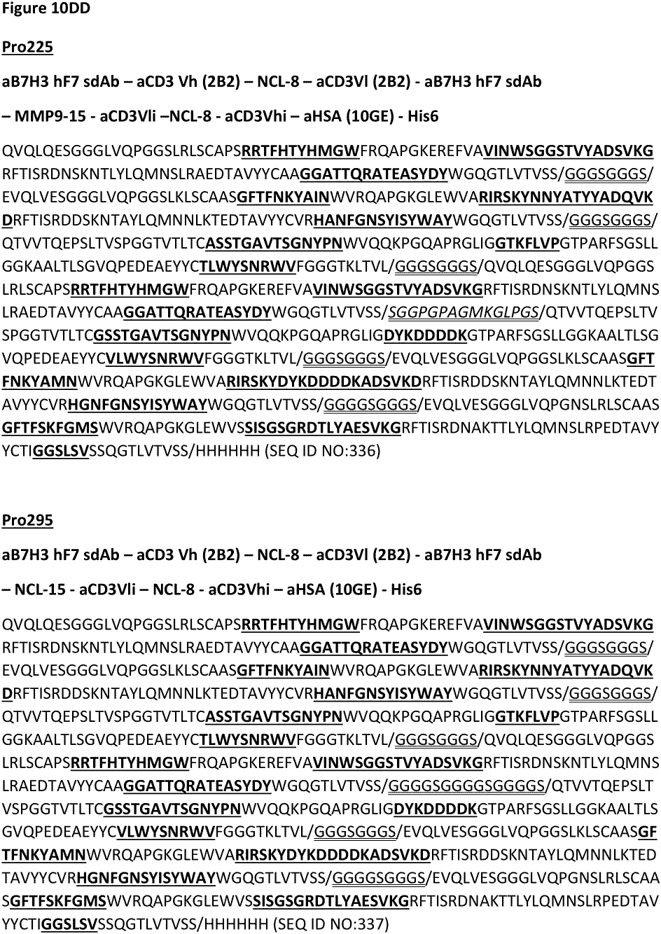

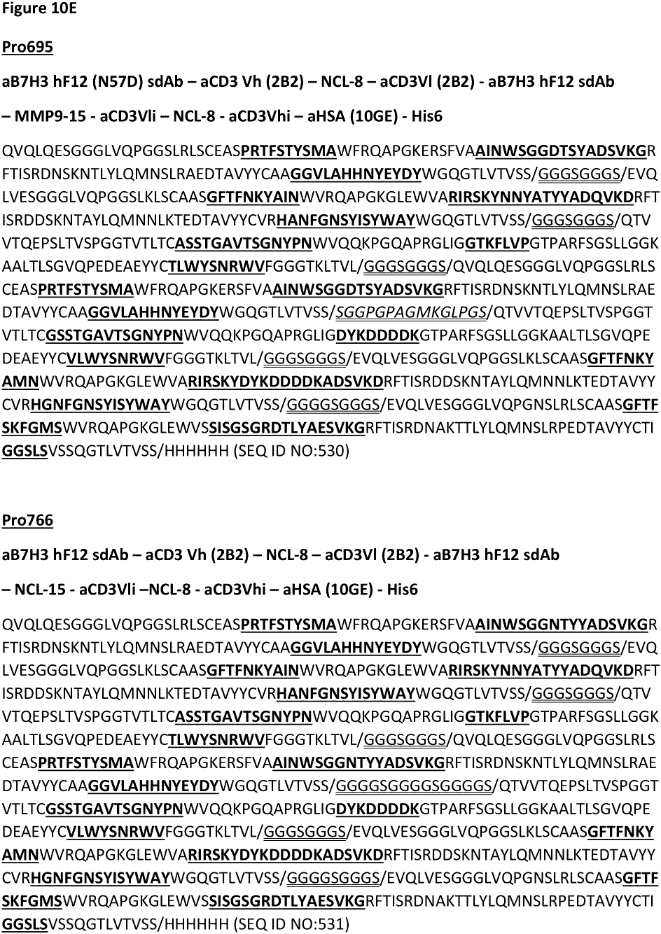

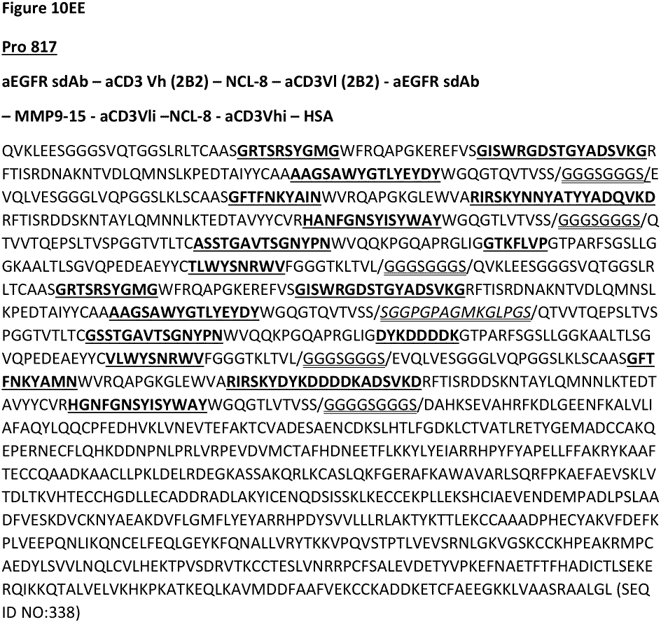

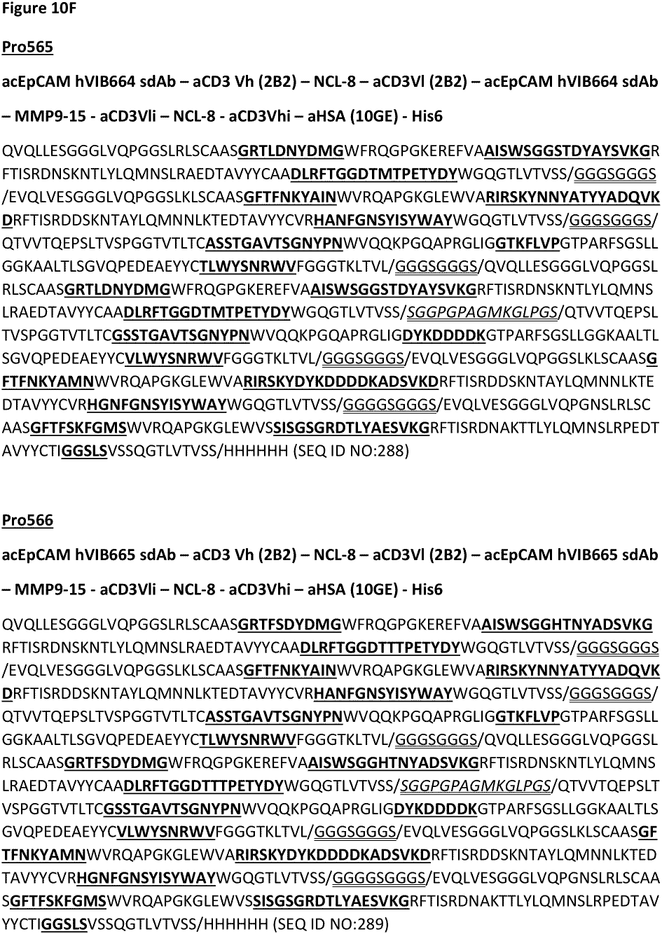

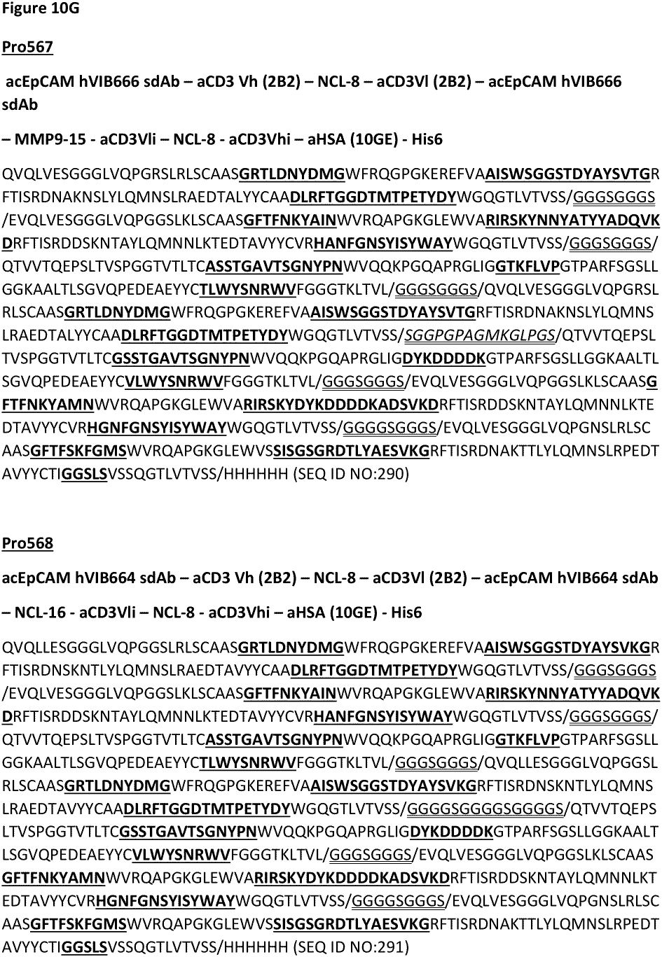

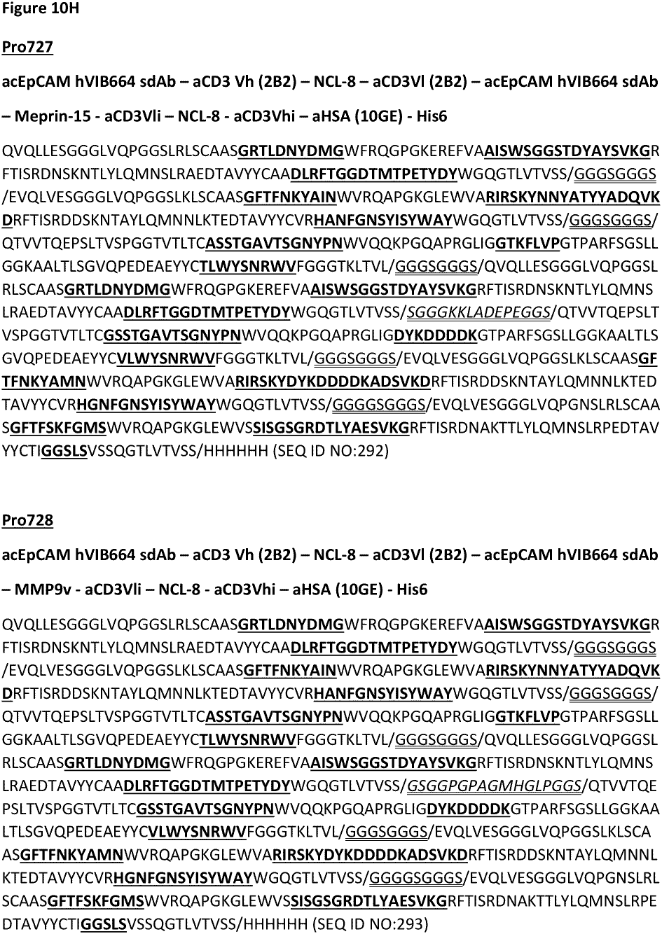

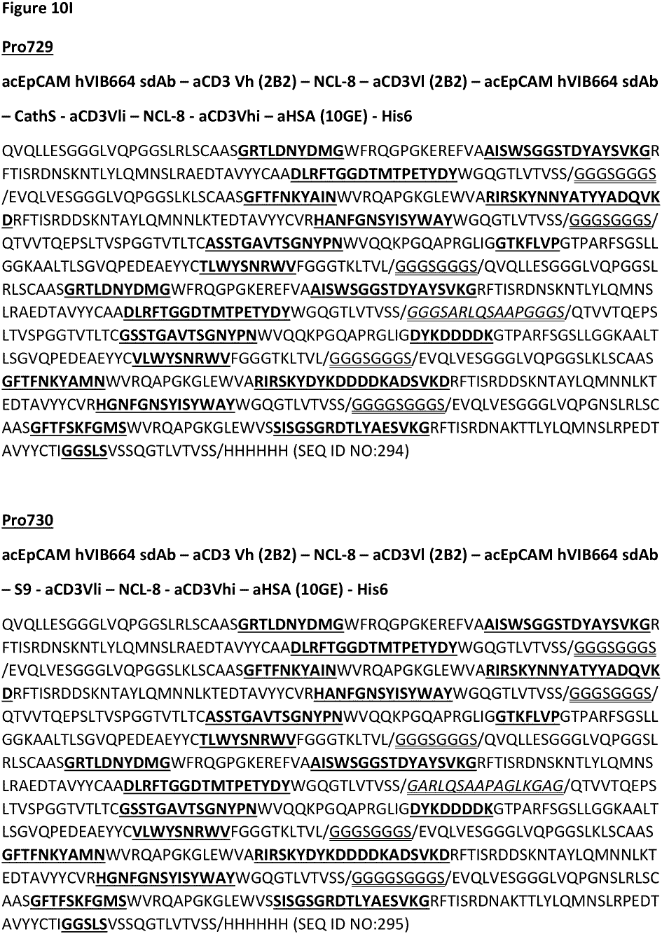

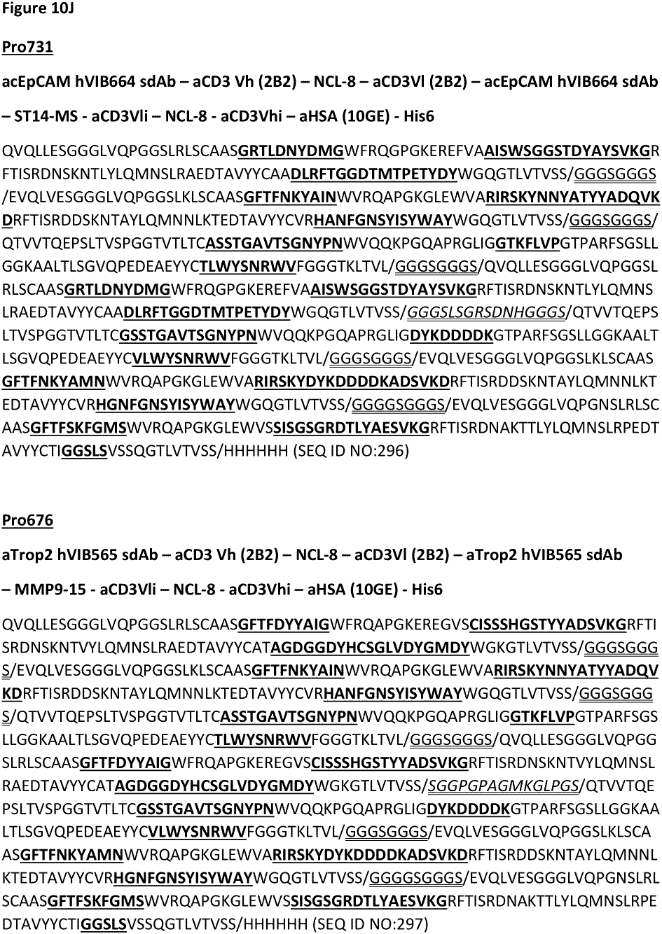

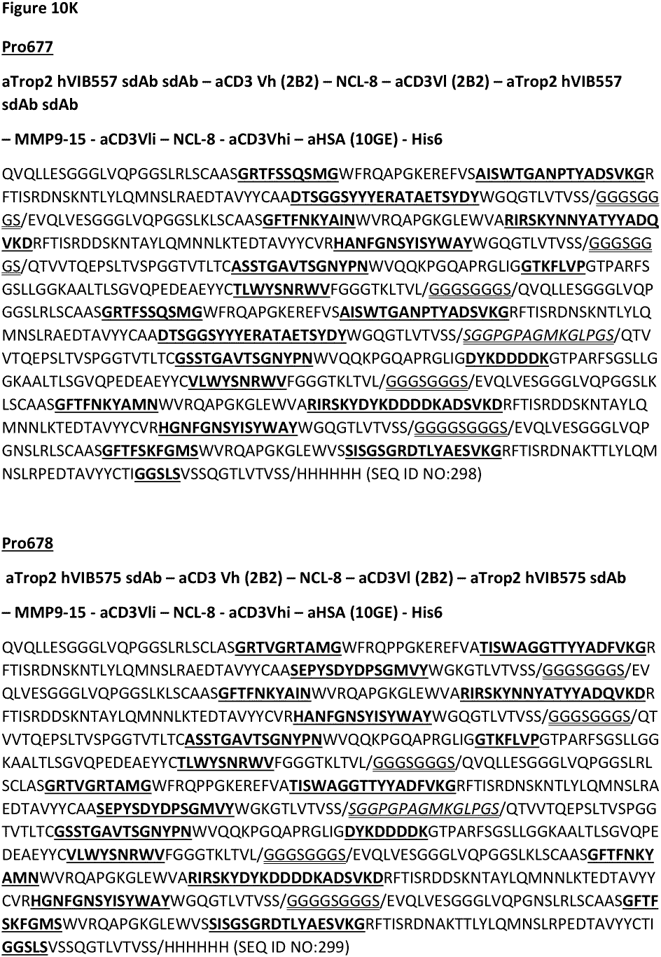

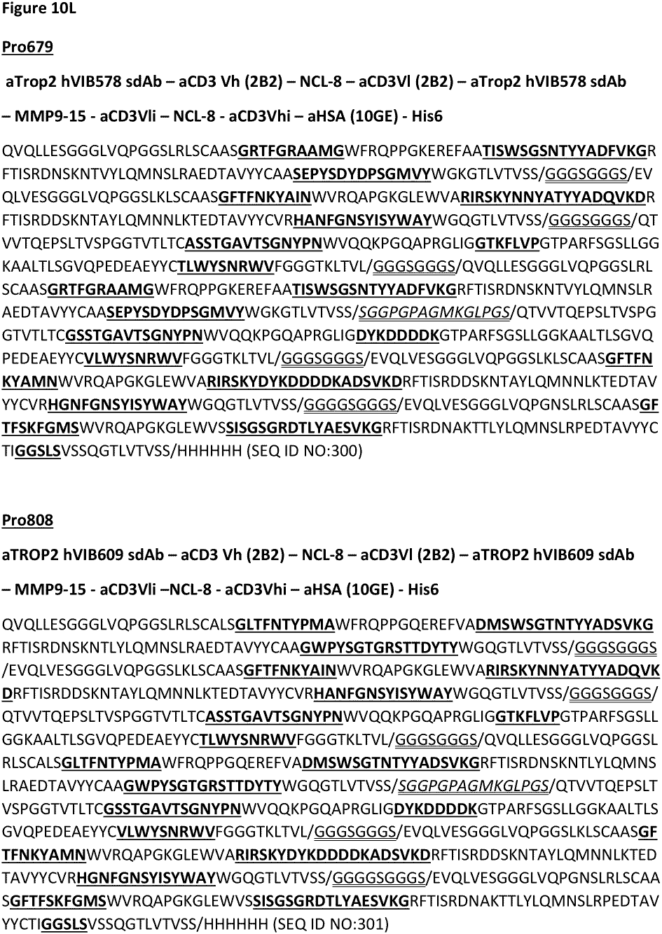

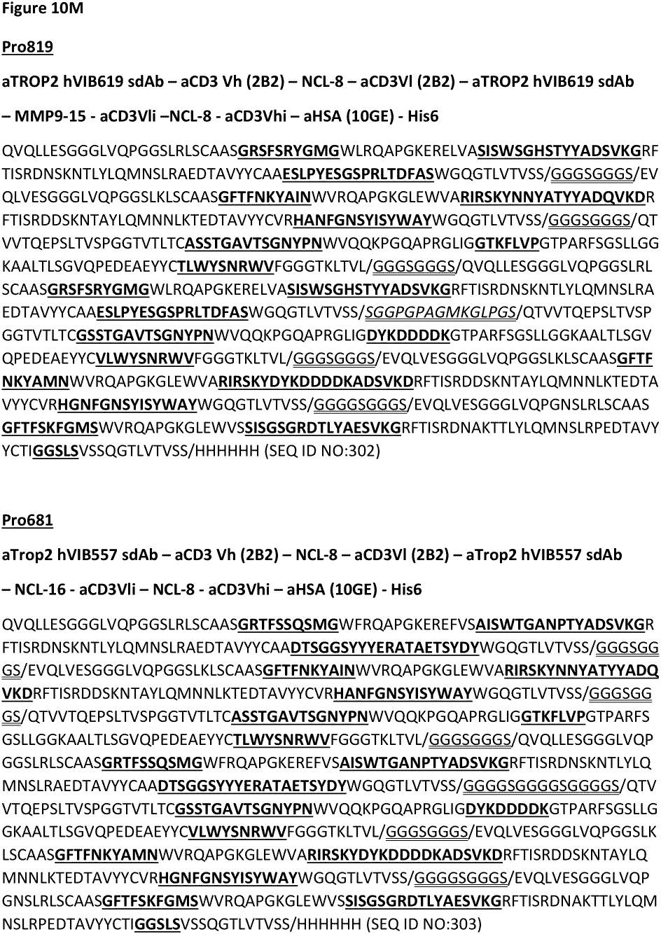

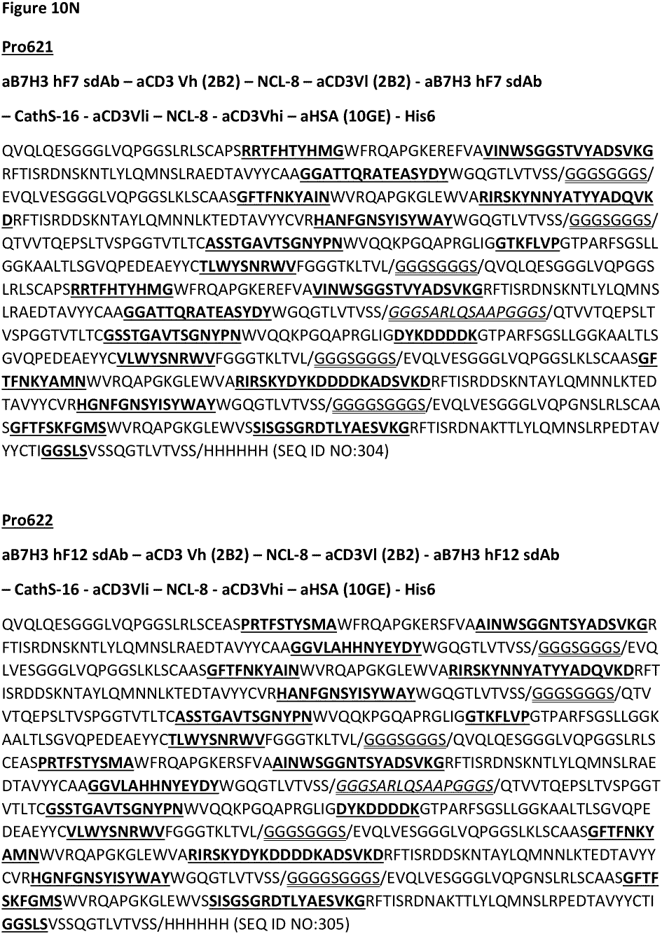

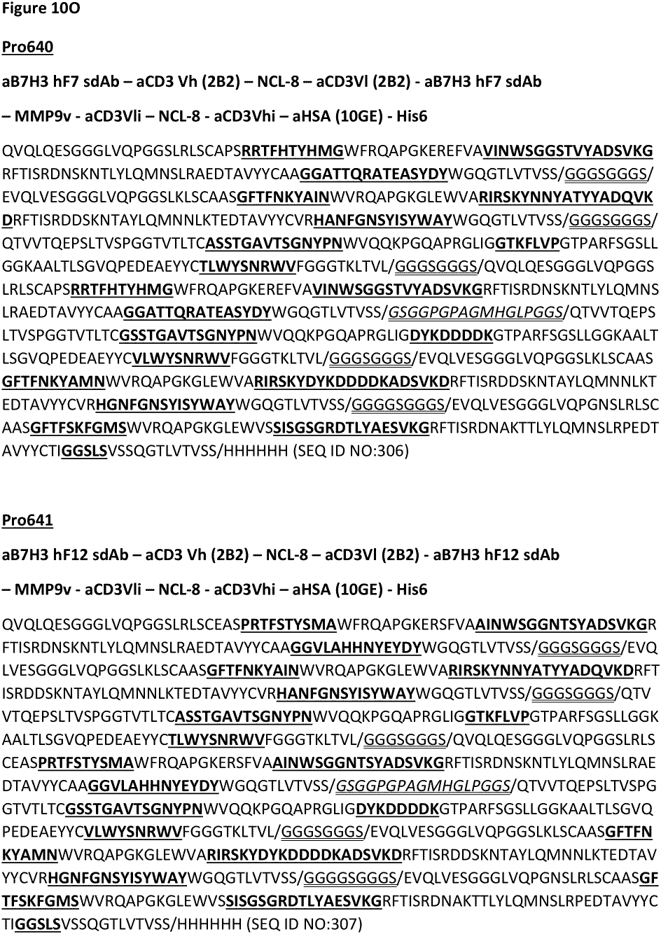

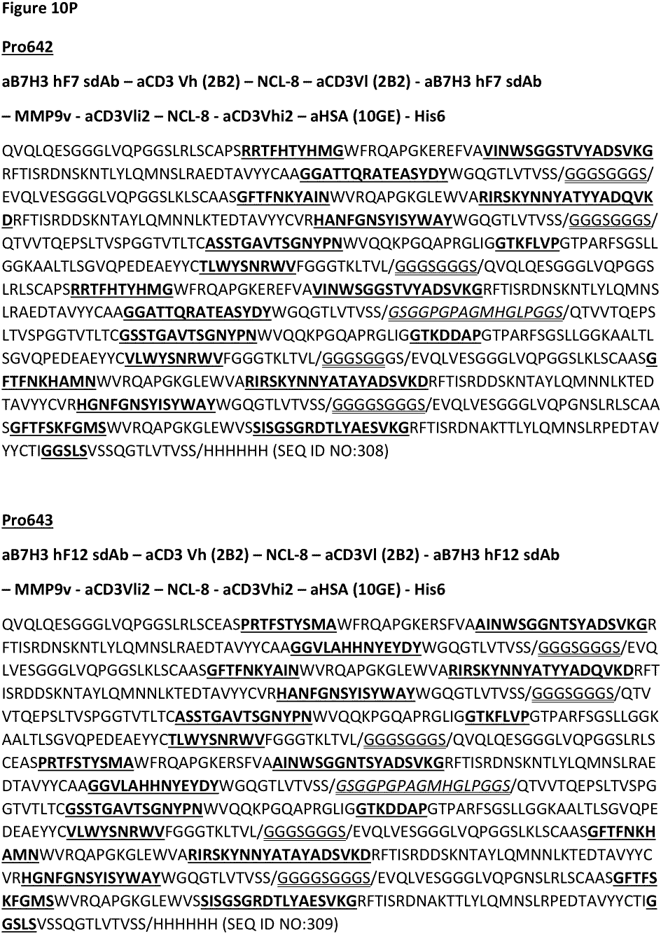

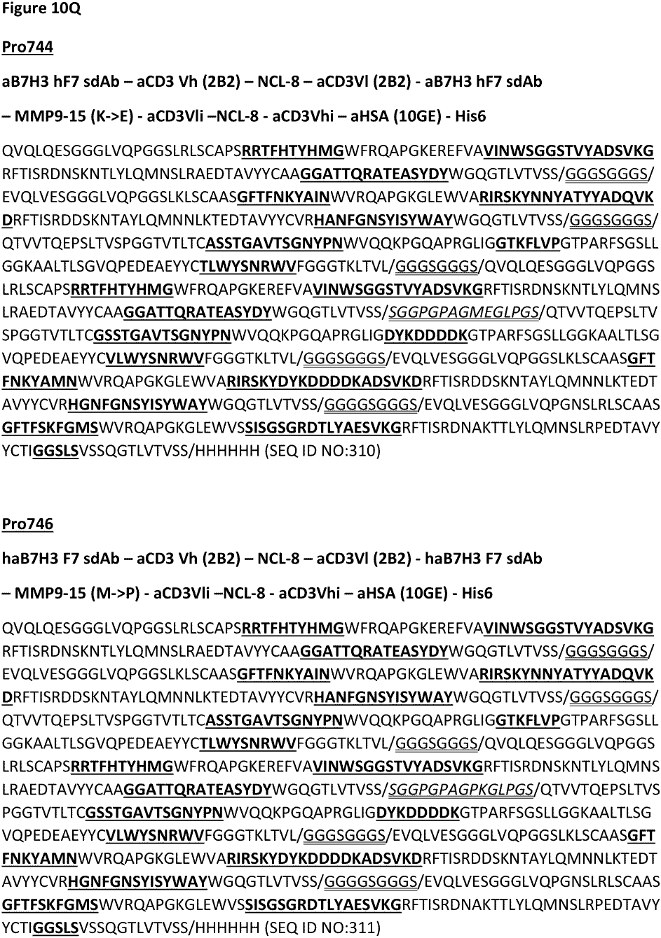

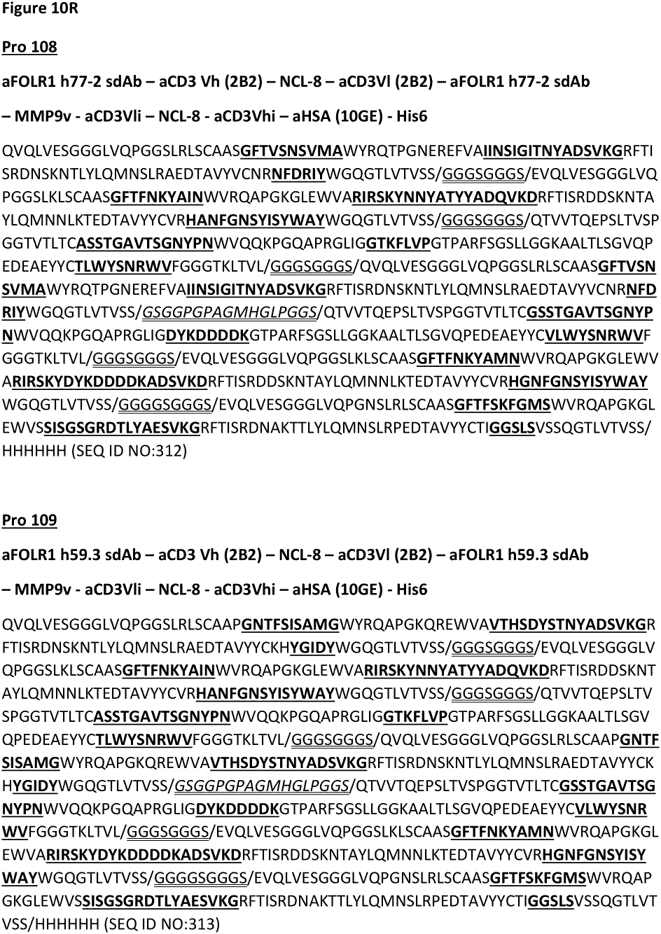

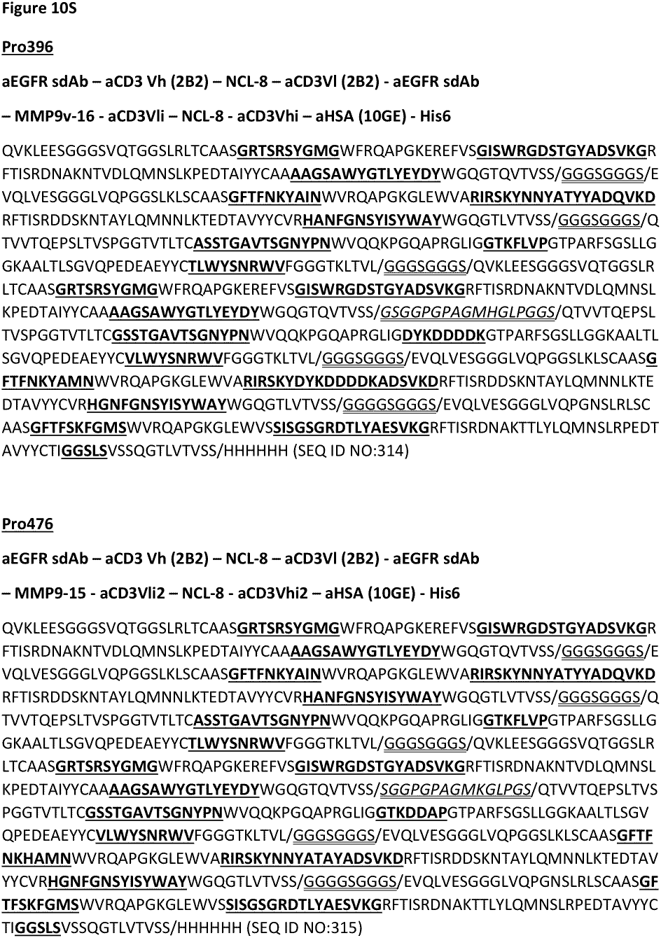

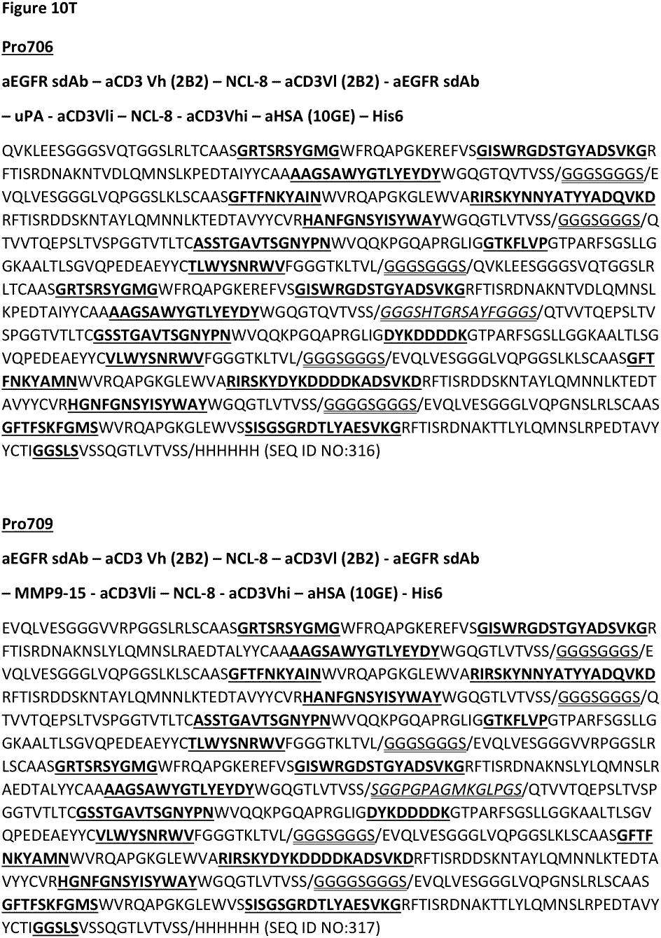

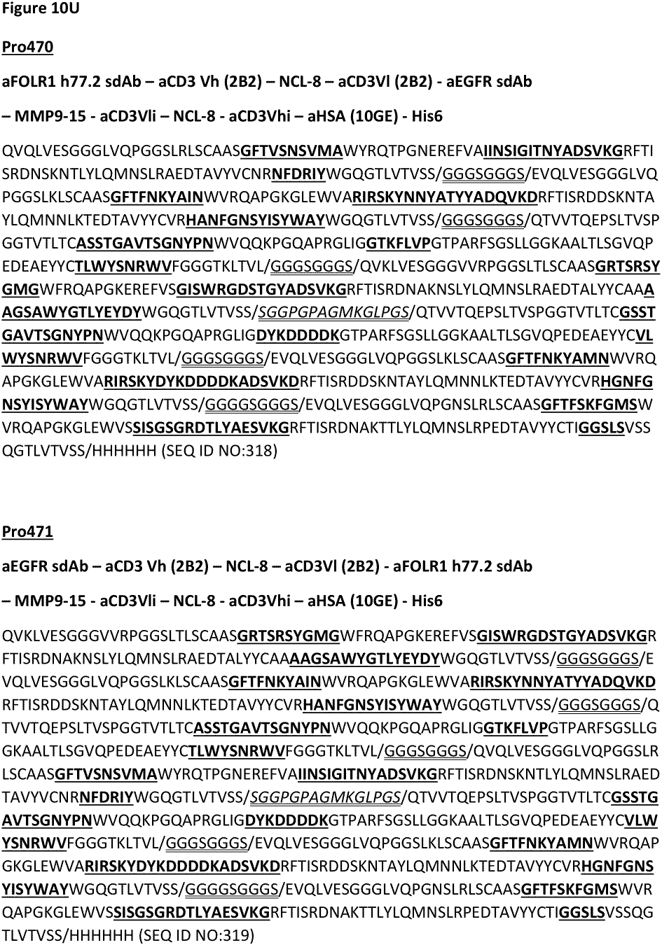

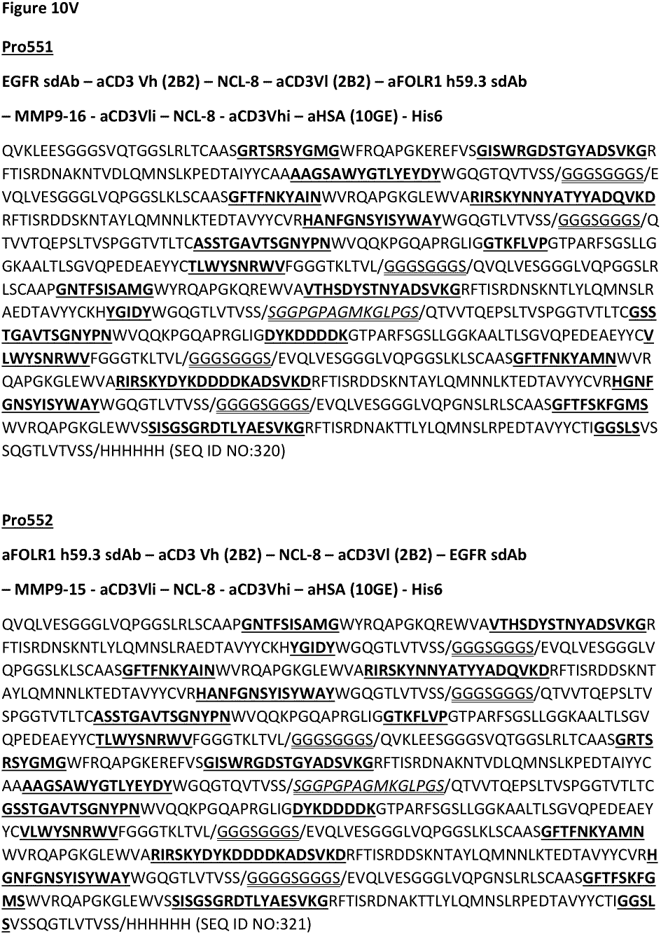

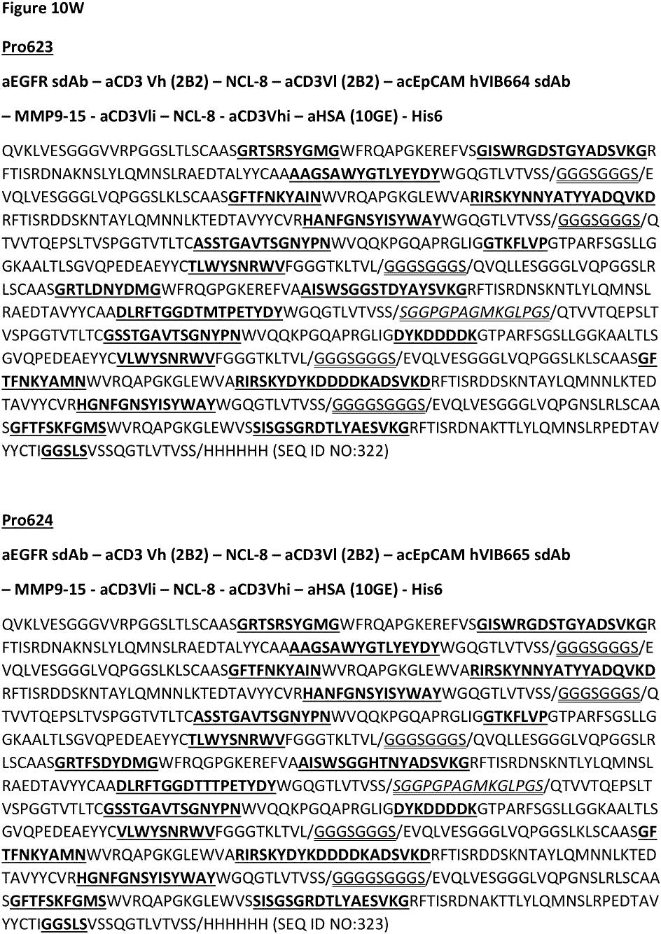

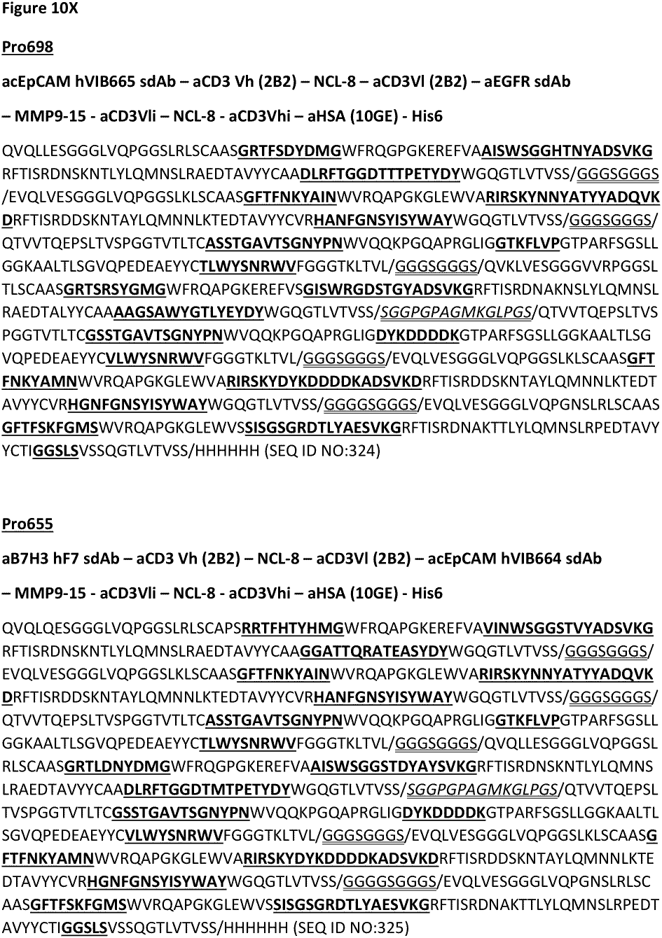

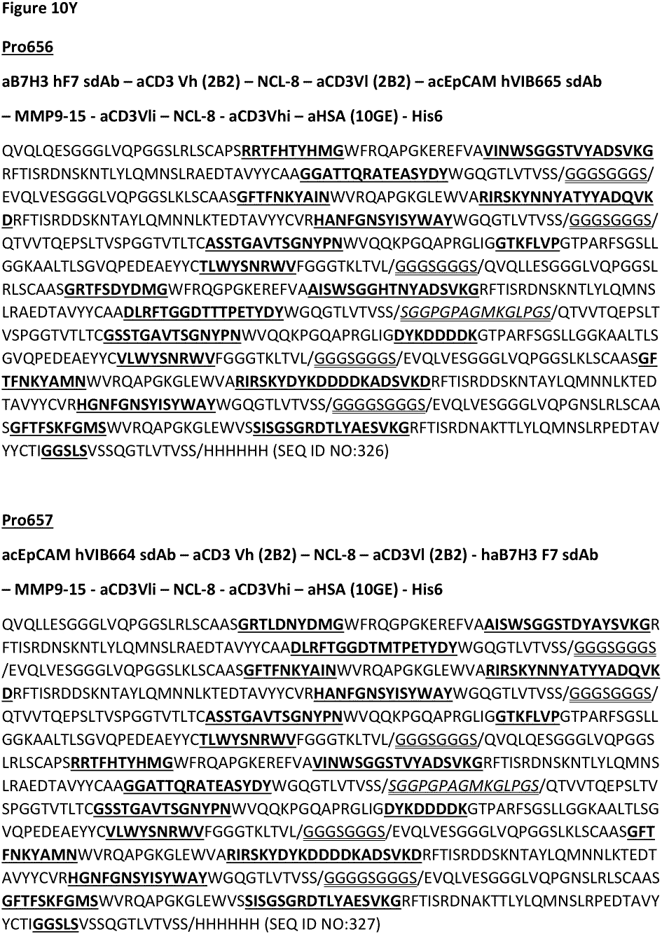

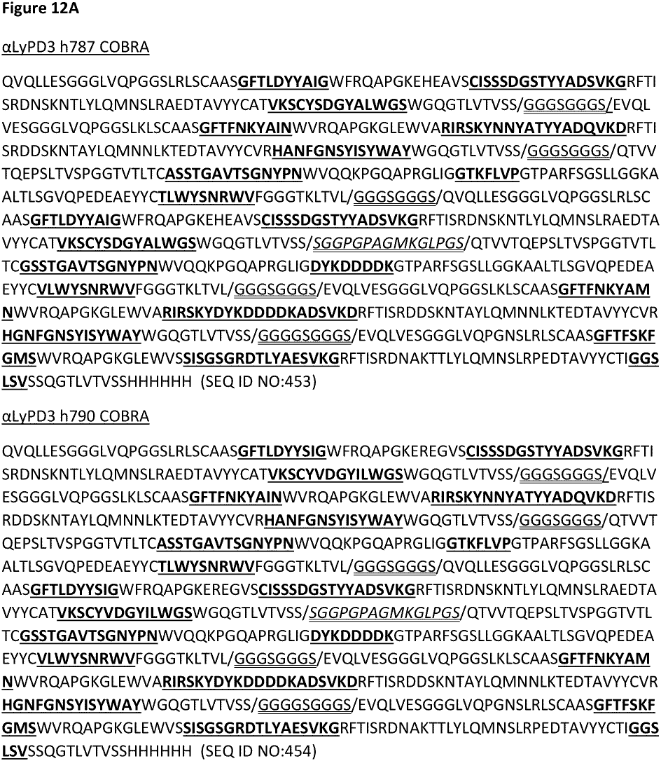

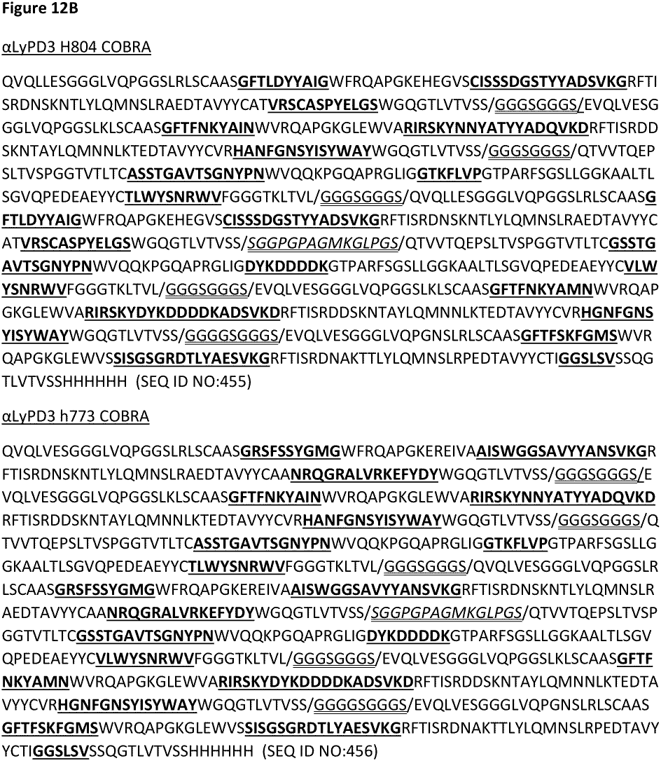

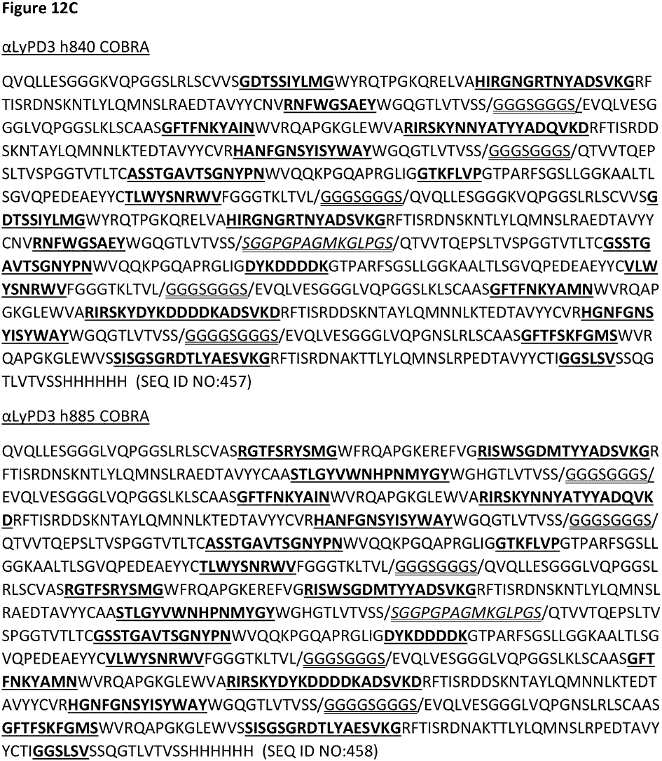

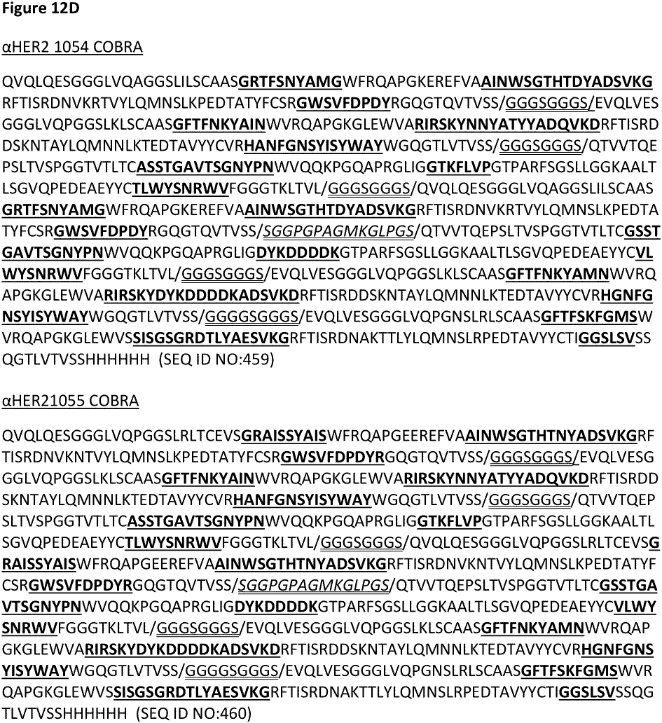

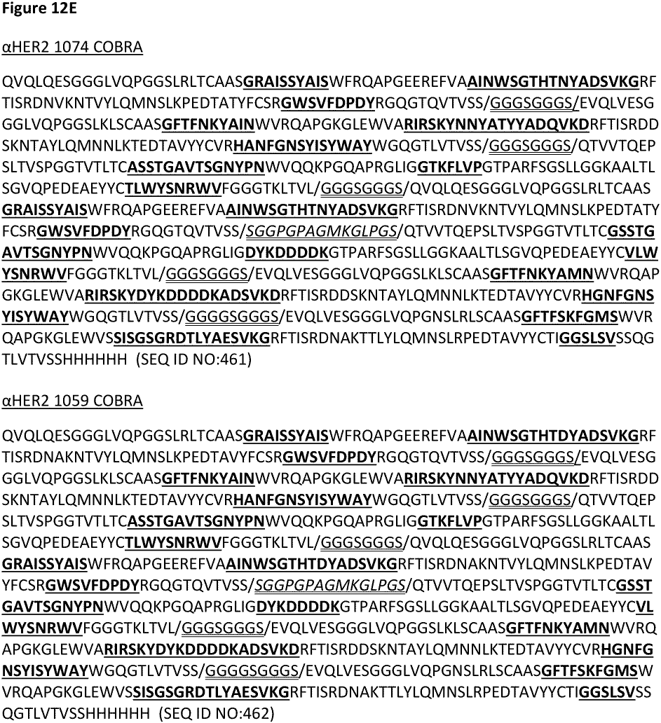

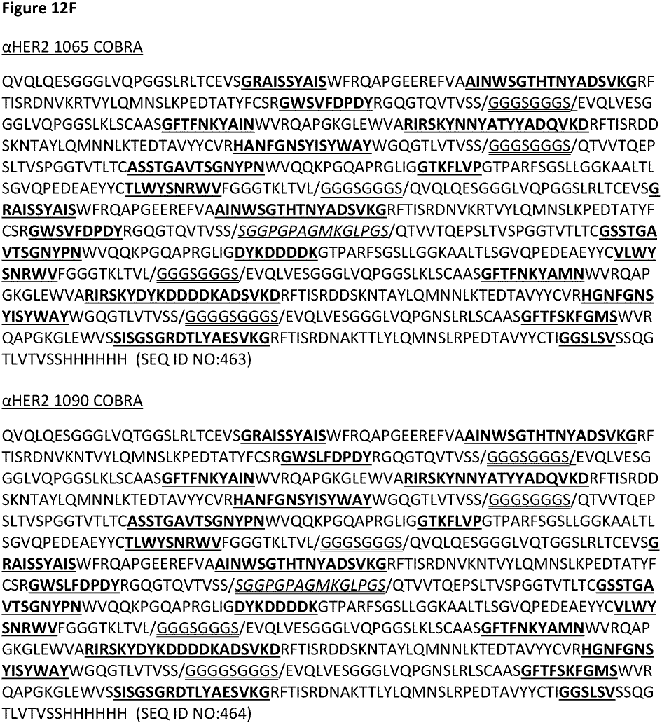

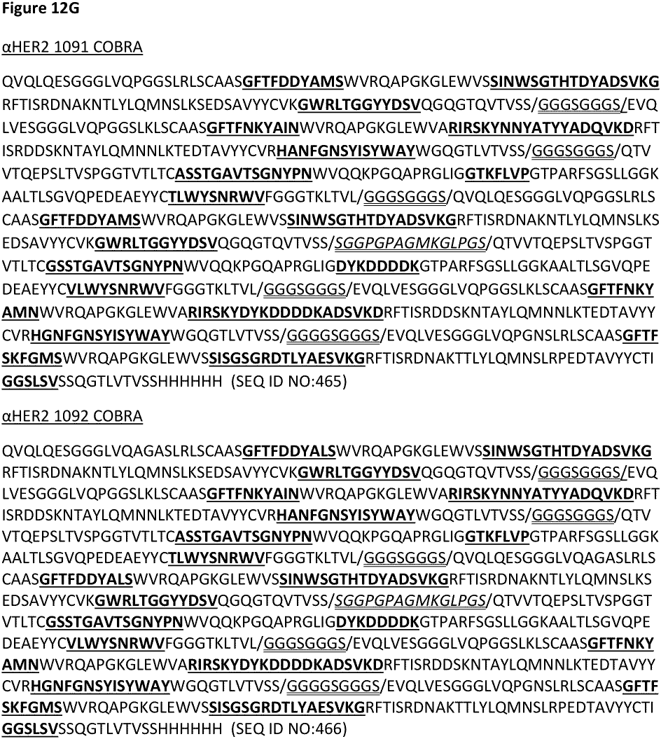

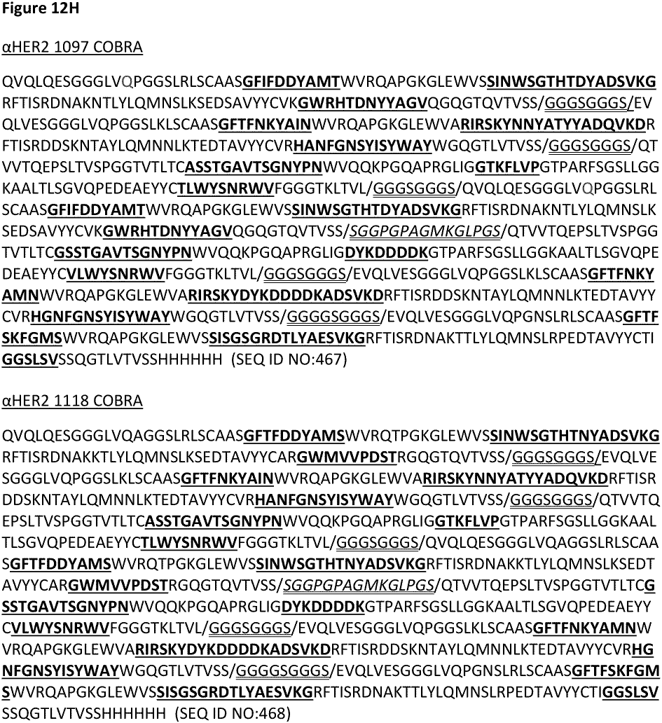

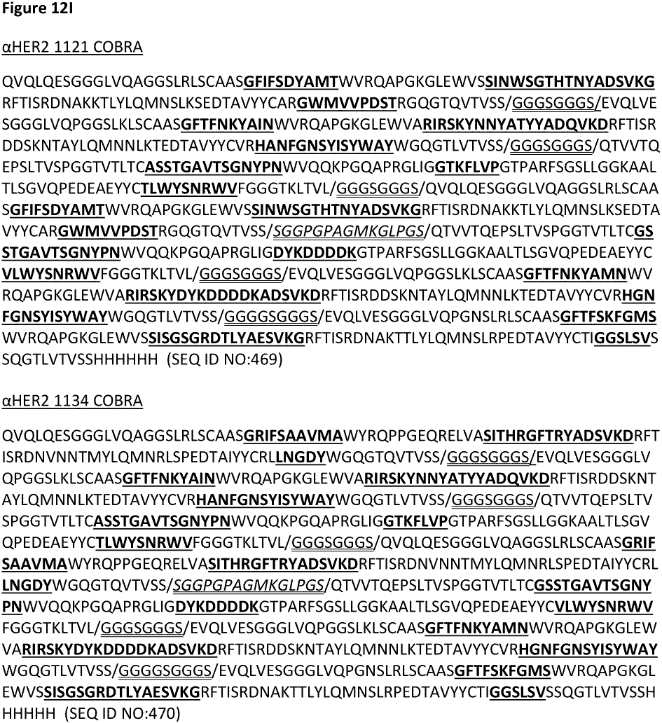

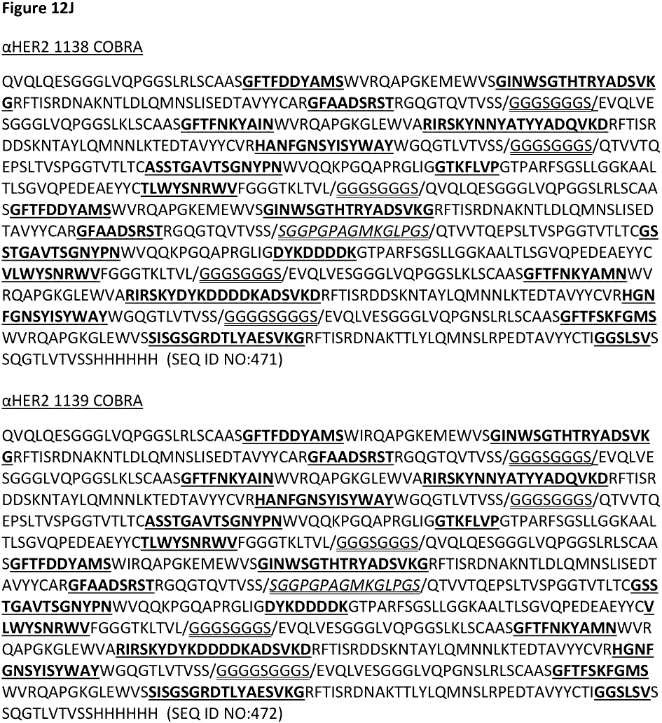

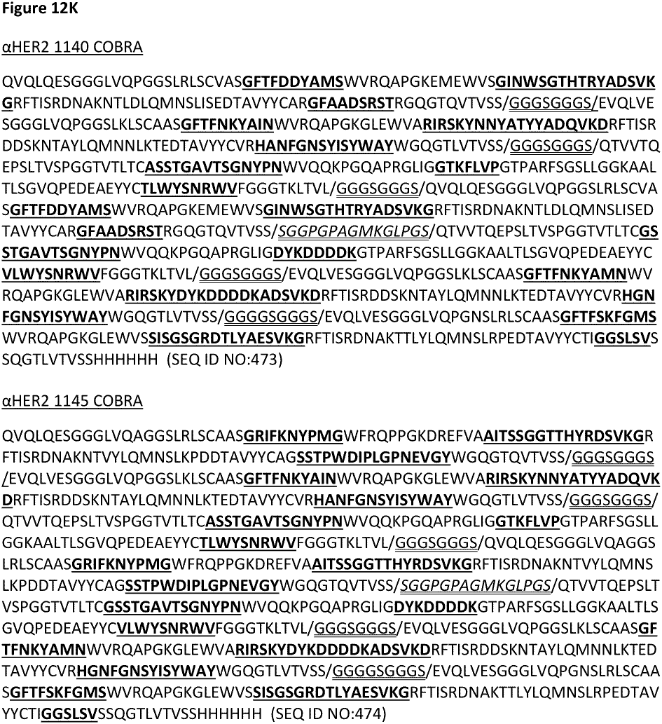

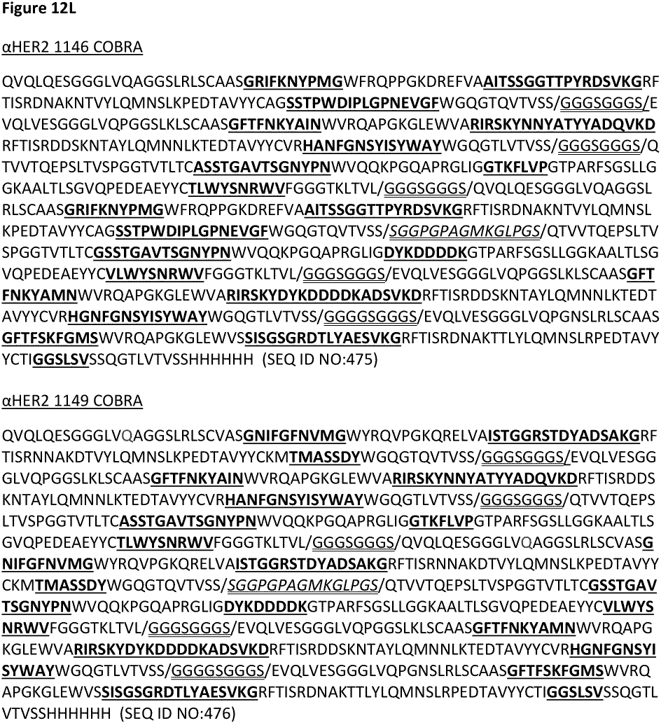

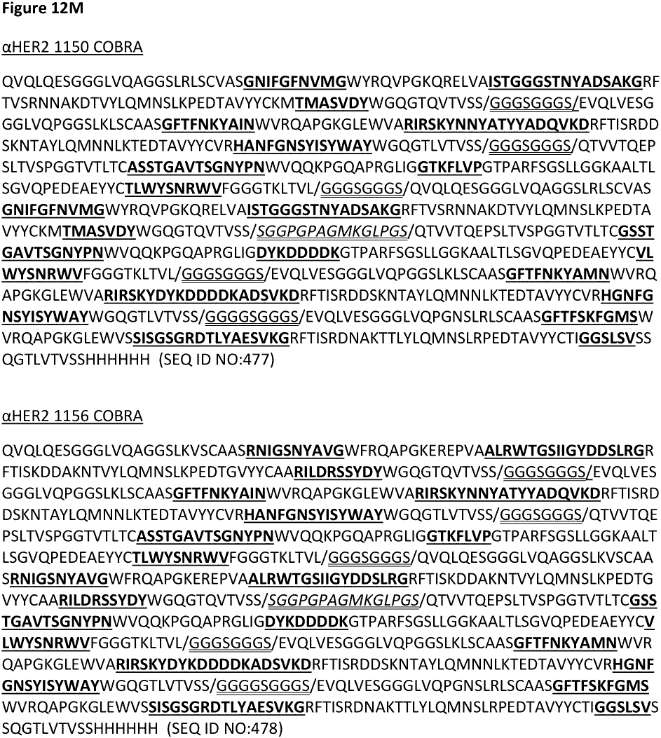

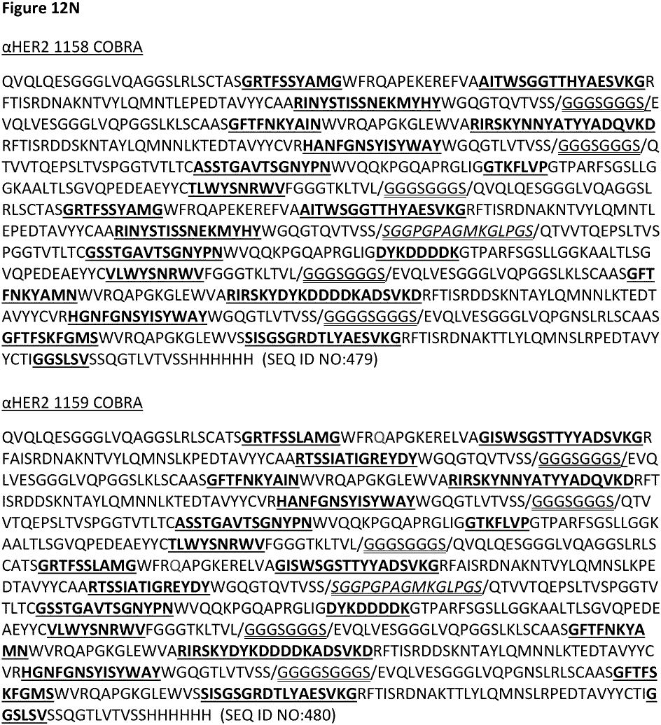

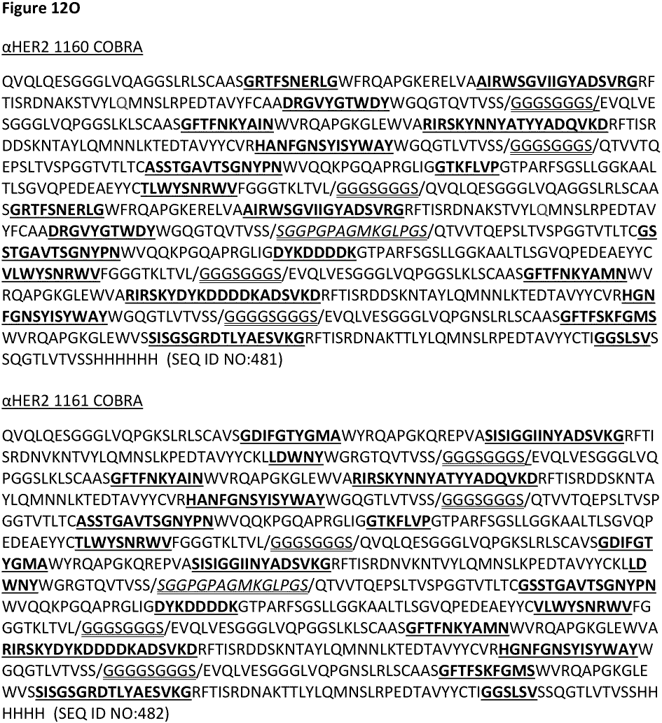

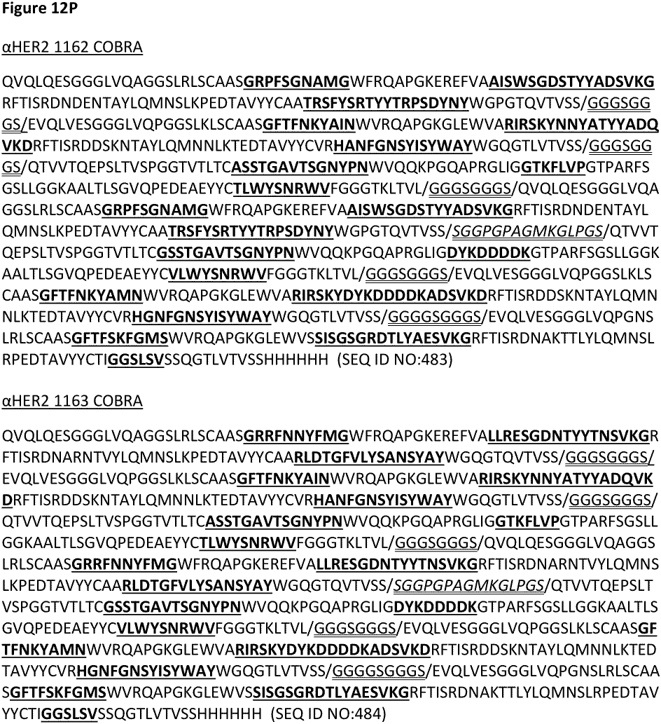

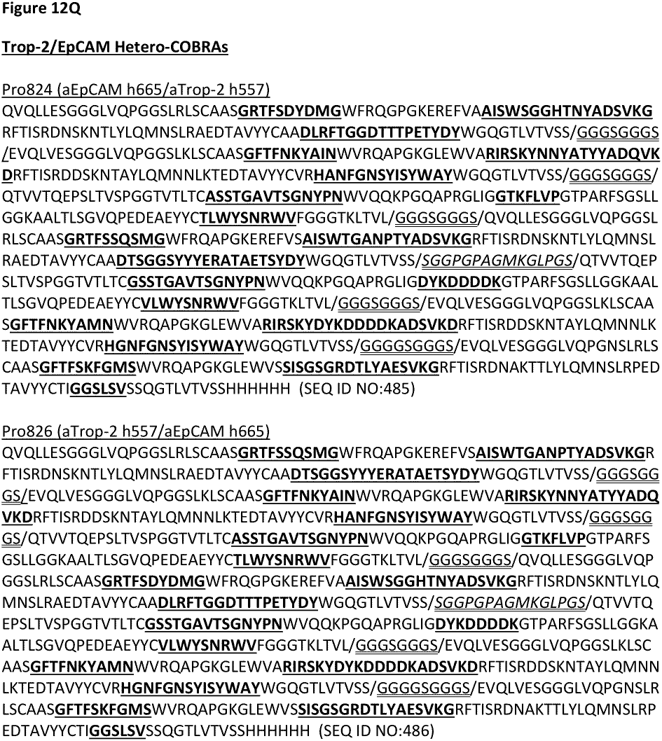

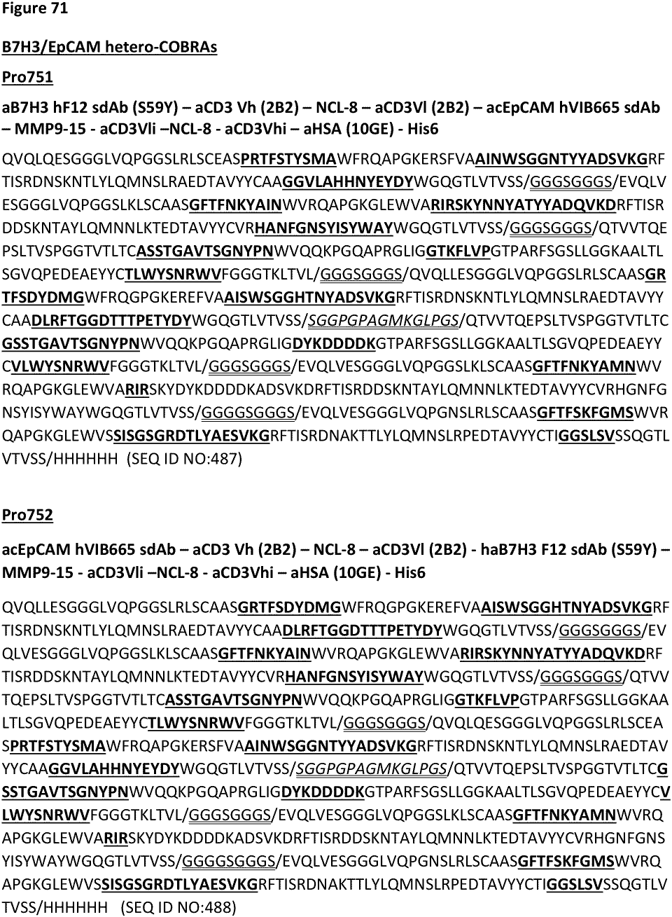

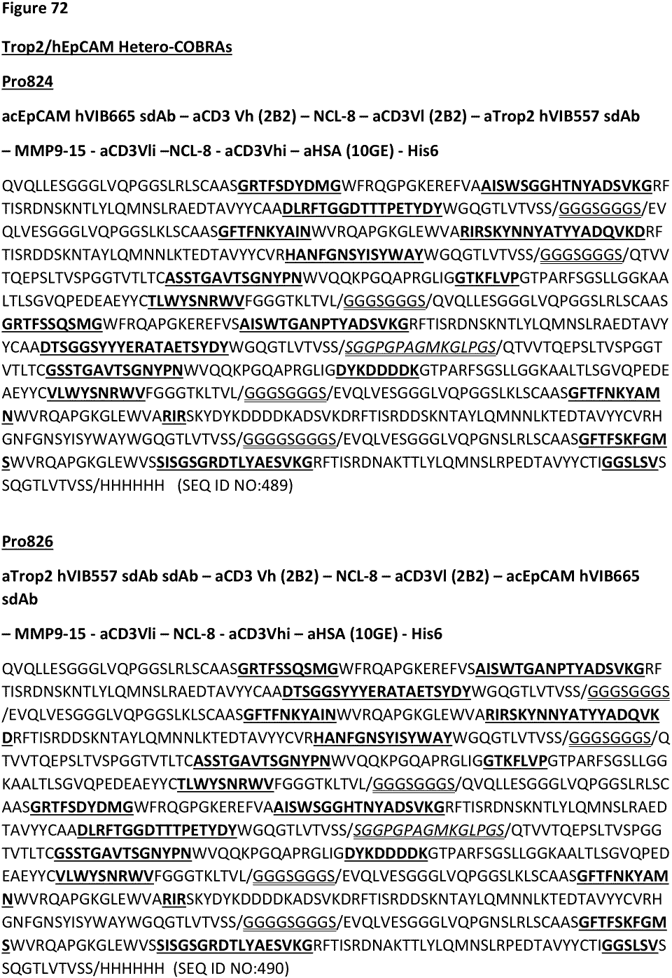

In some embodiments, the prodrug protein described herein is provided in the figures including FIGS. 9C-9V and the corresponding sequences set forth as SEQ ID NOS:413-452, which represent the exemplary proteins Pro225, Pro226, Pro233, Pro311, Pro312, Pro313, Pro246, Pro256, Pro420, Pro421, Pro393, Pro394, Pro395, Pro396, Pro429, Pro430, Pro431, Pro258, Pro221, Pro222, Pro223, Pro224, Pro254, Pro255, Pro262, Pro356, Pro359, Pro364, Pro388, Pro429, Pro430, Pro431, Pro432, Pro448, Pro449, Pro450, Pro451, Pro479, Pro480, and Pro495. In some embodiments, the prodrug protein described herein is provided in the figures including (i) FIG. 10A-10EE and the corresponding sequences set forth as SEQ ID NOS: 288-290, 291-302, 304-334, 336, 338 and 522-530, which represent the exemplary proteins Pro601, Pro602, V3, V4, Pro664, Pro665, Pro667, Pro694, Pro695, Pro565, Pro566, Pro567, Pro727, Pro728, Pro729, Pro730, Pro731, Pro676, Pro677, Pro678, Pro679, Pro808, Pro819, Pro621, Pro622, Pro640, Pro641, Pro642, Pro643, Pro744, Pro746, Pro108, Pro109, Pro396, Pro476, Pro706, Pro709, Pro470, Pro471, Pro551, Pro552, Pro623, Pro624, Pro698, Pro655, Pro656, Pro657, Pro658, Pro516, Pro517, Pro518, Pro519, Pro513, Pro186, Pro225, and Pro817, (ii) FIGS. 12A-12Q and the corresponding sequences set forth as SEQ ID NOS:453-486 which represent the exemplary proteins aLyPD3 h787 COBRA, aLyPD3 h790 COBRA, aLyPD3 h804 COBRA, aLyPD3 h773 COBRA, aLyPD3 h840 COBRA, aLyPD3 h885 COBRA, aHER2 1054 COBRA, aHER2 1055 COBRA, aHER2 1059 COBRA, aHER2 1065 COBRA, aHER2 1074 COBRA, aHER2 1090 COBRA, aHER2 1091 COBRA, aHER2 1092 COBRA, aHER2 1097 COBRA, aHER2 1118 COBRA, aHER2 1121 COBRA, aHER2 1134 COBRA, aHER2 1138 COBRA, aHER2 1139 COBRA, aHER2 1140 COBRA, aHER2 1145 COBRA, aHER2 1146, aHER2 1149 COBRA, aHER2 1150 COBRA, aHER2 1156 COBRA, aHER2 1158 COBRA, aHER2 1159 COBRA, aHER2 1160 COBRA, aHER2 1161 COBRA, aHER2 1162 COBRA, aHER2 1163 COBRA, Pro824, and Pro826, (iii) FIGS. 71-74 and and the corresponding sequences set forth as SEQ ID NOS:487-494 which represent the exemplary proteins Pro751, Pro752, Pro824, Pro826, Pro1109, Pro1111, Pro1117 and Pro1118.

In some embodiments, a COBRA construct is generated using the anti-HER2 VIB1058 sdABD (SEQ ID NO:536; e.g., an aHER2 1058 COBRA) or the hVIB1058 sdABD (SEQ ID NO:537; e.g., a humanized aHER2 1058 COBRA). In some such embodiments, the COBRA includes two copies of the VIB 1058 sdABD or hVIB1058 sdABD, e.g., a single targeting format 2 construct / “mono-specific COBRA.” In some such embodiments, the COBRA includes one copy of the VIB1058 sdABD or hVIB 1058 sdABD, e.g., a dual targeting format 2 Construct / “heteroCOBRA.”

### 1. Single Targeting Format 2 Constructs: “Mono-specific COBRAs”

In some embodiments, both of the αTTA domains bind to the same tumor target. Accordingly, in some embodiments, the prodrug protein comprises, from N- to C-terminal: (sdABD-TTA1)-domain linker-aVH-CNCL-aVL-domain linker-sdABD-TTA2)-CL-iVL-CNCL-iVH-domain linker-(sdABD-HSA). In this embodiment, the aVH, aVL, iVH, iVL have the sequences shown in FIGS. 7A-7B. In this embodiment, the two targeting domains bind to the same TTA, which can be EGFR, EpCAM, FOLR1, Trop2, CA9, B7H3, LyPD3 or HER2, the sequences for which are depicted in FIGS. 5A-5N.

In some embodiments, the sdABD-TTA1 is selected from the group consisting of a sdABD-B7H3, sdABD-CA9, sdABD-EGFR, sdABD-EpCAM, sdABD-HER2, sdABD-LyPD3, and sdABD-Trop2. In some embodiments, the sdABD-TTA2 is selected from the group consisting of a sdABD-B7H3, sdABD-CA9, sdABD-EGFR, sdABD-EpCAM, sdABD-HER2, sdABD-LyPD3, and sdABD-Trop2. In some embodiments, the sdABD-TTA1 and sdABD-TTA2 bind the same target antigen. In some embodiments, the sdABD-TTA1 and the sdABD-TTA2 bind the same target antigen but at different locations. In some embodiments, the sdABD-TTA1 and the sdABD-TTA2 bind the same target antigen but at the same location. In some embodiments, the sdABD-TTA1 and the sdABD-TTA2 have the same amino acid sequence. Any sequence of the sdABDs described herein can be the sequence of the sdABD-TTA1, the sdABD-TTA2, or both. In some embodiments, the sdCDR1, sdCDR2 and sdCDR3 of sdABD-TTA1 are the same as the the sdCDR1, sdCDR2 and sdCDR3 of sdABD-TTA2, respectively.

In some embodiments, exemplary mono-specific COBRAs bind to a tumor target selected from the group consisting of B7H3, CA9, EGFR, EpCAM, HER2, LyPD3 and Trop2. In some embodiments, a mono-specific COBRA that bind to B7H3 (e.g., human B7H3) includes any of the fusion proteins of FIG. 9C, such as Pro225 (SEQ ID NO:413) and Pro226 (SEQ ID NO:414); FIGS. 10A-10E, such as Pro601 (SEQ ID NO:522), Pro602 (SEQ ID NO:523), V3 (SEQ ID NO:524), V4 (SEQ ID NO:525), Pro664 (SEQ ID NO:526), Pro665 (SEQ ID NO:527), Pro667 (SEQ ID NO:528), Pro694 (SEQ ID NO:529), and Pro695 (SEQ ID NO:530); FIGS. 10O-10Q, such as Pro640 (SEQ ID NO:306), Pro641 (SEQ ID NO:307), Pro642 (SEQ ID NO:308), Pro643 (SEQ ID NO:309), Pro774 (SEQ ID NO:310) and Pro746 (SEQ ID NO:311); FIG. 10DD-10EE such as Pro225 (SEQ ID NO:336) and Pro817 (SEQ ID NO:338).

In some embodiments, a mono-specific COBRA that bind to CA9 (e.g., human CA9) includes any of the fusion proteins of FIG. 10Z-10BB such as Pro516 (SEQ ID NO:329), Pro517 (SEQ ID NO:330), Pro518 (SEQ ID NO:331), and Pro519 (SEQ ID NO:332).

In some embodiments, a mono-specific COBRA that bind to EGFR (e.g., human EGFR) includes any of the fusion proteins of FIGS. 10S-10T such as Pro396 (SEQ ID NO:314), Pro476 (SEQ ID NO:315), Pro706 (SEQ ID NO:316), and Pro709 (SEQ ID NO:317).

In some embodiments, a mono-specific COBRA that bind to EpCAM (e.g., human EpCAM) includes any of the fusion proteins of FIGS. 10F-10J such as Pro565 (SEQ ID NO:288), Pro566 (SEQ ID NO:289), Pro567 (SEQ ID NO:290), Pro727 (SEQ ID NO:292), Pro728 (SEQ ID NO:293), Pro729 (SEQ ID NO:294), Pro730 (SEQ ID NO:295), and Pro731 (SEQ ID NO:296).

In some embodiments, a mono-specific COBRA that bind to FOLR1 (e.g., human FOLR1) includes any of the fusion proteins of FIGS. 9D and 9E such as Pro311 (SEQ ID NO:416), Pro312 (SEQ ID NO:417), and Pro313 (SEQ ID NO:418).

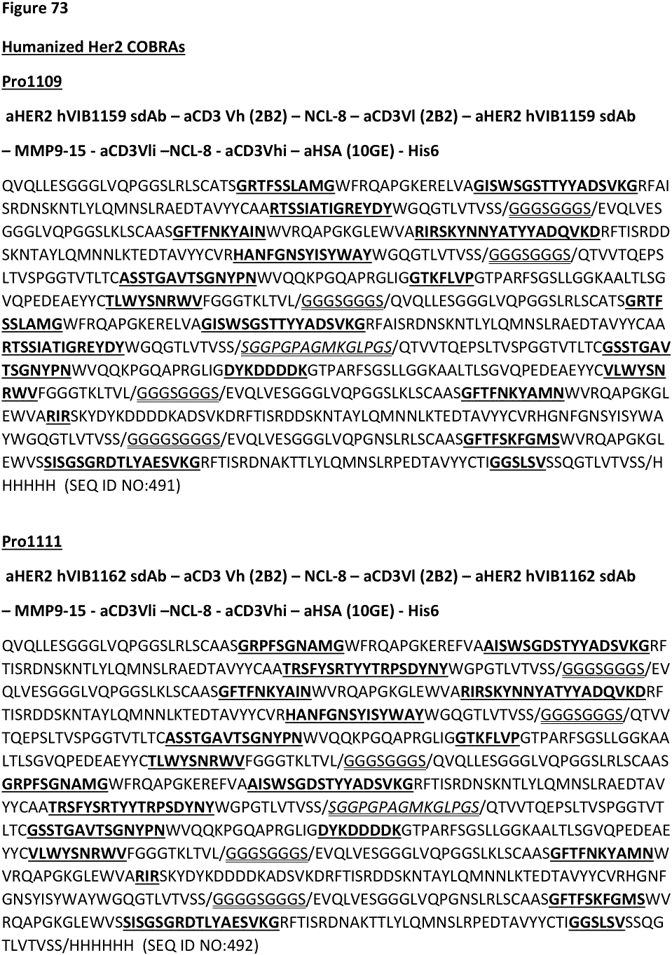

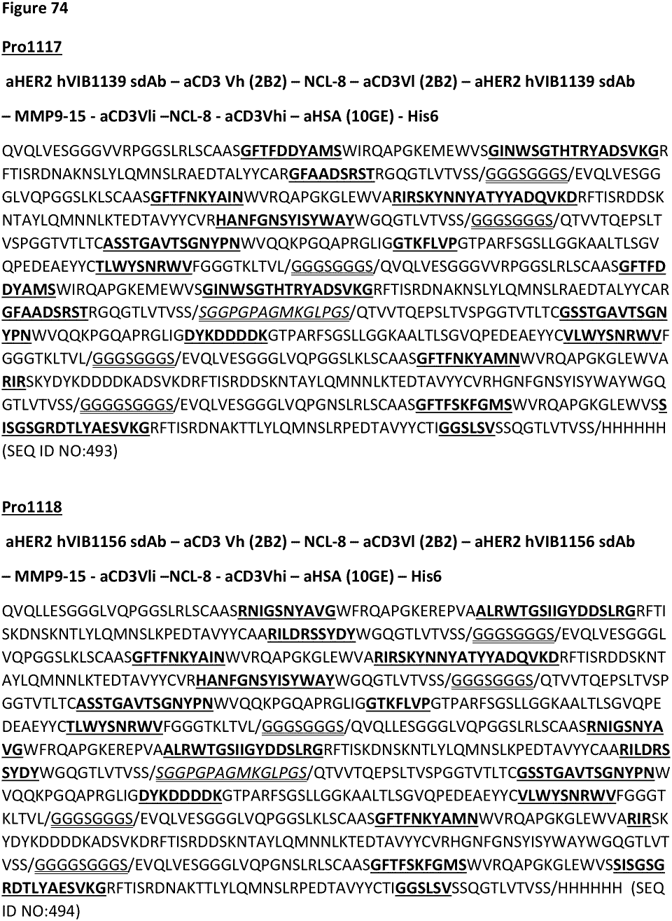

In some embodiments, a mono-specific COBRA that bind to HER2 (e.g., human HER2) includes any of the fusion proteins of FIGS. 12D-12P such as any one of SEQ ID NOS: 459-484; and FIGS. 73 and 74 such as Pro1109 (SEQ ID NO:491), Pro1111 (SEQ ID NO:492), Pro1117 (SEQ ID NO:493), and Pro1118 (SEQ ID NO:494).

In some embodiments, a mono-specific COBRA that bind to LyPD3 (e.g., human LyPD3) includes any of the fusion proteins of FIGS. 12A-12C such as any of SEQ ID NOS: 453-458.

In some embodiments, a mono-specific COBRA that bind to Trop2 (e.g., human Trop2) includes any of the fusion proteins of FIGS. 10J-10M such as Pro676 (SEQ ID NO:297), Pro677 (SEQ ID NO:298), Pro678 (SEQ ID NO:299), Pro679 (SEQ ID NO:300), Pro808 (SEQ ID NO:301), and Pro819 (SEQ ID NO:302).

### 2. Dual Targeting Format 2 Constructs: “HeteroCOBRAs”

In some embodiments, each of the αTTA domains bind to a different tumor target. Accordingly, in some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In this embodiment, the aVH, aVL, iVH, iVL have the sequences shown in FIGS. 5A-5B. In this embodiment, the two targeting domains bind to different TTAs.

In Format 2, preferred dual targeting constructs (sometimes referred to herein as “hetero-COBRAs”) include combinations that target EGFR and EpCAM, EGFR and Trop2, EGFR and FOLR1, EGFR and B7H3, EGFR and LyPD3, EGFR and HER2, EpCAM and FOLR1, EpCAM and B7H3, EpCAM and Trop2, EpCAM and LyPD3, EpCAM and HER2, FOLR1 and B7H3, FOLR1 and HER2, FOLR1 and Trop2, FOLR1 and LyPD3, B7H3 and HER2, B7H3 and Trop2, B7H3 and LyPD3, HER2 and Trop2, HER2 and LyPD3, and Trop2 and LyPD3. These sometimes are discussed herein as “EGFR X EpCAM”, etc., constructs.

In some embodiments, the sdABD-TTA1 is selected from the group consisting of a sdABD-B7H3, sdABD-CA9, sdABD-EGFR, sdABD-EpCAM, sdABD-HER2, sdABD-LyPD3, and sdABD-Trop2. In some embodiments, the sdABD-TTA2 is selected from the group consisting of a sdABD-B7H3, sdABD-CA9, sdABD-EGFR, sdABD-EpCAM, sdABD-HER2, sdABD-LyPD3, and sdABD-Trop2. In some embodiments, the sdABD-TTA1 and sdABD-TTA2 bind different target antigens.

In some embodiments, the sdABD-TTA1 is a sdABD-B7H3 and the sdABD-TTA2 is selected from the group consisting of a sdABD-CA9, sdABD-EGFR, sdABD-EpCAM, sdABD-HER2, sdABD-LyPD3, and sdABD-Trop2. In some embodiments, the sdABD-TTA1 is a sdABD-CA9 and the sdABD-TTA2 is selected from the group consisting of a sdABD-B7H3, sdABD-EGFR, sdABD-EpCAM, sdABD-HER2, sdABD-LyPD3, and sdABD-Trop2. In some embodiments, the sdABD-TTA1 is a sdABD-EGFR and the sdABD-TTA2 is selected from the group consisting of a sdABD-B7H3, sdABD-CA9, sdABD-EpCAM, sdABD-HER2, sdABD-LyPD3, and sdABD-Trop2. In some embodiments, the sdABD-TTA1 is a sdABD-EpCAM and the sdABD-TTA2 is selected from the group consisting of a sdABD-B7H3, sdABD-CA9, sdABD-EGFR, sdABD-HER2, sdABD-LyPD3, and sdABD-Trop2. In some embodiments, the sdABD-TTA1 is a sdABD-HER2 and the sdABD-TTA2 is selected from the group consisting of a sdABD-B7H3, sdABD-CA9, sdABD-EGFR, sdABD-EpCAM, sdABD-LyPD3, and sdABD-Trop2. In some embodiments, the sdABD-TTA1 is a sdABD-LyPD3 and the sdABD-TTA2 is selected from the group consisting of a sdABD-B7H3, sdABD-CA9, sdABD-EGFR, sdABD-EpCAM, sdABD-HER2, and sdABD-Trop2. In some embodiments, the sdABD-TTA1 is a sdABD-Trop2 and the sdABD-TTA2 is selected from the group consisting of a sdABD-B7H3, sdABD-CA9, sdABD-EGFR, sdABD-EpCAM, sdABD-HER2, and sdABD-LyPD3. Any sequence of a sdABD-TTA described herein such as those of a sdABD-B7H3, a sdABD-CA9, a sdABD-EGFR, a sdABD-EpCAM, a sdABD-HER2, a sdABD-LyPD3 and a sdABD-Trop2 can be used in a dual targeting format 2 construct or hetero-COBRA.

In many embodiments, the sdABD-TTA1 is selected from the group consisting of a sdABD-CA9, sdABD-EGFR, sdABD-EpCAM, sdABD-HER2, sdABD-LyPD3, and sdABD-Trop2, and the sdABD-TTA2 is a sdABD-B7H3. In many embodiments, the sdABD-TTA1 is selected from the group consisting of a sdABD-B7H3, sdABD-EGFR, sdABD-EpCAM, sdABD-HER2, sdABD-LyPD3, and sdABD-Trop2, and the sdABD-TTA2 is a sdABD-CA9. In many embodiments, the sdABD-TTA1 is selected from the group consisting of a sdABD-B7H3, sdABD-CA9, sdABD-EpCAM, sdABD-HER2, sdABD-LyPD3, and sdABD-Trop2, and the sdABD-TTA2 is a sdABD-EGFR. In many embodiments, the sdABD-TTA1 is selected from the group consisting of a sdABD-B7H3, sdABD-CA9, sdABD-EGFR, sdABD-HER2, sdABD-LyPD3, and sdABD-Trop2, and the sdABD-TTA2 is a sdABD-EpCAM. In many embodiments, the sdABD-TTA1 is selected from the group consisting of a sdABD-B7H3, sdABD-CA9, sdABD-EGFR, sdABD-EpCAM, sdABD-LyPD3, and sdABD-Trop2, and the sdABD-TTA2 is a sdABD-HER2. In many embodiments, the sdABD-TTA1 is selected from the group consisting of a sdABD-B7H3, sdABD-CA9, sdABD-EGFR, sdABD-EpCAM, sdABD-HER2, and sdABD-Trop2, and the sdABD-TTA2 is a sdABD-LyPD3. In many embodiments, the sdABD-TTA1 is selected from the group consisting of a sdABD-B7H3, sdABD-CA9, sdABD-EGFR, sdABD-EpCAM, sdABD-HER2, and sdABD-LyPD3, and the sdABD-TTA2 is a sdABD-Trop2. Any sequence of a sdABD-TTA described herein such as those of a sdABD-B7H3, a sdABD-CA9, a sdABD-EGFR, a sdABD-EpCAM, a sdABD-HER2, a sdABD-LyPD3 and a sdABD-Trop2 can be used in such dual targeting format 2 constructs or hetero-COBRAs.

### A. EGFR X EpCAM

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In this embodiment, the aVH, aVL, iVH, iVL have the sequences shown in FIGS. 7A-7B. In this embodiment, the two targeting domains bind to EGFR and EpCAM, and the sdABD-TTAs have the sequences in FIGS. 5A, 5D, 5E, and 75 and the sequences provided therein and the corresponding sequence listing. In this embodiment, some combinations of the EGFR sdABD and the EpCAM sdABD include:

| | | | | | |
|---|---|---|---|---|---|
| cross | EGFR1 | hEGFR1 | EGFR2 | EGFR2a | EGFR2d |
| EpCAM h13 | In either orientation | In either orientation | In either orientation | In either orientation | In either orientation |
| EpCAM h23 | In either orientation | In either orientation | In either orientation | In either orientation | In either orientation |
| EpCAM hVIB665 | In either orientation | In either orientation | In either orientation | In either orientation | In either orientation |
| EpCAM hVIB666 | In either orientation | In either orientation | In either orientation | In either orientation | In either orientation |
| EpCAM h664 | In either orientation | In either orientation | In either orientation | In either orientation | In either orientation |

In this case, “either orientation” means that either the EpCAM sdABD is N-terminal to the EGFR sdABD in the constructs of the invention or the EpCAM sdABD is C-terminal to it.

### B. EGFR X FOLR1

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiments, the aVH, aVL, iVH, iVL have the sequences shown in FIGS. 7A-7B. In some embodiments, the two targeting domains bind to EGFR and FOLR1, and the sdABD-TTAs have the sequences in FIGS. 5A-5B and the sequences provided therein and the corresponding sequence listing. In some embodiments, some combinations of the EGFR sdAb and the FOLR1 sdAb include:

| | | | | | |
|---|---|---|---|---|---|
| cross | EGFR1 | hEGFR1 | EGFR2 | EGFR2a | EGFR2d |
| FOLR1 h77-2 | In either orientation | In either orientation | In either orientation | In either orientation | In either orientation |
| FOLR1 h59.3 | In either orientation | In either orientation | In either orientation | In either orientation | In either orientation |
| FOLR h22-4 | In either orientation | In either orientation | In either orientation | In either orientation | In either orientation |

In this case, “either orientation” means that either the FOLR1 sdABD is N-terminal to the EGFR sdABD in the constructs of the invention or the FOLR1 sdABD is C-terminal to it.

### C. EGFR X B7H3

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In this embodiment, the aVH, aVL, iVH, iVL have the sequences shown in FIGS. 7A-7B. In some embodiments, the two targeting domains bind to EGFR and B7H3, and the sdABD-TTAs have the sequences in FIGS. 5A-5D and the sequences provided therein and the corresponding sequence listing. In some embodiments, some combinations of the EGFR sdABD and the B7H3 sdABD include:

| | | | | | |
|---|---|---|---|---|---|
| cross | EGFR1 | hEGFR1 | EGFR2 | EGFR2a | EGFR2d |
| B7H3 hF7 | In either orientation | In either orientation | In either orientation | In either orientation | In either orientation |
| B7H3 hF12 | In either orientation | In either orientation | In either orientation | In either orientation | In either orientation |
| B7H3 hF12 (N57Q) | In either orientation | In either orientation | In either orientation | In either orientation | In either orientation |
| B7H3 hF12 (N57E) | In either orientation | In either orientation | In either orientation | In either orientation | In either orientation |
| B7H3 hF12 (N57D) | In either orientation | In either orientation | In either orientation | In either orientation | In either orientation |
| B7H3 hF12 (S59A) | In either orientation | In either orientation | In either orientation | In either orientation | In either orientation |
| B7H3 hF12 (S59Y) | In either orientation | In either orientation | In either orientation | In either orientation | In either orientation |

In this case, “either orientation” means that either the B7H3 sdABD is N-terminal to the EGFR sdABD in the constructs of the invention or the B7H3 sdABD is C-terminal to it.

### D. EGFR X Trop2

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiments, the aVH, aVL, iVH, iVL have the sequences shown in FIG. 7. In some embodiments, the two targeting domains bind to EGFR and Trop2, and the sdABD-TTAs have the sequences in FIGS. 5A, 5E and 5F and the sequences provided therein and the corresponding sequence listing. In this embodiments, some combinations of the EGFR sdABD and the Trop2 sdABD include:

| | | | | | |
|---|---|---|---|---|---|
| cross | EGFR1 | hEGFR1 | EGFR2 | EGFR2a | EGFR2d |
| aTrop2 hVIB557 | In either orientation | In either orientation | In either orientation | In either orientation | In either orientation |
| aTrop2 hVIB565 | In either orientation | In either orientation | In either orientation | In either orientation | In either orientation |
| aTrop2 hVIB575 | In either orientation | In either orientation | In either orientation | In either orientation | In either orientation |
| aTrop2 hVIB578 | In either orientation | In either orientation | In either orientation | In either orientation | In either orientation |
| aTrop2 hVIB609 | In either orientation | In either orientation | In either orientation | In either orientation | In either orientation |
| aTrop2 hVIB619 | In either orientation | In either orientation | In either orientation | In either orientation | In either orientation |

In this case, “either orientation” means that either the Trop2 sdABD is N-terminal to the EGFR sdABD in the constructs of the invention or the Trop2 sdABD is C-terminal to it.

### E. EGFR X LyPD3

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiments, the aVH, aVL, iVH, iVL have the sequences shown in FIGS. 7A-7B. In some embodiments, the two targeting domains bind to EGFR and LyPD3, and the sdABD-TTAs have the sequences in FIGS. 5A, 5G and 5H and the sequences provided therein and the corresponding sequence listing. In this embodiment, some combinations of the EGFR sdABD and the Trop2 sdABD include:

| | | | | | |
|---|---|---|---|---|---|
| cross | EGFR1 | hEGFR1 | EGFR2 | EGFR2a | EGFR2d |
| aLyPD3 h790 | In either orientation | In either orientation | In either orientation | In either orientation | In either orientation |
| aLyPD3 h804 | In either orientation | In either orientation | In either orientation | In either orientation | In either orientation |
| aLyPD3 h773 | In either orientation | In either orientation | In either orientation | In either orientation | In either orientation |
| aLyPD3 h840 | In either orientation | In either orientation | In either orientation | In either orientation | In either orientation |
| aLyPD3 h885 | In either orientation | In either orientation | In either orientation | In either orientation | In either orientation |
| aLyPD3 h787 | In either orientation | In either orientation | In either orientation | In either orientation | In either orientation |

In this case, “either orientation” means that either the Trop2 sdABD is N-terminal to the EGFR sdABD in the constructs of the invention or the Trop2 sdABD is C-terminal to it.

### F. EGFR X HER2

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiments, the aVH, aVL, iVH, iVL have the sequences shown in FIGS. 7A-7B. In some embodiment, the two targeting domains bind to EGFR and HER2, and the sdABD-TTAs have the sequences in FIGS. 5A, 5H-5N and 75 and the sequences provided therein and the corresponding sequence listing. In this embodiment, some combinations of the EGFR sdABD and the HER2 sdABD include:

| | | | | | |
|---|---|---|---|---|---|
| Cross | EGFR1 | hEGFR1 | EGFR2 | EGFR2a | EGFR2d |
| aHER2 1054 | In either orientation | In either orientation | In either orientation | In either orientation | In either orientation |
| aHER2 1055 | In either orientation | In either orientation | In either orientation | In either orientation | In either orientation |
| aHER2 1058 | In either orientation | In either orientation | In either orientation | In either orientation | In either orientation |
| aHER2 1059 | In either orientation | In either orientation | In either orientation | In either orientation | In either orientation |
| aHER2 1065 | In either orientation | In either orientation | In either orientation | In either orientation | In either orientation |
| aHER2 1090 | In either orientation | In either orientation | In either orientation | In either orientation | In either orientation |
| aHER2 1091 | In either orientation | In either orientation | In either orientation | In either orientation | In either orientation |
| aHER2 1092 | In either orientation | In either orientation | In either orientation | In either orientation | In either orientation |
| aHER2 1097 | In either orientation | In either orientation | In either orientation | In either orientation | In either orientation |
| aHER2 1118 | In either orientation | In either orientation | In either orientation | In either orientation | In either orientation |
| aHER2 1121 | In either orientation | In either orientation | In either orientation | In either orientation | In either orientation |
| aHER2 1134 | In either orientation | In either orientation | In either orientation | In either orientation | In either orientation |
| aHER2 1138 | In either orientation | In either orientation | In either orientation | In either orientation | In either orientation |
| aHER2 1139 | In either orientation | In either orientation | In either orientation | In either orientation | In either orientation |
| aHER2 1140 | In either orientation | In either orientation | In either orientation | In either orientation | In either orientation |
| aHER2 1145 | In either orientation | In either orientation | In either orientation | In either orientation | In either orientation |
| aHER2 1146 | In either orientation | In either orientation | In either orientation | In either orientation | In either orientation |
| aHER2 1149 | In either orientation | In either orientation | In either orientation | In either orientation | In either orientation |
| aHER2 1150 | In either orientation | In either orientation | In either orientation | In either orientation | In either orientation |
| aHER2 1156 | In either orientation | In either orientation | In either orientation | In either orientation | In either orientation |
| aHER2 1158 | In either orientation | In either orientation | In either orientation | In either orientation | In either orientation |
| aHER2 1159 | In either orientation | In either orientation | In either orientation | In either orientation | In either orientation |
| aHER2 1160 | In either orientation | In either orientation | In either orientation | In either orientation | In either orientation |
| aHER2 1161 | In either orientation | In either orientation | In either orientation | In either orientation | In either orientation |
| aHER2 1162 | In either orientation | In either orientation | In either orientation | In either orientation | In either orientation |
| aHER2 1163 | In either orientation | In either orientation | In either orientation | In either orientation | In either orientation |
| aHER2 h1139 | In either orientation | In either orientation | In either orientation | In either orientation | In either orientation |
| aHER2 h1156 | In either orientation | In either orientation | In either orientation | In either orientation | In either orientation |
| aHER2 h1159 | In either orientation | In either orientation | In either orientation | In either orientation | In either orientation |
| aHER2 h1162 | In either orientation | In either orientation | In either orientation | In either orientation | In either orientation |
| aHER2 h1058 | In either orientation | In either orientation | In either orientation | In either orientation | In either orientation |
| aHER2 h074 | In either orientation | In either orientation | In either orientation | In either orientation | In either orientation |

In this case, “either orientation” means that either the HER2 sdABD is N-terminal to the EGFR sdABD in the constructs of the invention or C-terminal to it.

### G. EpCAM X FOLR1

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiments, the aVH, aVL, iVH, iVL have the sequences shown in FIGS. 7A-7B. In some embodiments, the two targeting domains bind to EpCAM and FOLR1, and the sdABD-TTAs have the sequences in FIGS. 5B, 5D, 5E, and 75 and the sequences provided therein and the corresponding sequence listing. In this embodiment, some combinations of the EpCAM sdABDs and the FOLR1 sdABDs include:

| | | | |
|---|---|---|---|
| cross | FOLR1h77-2 | FOLR1 h59.3 | FOLR h22-4 |
| EpCAM h13 | In either orientation | In either orientation | In either orientation |
| EpCAM h23 | In either orientation | In either orientation | In either orientation |
| EpCAM hVIB665 | In either orientation | In either orientation | In either orientation |
| EpCAM hVIB666 | In either orientation | In either orientation | In either orientation |
| EpCAM h664 | In either orientation | In either orientation | In either orientation |

In this case, “either orientation” means that either the EpCAM sdABD is N-terminal to the FOLR1 sdABD in the constructs of the invention or the EpCAM sdABD is C-terminal to it.

### H. EpCAM X B7H3

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiments, the aVH, aVL, iVH, iVL have the sequences shown in FIGS. 7A-7B. In some embodiments, the two targeting domains bind to EpCAM and B7H3, and the sdABD-TTAs have the sequences in FIGS. 5B-5E and 75 and the sequences provided therein and the corresponding sequence listing. In this embodiment, some combinations of the EpCAM sdABDs and the B7H3 sdABDs include:

| | | | | | |
|---|---|---|---|---|---|
| cross | EpCAM h13 | EpCAM h23 | EpCAM hVIB665 | EpCAM hVIB666 | EpCAM h664 |
| B7H3 hF7 | In either orientation | In either orientation | In either orientation | In either orientation | In either orientation |
| B7H3 hF12 | In either orientation | In either orientation | In either orientation | In either orientation | In either orientation |
| B7H3 hF12 (N57Q) | In either orientation | In either orientation | In either orientation | In either orientation | In either orientation |
| B7H3 hF12 (N57E) | In either orientation | In either orientation | In either orientation | In either orientation | In either orientation |
| B7H3 hF12 (N57D) | In either orientation | In either orientation | In either orientation | In either orientation | In either orientation |
| B7H3 hF12 (S59A) | In either orientation | In either orientation | In either orientation | In either orientation | In either orientation |
| B7H3 hF12 (S59Y) | In either orientation | In either orientation | In either orientation | In either orientation | In either orientation |

In this case, “either orientation” means that either the B7H3 sdABD is N-terminal to the EpCAM sdABD in the constructs of the invention or the B7H3 sdABD is C-terminal to it.

### I. EpCAM X Trop2

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiments, the aVH, aVL, iVH, iVL have the sequences shown in FIGS. 7A-7B. In some embodiments, the two targeting domains bind to EpCAM and Trop2, and the sdABD-TTAs have the sequences in FIGS. 5D, 5E, 5F, and 75 and the sequences provided therein and the corresponding sequence listing. In this embodiment, preferred combinations of the EpCAM sdABDs and the Trop2 sdABDs include:

| | | | | | |
|---|---|---|---|---|---|
| cross | EpCAM h13 | EpCAM h23 | EpCAM hVIB665 | EpCAM hVIB666 | EpCAM h664 |
| aTrop2 hVIB557 | In either orientation | In either orientation | In either orientation | In either orientation | In either orientation |
| aTrop2 hVIB565 | In either orientation | In either orientation | In either orientation | In either orientation | In either orientation |
| aTrop2 hVIB575 | In either orientation | In either orientation | In either orientation | In either orientation | In either orientation |
| aTrop2 hVIB578 | In either orientation | In either orientation | In either orientation | In either orientation | In either orientation |
| aTrop2 hVIB609 | In either orientation | In either orientation | In either orientation | In either orientation | In either orientation |
| aTrop2 hVIB619 | In either orientation | In either orientation | In either orientation | In either orientation | In either orientation |

In this case, “either orientation” which means that either the Trop2 sdABD is N-terminal to the EpCAM sdABD in the constructs of the invention or the Trop2 sdABD is C-terminal to it.

### J. EpCAM X LyPD3

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiments, the aVH, aVL, iVH, iVL have the sequences shown in FIGS. 7A-7B. In some embodiments, the two targeting domains bind to EpCAM and LyPD3, and the sdABD-TTAs have the sequences in FIGS. 5D, 5E, 5H and 75 and the sequences provided therein and the corresponding sequence listing. In this embodiment, some combinations of the LyPD3 sdABDs and the EpCAM sdABDs include:

| | | | | | |
|---|---|---|---|---|---|
| cross | EpCAM h13 | EpCAM h23 | EpCAM hVIB665 | EpCAM hVIB666 | EpCAM h664 |
| aLyPD3 h790 | In either orientation | In either orientation | In either orientation | In either orientation | In either orientation |
| aLyPD3 h804 | In either orientation | In either orientation | In either orientation | In either orientation | In either orientation |
| aLyPD3 h773 | In either orientation | In either orientation | In either orientation | In either orientation | In either orientation |
| aLyPD3 h840 | In either orientation | In either orientation | In either orientation | In either orientation | In either orientation |
| aLyPD3 h885 | In either orientation | In either orientation | In either orientation | In either orientation | In either orientation |
| aLyPD3 h787 | In either orientation | In either orientation | In either orientation | In either orientation | In either orientation |

In this case, “either orientation” which means that either the LyPD3 sdABD is N-terminal to the EpCAM sdABD in the constructs of the invention or C-terminal to it.

### K. EpCAM X HER2

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiments, the aVH, aVL, iVH, iVL have the sequences shown in FIGS. 7A-7B. In some embodiments, the two targeting domains bind to HER2 and EpCAM, and the sdABD-TTAs have the sequences in FIGS. 5D, 5E, 5H-5N, and 75 and the sequences provided therein and the corresponding sequence listing. In this embodiment, some combinations of the HER2 sdABDs and the EpCAM sdABDs include:

| | | | | | |
|---|---|---|---|---|---|
| cross | EpCAM h13 | EpCAM h23 | EpCAM hVIB665 | EpCAM hVIB666 | EpCAM h664 |
| aHER2 1054 | In either orientation | In either orientation | In either orientation | In either orientation | In either orientation |
| aHER2 1055 | In either orientation | In either orientation | In either orientation | In either orientation | In either orientation |
| aHER2 1058 | In either orientation | In either orientation | In either orientation | In either orientation | In either orientation |
| aHER2 1059 | In either orientation | In either orientation | In either orientation | In either orientation | In either orientation |
| aHER2 1065 | In either orientation | In either orientation | In either orientation | In either orientation | In either orientation |
| aHER2 1090 | In either orientation | In either orientation | In either orientation | In either orientation | In either orientation |
| aHER2 1091 | In either orientation | In either orientation | In either orientation | In either orientation | In either orientation |
| aHER2 1092 | In either orientation | In either orientation | In either orientation | In either orientation | In either orientation |
| aHER2 1097 | In either orientation | In either orientation | In either orientation | In either orientation | In either orientation |
| aHER2 1118 | In either orientation | In either orientation | In either orientation | In either orientation | In either orientation |
| aHER2 1121 | In either orientation | In either orientation | In either orientation | In either orientation | In either orientation |
| aHER2 1134 | In either orientation | In either orientation | In either orientation | In either orientation | In either orientation |
| aHER2 1138 | In either orientation | In either orientation | In either orientation | In either orientation | In either orientation |
| aHER2 1139 | In either orientation | In either orientation | In either orientation | In either orientation | In either orientation |
| aHER2 1140 | In either orientation | In either orientation | In either orientation | In either orientation | In either orientation |
| aHER2 1145 | In either orientation | In either orientation | In either orientation | In either orientation | In either orientation |
| aHER2 1146 | In either orientation | In either orientation | In either orientation | In either orientation | In either orientation |
| aHER2 1149 | In either orientation | In either orientation | In either orientation | In either orientation | In either orientation |
| aHER2 1150 | In either orientation | In either orientation | In either orientation | In either orientation | In either orientation |
| aHER2 1156 | In either orientation | In either orientation | In either orientation | In either orientation | In either orientation |
| aHER2 1158 | In either orientation | In either orientation | In either orientation | In either orientation | In either orientation |
| aHER2 1159 | In either orientation | In either orientation | In either orientation | In either orientation | In either orientation |
| aHER2 1160 | In either orientation | In either orientation | In either orientation | In either orientation | In either orientation |
| aHER2 1161 | In either orientation | In either orientation | In either orientation | In either orientation | In either orientation |
| aHER2 1162 | In either orientation | In either orientation | In either orientation | In either orientation | In either orientation |
| aHER2 1163 | In either orientation | In either orientation | In either orientation | In either orientation | In either orientation |
| aHER2 h1139 | In either orientation | In either orientation | In either orientation | In either orientation | In either orientation |
| aHER2 h1156 | In either orientation | In either orientation | In either orientation | In either orientation | In either orientation |
| aHER2 h1159 | In either orientation | In either orientation | In either orientation | In either orientation | In either orientation |
| aHER2 h1162 | In either orientation | In either orientation | In either orientation | In either orientation | In either orientation |
| aHER2 h1058 | In either orientation | In either orientation | In either orientation | In either orientation | In either orientation |
| aHER2 1074 | In either orientation | In either orientation | In either orientation | In either orientation | In either orientation |

In this case, “EO” is “either orientation” which means that either the LyPD3 sdABD is N-terminal to the EpCAM sdABD in the constructs of the invention or C-terminal to it.

### 1. FOLR1 X B7H3

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiments, the aVH, aVL, iVH, iVL have the sequences shown in FIGS. 7A-7B. In some embodiments, the two targeting domains bind to FOLR1 and B7H3, and the sdABD-TTAs have the sequences in FIGS. 5B-5D and the sequences provided therein and the corresponding sequence listing. In this embodiment, some combinations of the FOLR1 sdABDs and the B7H3 sdABDs include:

| | | | |
|---|---|---|---|
| cross | FOLR1h77-2 | FOLR1 h59.3 | FOLR h22-4 |
| B7H3 hF7 | In either orientation | In either orientation | In either orientation |
| B7H3 hF12 | In either orientation | In either orientation | In either orientation |
| B7H3 hF12 (N57Q) | In either orientation | In either orientation | In either orientation |
| B7H3 hF12 (N57E) | In either orientation | In either orientation | In either orientation |
| B7H3 hF12 (N57D) | In either orientation | In either orientation | In either orientation |
| B7H3 hF12 (S59A) | In either orientation | In either orientation | In either orientation |
| B7H3 hF12 (S59Y) | In either orientation | In either orientation | In either orientation |

In this case, “either orientation” means that either the B7H3 sdABD is N-terminal to the FOLR1 sdABD in the constructs of the invention or C-terminal to it.

### M. FOLR1 X HER2

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiments, the aVH, aVL, iVH, iVL have the sequences shown in FIGS. 7A-7B. In some embodiments, the two targeting domains bind to FOLR1 and HER2, and the sdABD-TTAs have the sequences in FIGS. 5B, 5H-5N, and 75 and the sequences provided therein and the corresponding sequence listing. In this embodiment, some combinations of the FOLR1 sdABDs and the HER2 sdABDs include:

| | | | |
|---|---|---|---|
| Cross | FOLR1h77-2 | FOLR1 h59.3 | FOLR h22-4 |
| aHER2 1054 | In either orientation | In either orientation | In either orientation |
| aHER2 1055 | In either orientation | In either orientation | In either orientation |
| aHER2 1058 | In either orientation | In either orientation | In either orientation |
| aHER2 1059 | In either orientation | In either orientation | In either orientation |
| aHER2 1065 | In either orientation | In either orientation | In either orientation |
| aHER2 1090 | In either orientation | In either orientation | In either orientation |
| aHER2 1091 | In either orientation | In either orientation | In either orientation |
| aHER2 1092 | In either orientation | In either orientation | In either orientation |
| aHER2 1097 | In either orientation | In either orientation | In either orientation |
| aHER2 1118 | In either orientation | In either orientation | In either orientation |
| aHER2 1121 | In either orientation | In either orientation | In either orientation |
| aHER2 1134 | In either orientation | In either orientation | In either orientation |
| aHER2 1138 | In either orientation | In either orientation | In either orientation |
| aHER2 1139 | In either orientation | In either orientation | In either orientation |
| aHER2 1140 | In either orientation | In either orientation | In either orientation |
| aHER2 1145 | In either orientation | In either orientation | In either orientation |
| aHER2 1146 | In either orientation | In either orientation | In either orientation |
| aHER2 1149 | In either orientation | In either orientation | In either orientation |
| aHER2 1150 | In either orientation | In either orientation | In either orientation |
| aHER2 1156 | In either orientation | In either orientation | In either orientation |
| aHER2 1158 | In either orientation | In either orientation | In either orientation |
| aHER2 1159 | In either orientation | In either orientation | In either orientation |
| aHER2 1160 | In either orientation | In either orientation | In either orientation |
| aHER2 1161 | In either orientation | In either orientation | In either orientation |
| aHER2 1162 | In either orientation | In either orientation | In either orientation |
| aHER2 1163 | In either orientation | In either orientation | In either orientation |
| aHER2 h1139 | In either orientation | In either orientation | In either orientation |
| aHER2 h1156 | In either orientation | In either orientation | In either orientation |
| aHER2 h1159 | In either orientation | In either orientation | In either orientation |
| aHER2 h1162 | In either orientation | In either orientation | In either orientation |
| aHER2 h1058 | In either orientation | In either orientation | In either orientation |
| aHER2 1074 | In either orientation | In either orientation | In either orientation |

In this case, “either orientation” means that either the HER2 sdABD is N-terminal to the FOLR1 sdABD in the constructs of the invention or the HER2 sdABD is C-terminal to it.

### N. FOLR1 X Trop2

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiments, the aVH, aVL, iVH, iVL have the sequences shown in FIGS. 7A-7B. In some embodiments, the two targeting domains bind to FOLR1 and Trop2, and the sdABD-TTAs have the sequences in FIGS. 5B, 5E and 5F and the sequences provided therein and the corresponding sequence listing. In this embodiment, preferred combinations of the FOLR1 sdABDs and the Trop2 sdABDs include:

| | | | |
|---|---|---|---|
| cross | FOLR1h77-2 | FOLR1 h59.3 | FOLR h22-4 |
| aTrop2 hVIB557 | In either orientation | In either orientation | In either orientation |
| aTrop2 hVIB565 | In either orientation | In either orientation | In either orientation |
| aTrop2 hVIB575 | In either orientation | In either orientation | In either orientation |
| aTrop2 hVIB578 | In either orientation | In either orientation | In either orientation |
| aTrop2 hVIB609 | In either orientation | In either orientation | In either orientation |

In this case, “either orientation” means that either the Trop2 sdABD is N-terminal to the FOLR1 sdABD in the constructs of the invention or the Trop2 sdABD is C-terminal to it.

### O. FOLR1 X LyPD3

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiments, the aVH, aVL, iVH, iVL have the sequences shown in FIGS. 7A-7B. In some embodiments, the two targeting domains bind to FOLR1 and LyPD3, and the sdABD-TTAs have the sequences in FIGS. 5B, 5G and 5H and the sequences provided therein and the corresponding sequence listing. In this embodiment, some combinations of the FOLR1sdABDs and the LyPD3 sdABDs include:

| | | | |
|---|---|---|---|
| Cross | FOLR1h77-2 | FOLR1 h59.3 | FOLR h22-4 |
| aLyPD3 h790 | In either orientation | In either orientation | In either orientation |
| aLyPD3 h804 | In either orientation | In either orientation | In either orientation |
| aLyPD3 h773 | In either orientation | In either orientation | In either orientation |
| aLyPD3 h840 | In either orientation | In either orientation | In either orientation |
| aLyPD3 h885 | In either orientation | In either orientation | In either orientation |
| aLyPD3 h787 | In either orientation | In either orientation | In either orientation |

In this case, “either orientation” means that either the LyPD3 sdABD is N-terminal to the FOLR1 sdABD in the constructs of the invention or the LyPD3 sdABD is C-terminal to it.

### P. B7H3 X HER2

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiments, the aVH, aVL, iVH, iVL have the sequences shown in FIGS. 7A-7B. In some embodiments, the two targeting domains bind to B7H3 and HER2, and the sdABD-TTAs have the sequences in FIGS. 5C, 5D, 5H-5N, and 75 and the sequences provided therein and the corresponding sequence listing. In this embodiment, some combinations of the B7H3 sdABDs and the HER2 sdABDs include:

| | | | | | | | |
|---|---|---|---|---|---|---|---|
| Cross | B7H3 hF7 | B7H3 hF12 | B7H3 hF12 (N57Q) | B7H3 hF12 (N57E) | B7H3 hF12 (N57D) | B7H3 hF12 (S59A) | B7H3 hF12 (S59Y) |
| aHER2 054 | In either orientation | In either orientation | In either orientation | In either orientation | In either orientation | In either orientation | In either orientation |
| aHER2 1055 | In either orientation | In either orientation | In either orientation | In either orientation | In either orientation | In either orientation | In either orientation |
| aHER2 1058 | In either orientation | In either orientation | In either orientation | In either orientation | In either orientation | In either orientation | In either orientation |
| aHER2 1059 | In either orientation | In either orientation | In either orientation | In either orientation | In either orientation | In either orientation | In either orientation |
| aHER2 1065 | In either orientation | In either orientation | In either orientation | In either orientation | In either orientation | In either orientation | In either orientation |
| aHER2 1090 | In either orientation | In either orientation | In either orientation | In either orientation | In either orientation | In either orientation | In either orientation |
| aHER2 1091 | In either orientation | In either orientation | In either orientation | In either orientation | In either orientation | In either orientation | In either orientation |
| aHER2 1092 | In either orientation | In either orientation | In either orientation | In either orientation | In either orientation | In either orientation | In either orientation |
| aHER2 1097 | In either orientation | In either orientation | In either orientation | In either orientation | In either orientation | In either orientation | In either orientation |
| aHER2 1118 | In either orientation | In either orientation | In either orientation | In either orientation | In either orientation | In either orientation | In either orientation |
| aHER2 1121 | In either orientation | In either orientation | In either orientation | In either orientation | In either orientation | In either orientation | In either orientation |
| aHER2 1134 | In either orientation | In either orientation | In either orientation | In either orientation | In either orientation | In either orientation | In either orientation |
| aHER2 1138 | In either orientation | In either orientation | In either orientation | In either orientation | In either orientation | In either orientation | In either orientation |
| aHER2 1139 | In either orientation | In either orientation | In either orientation | In either orientation | In either orientation | In either orientation | In either orientation |
| aHER2 1140 | In either orientation | In either orientation | In either orientation | In either orientation | In either orientation | In either orientation | In either orientation |
| aHER2 1145 | In either orientation | In either orientation | In either orientation | In either orientation | In either orientation | In either orientation | In either orientation |
| aHER2 1146 | In either orientation | In either orientation | In either orientation | In either orientation | In either orientation | In either orientation | In either orientation |
| aHER2 1149 | In either orientation | In either orientation | In either orientation | In either orientation | In either orientation | In either orientation | In either orientation |
| aHER2 1150 | In either orientation | In either orientation | In either orientation | In either orientation | In either orientation | In either orientation | In either orientation |
| aHER2 1156 | In either orientation | In either orientation | In either orientation | In either orientation | In either orientation | In either orientation | In either orientation |
| aHER2 1158 | In either orientation | In either orientation | In either orientation | In either orientation | In either orientation | In either orientation | In either orientation |
| aHER2 1159 | In either orientation | In either orientation | In either orientation | In either orientation | In either orientation | In either orientation | In either orientation |
| aHER2 1160 | In either orientation | In either orientation | In either orientation | In either orientation | In either orientation | In either orientation | In either orientation |
| aHER2 1161 | In either orientation | In either orientation | In either orientation | In either orientation | In either orientation | In either orientation | In either orientation |
| aHER2 1162 | In either orientation | In either orientation | In either orientation | In either orientation | In either orientation | In either orientation | In either orientation |
| aHER2 1163 | In either orientation | In either orientation | In either orientation | In either orientation | In either orientation | In either orientation | In either orientation |
| aHER2 h1139 | In either orientation | In either orientation | In either orientation | In either orientation | In either orientation | In either orientation | In either orientation |
| aHER2 h1156 | In either orientation | In either orientation | In either orientation | In either orientation | In either orientation | In either orientation | In either orientation |
| aHER2 h1159 | In either orientation | In either orientation | In either orientation | In either orientation | In either orientation | In either orientation | In either orientation |
| aHER2 h1162 | In either orientation | In either orientation | In either orientation | In either orientation | In either orientation | In either orientation | In either orientation |
| aHER2 h1058 | In either orientation | In either orientation | In either orientation | In either orientation | In either orientation | In either orientation | In either orientation |
| aHER2 1074 | In either orientation | In either orientation | In either orientation | In either orientation | In either orientation | In either orientation | In either orientation |

In this case, “either orientation” means that either the HER2 sdABD is N-terminal to the B7H3 sdABD in the constructs of the invention or the HER2 sdABD is C-terminal to it.

### Q. B7H3 X Trop2

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiment, the aVH, aVL, iVH, iVL have the sequences shown in FIGS. 7A-7B. In some embodiment, the two targeting domains bind to B7H3 and Trop2, and the sdABD-TTAs have the sequences in FIGS. 5B-5F and the sequences provided therein and the corresponding sequence listing. In this embodiment, some combinations of the B7H3 sdABDs and the Trop2 sdABDs include:

| | | | | | | |
|---|---|---|---|---|---|---|
| Cross | aTrop2 hVIB557 | aTrop2 hVIB565 | aTrop2 hVIB575 | aTrop2 hVIB578 | aTrop2 hVIB609 | aTrop2 hVIB619 |
| B7H3 hF7 | In either orientation | In either orientation | In either orientation | In either orientation | In either orientation | In either orientation |
| B7H3 hF12 | In either orientation | In either orientation | In either orientation | In either orientation | In either orientation | In either orientation |
| B7H3 hF12 (N57Q) | In either orientation | In either orientation | In either orientation | In either orientation | In either orientation | In either orientation |
| B7H3 hF12 (N57E) | In either orientation | In either orientation | In either orientation | In either orientation | In either orientation | In either orientation |
| B7H3 hF12 (N57D) | In either orientation | In either orientation | In either orientation | In either orientation | In either orientation | In either orientation |
| B7H3 hF12 (S59A) | In either orientation | In either orientation | In either orientation | In either orientation | In either orientation | In either orientation |
| B7H3 hF12 (S59Y) | In either orientation | In either orientation | In either orientation | In either orientation | In either orientation | In either orientation |

In this case, “either orientation” means that either the Trop2 sdABD is N-terminal to the B7H3 sdABD in the constructs of the invention or the Trop2 sdABD is C-terminal to it.

### R. B7H3 X LyPD3

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiments, the aVH, aVL, iVH, iVL have the sequences shown in FIGS. 7A-7B. In some embodiment, the two targeting domains bind to B7H3 and LyPD3, and the sdABD-TTAs have the sequences in FIGS. 5B-5D, 5G, and 5H and the sequences provided therein and the corresponding sequence listing. In this embodiment, some combinations of the B7H3 sdABDs and the LyPD3 sdABDs include:

| | | | | | | |
|---|---|---|---|---|---|---|
| Cross | aLyPD3 h787 | aLyPD3 h790 | aLyPD3 h804 | aLyPD3 h773 | aLyPD3 h840 | aLyPD3 h885 |
| B7H3 hF7 | In either orientation | In either orientation | In either orientation | In either orientation | In either orientation | In either orientation |
| B7H3 hF12 | In either orientation | In either orientation | In either orientation | In either orientation | In either orientation | In either orientation |
| B7H3 hF12 (N57Q) | In either orientation | In either orientation | In either orientation | In either orientation | In either orientation | In either orientation |
| B7H3 hF12 (N57E) | In either orientation | In either orientation | In either orientation | In either orientation | In either orientation | In either orientation |
| B7H3 hF12 (N57D) | In either orientation | In either orientation | In either orientation | In either orientation | In either orientation | In either orientation |
| B7H3 hF12 (S59A) | In either orientation | In either orientation | In either orientation | In either orientation | In either orientation | In either orientation |
| B7H3 hF12 (S59Y) | In either orientation | In either orientation | In either orientation | In either orientation | In either orientation | In either orientation |

In this case, “either orientation” means that either the LyPD3 sdABD is N-terminal to the B7H3 sdABD in the constructs of the invention or the LyPD3 sdABD is C-terminal to it.

### S. HER2 X Trop2

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiment, the aVH, aVL, iVH, iVL have the sequences shown in FIGS. 7A-7B. In some embodiment, the two targeting domains bind to HER2 and Trop2, and the sdABD-TTAs have the sequences in FIGS. 5E, 5F, 5H-5N, and 75 and the sequences provided therein and the corresponding sequence listing. In this embodiment, some combinations of the HER2 sdABDs and the Trop2 sdABDs include.

| | | | | | | |
|---|---|---|---|---|---|---|
| Cross | aTrop2 hVIB557 | aTrop2 hVIB565 | aTrop2 hVIB575 | aTrop2 hVIB578 | aTrop2 hVIB609 | aTrop2 hVIB619 |
| aHER2 1054 | In either orientation | In either orientation | In either orientation | In either orientation | In either orientation | In either orientation |
| aHER2 1055 | In either orientation | In either orientation | In either orientation | In either orientation | In either orientation | In either orientation |
| aHER2 1058 | In either orientation | In either orientation | In either orientation | In either orientation | In either orientation | In either orientation |
| aHER2 1059 | In either orientation | In either orientation | In either orientation | In either orientation | In either orientation | In either orientation |
| aHER2 1065 | In either orientation | In either orientation | In either orientation | In either orientation | In either orientation | In either orientation |
| aHER2 1090 | In either orientation | In either orientation | In either orientation | In either orientation | In either orientation | In either orientation |
| aHER2 1091 | In either orientation | In either orientation | In either orientation | In either orientation | In either orientation | In either orientation |
| aHER2 1092 | In either orientation | In either orientation | In either orientation | In either orientation | In either orientation | In either orientation |
| aHER2 1097 | In either orientation | In either orientation | In either orientation | In either orientation | In either orientation | In either orientation |
| aHER2 1118 | In either orientation | In either orientation | In either orientation | In either orientation | In either orientation | In either orientation |
| aHER2 1121 | In either orientation | In either orientation | In either orientation | In either orientation | In either orientation | In either orientation |
| aHER2 1134 | In either orientation | In either orientation | In either orientation | In either orientation | In either orientation | In either orientation |
| aHER2 1138 | In either orientation | In either orientation | In either orientation | In either orientation | In either orientation | In either orientation |
| aHER2 1139 | In either orientation | In either orientation | In either orientation | In either orientation | In either orientation | In either orientation |
| aHER2 1140 | In either orientation | In either orientation | In either orientation | In either orientation | In either orientation | In either orientation |
| aHER2 1145 | In either orientation | In either orientation | In either orientation | In either orientation | In either orientation | In either orientation |
| aHER2 1146 | In either orientation | In either orientation | In either orientation | In either orientation | In either orientation | In either orientation |
| aHER2 1149 | In either orientation | In either orientation | In either orientation | In either orientation | In either orientation | In either orientation |
| aHER2 1150 | In either orientation | In either orientation | In either orientation | In either orientation | In either orientation | In either orientation |
| aHER2 1156 | In either orientation | In either orientation | In either orientation | In either orientation | In either orientation | In either orientation |
| aHER2 1158 | In either orientation | In either orientation | In either orientation | In either orientation | In either orientation | In either orientation |
| aHER2 1159 | In either orientation | In either orientation | In either orientation | In either orientation | In either orientation | In either orientation |
| aHER2 1160 | In either orientation | In either orientation | In either orientation | In either orientation | In either orientation | In either orientation |
| aHER2 1161 | In either orientation | In either orientation | In either orientation | In either orientation | In either orientation | In either orientation |
| aHER2 1162 | In either orientation | In either orientation | In either orientation | In either orientation | In either orientation | In either orientation |
| aHER2 1163 | In either orientation | In either orientation | In either orientation | In either orientation | In either orientation | In either orientation |
| aHER2 h1139 | In either orientation | In either orientation | In either orientation | In either orientation | In either orientation | In either orientation |
| aHER2 h1156 | In either orientation | In either orientation | In either orientation | In either orientation | In either orientation | In either orientation |
| aHER2 h1159 | In either orientation | In either orientation | In either orientation | In either orientation | In either orientation | In either orientation |
| aHER2 h1162 | In either orientation | In either orientation | In either orientation | In either orientation | In either orientation | In either orientation |
| aHER2 h1058 | In either orientation | In either orientation | In either orientation | In either orientation | In either orientation | In either orientation |
| aHER2 1074 | In either orientation | In either orientation | In either orientation | In either orientation | In either orientation | In either orientation |

In this case, “either orientation” means that either the HER2 sdABD is N-terminal to the Trop2 sdABD in the constructs of the invention or the HER2 sdABD is C-terminal to it.

### T. HER2 X LyPD3

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiments, the aVH, aVL, iVH, iVL have the sequences shown in FIGS. 7A-7B. In some embodiments, the two targeting domains bind to HER2 and LyPD3, and the sdABD-TTAs have the sequences in FIGS. 5G, 5H-5N and 75 and the sequences provided therein and the corresponding sequence listing. In this embodiment, some combinations of the HER2 sdABDs and the LyPD3 sdABDs include:

| | | | | | | |
|---|---|---|---|---|---|---|
| cross | aLyPD3 h790 | aLyPD3 h790 | aLyPD3 h790 | aLyPD3 h790 | aLyPD3 h790 | aLyPD3 h790 |
| aHER2 1054 | In either orientation | In either orientation | In either orientation | In either orientation | In either orientation | In either orientation |
| aHER2 1055 | In either orientation | In either orientation | In either orientation | In either orientation | In either orientation | In either orientation |
| aHER2 1058 | In either orientation | In either orientation | In either orientation | In either orientation | In either orientation | In either orientation |
| aHER2 1059 | In either orientation | In either orientation | In either orientation | In either orientation | In either orientation | In either orientation |
| aHER2 1065 | In either orientation | In either orientation | In either orientation | In either orientation | In either orientation | In either orientation |
| aHER2 1090 | In either orientation | In either orientation | In either orientation | In either orientation | In either orientation | In either orientation |
| aHER2 1091 | In either orientation | In either orientation | In either orientation | In either orientation | In either orientation | In either orientation |
| aHER2 1092 | In either orientation | In either orientation | In either orientation | In either orientation | In either orientation | In either orientation |
| aHER2 1097 | In either orientation | In either orientation | In either orientation | In either orientation | In either orientation | In either orientation |
| aHER2 1118 | In either orientation | In either orientation | In either orientation | In either orientation | In either orientation | In either orientation |
| aHER2 1121 | In either orientation | In either orientation | In either orientation | In either orientation | In either orientation | In either orientation |
| aHER2 1134 | In either orientation | In either orientation | In either orientation | In either orientation | In either orientation | In either orientation |
| aHER2 1138 | In either orientation | In either orientation | In either orientation | In either orientation | In either orientation | In either orientation |
| aHER2 1139 | In either orientation | In either orientation | In either orientation | In either orientation | In either orientation | In either orientation |
| aHER2 1140 | In either orientation | In either orientation | In either orientation | In either orientation | In either orientation | In either orientation |
| aHER2 1145 | In either orientation | In either orientation | In either orientation | In either orientation | In either orientation | In either orientation |
| aHER2 1146 | In either orientation | In either orientation | In either orientation | In either orientation | In either orientation | In either orientation |
| aHER2 1149 | In either orientation | In either orientation | In either orientation | In either orientation | In either orientation | In either orientation |
| aHER2 1150 | In either orientation | In either orientation | In either orientation | In either orientation | In either orientation | In either orientation |
| aHER2 1156 | In either orientation | In either orientation | In either orientation | In either orientation | In either orientation | In either orientation |
| aHER2 1158 | In either orientation | In either orientation | In either orientation | In either orientation | In either orientation | In either orientation |
| aHER2 1159 | In either orientation | In either orientation | In either orientation | In either orientation | In either orientation | In either orientation |
| aHER2 1160 | In either orientation | In either orientation | In either orientation | In either orientation | In either orientation | In either orientation |
| aHER2 1161 | In either orientation | In either orientation | In either orientation | In either orientation | In either orientation | In either orientation |
| aHER2 1162 | In either orientation | In either orientation | In either orientation | In either orientation | In either orientation | In either orientation |
| aHER2 1163 | In either orientation | In either orientation | In either orientation | In either orientation | In either orientation | In either orientation |
| aHER2 h1139 | In either orientation | In either orientation | In either orientation | In either orientation | In either orientation | In either orientation |
| aHER2 h1156 | In either orientation | In either orientation | In either orientation | In either orientation | In either orientation | In either orientation |
| aHER2 h1159 | In either orientation | In either orientation | In either orientation | In either orientation | In either orientation | In either orientation |
| aHER2 h1162 | In either orientation | In either orientation | In either orientation | In either orientation | In either orientation | In either orientation |
| aHER2 h1058 | In either orientation | In either orientation | In either orientation | In either orientation | In either orientation | In either orientation |
| aHER2 1074 | In either orientation | In either orientation | In either orientation | In either orientation | In either orientation | In either orientation |

In this case, “either orientation” means that either the HER2 sdABD is N-terminal to the Trop2 sdABD in the constructs of the invention or C-terminal to it.

### U. Trop2 X LyPD3

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In some embodiments, the aVH, aVL, iVH, iVL have the sequences shown in FIGS. 7A-7B. In some embodiments, the two targeting domains bind to Trop2 and LyPD3, and the sdABD-TTAs have the sequences in FIGS. 5E-5G and 5H and the sequences provided therein and the corresponding sequence listing. In this embodiment, some combinations of the Trop2 sdABDs and the LyPD3 sdABDs include:

| | | | | | | |
|---|---|---|---|---|---|---|
| cross | aTrop2 hVIB557 | aTrop2 hVIB565 | aTrop2 hVIB575 | aTrop2 hVIB578 | aTrop2 hVIB609 | aTrop2 hVIB619 |
| aLyPD3 h787 | In either orientation | In either orientation | In either orientation | In either orientation | In either orientation | In either orientation |
| aLyPD3 h790 | In either orientation | In either orientation | In either orientation | In either orientation | In either orientation | In either orientation |
| aLyPD3 h804 | In either orientation | In either orientation | In either orientation | In either orientation | In either orientation | In either orientation |
| aLyPD3 h773 | In either orientation | In either orientation | In either orientation | In either orientation | In either orientation | In either orientation |
| aLyPD3 h840 | In either orientation | In either orientation | In either orientation | In either orientation | In either orientation | In either orientation |
| aLyPD3 h885 | In either orientation | In either orientation | In either orientation | In either orientation | In either orientation | In either orientation |

In this case, “either orientation” means that either the Trop2 sdABD is N-terminal to the LyPD3 sdABD in the constructs of the invention or the Trop2 sdABD is C-terminal to it.

In some embodiments, the prodrug protein comprises, from N- to C-terminal: (sdABD-TTA1)-domain linker-aVH-CNCL-aVL-domain linker-(sdABD-TTA2)-CL-iVL-CNCL-iVH-domain linker-(sdABD-HSA). In this embodiment, the aVH, aVL, iVH, iVL have the sequences shown in FIGS. 7A-7B. In some embodiments, the two targeting domains bind to the same TTA, which can be EGFR, FOLR1, B7H3, CA9, Trop2, LyPD3, HER2 or EpCAM, the sequences for which are depicted in FIGS. 5A-M and 75, and the CCL and CL is selected from a linker that is cleaved by MMP9 or meprin, and the sdABD(½) has SEQ ID NO:249.

In Format 2, a preferred domain linker is SEQ ID NO:287 (which also serves as a preferred constrained non cleavable linker).

### B. Cleavable Formats With Dual Targeting

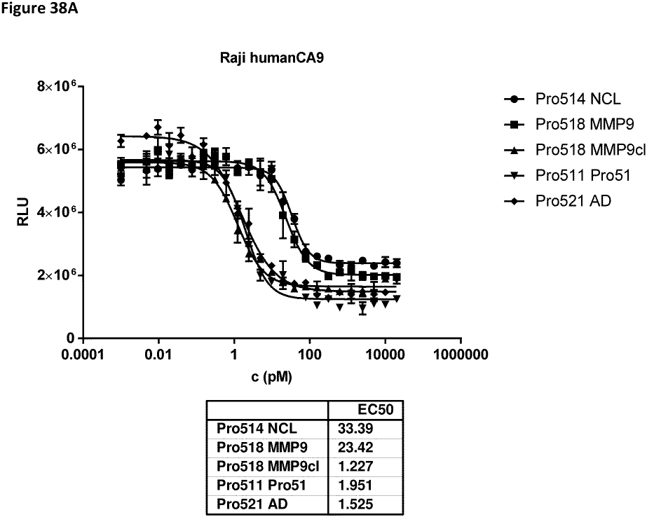

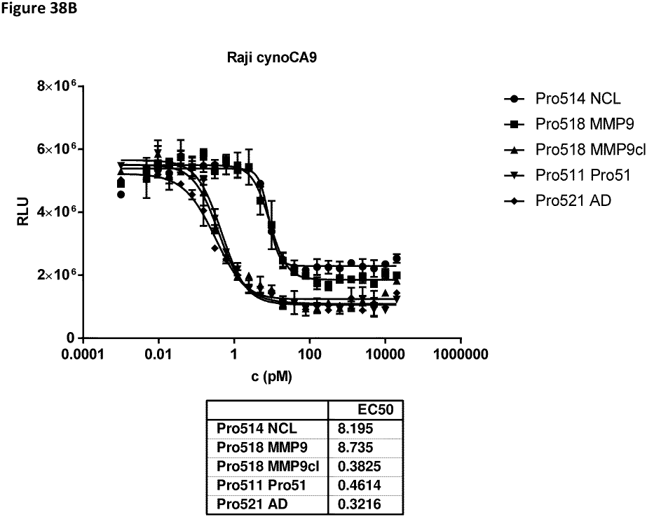

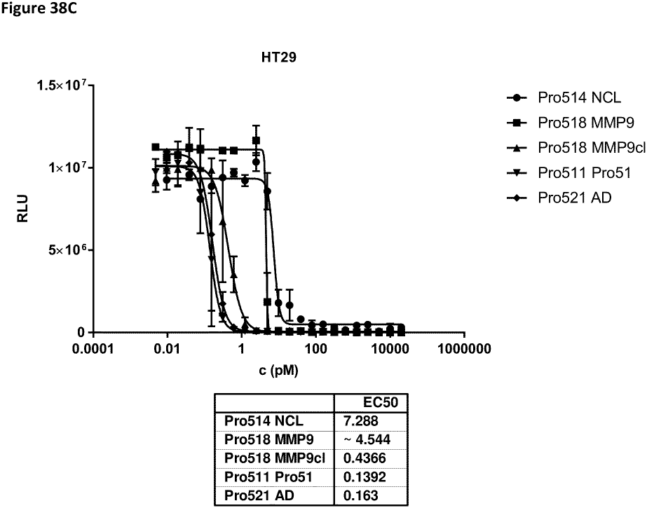

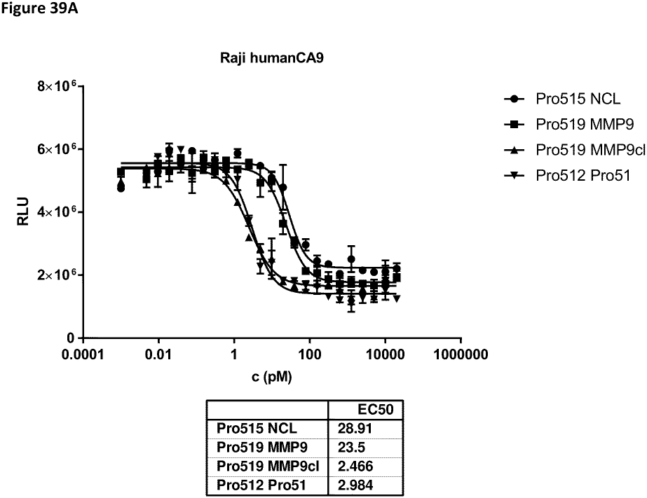

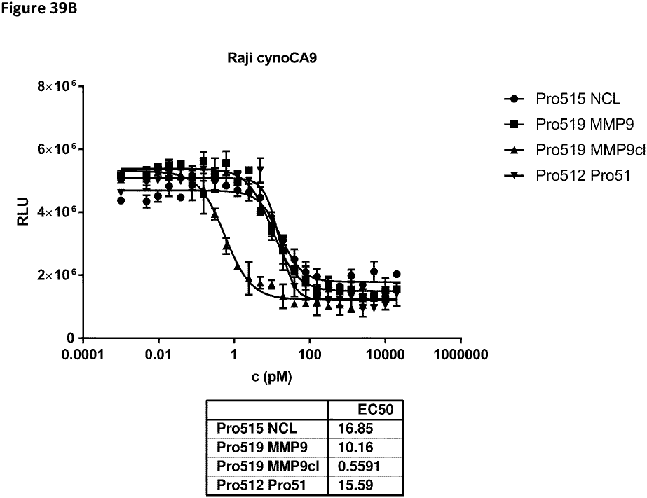

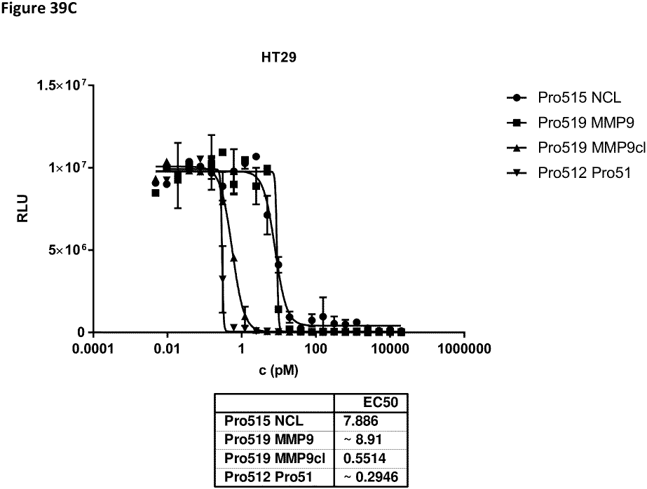

The invention provides non-isomerizing cleavable formats of the “format 1” type in FIG. 1. In this embodiment, the constrained Fv domain comprise VH and VL domains that are linked using constrained cleavable linkers and the constrained pseudo Fv domain uses constrained non-cleavable linkers. For ease of discussion, both of these are referred to herein as “constrained”, but as discussed above and shown in Figure 37, Figure 38 and Figure 39 of WO2019/051102, only one of these needs to be constrained, although generally, when both linkers are constrained, the protein has better expression.

All constructs in Format 1 (as well as the other formats) also have a cleavable linker (CL) that is cleaved by a human tumor protease.

The invention provides prodrug proteins, comprising, from N- to C-terminal, (sdABD-TTA1)-domain linker-constrained Fv domain-domain linker-(sdABD-TTA2)-CL-constrained pseudo Fv domain-domain linker-(sdABD-HSA).

As will be appreciated by those in the art, the order of the VH and VL in either a constrained Fv domain or a constrained pseudo Fv domain can be either (N- to C-terminal) VH-linker-VL or VL-linker-VH.

Thus, in one embodiment, the prodrug protein comprises, from N- to C-terminal: (sdABD-TTA1)-domain linker-aVH-CCL-aVL-domain linker-(sdABD-TTA2)-CL-iVL-CNCL-iVH-domain linker-(sdABD-HSA).

Thus, in one embodiment, the prodrug protein comprises, from N- to C-terminal: (sdABD-TTA1)-domain linker-aVH-CCL-aVL-domain linker-(sdABD-TTA2)-CL-iVH-CCL-iVL-domain linker-sdABD-HSA.

Thus, in one embodiment, the prodrug protein comprises, from N- to C-terminal: (sdABD-TTA1)-domain linker-aVL-CCL-aVH-domain linker-(sdABD-TTA2)-CL-iVL-CCL-iVH-domain linker-(sdABD-HSA).

Thus, in one embodiment, the prodrug protein comprises, from N- to C-terminal: (sdABD-TTA1)-domain linker-aVL-CCL-aVH-domain linker-(sdABD-TTA2)-CL-iVH-CCL-iVL-domain linker-(sdABD-HSA).

In some embodiments, the prodrug construct comprises sdABD(TTA1)-domain linker-aVH-CCL-aVL-domain linker-sdABD(TTA2)-CL-iVL-CNCL-iVH-NCL-sdABD(½). In this embodiment, the aVH, aVL, iVH and iVL have the sequences shown in FIGS. 7A-7B.

In some embodiments, the prodrug construct comprises sdABD(TTA1)-domain linker-aVH-CCL-aVL-domain linker-sdABD(TTA2)-CL-iVL-CNCL-iVH-domain linker-sdABD(½). In this embodiment, the aVH, aVL, iVH, iVL have the sequences shown in FIGS. 7A-7B. In this embodiment, the two targeting domains bind to the same TTA, which can be EGFR, EpCAM, FOLR1, Trop2, CA9, LyPD3, HER2 or B7H3, the sequences for which are depicted in FIGS. 5A-5N and 75.

In some embodiments, the prodrug construct comprises sdABD(TTA1)-domain linker-aVH-CCL-aVL-domain linker-sdABD(TTA2)-CL-iVL-CNCL-iVH-domain linker-sdABD(½). In this embodiment, the aVH, aVL, iVH, iVL have the sequences shown in FIGS. 7A-7B. In this embodiment, the two targeting domains bind to different TTAs.

In some embodiments, the prodrug construct comprises sdABD(TTA1)-domain linker-aVH-CCL-aVL-domain linker-sdABD(TTA2)-CL-iVL-CNCL-iVH-domain linker-sdABD(½). In this embodiment, the aVH, aVL, iVH, iVL have the sequences shown in FIGS. 7A-7B. In this embodiment, the two targeting domains bind to EGFR and EpCAM, and the sdABD-TTAs have the sequences in FIGS. 5A-5N and 75.

In some embodiments, the prodrug construct comprises sdABD(TTA1)-domain linker-aVH-CCL-aVL-domain linker-sdABD(TTA2)-CL-iVL-CNCL-iVH-domain linker-sdABD(½). In this embodiment, the aVH, aVL, iVH, iVL have the sequences shown in FIGS. 7A-7B. In this embodiment, the two targeting domains bind to EGFR and FOLR1, and the sdABD-TTAs have the sequences in FIGS. 5A-5N and 75.

In some embodiments, the prodrug construct comprises sdABD(TTA1)-domain linker-aVH-CCL-aVL-domain linker-sdABD(TTA2)-CL-iVL-CNCL-iVH-domain linker-sdABD(½). In this embodiment, the aVH, aVL, iVH, iVL have the sequences shown in FIGS. 7A-7B. In this embodiment, the two targeting domains bind to EGFR and B7H3, and the sdABD-TTAs have the sequences in FIGS. 5A-5N and 75.

In some embodiments, the prodrug construct comprises sdABD(TTA1)-domain linker-aVH-CCL-aVL-domain linker-sdABD(TTA2)-CL-iVL-CNCL-iVH-domain linker-sdABD(½). In this embodiment, the aVH, aVL, iVH, iVL have the sequences shown in FIGS. 7A-7B. In this embodiment, the two targeting domains bind to EpCAM and FOLR1, and the sdABD-TTAs have the sequences in FIGS. 5A-5N and 75.

In some embodiments, the prodrug construct comprises sdABD(TTA1)-domain linker-aVH-CCL-aVL-domain linker-sdABD(TTA2)-CL-iVL-CNCL-iVH-domain linker-sdABD(½). In this embodiment, the aVH, aVL, iVH, iVL have the sequences shown in FIGS. 7A-7B. In this embodiment, the two targeting domains bind to EpCAM and B7H3, and the sdABD-TTAs have the sequences in FIGS. 5A-5N and 75.

In some embodiments, the prodrug construct comprises sdABD(TTA1)-domain linker-aVH-CCL-aVL-domain linker-sdABD(TTA2)-CL-iVL-CNCL-iVH-domain linker-sdABD(½). In this embodiment, the aVH, aVL, iVH, iVL have the sequences shown in FIGS. 7A-7B. In this embodiment, the two targeting domains bind to B7H3 and FOLR1, and the sdABD-TTAs have the sequences in FIGS. 5A-5N and 75.

In some embodiments, the prodrug construct comprises sdABD(TTA1)-domain linker-aVH-CCL-aVL-domain linker-sdABD(TTA2)-CL-iVL-CNCL-iVH-domain linker-sdABD(½). In this embodiment, the aVH, aVL, iVH, iVL have the sequences shown in FIG. 7. In this embodiment, the two targeting domains bind to the same TTA, which can be EGFR, FOLR1, B7H3, Trop2, CA9, LyPD3, HER2 or EpCAM, the sequences for which are depicted in FIG. 5, and the CCL and CL is selected from a linker that is cleaved by MMP9 or meprin, and the sdABD(½) has SEQ ID NO:245 or SEQ ID NO:249.

In Format 1, a preferred domain linker is SEQ ID NO:287 (which also serves as a preferred constrained non cleavable linker).

### C. Single TTA Constructs

As is shown in FIG. 4, “format 4” constructs are also included in the compositions of the invention, that are similar to Format 2 constructs but without a second TTA ABD. In this embodiment, it is understood that the “non-cleavable” applies only to the linkage of the constrained Fv domain, as there is the activating cleavage site in the prodrug construct. In this embodiment, the constrained Fv domain comprise VH and VL domains that are linked using constrained non-cleavable linkers and the constrained pseudo Fv domain uses constrained non-cleavable linkers.

As will be appreciated by those in the art, the order of the VH and VL in either a constrained Fv domain or a constrained pseudo Fv domain can be either (N- to C-terminal) VH-linker-VL or VL-linker-VH.

The invention provides prodrug proteins, comprising, from N- to C-terminal: (sdABD-TTA)-domain linker-constrained Fv domain-cleavable linker-(sdABD-HSA)-constrained pseudo Fv domain. (Note that for all constructs for this format, the sdABD-HSA does not generally have a His6 tag, although it can be included).

As will be appreciated by those in the art, the order of the VH and VL in either a constrained Fv domain or a constrained pseudo Fv domain can be either (N- to C-terminal) VH-linker-VL or VL-linker-VH.

Thus, in one embodiment, the prodrug protein comprises, from N- to C-terminal: (sdABD-TTA)-domain linker-aVH-CNCL-aVL-CL-(sdABD-HSA)-domain linker-iVL-CNCL-iVH.

Thus, in one embodiment, the prodrug protein comprises, from N- to C-terminal: (sdABD-TTA)-domain linker-aVH-CNCL-aVL-CL-(sdABD-HSA)-domain linker-iVH-CNCL-iVL.

Thus, in one embodiment, the prodrug protein comprises, from N- to C-terminal: (sdABD-TTA)-domain linker-aVL-CNCL-aVH-CL-(sdABD-HSA)-domain linker-iVH-CNCL-iVL.

Thus, in one embodiment, the prodrug protein comprises, from N- to C-terminal: (sdABD-TTA)-domain linker-aVL-CNCL-aVH-CL-(sdABD-HSA)-domain linker-iVL-CNCL-iVH.

Thus, in one embodiment, the prodrug protein comprises, from N- to C-terminal: (sdABD-TTA)-domain linker-aVH-CNCL-aVL-CL-(sdABD-HSA)-domain linker-iVL-CNCL-iVH. In this embodiment, the aVH, aVL, iVH, iVL have the sequences shown in FIGS. 7A-7B. In this embodiment, the targeting domain binds to a TTA which can be EGFR, EpCAM, FOLR1, Trop2, CA9, LyPD3, HER2 or B7H3, the sequences for which are depicted in FIGS. 5A-5N and 75.

### D. Two Protein Compositions

In some embodiments, the compositions of the invention comprise two different molecules, sometimes referred to as “hemi-COBRAs™”, or “hemi-constructs”, that in the absence of cleavage, intramolecularly associate to form pseudo-Fvs. In the presence of the protease, the cleavage sites are cleaved, releasing the inert variable domains, and the protein pair then forms an active antigen binding domain to CD3, as generally depicted in FIG. 3.

What is important in the design of the hemi-constructs is that the active variable domain and the sdABD-TTA remain together after cleavage, such that the two cleaved portions are held together by the tumor antigen receptor on the tumor surface and then can form an active anti-CD3 binding domain.

There are two different general Format 3 constructs, those wherein each member of the pair has a single sdABD-TTA (FIG. 3A) and those with two different sdABD-TTAs, each to a different TTA (FIG. 3B).

### 1. Hemi-COBRA™ Constructs With Single TTA Binding Domains (Format 3A)

In some embodiments, the first hemi-COBRA™ has, from N- to C-terminal, sdABD(TTA1)-domain linker-aVH-CL-iVL-domain linker- sdABD(½) and the second has sdABD(½)-domain linker-iVH-CL-aVL-domain linker-sdABD(TTA2). In this embodiment, the aVH, aVL, iVH, iVL and sdABD(½) have the sequences shown in FIGS. 6 and 7, and the sdABD-TTAa bind to human EGFR, EpCAM, Trop2, CA9, LyPD3, HER2, FOLR1 and/or B7H3, and has a sequence depicted in FIGS. 5A-5N and 75.

### 2. Hemi-COBRA™ Constructs With Dual TTA ABDs

In some embodiments, the paired pro-drug constructs can have two sdABD-TTA binding domains per construct, as is shown in FIG. 3B. In this embodiments, the first member of the pair comprises, from N- to C-terminal, sdABD-TTA1-domain linker-sdABD-TTA2-domain linker-aVH-CL-iVL-domain linker-sdABD(HAS), and the second member comprises, from N- to C-terminal, sdABD-TTA1-domain linker-sdABD-TTA2-aVL-CL-iVH-domain linker-(sdABD-HSA).

The two sdABD-TTAs on each member of the pair are different, but generally both members (hemi-COBRAs™) have the same two sdABD-TTAs, e.g. both have EGFR and FOLR1 or EGFR and B7H3, etc.

The two sdABD-TTAs are in some embodiments selected from the ones shown in FIGS. 5A-5N and 75.

### IV. Methods of Making the Compositions of the Invention

The pro-drug compositions of the invention are made as will generally be appreciated by those in the art and outlined below.

The invention provides nucleic acid compositions that encode the pro-drug compositions of the invention. As will be appreciated by those in the art, the nucleic acid compositions will depend on the format of the pro-drug polypeptide(s). Thus, for example, when the format requires two amino acid sequences, such as the “format 3” constructs, two nucleic acid sequences can be incorporated into one or more expression vectors for expression. Similarly, prodrug constructs that are a single polypeptide (formats 1, 2 and 4), need a single nucleic acid in a single expression vector for production.

As is known in the art, the nucleic acids encoding the components of the invention can be incorporated into expression vectors as is known in the art, and depending on the host cells used to produce the prodrug compositions of the invention. Generally the nucleic acids are operably linked to any number of regulatory elements (promoters, origin of replication, selectable markers, ribosomal binding sites, inducers, etc.). The expression vectors can be extra-chromosomal or integrating vectors.

The nucleic acids and/or expression vectors of the invention are then transformed into any number of different types of host cells as is well known in the art, including mammalian, bacterial, yeast, insect and/or fungal cells, with mammalian cells (e.g. CHO cells, 293 cells), finding use in many embodiments.

The prodrug compositions of the invention are made by culturing host cells comprising the expression vector(s) as is well known in the art. Once produced, traditional antibody purification steps are done, including an protein A affinity chromatography step and/or an ion exchange chromatography step.

### V. Formulation and Administration of the Pro-Drug Compositions

Formulations of the pro-drug compositions used in accordance with the present invention are prepared for storage by mixing the pro-drugs (single proteins in the case of formats 1, 2 and 4 and two proteins in the case of format 3) having the desired degree of purity with optional pharmaceutically acceptable carriers, excipients or stabilizers (as generally outlined in Remington’s Pharmaceutical Sciences 16th edition, Osol, A. Ed. [1980]), in the form of lyophilized formulations or aqueous solutions.

The pro-drug compositions of the invention are administered to a subject, in accord with known methods, such as intravenous administration as a bolus or by continuous infusion over a period of time.

The pro-drug compositions of the invention are useful in the treatment of cancer. Provided herein are methods of treating cancer in a patient comprising any of the pro-drug compositions described. Described herein is a pro-drug composition for use as a medicament. Provided is a pharmaceutical composition for treating cancer comprising any of the pro-drug compositions described. Provided is a pharmaceutical composition comprising any of the pro-drug compositions described for treating cancer in a patient in need thereof. Provided is a pro-drug composition as described for the treatment or for the use in a method for treating cancer. Provided is a pro-drug composition described herein for treating cancer in a patient in need thereof. Provided is the use of a pro-drug composition in the manufacture of a medicament for the treatment of cancer.

### VI. Exemplary Embodiments

The present invention provides a number of different protein compositions for the treatment of cancer. Accordingly, in one aspect, the invention provides “Format 2” proteins comprising, from N- to C-terminal: a first single domain antigen binding domain (sdABD) that binds to a human tumor target antigen (TTA) (sdABD-TTA); b) a domain linker; c) a constrained Fv domain comprising: i) a variable heavy domain comprising a vhCDR1, vhCDR2 and vhCDR3; ii) a constrained non-cleavable linker (CNCL); and iii) a variable light domain comprising vlCDR1, vlCDR2 and vlCDR3; d) a second domain linker; e) a second sdABD-TTA; f) a cleavable linker (CL); g) a constrained pseudo Fv domain comprising: i) a pseudo light variable domain; ii) a constrained non-cleavable linker (CNCL); and iii) a pseudo heavy variable domain; h) a third domain linker; and i) a third sdABD that binds to human serum albumin; wherein the variable heavy domain and the variable light domain are capable of binding human CD3 but the constrained Fv domain does not bind CD3; the variable heavy domain and the pseudo variable light domain intramolecularly associate to form an inactive Fv; and the variable light domain and the pseudo variable heavy domain intramolecularly associate to form an inactive Fv. In some embodiments, the human tumor target antigen is B7H3.

In a further aspect, the invention provides proteins comprising, from N- to C-terminal: a first single domain antigen binding domain (sdABD) that binds to a human tumor target antigen (TTA) (sdABD-TTA) comprising sdFR1-sdCDR1-sdFR2-sdCDR2-sdFR3-sdCDR3-sdFR4; b) a first domain linker; c) a constrained Fv domain comprising: i) a variable heavy domain comprising vhFR1-vhCDR1-vhFR2-vhCDR2-vhFR3-vhCDR3-vhFR4; ii) a constrained non-cleavable linker (CNCL); and iii) a variable light domain comprising vlFR1-vlCDR1-vlFR2-vlCDR2-vlFR3-vlCDR3-vlFR4; d) a second domain linker; e) a second sdABD-TTA; f) a cleavable linker (CL); g) a constrained pseudo Fv domain comprising: i) a pseudo light variable domain comprising sdFR1-sdCDR1-sdFR2-sdCDR2-sdFR3-sdCDR3-sdFR4; ii) a constrained non-cleavable linker (CNCL); and iii) a pseudo heavy variable domain comprising vlFR1-vlCDR1-vlFR2-vlCDR2-vlFR3-vlCDR3-vlFR4; h) a third domain linker; and i) a third sdABD that binds to human serum albumin comprising sdFR1-sdCDR1-sdFR2-sdCDR2-sdFR3-sdCDR3-sdFR4; wherein the variable heavy domain and the variable light domain are capable of binding human CD3 but the constrained Fv domain does not bind CD3; the variable heavy domain and the pseudo variable light domain intramolecularly associate to form an inactive Fv; and the variable light domain and the pseudo variable heavy domain intramolecularly associate to form an inactive Fv. In some embodiments, the human tumor target antigen is B7H3.

In some embodiments of Format 2 proteins, the variable heavy domain is N-terminal to the variable light domain and the pseudo light variable domain is N-terminal to the pseudo variable heavy domain. In some embodiments, the variable heavy domain is N-terminal to the variable light domain and the pseudo variable light domain is C-terminal to the pseudo variable heavy domain. In some embodiments, the variable heavy domain is C-terminal to the variable light domain and the pseudo variable light domain is N-terminal to the pseudo variable heavy domain. In some embodiments, the variable heavy domain is C-terminal to the variable light domain and the pseudo variable light domain is C-terminal to the pseudo variable heavy domain.

In some embodiments of Format 2 proteins, the first sdABDTTA and the second sdABDTTA are the same. In some embodiments, the first sdABDTTA and the second sdABDTTA are different. In these embodiments, the sdABD-TTAs are selected from those depicted in FIG. 7, including SEQ ID NO:1, SEQ ID NO:5, SEQ ID NO:9, SEQ ID NO:13; SEQ ID NO:17; SEQ ID NO:21, SEQ ID NO:25, SEQ ID NO:29, SEQ ID NO:33, SEQ ID NO:37, SEQ ID NO:41, SEQ ID NO:45, SEQ ID NO:49, SEQ ID NO:53, SEQ ID NO:57, SEQ ID NO:61, SEQ ID NO:65, SEQ ID NO:69, SEQ ID NO:73,77, SEQ ID NO:81, SEQ ID NO:85, SEQ ID NO:89, SEQ ID NO:93, SEQ ID NO:97, SEQ ID NO:101, SEQ ID NO:105, SEQ ID NO:109 and SEQ ID NO:113.

In some embodiments of Format 2 proteins, the pseudo heavy variable domain of the constrained pseudo Fv domain is selected from the group of SEQ ID NO:146 (V_{Hi}), SEQ ID NO:150 (V_{Hi2}) and SEQ ID NO:154 (VHiGL4), as shown in FIG. 7. In some embodiments, the pseudo light variable domain of the constrained pseudo Fv domain is selected from the group of SEQ ID NO:130 (V_{Li}), SEQ ID NO:134 (V_{Li2}) and SEQ ID NO:138 (V_{LiGL}), as shown in FIG. 7.

In a further aspect, the invention provides “Format 1” proteins comprising, from N- to C-terminal: a) a first sdABD-TTA; b) a first domain linker; c) a constrained Fv domain comprising: i) a first variable heavy domain comprising a vhCDR1, vhCDR2 and vhCDR3; ii) a constrained cleavable linker (CCL); and iii) a first variable light domain comprising vlCDR1, vlCDR2 and vlCDR3; d) a second domain linker; e) a second sdABD-TTA; f) a cleavable linker (CL); g) a constrained pseudo Fv domain comprising: i) a first pseudo light variable domain; ii) a constrained non-cleavable linker (CNCL); and iii) a first pseudo heavy variable domain; h) a third domain linker; and i) a third sdABD that binds to human serum albumin; wherein the first variable heavy domain and the first variable light domain are capable of binding human CD3 but the constrained Fv domain does not bind CD3; wherein the first variable heavy domain and the first pseudo variable light domain intramolecularly associate to form an inactive Fv; and wherein the first variable light domain and the first pseudo variable heavy domain intramolecularly associate to form an inactive Fv. In an additional aspect, the invention provides “Format 4” proteins comprising, from N- to C-terminal: a) a single domain antigen binding domain (sdABD) that binds to a human tumor target antigen (TTA) (sdABD-TTA); b) a first domain linker; c) a constrained Fv domain comprising: i) a first variable heavy domain comprising a vhCDR1, vhCDR2 and vhCDR3; ii) a constrained non-cleavable linker (CNCL); and iii) a first variable light domain comprising vlCDR1, vlCDR2 and vlCDR3; d) a cleavable linker (CL); e) a second sdABD that binds to human serum albumin; f) a domain linker; g) a constrained pseudo Fv domain comprising: i) a first pseudo light variable domain; ii) a constrained non-cleavable linker (CNCL); and iii) a first pseudo heavy variable domain; wherein the first variable heavy domain and the first variable light domain are capable of binding human CD3 but the constrained Fv domain does not bind CD3; wherein the first variable heavy domain and the first pseudo variable light domain intramolecularly associate to form an inactive Fv; and wherein the first variable light domain and the first pseudo variable heavy domain intramolecularly associate to form an inactive Fv.

In a further aspect to the Format 1, Format 2 and Format 4 proteins listed above, the first variable heavy domain is N-terminal to the first variable light domain and the pseudo light variable domain is N-terminal to the pseudo variable heavy domain.

In a further aspect to the Format 1, Format 2 and Format 4 proteins listed above, the first variable heavy domain is N-terminal to the first variable light domain and the pseudo variable heavy domain is N-terminal to the pseudo variable light domain.

In a further aspect to the Format 1, Format 2 and Format 4 proteins listed above, the first variable light domain is N-terminal to the first variable heavy domain and the pseudo light variable domain is N-terminal to the pseudo variable heavy domain.

In a further aspect to the Format 1, Format 2 and Format 4 proteins listed above, the first variable light domain is N-terminal to the first variable heavy domain and the pseudo variable heavy domain is N-terminal to the pseudo variable light domain.

In an additional aspect, the invention provides Format 1 and 2 proteins wherein the first and second TTA are the same. In a further aspect, the invention provides Format 1 and 2 proteins wherein the first and second TTA are different.

In an additional aspect, the invention provides Format 1, 2 and 4 proteins wherein the first and second TTA are selected from EGFR, EpCAM, FOLR1, Trop2, ca9 and B7H3. These sequences can be selected from the group consisting of SEQ ID NO:1, SEQ ID NO:5, SEQ ID NO:9, SEQ ID NO:13; SEQ ID NO:17; SEQ ID NO:21, SEQ ID NO:25, SEQ ID NO:29, SEQ ID NO:33, SEQ ID NO:37, SEQ ID NO:41, SEQ ID NO:45, SEQ ID NO:49, SEQ ID NO:53, SEQ ID NO:57, SEQ ID NO:61, SEQ ID NO:65, SEQ ID NO:69, SEQ ID NO:73,77, SEQ ID NO:81, SEQ ID NO:85, SEQ ID NO:89, SEQ ID NO:93, SEQ ID NO:97, SEQ ID NO:101, SEQ ID NO: 105, SEQ ID NO: 109 and SEQ ID NO:113.

In a further aspect, the invention provides Format 1, 2 and 4 proteins wherein the half-life extension domain has SEQ ID NO: 117 (aHSA (10GE)) and SEQ ID NO: 121 (aHSA with His tag).

In an additional aspect, the invention provides Format 1, 2 and 4 proteins wherein the cleavable linker is cleaved by a human protease selected from the group consisting of MMP2, MMP9, Meprin A, Meprin B, Cathepsin S, Cathepsin K, Cathespin L, GranzymeB, uPA, Kallekriein7, matriptase and thrombin, or others as depicted in FIG. 6.

In a further aspect, the invention provides a protein selected from the group consisting of Pro186, Pro225, Pro226, Pro233, Pro262, Pro311, Pro312, Pro313,Pro356, Pro359, Pro364, Pro388, Pro448, Pro449, Pro450, Pro451, Pro495, Pro246, Pro254, Pro255, Pro256, Pro420, Pro421, Pro432, Pro479, Pro480, Pro187, Pro221, Pro222, Pro223, Pro224, Pro393, Pro394, Pro395, Pro396, Pro429, Pro430, Pro431, Pro601, Pro602, V3 and V4, Pro664, Pro665, Pro667, Pro694, Pro695, Pro565, Pro566, Pro567, Pro727, Pro728, Pro729, Pro730, Pro731, Pro676, Pro677, Pro678, Pro679, Pro808, Pro819, Pro621, Pro622, Pro640, Pro641, Pro642, Pro643, Pro744, Pro746, Pro638, Pro639, Pro396, Pro476, Pro706, Pro709, Pro470, Pro471, Pro551, Pro552, Pro623, Pro624, Pro698, Pro655, Pro656, Pro657, Pro658, Pro516, Pro517, Pro518 and Pro519.

In an additional aspect, the invention provides nucleic acids encoding a Format 1, Format 2 or Format 4 protein as described herein, as well as expression vectors and host cells comprising the nucleic acids encoding the protein.

In a further aspect, the invention provides methods of making the proteins of the invention and methods of treating patients in need thereof.

In an additional aspect, the invention provides compositions comprising “Format 3A” pairs of pro-drug proteins, comprising: a) a first protein comprising, from N- to C-terminal: i) a first sdABD-TTA; ii) a first domain linker; iii) a pseudo Fv domain comprising, from N- to C-terminal: 1) a variable heavy chain comprising a vhCDR1, vhCDR2 and vhCDR3; 2) a cleavable linker; and 3) a first pseudo variable light domain comprising iVLCDR1, iVLCDR2 and iVLCDR3; iv) a second domain linker; v) a sdABD-HSA; a) a second protein comprising, from N- to C-terminal: i) a third sdABD that binds to a human tumor target antigen; ii) a third domain linker; iii) a pseudo Fv domain comprising, from N- to C-terminal: 1) a variable light chain comprising a VLCDR1, VLCDR2 and VLCDR3; 2) a cleavable linker; and 3) a first pseudo variable heavy domain comprising iVHCDR1, iVHCDR2 and iVHCDR3; iv) a fourth domain linker; v) a sdABD-HSA; wherein the first variable heavy domain and the first variable light domain are capable of binding human CD3 when associated; wherein the first variable heavy domain and the first pseudo variable light domain intermolecularly associate to form an inactive Fv; wherein the first variable light domain and the first pseudo variable heavy domain intermolecularly associate to form an inactive Fv; and wherein the first and third sdABD are selected from the group consisting of SEQ ID NO:1, SEQ ID NO:5, SEQ ID NO:9, SEQ ID NO:13; SEQ ID NO:17; SEQ ID NO:21, SEQ ID NO:25, SEQ ID NO:29, SEQ ID NO:33, SEQ ID NO:37, SEQ ID NO:41, SEQ ID NO:45, SEQ ID NO:49, SEQ ID NO:53, SEQ ID NO:57, SEQ ID NO:61, SEQ ID NO:65, SEQ ID NO:69, SEQ ID NO:73,77, SEQ ID NO:81, SEQ ID NO:85, SEQ ID NO:89, SEQ ID NO:93, SEQ ID NO:97, SEQ ID NO:101, SEQ ID NO: 105, SEQ ID NO: 109 and SEQ ID NO:113.

In a further aspect, the invention provides compositions comprising “Format 3B” pairs of pro-drug proteins, comprising a) a first protein comprising, from N- to C-terminal: i) a first sdABD-TTA; ii) a first domain linker; iii) a second sdABD-TTA; iv) a second domain linker; iii) a pseudo Fv domain comprising, from N- to C-terminal: 1) a variable heavy chain comprising a vhCDR1, vhCDR2 and vhCDR3; 2) a cleavable linker; and 3) a first pseudo variable light domain comprising iVLCDR1, iVLCDR2 and iVLCDR3; iv) a third domain linker; and v) a sdABD-HSA; a) a first second protein comprising, from N- to C-terminal: i) a third sdABD-TTA; ii) a fourth domain linker; iii) a fourth sdABD-TTA; iv) a fifth domain linker; iii) a pseudo Fv domain comprising, from N- to C-terminal: 1) a variable light chain comprising a VLCDR1, VLCDR2 and VLCDR3; 2) a cleavable linker; and 3) a first pseudo variable heavy domain comprising iVHCDR1, iVHCDR2 and iVHCDR3; iv) a sixth domain linker; v) a sdABD-HSA; wherein the first variable heavy domain and the first variable light domain are capable of binding human CD3 when associated; wherein the first variable heavy domain and the first pseudo variable light domain intermolecularly associate to form an inactive Fv; and wherein the first variable light domain and the first pseudo variable heavy domain intermolecularly associate to form an inactive Fv.

In an additional aspect, Format 3A and Format 3B proteins have sdABD-HSA that have SEQ ID NO: 117 or SEQ ID NO: 121. In a further aspect, Format 3A and Format 3B proteins have sdABD-TTA that binds to a TTA selected from EGFR, EpCAM, Trop2, CA9, FOLR1 and B7H3. The sdABD-TTAs can be selected from the group consisting of SEQ ID NO:1, SEQ ID NO:5, SEQ ID NO:9, SEQ ID NO:13; SEQ ID NO:17; SEQ ID NO:21, SEQ ID NO:25, SEQ ID NO:29, SEQ ID NO:33, SEQ ID NO:37, SEQ ID NO:41, SEQ ID NO:45, SEQ ID NO:49, SEQ ID NO:53, SEQ ID NO:57, SEQ ID NO:61, SEQ ID NO:65, SEQ ID NO:69, SEQ ID NO:73,77, SEQ ID NO:81, SEQ ID NO:85, SEQ ID NO:89, SEQ ID NO:93, SEQ ID NO:97, SEQ ID NO:101, SEQ ID NO: 105, SEQ ID NO: 109 and SEQ ID NO:113.

In an additional aspect, the invention provides sdABDs that bind to human Trop2, having a sequence selected from SEQ ID NO:77, SEQ ID NO:81, SEQ ID NO:85, SEQ ID NO:89 and SEQ ID NO:93. In a further aspect, the invention provides sdABDs that bind to human B7H3 having a sequence selected from SEQ ID NO:41, SEQ ID NO:45, SEQ ID NO:49, SEQ ID NO:53 and SEQ ID NO:57. In an additional aspect, the invention provides sdABDs that bind to human CA9 having a sequence selected from SEQ ID NO:101, SEQ ID NO: 105, SEQ ID NO: 109 and SEQ ID NO:113. In a further aspect the invention provides sdABDs that bind to human EpCAM having a sequence selected from SEQ ID NO:69 and SEQ ID NO:73.

In some aspects, provided herein is a fusion protein comprising, from N- to C-terminal: (a) a first sdABD that binds a tumor target antigen (sdABD-TTA); (b) a first domain linker; (c) a constrained Fv domain comprising:(i) a first variable heavy domain comprising a vhCDR1, vhCDR2 and vhCDR3; (ii) a constrained non-cleavable linker (CNCL); and(iii) a first variable light domain comprising vlCDR1, vlCDR2 and vlCDR3; (d) a second domain linker; (e) a second sdABD-TTA; (f) a cleavable linker (CL); (g) a constrained pseudo Fv domain comprising: (i) a first pseudo light variable domain; (ii) a non-cleavable linker (NCL); and (iii) a first pseudo heavy variable domain; (h) a third domain linker; and (i) a third sdABD that binds to human serum albumin (sdABD-HSA); wherein said first variable heavy domain and said first variable light domain are capable of binding human CD3 but the constrained Fv domain does not bind CD3; the first variable heavy domain and the first pseudo variable light domain intramolecularly associate to form an inactive Fv; and the first sdABD-TTA and the second sdABD-TTA bind the same TTA selected from the group consisting of B7H3, CA9, EGFR, EpCAM, FOLR1, HER2, LyPD3, and Trop2. In some embodiments, the first and/or second sdABD-TTA can be any sdABD-TTA disclosed herein.

In some aspects, provided herein is a fusion protein comprising, from N- to C-terminal: (a) a first sdABD that binds a tumor target antigen (sdABD-TTA); (b) a first domain linker; (c) a constrained Fv domain comprising:(i) a first variable heavy domain comprising a vhCDR1, vhCDR2 and vhCDR3; (ii) a constrained non-cleavable linker (CNCL); and(iii) a first variable light domain comprising vlCDR1, vlCDR2 and vlCDR3; (d) a second domain linker; (e) a second sdABD-TTA; (f) a cleavable linker (CL); (g) a constrained pseudo Fv domain comprising: (i) a first pseudo light variable domain; (ii) a non-cleavable linker (NCL); and (iii) a first pseudo heavy variable domain; (h) a third domain linker; and (i) a third sdABD that binds to human serum albumin (sdABD-HSA); wherein said first variable heavy domain and said first variable light domain are capable of binding human CD3 but the constrained Fv domain does not bind CD3; the first variable heavy domain and the first pseudo variable light domain intramolecularly associate to form an inactive Fv; and the first sdABD-TTA bind a TTA selected from the group consisting of B7H3, CA9, EGFR, EpCAM, FOLR1, HER2, LyPD3, and Trop2 and the second sdABD-TTA bind a different TTA selected from the group consisting of B7H3, CA9, EGFR, EpCAM, FOLR1, HER2, LyPD3, and Trop2. In some embodiments, the first and/or second sdABD-TTA can be any sdABD-TTA disclosed herein.

A fusion protein comprising an amino acid sequence consisting of any one selected from the group consisting of SEQ ID NO:288 (Pro565), SEQ ID NO:289 (Pro566), SEQ ID NO:290 (Pro567), SEQ ID NO:292 (Pro727), SEQ ID NO:293 (Pro728), SEQ ID NO:294 (Pro729), SEQ ID NO:295 (Pro730), SEQ ID NO:296 (Pro731), SEQ ID NO:297 (Pro676), SEQ ID NO:298 (Pro677), SEQ ID NO:299 (Pro678), SEQ ID NO:300 (Pro679), SEQ ID NO:301 (Pro808), SEQ ID NO:302 (Pro819), SEQ ID NO:304 (Pro621), SEQ ID NO:305 (Pro622), SEQ ID NO:306 Pro640, SEQ ID NO:307 (Pro641), SEQ ID NO:308 (Pro642), SEQ ID NO:309 (Pro643), SEQ ID NO:310 (Pro744), SEQ ID NO:311 (Pro746), SEQ ID NO:312 (Pro108), SEQ ID NO:313 (Pro109), SEQ ID NO:314 (Pro396,) SEQ ID NO:315 (Pro476), SEQ ID NO:316 (Pro706), SEQ ID NO:317 (Pro709), SEQ ID NO:318 (Pro470), SEQ ID NO:319 (Pro471), SEQ ID NO:320 (Pro551), SEQ ID NO:321 (Pro552), SEQ ID NO:322 (Pro623), SEQ ID NO:323 (Pro624), SEQ ID NO:324 (Pro698), SEQ ID NO:325 (Pro655), SEQ ID NO:326 (Pro656), SEQ ID NO:327 (Pro657), SEQ ID NO:328 (Pro658), SEQ ID NO:329 (Pro516), SEQ ID NO:330 (Pro517), SEQ ID NO:331 (Pro518), SEQ ID NO:332 (Pro519), SEQ ID NO:333 (Pro513), SEQ ID NO:336 (Pro225), SEQ ID NO:338 (Pro817), SEQ ID NO:416 (Pro311), SEQ ID NO:417 (Pro312), SEQ ID NO:418 (Pro313), SEQ ID NO:419 (Pro246), SEQ ID NO:420 (Pro256), SEQ ID NO:421 (Pro420), SEQ ID NO:422 (Pro421), SEQ ID NO:487 (Pro751), SEQ ID NO:488 (Pro752), SEQ ID NO:489 (Pro824), and SEQ ID NO:490 (Pro826) SEQ ID NO:522 (Pro601), SEQ ID NO:523 (Pro602), SEQ ID NO:524 (V3), SEQ ID NO:525 (V4), SEQ ID NO:526 (Pro664), SEQ ID NO:527 (Pro665), SEQ ID NO:528 (Pro667), SEQ ID NO:529 (Pro694), SEQ ID NO:530 (Pro695), and SEQ ID NO:531 (Pro565).

In a further aspect, the invention provides nucleic acid compositions comprising first nucleic acids that encode the first protein members of the prodrug pair and second nucleic acids that encode the second protein members of the pairs, and expression vectors and host cells containing the nucleic acids.

### EXAMPLES

### Example 1: Pro Construct Construction and Purification

### Transfections

Each protein (e.g. single proteins for Formats 1, 2 and 4) or pairs of constructs (Format 3) were expressed from a separate expression vector (pcdna3.4 derivative). Equal amounts of plasmid DNA that encoded the pair of hemi-cobra or single chain constructs were mixed and transfected to Expi293 cells following the manufacture’s transfection protocol. Conditioned media was harvested 5 days post transfection by centrifugation (6000 rpm x 25′) and filtration (0.2 uM filter). Protein expression was confirmed by SDS-PAGE. Constructs were purified and the final buffer composition was: 25 mM Citrate, 75 mM Arginine, 75 mM NaCl, 4% Sucrose, pH 7. The final preparations were stored at -80° C.

### Activation of MMP9

Recombinant human (rh) MMP9 was activated according to the following protocol. Recombinant human MMP-9 (R&D # 911-MP-010) is at 0.44 mg/ml (4.7 uM). p-aminophenylmercuric acetate (APMA) (Sigma) is prepared at the stock concentration of 100 mM in DMSO. Assay buffer is 50 mM Tris pH 7.5, 10 mM CaCl2, 150 mM NaCl, 0.05% Brij-35.
Dilute rhMMP9 with assay buffer to ~100 ug/ml (25 ul hMMP9 + 75 uL assay buffer)
Add p-aminophenylmercuric acetate (APMA) from 100 mM stock in DMSO to a final concentration of 1 mM (1 uL to 100 uL)
Incubate at 37° C. for 24 hrs
Dilute MMP9 to 10 ng/ul (add 900 ul of assay buffer to 100 ul of activated solution)

The concentration of the activated rhMMP9 is ~ 100 nM.

### Cleavage of Constructs for TDCC Assays

To cleave the constructs, 100 ul of the protein sample at 1 mg/ml concentration (10.5 uM) in the formulation buffer (25 mM Citric acid, 75 mM L-arginine, 75 mM NaCl, 4% sucrose) was supplied with CaCl₂ up to 10 mM. Activated rhMMP9 was added to the concentration 20-35 nM. The sample was incubated at room temperature overnight (16-20 hrs). The completeness of cleavage was verified using SDS PAGE (10-20% TG, TG running buffer, 200 v, 1 hr). Samples were typically 98% cleaved.

### Example 2: T Cell Dependent Cellular Cytotoxicity (TDCC) Assays

Firefly Luciferase transduced HT-29 cells were grown to approximately 80% confluency and detached with Versene (0.48 mM EDTA in PBS - Ca - Mg). Cells were centrifuged and resuspended in TDCC media (5% Heat Inactivated FBS in RPMI 1640 with HEPES, GlutaMax, Sodium Pyruvate, Non-essential amino acids, and β-mercaptoethanol). Purified human Pan-T cells were thawed, centrifuged and resuspended in TDCC media.

A coculture of HT-29_Luc cells and T cells was added to 384-well cell culture plates. Serially diluted COBRAs were then added to the coculture and incubated at 37° C. for 48 hours. Finally, an equal volume of SteadyGlo luciferase assay reagent was added to the plates and incubated for 20 minutes. The plates were read on the Perkin Elmer Envision with an exposure time of 0.1 s/well. Total luminescence was recorded and data were analyzed on GraphPad Prism 7 or Version 8.3.1 (depending on timing).

### Example 3: General Protocol Design of the In Vivo Adoptive T Cell Transfer Efficacy Model

These protocols were used in many of the experiments of the figures.

### Protocol 1:

Tumor cells were implanted subcutaneous (SC) in the right flank of NSG (NOD.Cg-Prkdcscid Il2rgtm1Wjl/SzJ) mice (The Jackson Laboratory, Cat. No. 005557) and allowed to grow until an established tumor with a mean volume of around 200 mm³ was reached. In parallel human T cells were cultured in T cell media (X-VIVO 15 [Lonza, Cat.No. 04-418Q], 5% Human Serum, 1% Penicillin/Streptomycin, 0.01 mM 2-Mercaptoethanol) in a G-Rex100M gas permeable flask (Wilson Wolf Cat. No. 81100S) with MACSiBeads from the T Cell Activation/Expansion Kit (Miltenyi Cat. No. 130-091-441) for around 10 days and supplemented with recombinant human IL-2 protein. Tumor growth in mice and human T cell activation/expansion were coordinated so that on Day 0 of the study mice were randomized into groups (N=6) based on tumor size; each were then injected intravenous (IV) with 2.5×10⁶ cultured human T cells and administered the first dose of the COBRA or control molecules. Mice were dosed every 3 days for 7 doses (Days 0, 3, 6, 9, 12, 15 and 18) and then followed for an additional 2-3 weeks until tumors reached >2000 mm³ in volume or the study was terminated. Tumor volumes were measured every 3 days.

### Protocol 2 for Human PBMC Engraftment Model

NSG-β2M-/- mice (Jackson) were engrafted with i.v. with human PBMC; 3d post engraftment, mice were implanted with tumor cell lines subcutaneously. Once tumor growth was established, mice were randomized based on tumor volume, and test articles were dosed i.v. as indicated. Tumor volume was assessed by caliper measurement. Plasma was collected 4h post-dose to assess cytokine levels (MesoScale Discovery) and liver enzyme elevations.

Note that the key difference between the two protocols is that human T cells are injected at the same time as the first COBRA dose, while in Protocol 2 human PBMC are put in at the same time as the tumor cells and the COBRA injections start about 10 days later.

### Example 4: In Vivo Activity With EGFR/MMP9 Hemi-COBRA Pair Pro77 and Pro53

5 × 10⁶ LoVo cells or 5 × 10⁶ HT29 cells were implanted subcutaneous in the right flank of NSG (NOD.Cg-Prkdcscid Il2rgtm1Wjl/SzJ) mice (The Jackson Laboratory, Cat. No. 005557) and allowed to grow until tumors were established. In parallel human T cells were cultured in T cell media (X-VIVO 15 [Lonza, Cat.No. 04-418Q], 5% Human Serum, 1% Penicillin/Streptomycin, 0.01 mM 2-Mercaptoethanol) in a G-Rex100M gas permeable flask (Wilson Wolf Cat. No. 81100S) with MACSiBeads from the T Cell Activation/Expansion Kit (Miltenyi Cat. No. 130-091-441) for 10 days and supplemented with recombinant human IL-2 protein. Tumor growth in mice and human T cell activation/expansion were coordinated so that on Day 0 of the study mice were randomized into groups (N=6) based on tumor size; each were then injected intravenous (IV) with 2.5×10⁶ cultured human T cells and administered the first dose of the COBRA or control molecules. Mice were dosed every 3 days for 7 doses (Days 0, 3, 6, 9, 12, 15 and 18) and then followed until tumors reach >2000 mm³ in volume or the study was terminated. Groups received 0.2 mg/kg (mpk) of the anti-EGFR x CD3 positive control Pro51 bispecific antibody (bsAb), 0.5 mpk of the negative control anti-hen egg lysozyme (HEL) x CD3 bsAb Pro98, 0.5 mpk each of the MMP9 cleavable linker containing anti-EGFR hemi-COBRA pair Pro77 and Pro53, or 0.5 mpk each of the non-cleavable(NCL) linker containing anti-EGFR hemi-COBRA pair Pro74 and Pro72. Tumor volumes were measured every 3 days.

### Example 5: In Vivo Activity With EGFR/MMP9 COBRA Pro140

5 × 10⁶ LoVo cells or 5 × 10⁶ HT29 cells were implanted subcutaneous in the right flank of NSG (NOD.Cg-Prkdcscid Il2rgtm1Wjl/SzJ) mice (The Jackson Laboratory, Cat. No. 005557) and allowed to grow until tumors were established. In parallel human T cells are cultured in T cell media (X-VIVO 15 [Lonza, Cat.No. 04-418Q], 5% Human Serum, 1% Penicillin/Streptomycin, 0.01 mM 2-Mercaptoethanol) in a G-Rex100M gas permeable flask (Wilson Wolf Cat. No. 81100S) with MACSiBeads from the T Cell Activation/Expansion Kit (Miltenyi Cat. No. 130-091-441) for 10 days and supplemented with recombinant human IL-2 protein. Tumor growth in mice and human T cell activation/expansion were coordinated so that on Day 0 of the study mice were randomized into groups (N=6) based on tumor size; each were then injected intravenous (IV) with 2.5×10⁶ cultured human T cells and administered the first dose of the COBRA or control molecules. Mice were dosed every 3 days for 7 doses (Days 0, 3, 6, 9, 12, 15 and 18) and then followed until tumors reach >2000 mm³ in volume or the study was terminated. Groups received 0.2 mpk of the anti-EGFR x CD3 positive control Pro51 bispecific antibody (bsAb), 0.5 mpk of the negative control anti-hen egg lysozyme (HEL) x CD3 bsAb Pro98, or 0.5 mpk of the MMP9 cleavable linker containing anti-EGFR COBRA Pro140. Tumor volumes were measured every 3 days.

### Example 6: In Vivo Activity With EGFR/MMP9 COBRA Pro186

5 × 10⁶ HT29 cells were implanted subcutaneous in the right flank of NSG (NOD.Cg-Prkdcscid Il2rgtm1Wjl/SzJ) mice (The Jackson Laboratory, Cat. No. 005557) and allowed to grow until tumors were established. In parallel human T cells are cultured in T cell media (X-VIVO 15 [Lonza, Cat.No. 04-418Q], 5% Human Serum, 1% Penicillin/Streptomycin, 0.01 mM 2-Mercaptoethanol) in a G-Rex100M gas permeable flask (Wilson Wolf Cat. No. 81100S) with MACSiBeads from the T Cell Activation/Expansion Kit (Miltenyi Cat. No. 130-091-441) for 10 days and supplemented with recombinant human IL-2 protein. Tumor growth in mice and human T cell activation/expansion were coordinated so that on Day 0 of the study mice were randomized into groups (N=6) based on tumor size; each were then injected intravenous (IV) with 2.5×10⁶ cultured human T cells and administered the first dose of the COBRA or control molecules. Mice were dosed every 3 days for 7 doses (Days 0, 3, 6, 9, 12, 15 and 18) and then followed until tumors reach >2000 mm³ in volume or the study was terminated. Groups received 0.1 mg/kg (mpk) of the anti-EGFR x CD3 positive control Pro51 bispecific antibody (bsAb), 0.3 mpk of the of the non-cleavable (NCL) control linker containing anti-EGFR COBRA Pro214, 0.1 or 0.3 mpk of the MMP9 cleavable linker containing anti-EGFR COBRA Pro140, or 0.1 or 0.3 mpk of the MMP9 cleavable linker containing anti-EGFR COBRA Pro186. Tumor volumes were measured every 3 days.

### Example 7A: Successful Humanization of anti-EGFR Sequences

The results are shown below.

| | | | |
|---|---|---|---|
| Molecule | KD (M) | Kon (⅟Ms) | Kdis(⅟s) |
| Pro22 (parental EGFR) | 2.58E-09M/2.6 nM | 2.05E+05 | 5.27E-04 |
| Pro90 (hEGFR1) | 2.00E-09M/2.0 nM | 2.21E+05 | 4.40E-04 |
| Pro48 (EGFR2) | 2.89E-09M/2.9 nM | 6.09E+05 | 1.76E-03 |
| Pro137 (hEGFR2) | 4.36E-09M/4.4. nM | 5.85E+05 | 2.55E-03 |
| Pro51 (hEGFR2) | 3.27E-09M/3.2 nM | 6.45E+05 | 2.11E-03 |
| Pro201 (hEGFR2 with 2 binding sites) | 2.25E-12M/2.3 pM | 1.55E+06 | 3.48E-06 |

These results show both that the humanization of the EGFR binding domains was successful, and that there is strong avidity to the target EGFR when two binding sites are on the molecule.

### Example 7B: Successful Humanization of EpCAM sdABDs

The results are shown below.

| Clone | Human binding affinity (nM) | Cyno binding affinity (nM) | Cyno/Human cross reactivity |
|---|---|---|---|
| VIB-13 | 2.3 | 11.6 | 5 |
| hVIB-13 | 2.8 | 12.7 | 4.5 |
| VIB-23 | 4.2 | 46.7 | 11.1 |
| hVIB-23 | 4.1 | 58.8 | 12.6 |

These results show both that the humanization of the EpCAM binding domains was successful.

### Example 8: COBRA™: A Novel Conditionally Active Bispecific Antibody that Regresses Established Solid Tumors in Mice - Mono-specific and Hetero-specific COBRAs

Despite clinical success with bispecific antibodies (bsAbs) targeting hematological malignancies (e.g. blinatumomab, a CD19xCD3 bsAb), efficacy in solid tumor indications remains a significant challenge. Because T cell redirecting bsAbs are so potent, even very low levels of cell surface target antigen expression on normal tissues may quickly become a safety liability and severely restrict the dose levels that can be achieved in patients. This limits the likelihood of reaching efficacious concentrations and reduces the therapeutic potential of these highly active molecules. Additionally, identifying “clean” target antigens that are uniquely expressed on the tumor and not on normal tissues has been very difficult at best.

To overcome these challenges, we have developed a novel recombinant bsAb platform called COBRA™ (Conditional Bispecific Redirected Activation). COBRAs are engineered to enable targeting of more widely expressed and validated tumor cell surface antigens by focusing T cell engagement within the tumor microenvironment. COBRA molecules are designed to bind to target antigen, which may be expressed on both tumor and normal cells, yet not engage T cells unless exposed to a proteolytic microenvironment, which is common in tumors but not in normal healthy tissues. Once bound to the tumor target antigen, protease-dependent linker cleavage allows COBRAs to convert an inactive anti-CD3 scFv to an active anti-CD3 scFv binding domain. Upon conversion, COBRAs are then able to simultaneously co-engage T cells and target antigen, resulting in a potent cytolytic T cell response against the tumor cells. In addition, COBRAs are designed with a half-life extension moiety that is removed from the active molecule upon proteolytic cleavage. This allows for a sustained presence in the circulation of the inactive COBRA prior to tumor target binding, and more rapid clearance of unbound active COBRA molecules, thereby decreasing the potential for cytotoxic activity in normal tissues.

Here we have revealed the novel design of the COBRA molecule and demonstrate its ability to engage CD3 and Epidermal Growth Factor Receptor (EGFR) to elicit potent cytotoxic activity in T cell culture and in human T cell implanted tumor-bearing mice. We have reported low-to-sub-picomolar T cell activation and cytotoxicity in vitro, and COBRA linker cleavage dependent T cell mediated regression of established solid tumor xenografts in NSG mice in vivo.

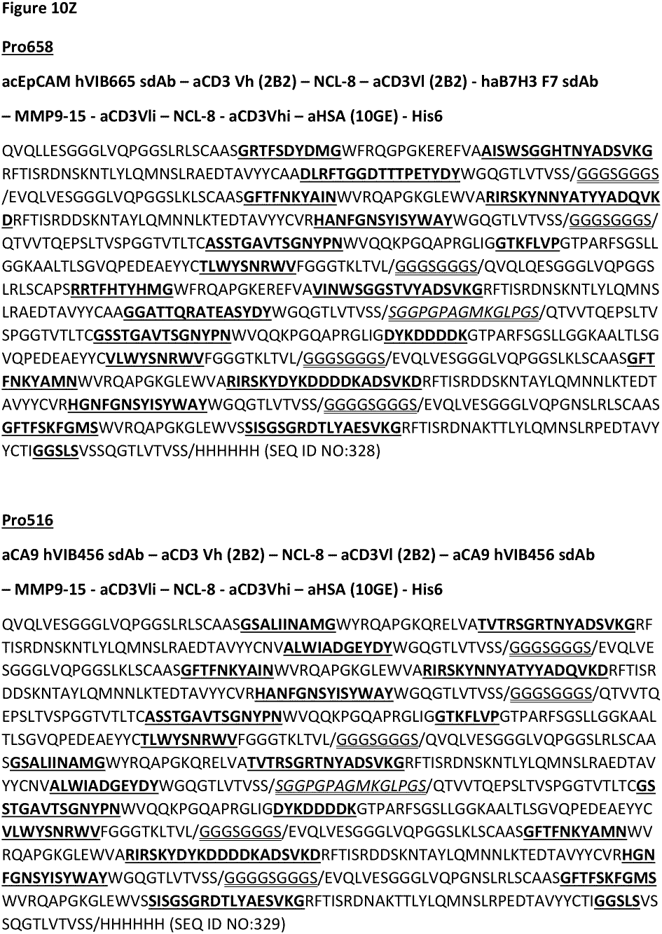

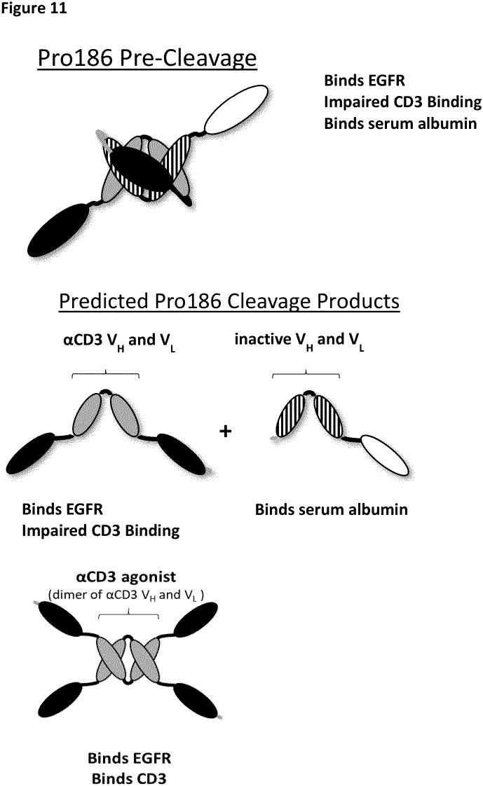

FIGS. 11A-11C illustrate the COBRA design and the predicted folding mechanism. FIG. 11A depicts a schematic of the PRO186 COBRA. FIG. 11B shows the predicted COBRA folding. The COBRA includes inactive VH and VL paired with anti-CD3 VH and VL domains. The uncleaved PRO186 COBRA binds EGFR, has impaired CD3 binding, and binds serum albumin. FIG. 11C shows an analytical size exclusion chromatogram of PRO186. The data shows that the uncleaved PRO186 folds into a single structure.

FIG. 11A-FIG. 11D illustrates the COBRA design and the predicted folding mechanism, with the predicted structure of the uncleaved molecule on the left, which still binds tumor antigen (EGFR, in the case of the MVC-101), has impaired CD3 binding and binds human serum albumin. The middle shows the predicted cleavage products and the left shows the active dimer.

FIG. 12A-FIG. 12Q depicts additional sequences of some COBRAs of the present invention.

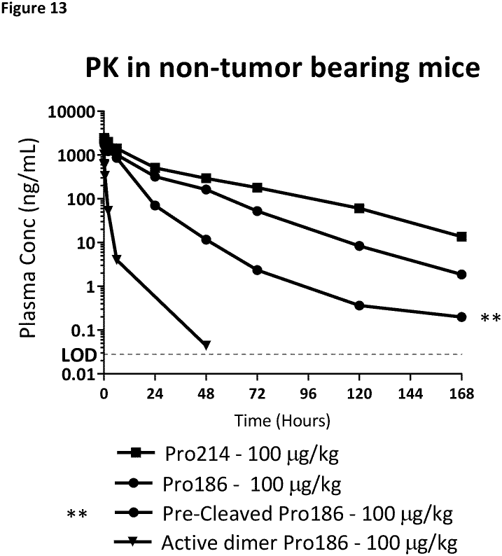

FIG. 13 shows that the format 2 constructs of the invention, once cleaved and dimerized, clear quickly from injected mice.

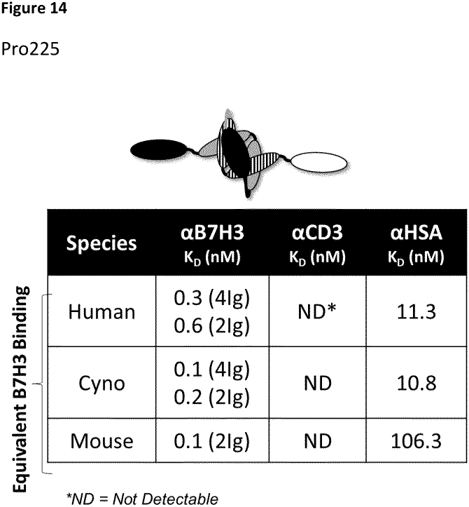

FIG. 14 shows the binding kinetics of Pro225.

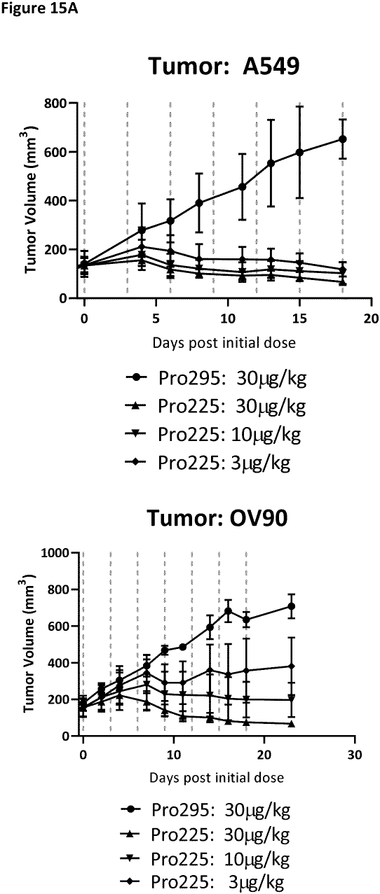

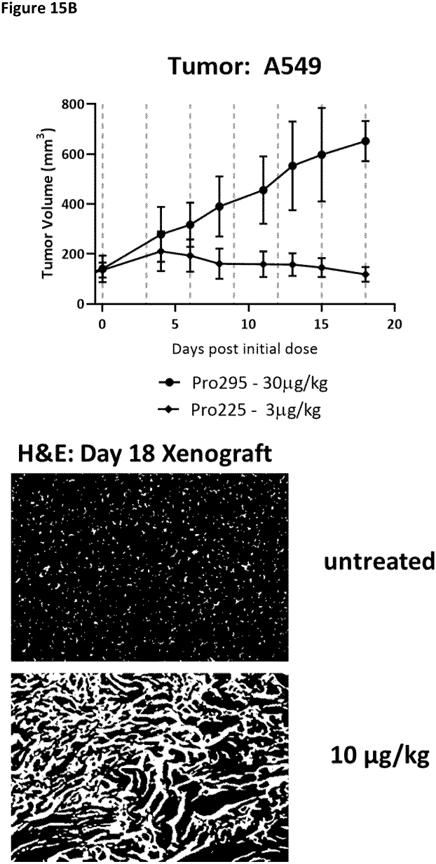

FIGS. 15A and 15B shows that format 2 constructs, in this case Pro225, regresses established solid tumors in mice.

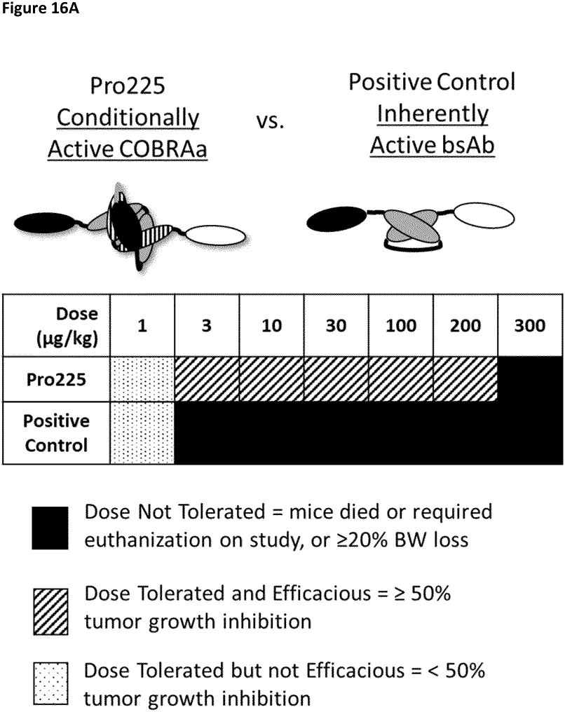

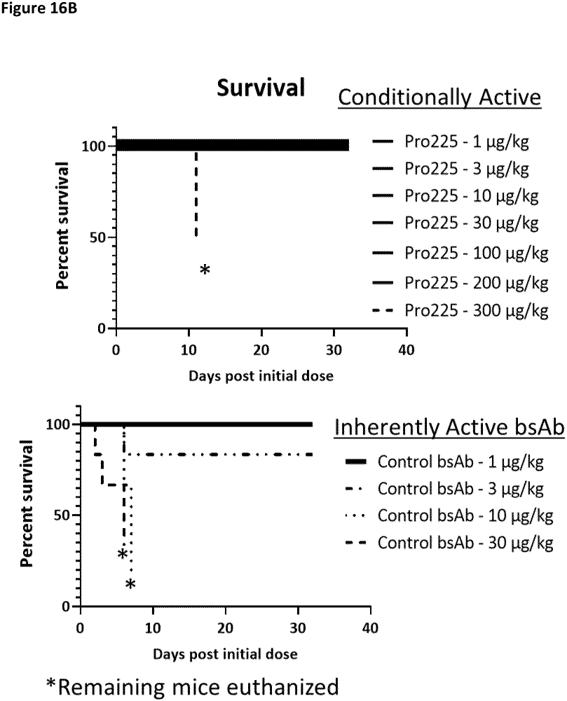

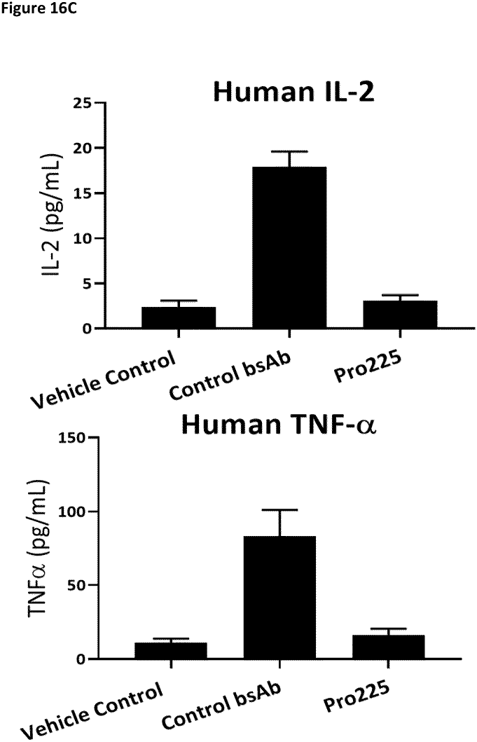

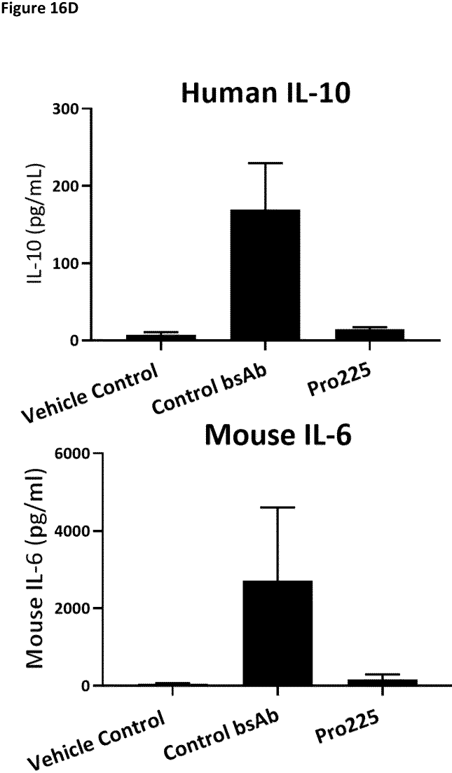

FIGS. 16A and 16B shows that the format 2 constructs of the invention, in this case Pro225, shows increased tolerability relative to inherently active T cell engagers. FIGS. 16C and 16D show that treatment with Pro225 results in lower cytokine release in mice, compared to an inherently active bispecific. Pro 225 does not induce IL2, TNFa, and IL10 in NHP and mouse IL6 in mice in comparison to inherently active T cell engagers

FIG. 17 shows the efficacy of a number of format 2 constructs of the invention in a T cell Dependent Cellular Cytotoxicity (TDCC) assay as outlined in Example 2. Pro233 is an aEGFR construct with an MMP9 cleavage site; Pro565 is an aEpCAM (h664) construct with an MMP9 cleavage site; Pro566 is an aEpCAM (h665) construct with an MMP9 cleavage site; Pro623 is a heteroCOBRA of aEGFR and aEpCAM (h664) and an MMP9 site; and Pro624 is a heteroCOBRA of aEGFR and aEpCAM (h665) and an MMP9 site.

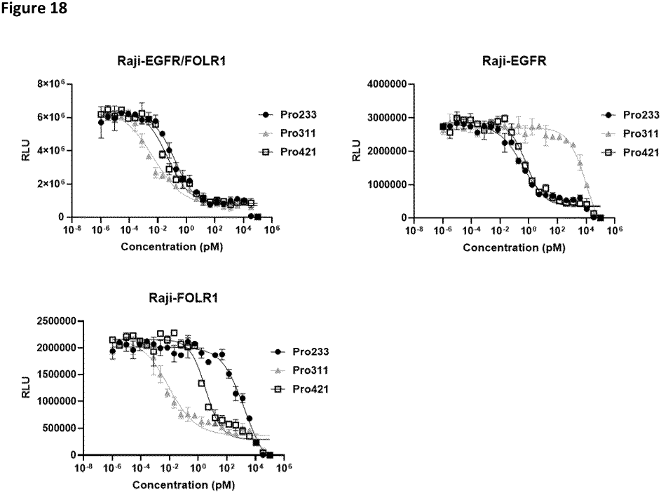

FIG. 18 shows the efficacy of a number of format 2 constructs of the invention in a TDCC assay as outlined in Example 2. Pro233 is an aEGFR construct with an MMP9 cleavage site; Pro311 is an aFOLR1 construct with an MMP9 cleavage site; and Pro421 is a heteroCOBRA of aEGFR and aFOLR1 and an MMP9 site.

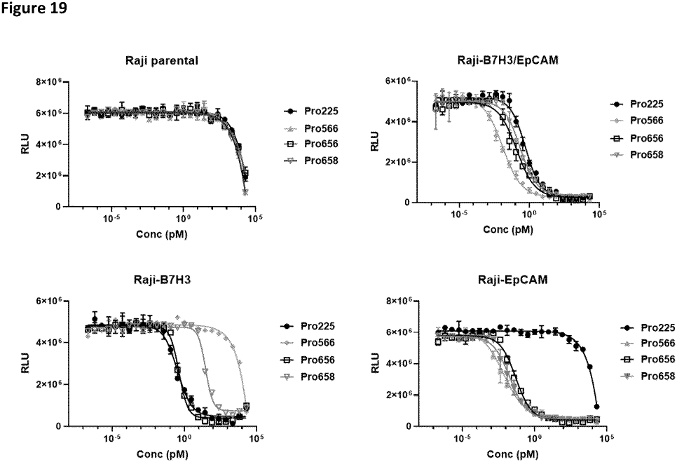

FIG. 19 shows the efficacy of a number of format 2 constructs of the invention in a TDCC assay as outlined in Example 2. Pro225 is an aB7H3 construct with an MMP9 cleavage site; Pro566 is an aEpCAM construct with an MMP9 cleavage site; Pro656 is a heteroCOBRA of aB7H3 and aEpCAM and an MMP9 site; and Pro658 is a heteroCOBRA of aEpCAM and aB7H3 and an MMP9 site.

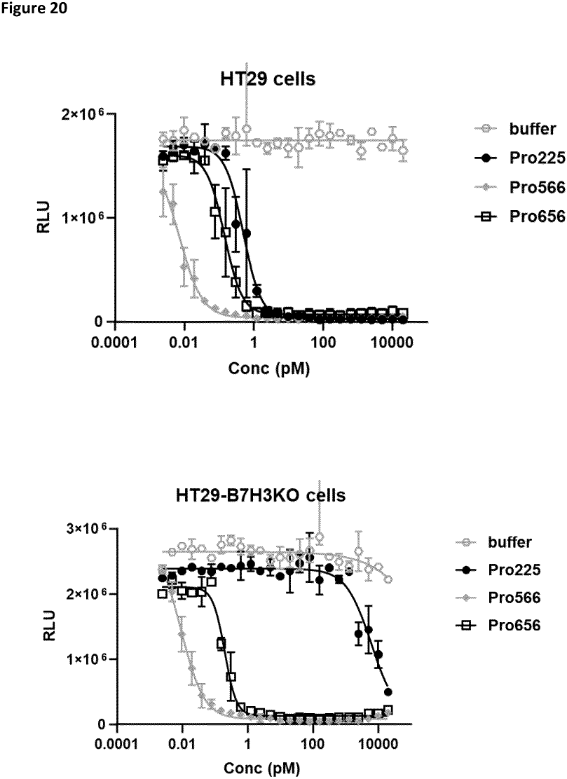

FIG. 20 shows the efficacy of a number of format 2 constructs of the invention in a TDCC assay as outlined in Example 2 on two different cell lines. Pro225 is an aB7H3 construct with an MMP9 cleavage site; Pro566 is an aEpCAM construct with an MMP9 cleavage site; and Pro656 is a heteroCOBRA of aB7H3 and aEpCAM and an MMP9 site. HT29 is an epithelial cell line that, unlike Raji cell lines, make good xenografts in mice. HT29 expresses both target genes, (B7H3 and EpCAM), and in this case, the B7H3 expression was knocked out using CRISPR. Thus, the heteroCOBRA and the EpCAM single targeting COBRA killed both, while the B7H3 single targeting COBRA did not.

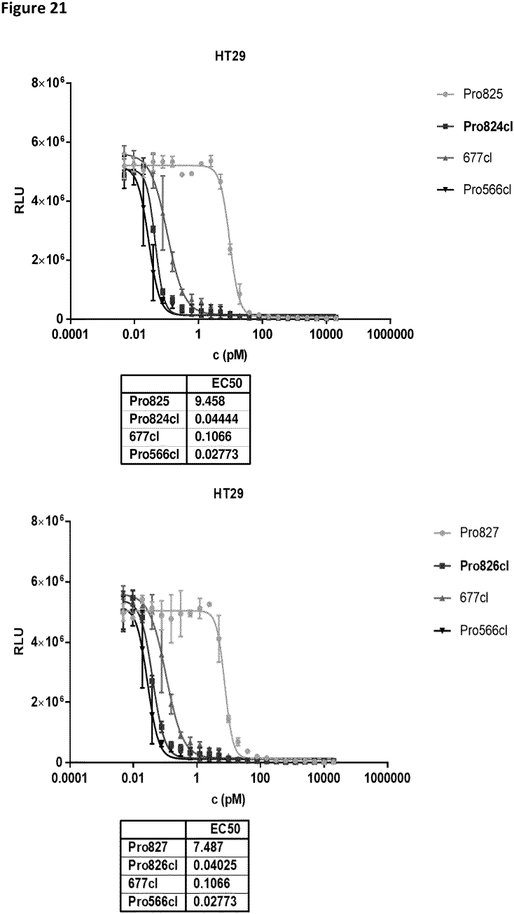

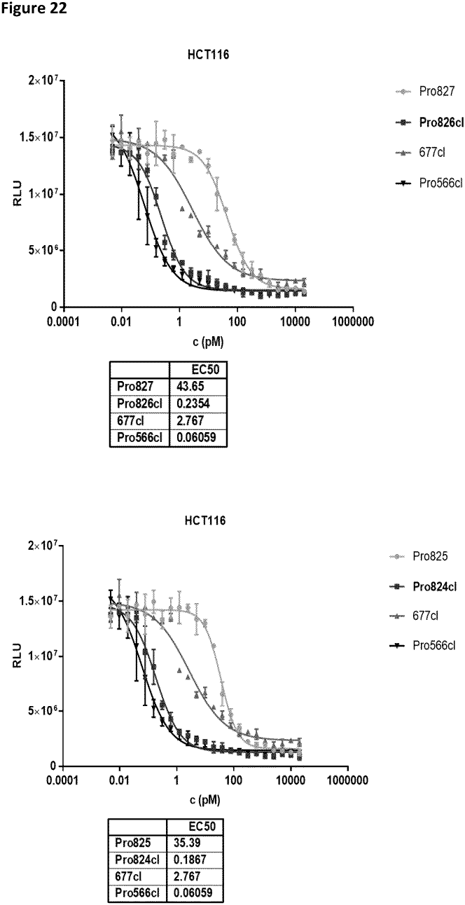

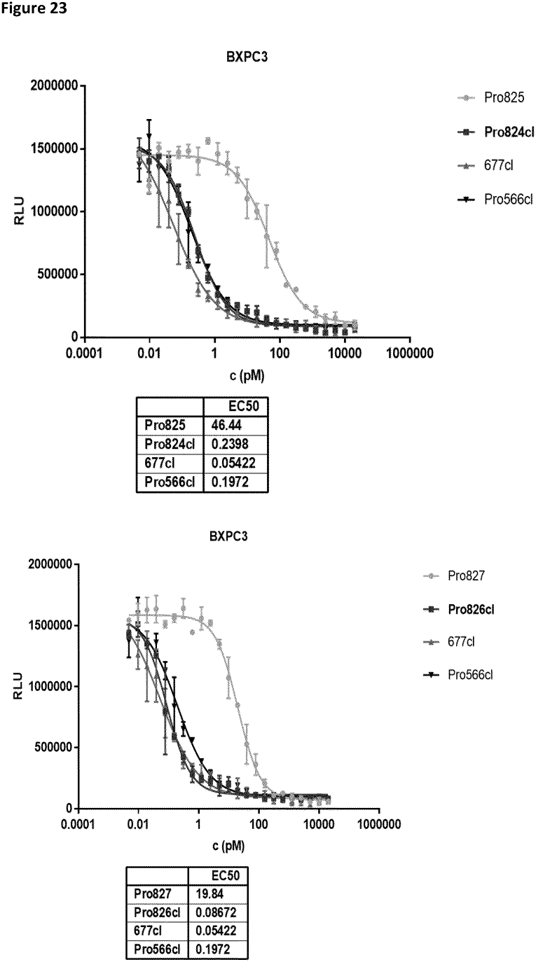

FIG. 21 shows the efficacy of a number of format 2 constructs of the invention in a TDCC assay as outlined in Example 2 on the HT29 cell line that has high EpCAM expression and low Trop2 expression. Pro824 is an aEpCAM X aTrop2 (with an MMP9 linker) heteroCOBRA. Pro825 is an aEpCAM X aTrop2 heteroCOBRA with a NCL (non-cleavable control). Pro826 is an aTrop2 X aEpCAM HeteroCOBRA with an MMP9 linker. Pro827 is an aTrop2 X aEpCAM HeteroCOBRA with a NCL (non-cleavable control). Pro677 is an aTrop2/MMP9 COBRA and Pro566 is an aEpCAM/MMP9 COBRA. As the levels of the two antigens vary, the heteroCOBRAs maintain good killing while the killing with the monospecific COBRAs varies. The monospecific COBRAs don’t kill as well when the the expression level of their specific antigen drops (in this case Trop2); the same is true for FIGS. 22 and 23.

FIG. 22 shows the efficacy of a number of format 2 constructs of the invention in a TDCC assay as outlined in Example 2 on the HT116 cell line that has high EpCAM expression and very low Trop2 expression. Pro824 is an aEpCAM X aTrop2 (with an MMP9 linker) heteroCOBRA. Pro825 is an aEpCAM X aTrop2 heteroCOBRA with a NCL (non-cleavable control). Pro826 is an aTrop2 X aEpCAM HeteroCOBRA with an MMP9 linker. Pro827 is an aTrop2 X aEpCAM HeteroCOBRA with a NCL (non-cleavable control). Pro677 is an aTrop2/MMP9 COBRA and Pro566 is aEpCAM/MMP9 COBRA.

FIG. 23 shows the efficacy of a number of format 2 constructs of the invention in a TDCC assay as outlined in Example 2 on the BXPC3 cell line that has medium EpCAM expression and high Trop2 expression. Pro824 is an aEpCAM X aTrop2 (with an MMP9 linker) heteroCOBRA. Pro825 is an aEpCAM X aTrop2 heteroCOBRA with a NCL (non-cleavable control). Pro826 is an aTrop2 X aEpCAM HeteroCOBRA with an MMP9 linker. Pro827 is an aTrop2 X aEpCAM HeteroCOBRA with a NCL (non-cleavable control). Pro677 is an aTrop2/MMP9 COBRA and Pro566 is an aEpCAM/MMP9 COBRA.

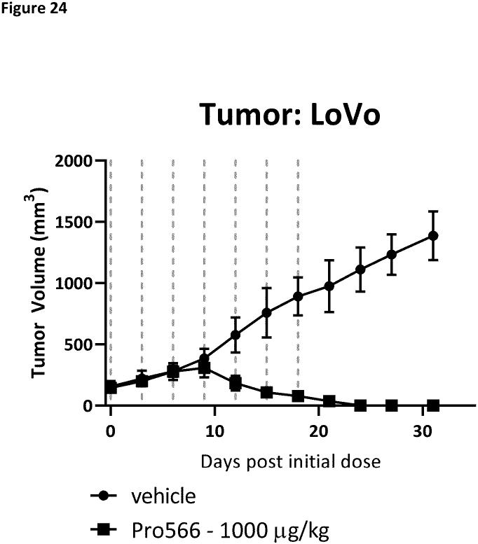

FIG. 24 shows the in vivo efficacy of an aEpCAM COBRA with an MMP9 cleavage site using Protocol 2 of Example 3. Pro566 showed efficacy on LoVo tumors, as well as HT29, BxPC3 and SW403 tumor xenografts.

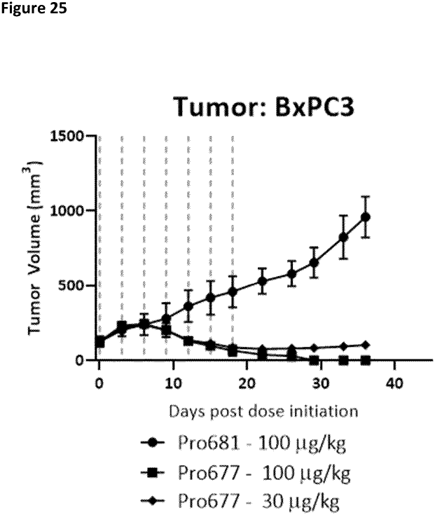

FIG. 25 shows the in vivo efficacy of an aTrop2 COBRA with an MMP9 cleavage site using Protocol 2 of Example 3. Pro677 showed efficacy on BxPC3 tumors, as well as HCC827 tumor xenografts.

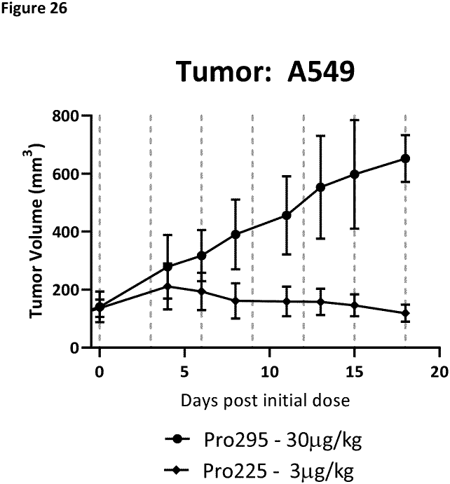

FIG. 26 shows the in vivo efficacy of an aB7H3 COBRA with an MMP9 cleavage site using Protocol 3 of Example 3. Pro225 showed efficacy on A549 tumors.

Conclusions: We have designed a multivalent sdAb-diabody fusion which converts into a highly potent bispecific redirected T-cell therapeutic upon proteolytic action. In vitro assay demonstrated that protease dependent linker cleavage increased potency of T cell-mediated killing by 200-fold, thus yielding a therapeutic with sub-picomolar potency. Administration of PRO186 (Pro186) in mice with established xenografts resulted in protease cleavage dependent T cell-mediated tumor regressions in multiple tumor models. PRO186 displayed (1) extended half-life in vivo upon administration and (2) rapid clearance post proteolytic activation, thereby demonstrating PRO186 to be a therapeutic with improved safety profile over conventional T-cell redirected bispecifics.

### Example 9: Anti-HER2 Mono-specific COBRAs Killed Tumor Cell Lines Conditionally in TDCC experiments

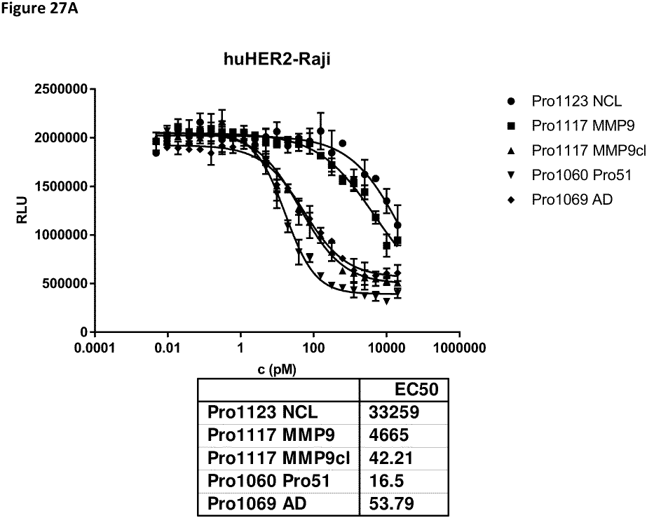

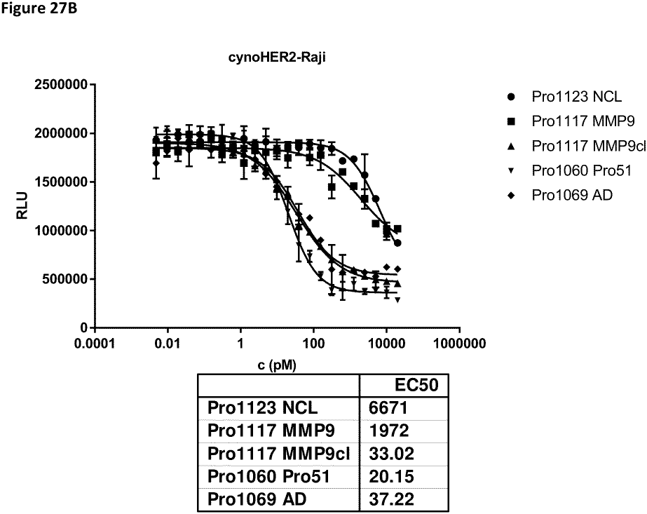

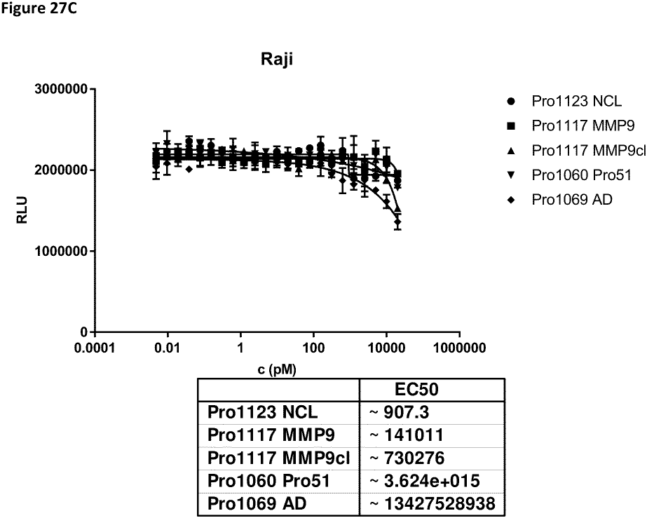

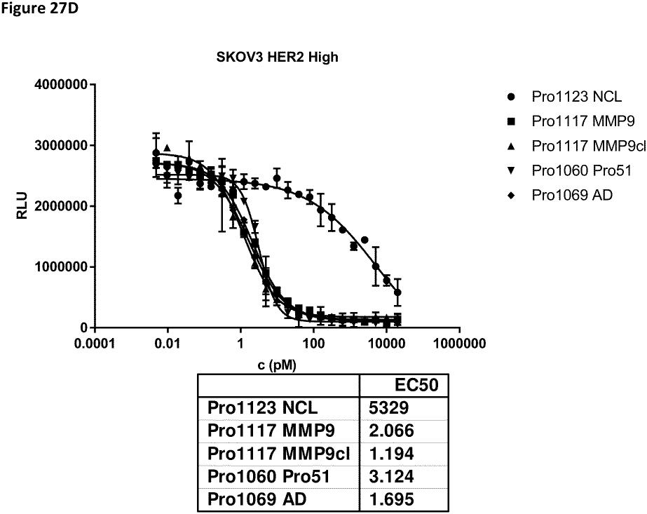

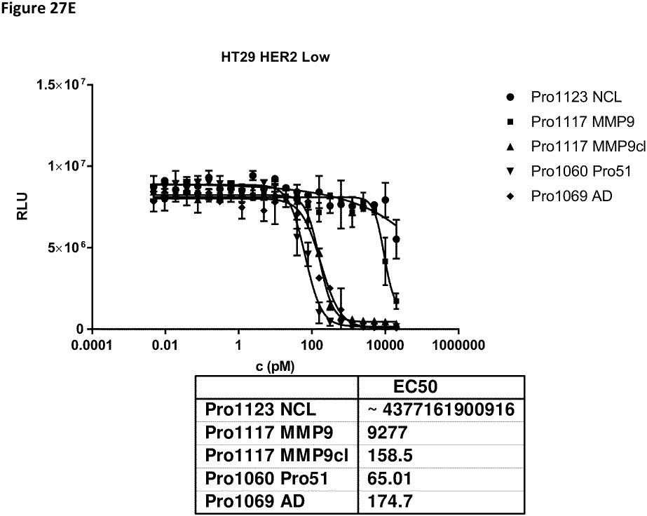

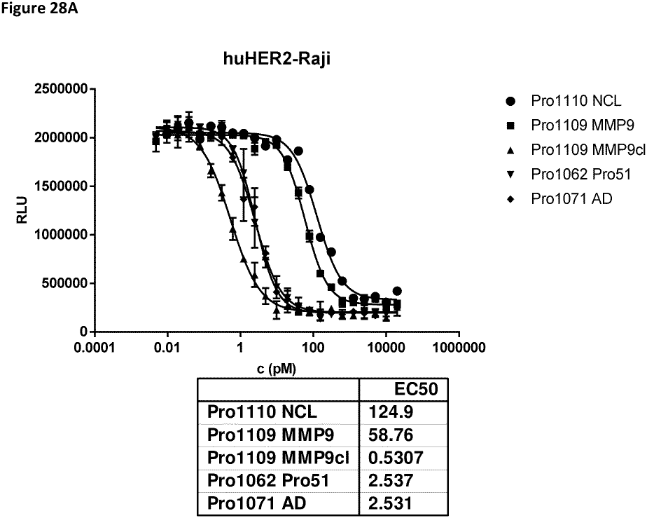

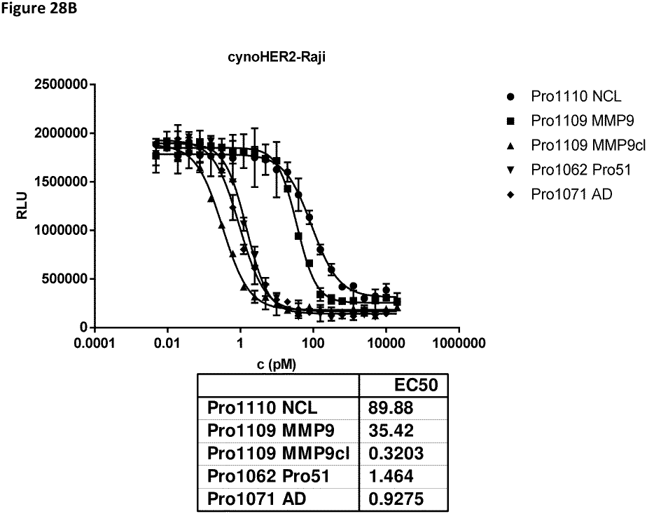

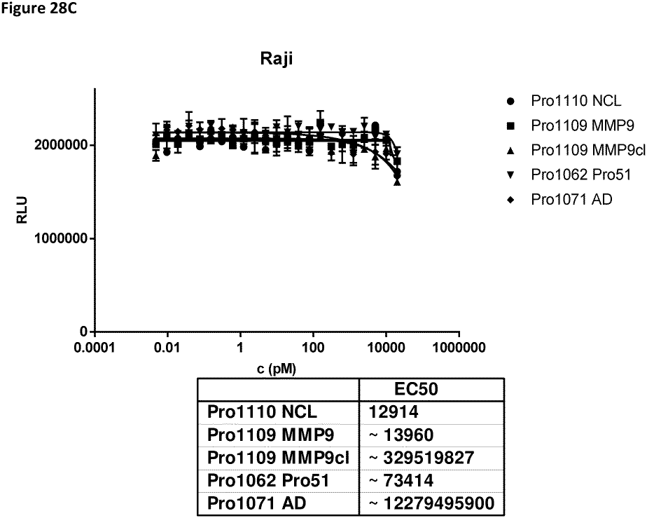

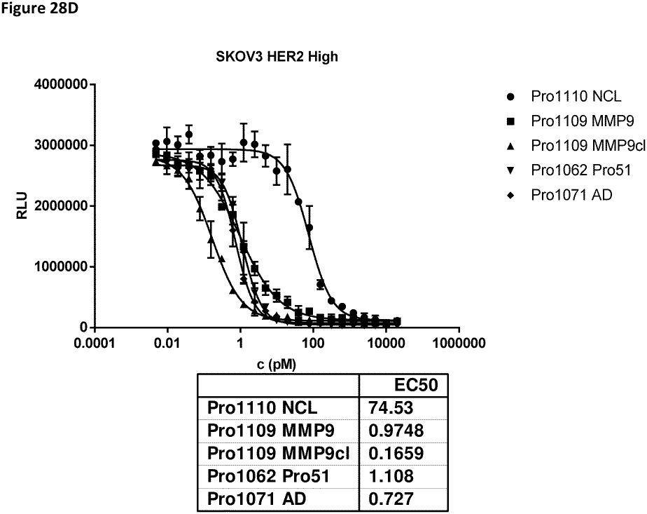

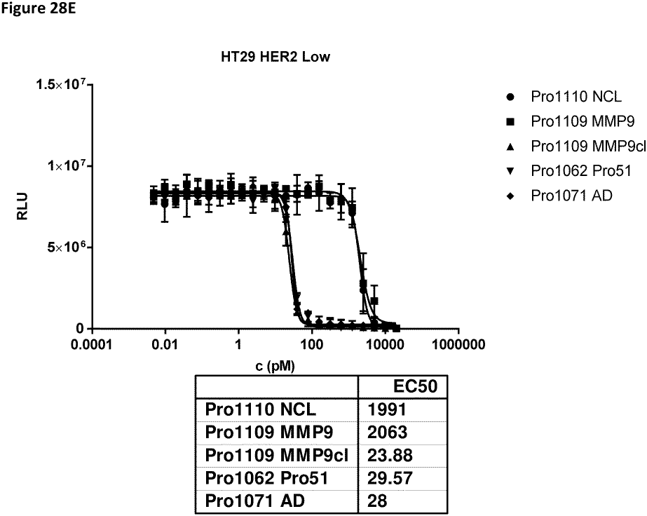

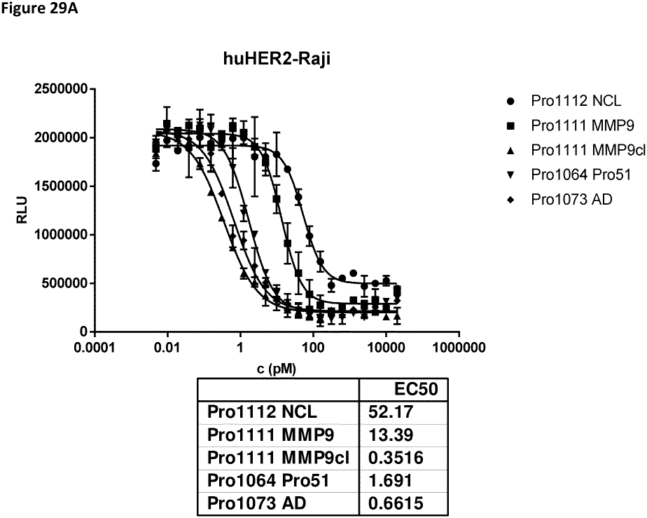

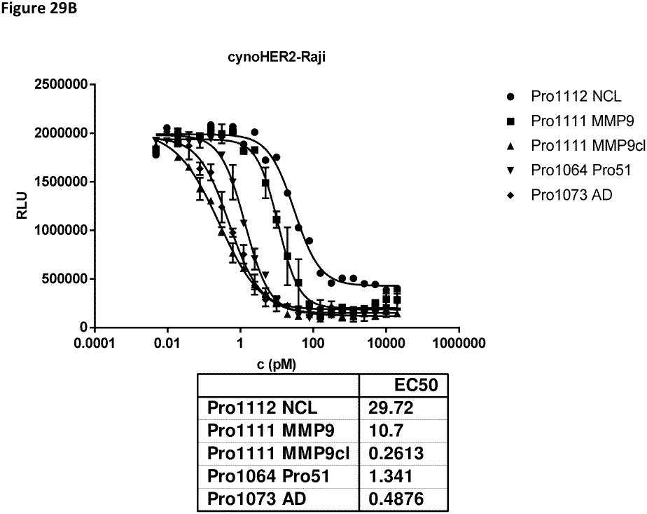

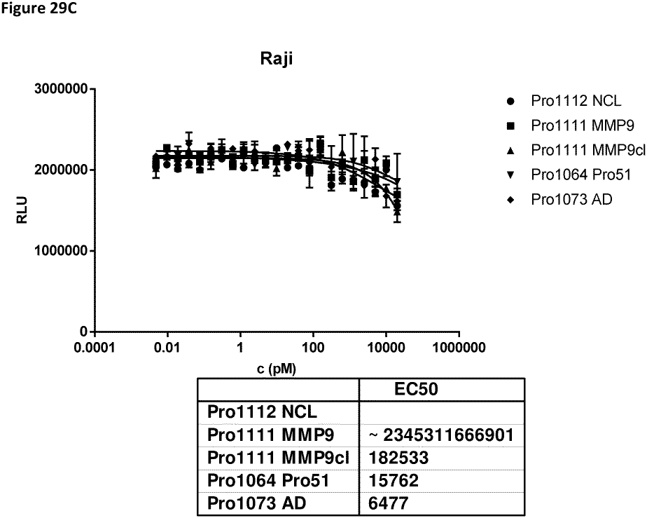

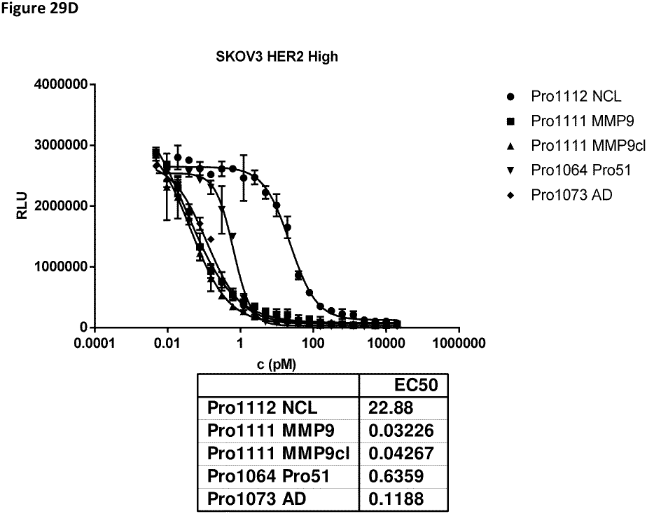

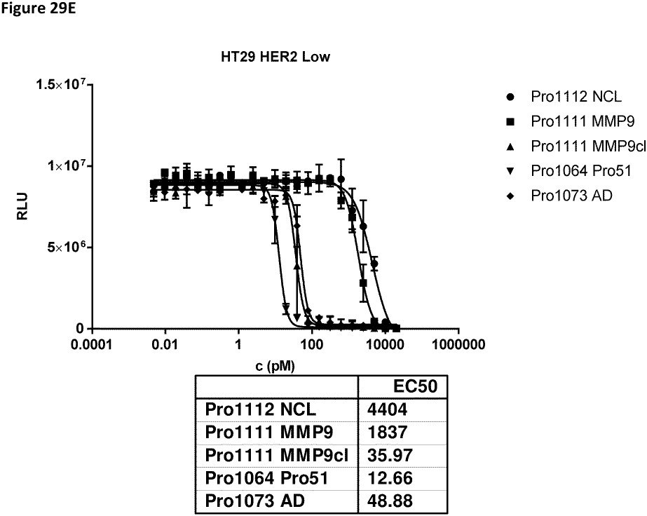

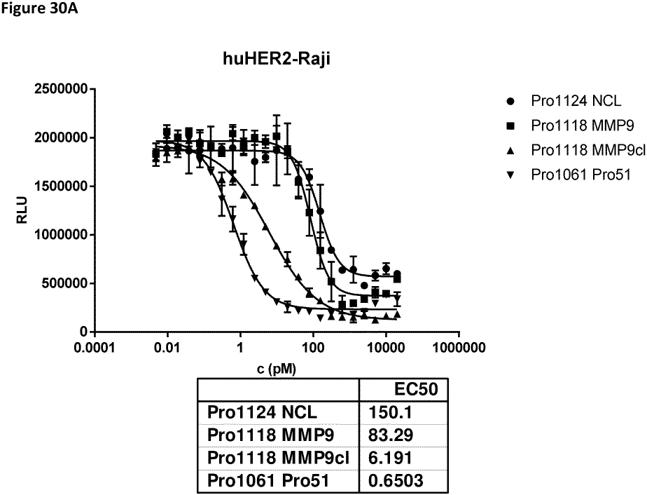

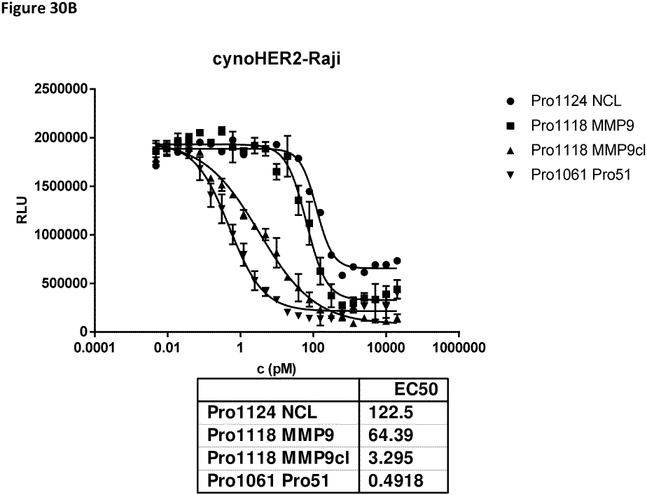

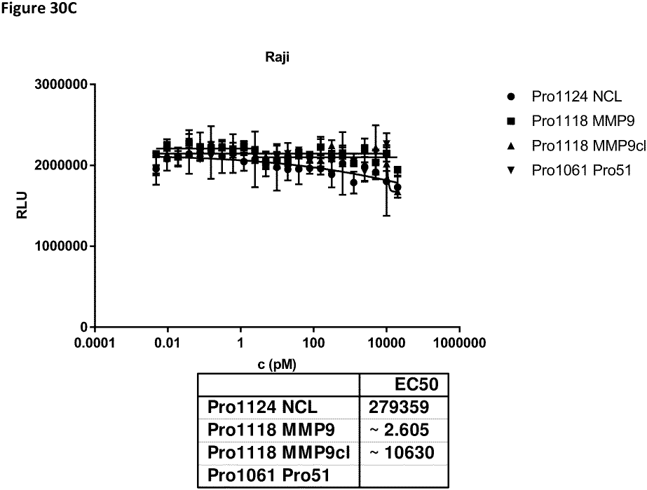

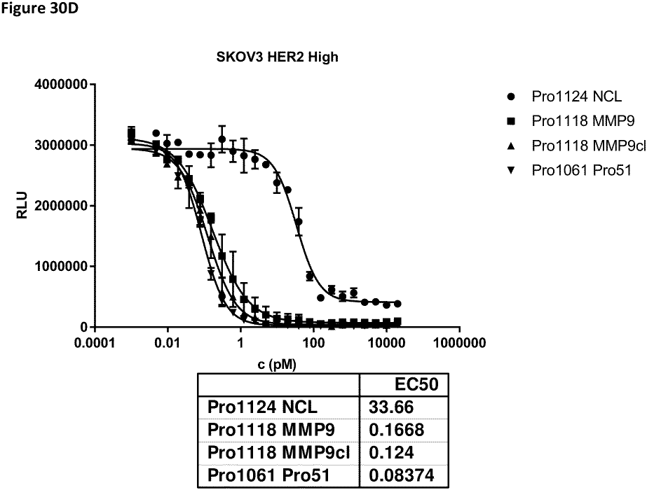

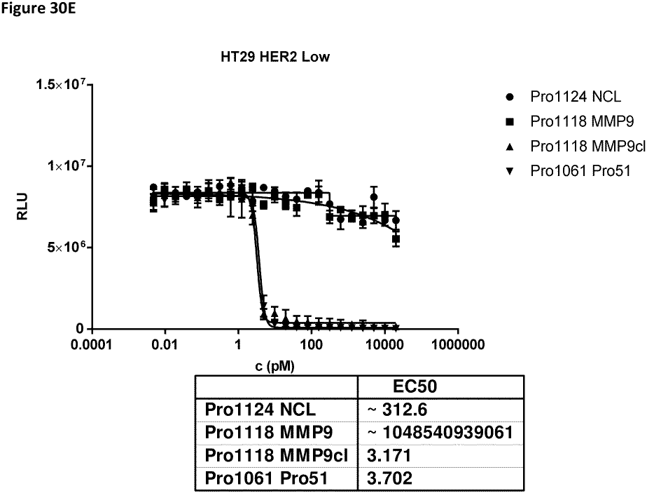

Human HER2-Raji cells, cynomolgus monkey (cyno) HER2-Raji cells, SKOV3 cells (low expressing HER2 cells), Raji-paternal cells, HT29 cells (high expressing HER2 cells) were tested with various fusion proteins: Pro1123 NCL, Pro1117 MMP9, Pro 1117 MMP9cl, Pro1060 Pro51, and Pro1069 AD (FIGS. 27A-27E); Pro1110 NCL, Pro1109 MMP9, Pro 1109 MMP9cl, Pro 1062 Pro51 and Pro1071 AD (FIGS. 28A-28E); Pro1112 NCL, Pro1111 MMP9, Pro 1111 MMP9cl, Pro1064 Pro51 and Pro1073 AD (FIGS. 29A-29E); Pro1124 NCL, Pro1118 MMP9, Pro 1118 MMP9cl, Pro1061 Pro51, and Pro1069 AD (FIGS. 30A-30E). The results showed that monospecific COBRAs comprising aHER2 sdABDs (aHer2 h1139, h1159, h1162, and h1156) were able to kill tumor cell lines conditionally in TDCC assays.

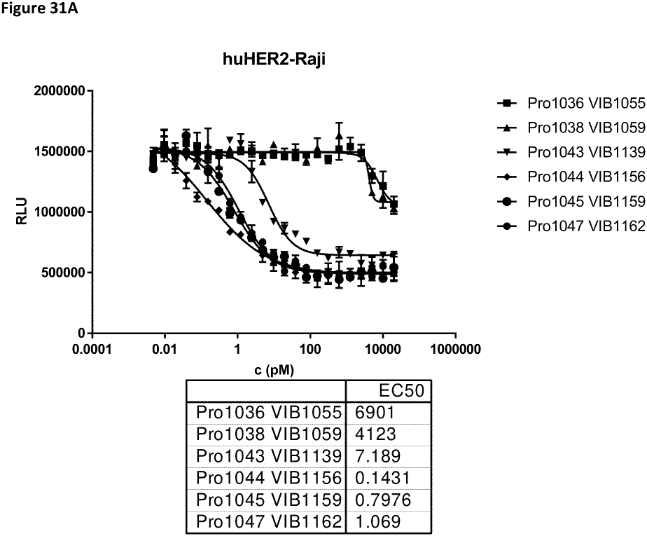

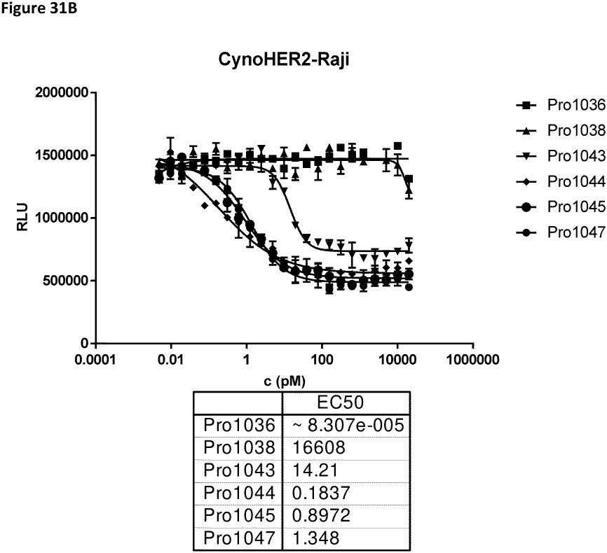

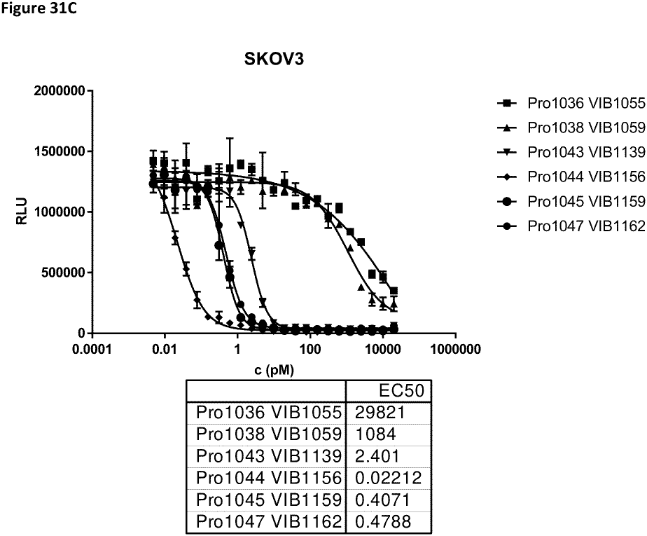

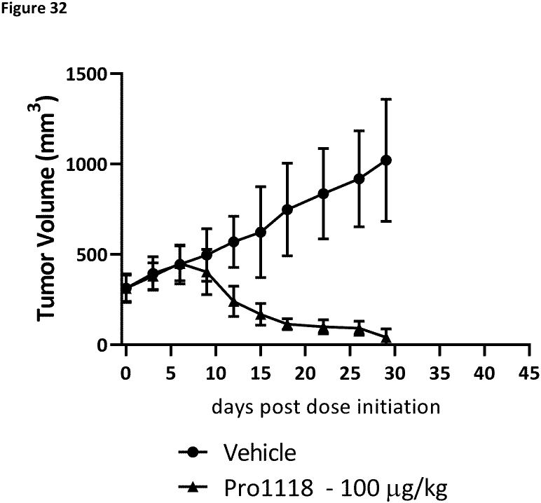

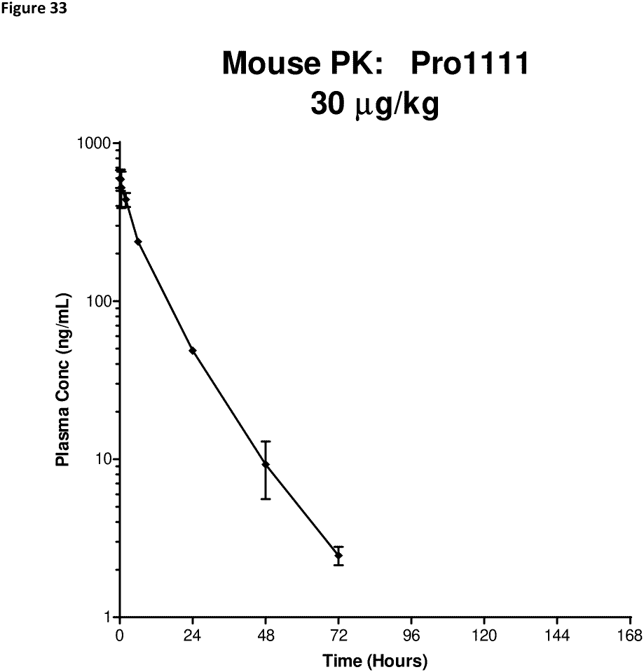

aHER2 fusion proteins in a Pro51 format and containing one aHER2 sdABD such as either the VIB1139 HER2 sdABD, the VIB1156 HER2 sdABD, the VIB1159 HER2 sdABD or the VIB1162 HER2 sdABD demonstrated good activity against human and cross-reactivity with cynomolgus in TDCC experiments (FIGS. 31A-31C). Additionally, aHER2 mono-specific COBRAs of Format 2 and comprising an MMP9 cleavage linker (HER2/MMP9 COBRA) were able to regress established tumor xenografts (FIG. 32), in particular, Pro1118 was administered to the mice at a dose of 100 ug/kg. FIG. 33 depicts a graph showing mouse PK data of a monospecific HER2 COBRA comprising an MMP9 cleavable linker. The results indicate that Pro1111 activity is consistent with murine HER2 binding.

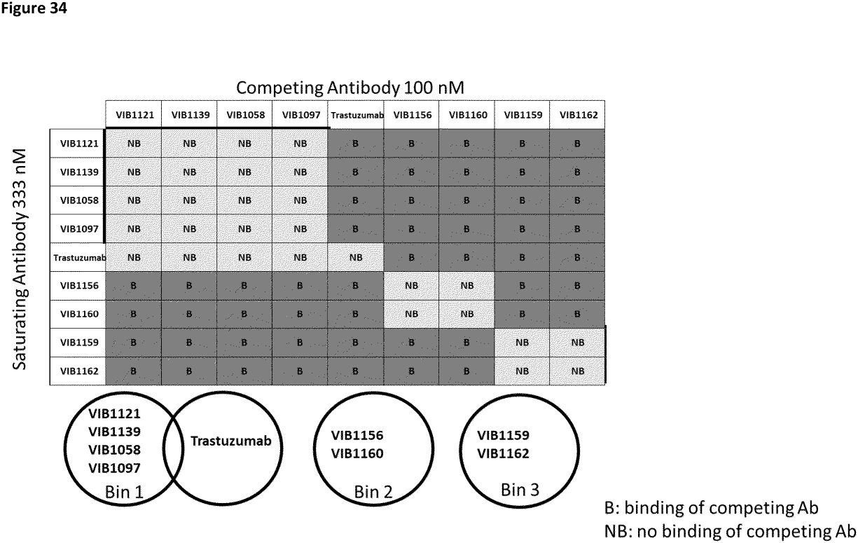

The epitope binning experiments of various HER2 sdAbs were performed as understood by those skilled in the art. Competing antibodies at 100 nM were tested with saturating antibodies at 333 nM. The tested competing antibodies were: Pro1118, Pro1111, trastuzumab, and pertuzumab. The saturating tested antibodies were: the VIB1121 HER2 sdABD, the VIB1139 HER2 sdABD, the VIB1058 HER2 sdABD, the VIB1097 HER2 sdABD, trastuzumab, the VIB1156 HER2 sdABD, the VIB1160 HER2 sdABD, the VIB1159 HER2 sdABD, and the VIB1162 HER2 sdABD (FIG. 34).

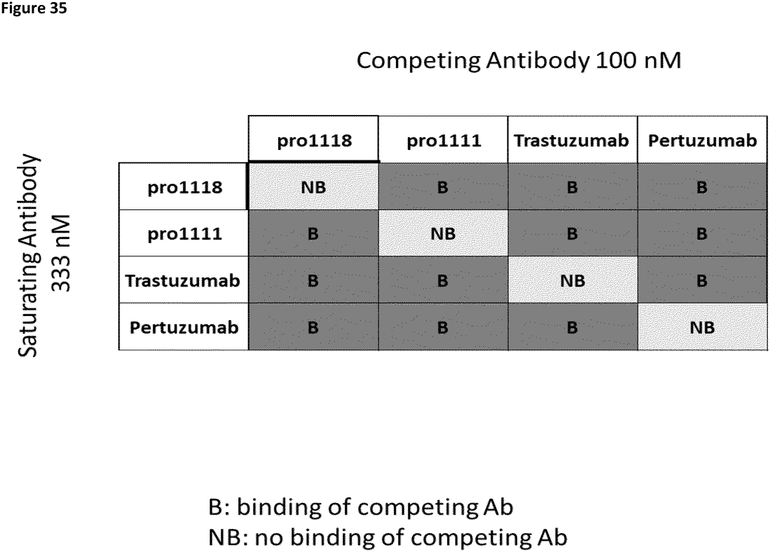

The epitope binning experiments of various HER2 sdAbs were performed as recognized by those skilled in the art. Competing antibodies at 100 nM were tested with saturating antibodies at 333 nM. The tested antibodies were: Pro1118, Pro1111, trastuzumab, and pertuzumab. “B” indicates binding of competing Ab and “NB” indicates no binding of competing Ab (FIG. 35).

The amino acids locations and sequences from the epitope mapping analysis of HER2 sdAb h1156 (Pro1061) and HER2 sdAb h1162 (Pro1064) were identified using HDX (hydrogen-deuterium exchange), performed as recognized by those skilled in the art (FIG. 36). Human Her2 protein showed significant reduction in deuterium uptakes upon binding to protein ligand Pro1061 at sequences AA147-148, WK, and AA157-161, LALTL, which could be assigned as the epitope on Human Her2 protein targeted by protein ligand Pro1061. Human Her2 protein showed moderate reduction in deuterium uptakes upon binding to protein ligand Pro1064 at sequences AA464-472, FRNPHQALL, which could be assigned as the epitope on Human Her2 protein targeted by protein ligand Pro1064.

The binding affinities of HER2 sdAbs in Pro51 format were determined. Various sdAb and fusion proteins combinations were assessed with targets from human, cynomolgus monkey and mouse. The combinations were the following: 1055 and Pro1036; 1058 and Pro1037; 1059 and Pro1038; 1091 and Pro1039; 1092 and Pro1040; 1097 and Pro1041; 1121 and Pro1042; 1139 and Pro1043; 1156 and Pro1044; 1159 and Pro1045; 1160 and Pro1046; 1162 and Pro1047; h1058 and Pro1056; h1092 and Pro1057; h1097 and Pro1058; h1121 and Pro1059; h1139 and Pro1060; h1156 and Pro1061; h1159 and Pro1062; h1160 and Pro1063; and h1162 and Pro1064 (FIG. 37).

### Example 10: Anti-CA9 Monospecific COBRAs Killed Tumor Cell Lines Conditionally in TDCC experiments

Human CA9-Raji cells, cyno CA9-Raji cells, and HT29-parental cells were tested with various fusion proteins: Pro514 NCL, Pro518 MMP9, Pro518 MMP9cl, Pro511 Pro51, and Pro521 AD (FIGS. 38A-38C). Monospecific COBRAs targeting CA9 such as those comprising aCA9 sdABDs (aCA9 h407) were able to kill human or cyno CA9 expressing tumor cell lines conditionally.

Human CA9-Raji cells, cyno CA9-Raji cells, and HT29-parental cells were tested with various fusion proteins: Pro515 NCL, Pro519 MMP9, Pro519 MMP9cl, and Pro512 Pro51. Monospecific COBRAs targeting CA9 such as those comprising aCA9 sdABDs (aCA9 h445) were able to kill human or cyno CA9 expressing tumor cell lines conditionally (FIGS. 39A-39C).

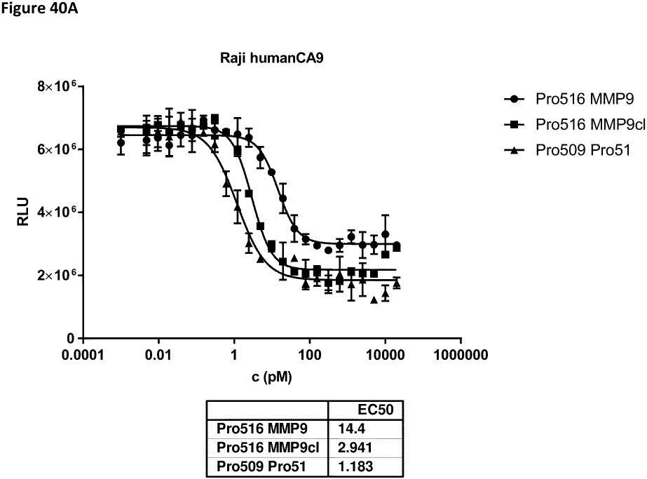

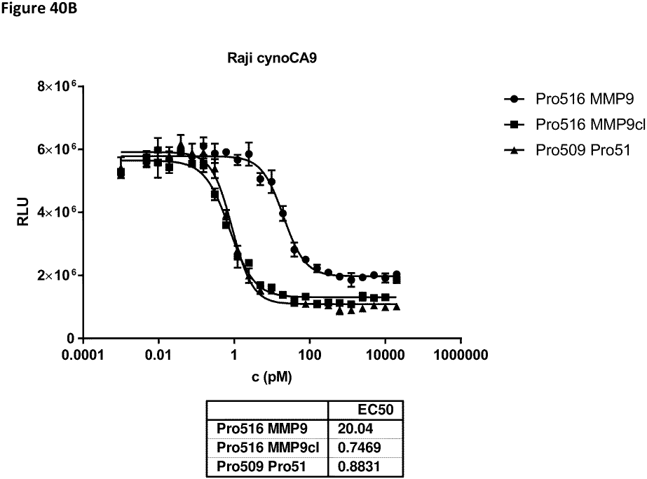

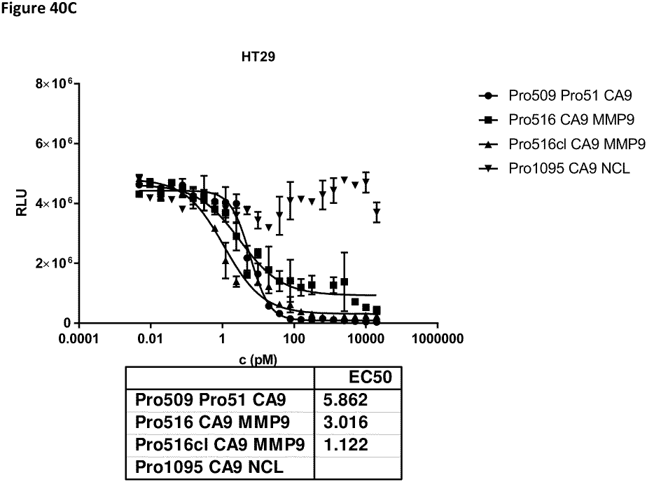

Human CA9-Raji cells, cyno CA9-Raji cells, and HT29-parental cells were tested with various fusion proteins: Pro1095 NCL, Pro516 MMP9, Pro516 MMP9cl, and Pro509 Pro51. Monospecific COBRAs targeting CA9 such as those comprising aCA9 sdABDs (aCA9 h456) were able to kill human or cyno CA9 expressing tumor cell lines conditionally (FIGS. 40A-40C).

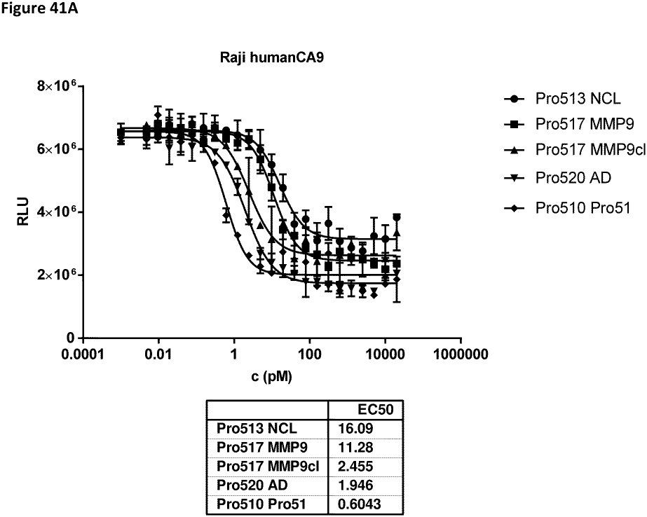

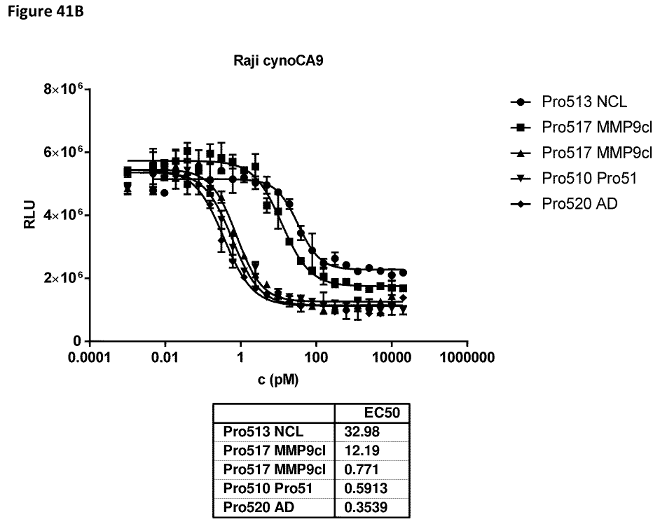

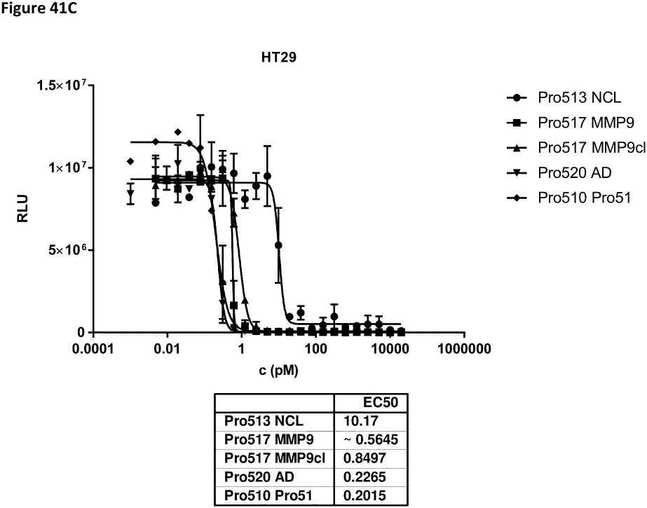

Human CA9-Raji cells, cyno CA9-Raji cells, and HT29-parental cells were tested with various fusion proteins: Pro513 NCL, Pro517 MMP9, Pro517 MMP9cl, Pro520 AD and Pro510 Pro51. Monospecific COBRAs targeting CA9 such as those comprising aCA9 sdABDs (aCA9 h4) were able to kill human or cyno CA9 expressing tumor cell lines conditionally (FIGS. 41A-41C).

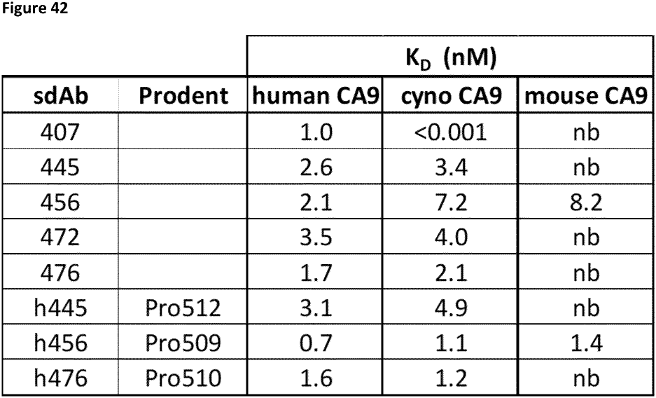

FIG. 42 is a table depicting the binding affinities of CA9 sdAbs in a Pro51 format. Various sdAb and combination of sdAb and fusion proteins were assessed in human, cyno and mouse. The sdAbs were the following: 407, 445, 456, 472 and 476 and the combinations were the following: h445 and Pro512; h456 and Pro509; and h476 and Pro510.

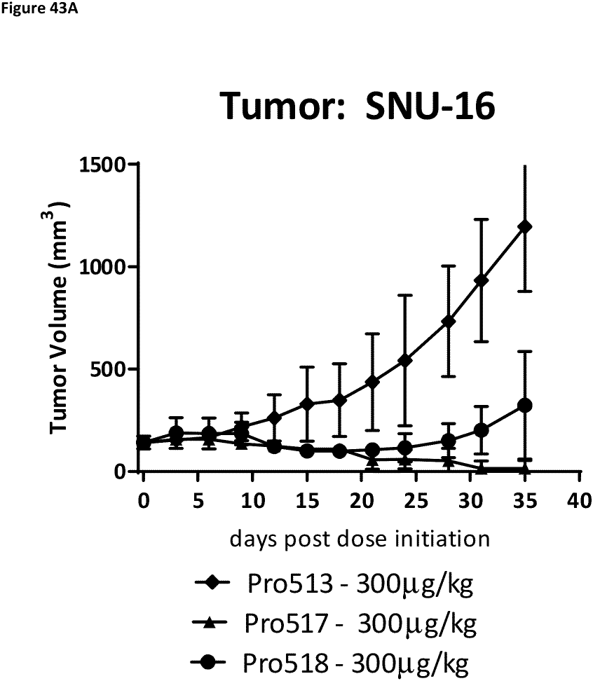

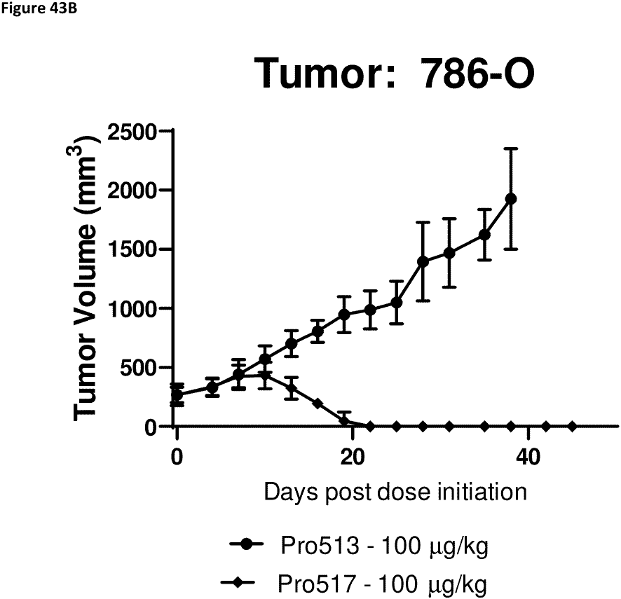

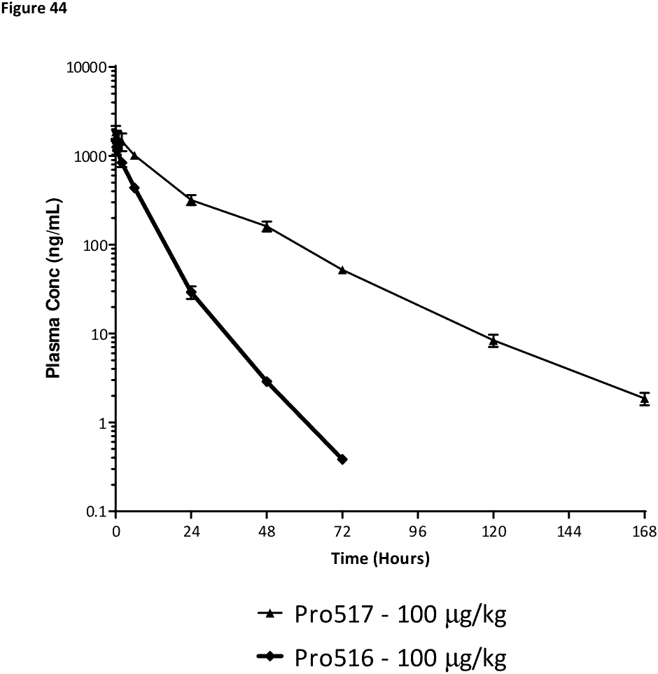

CA9 monospecific COBRAs of Format 2 and comprising an MMP9 cleavage linker (CA9/MMP9 COBRA) were able to regress established tumor xenografts. FIGS. 43A-43B are series of graphs demonstrating that CA9/MMP9 COBRAs regressed in established tumor xenograft models. Tumor SNU-16 in presence of Pro513, Pro517 and Pro518, all at a dose of 300 ug/kg. Tumor 786-O in presence of Pro513 and Pro517, all at a dose of 100ug/kg. FIG. 44 is a graph showing mouse PK data of CA9/MMP9 COBRA which is consistent with murine target binding for Pro516. Pro517 and Pro516 were used at dose of 100 ug/kg.

### Example 11: EGFR/EpCAM Hetero-COBRAs Induced TDCC of Cells expressing EGFR and EpCAM

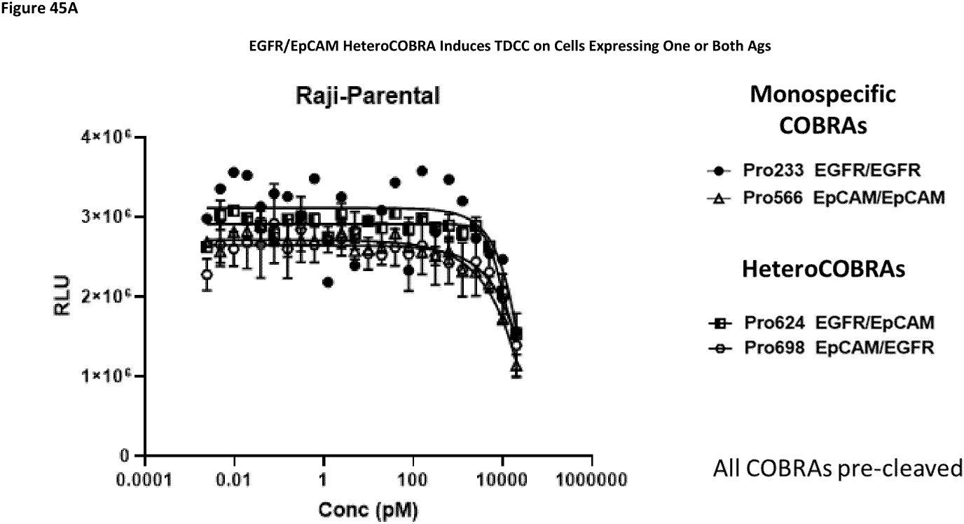

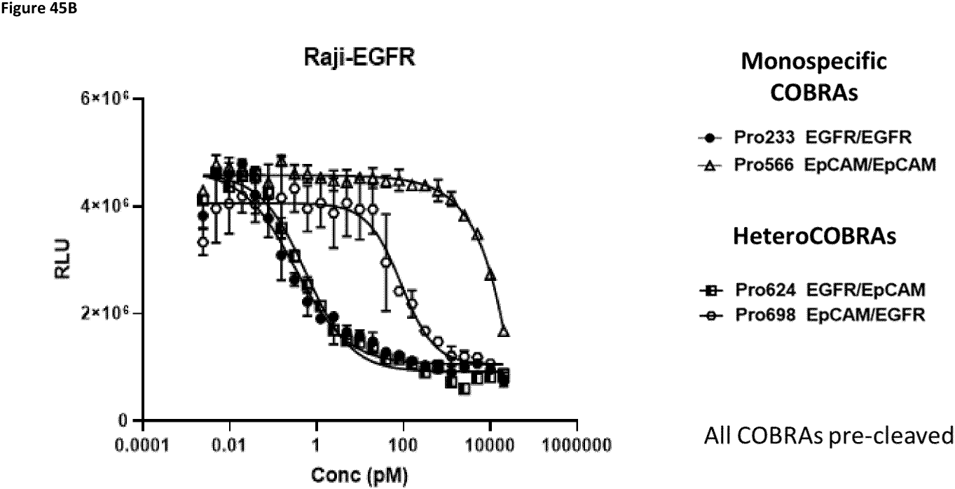

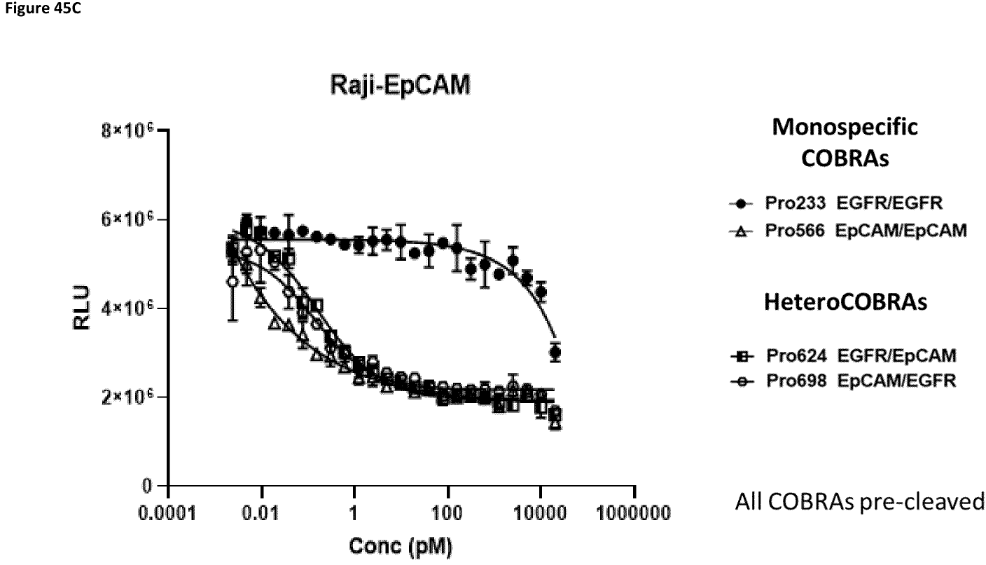

Raji-parental cells (FIG. 45A), Raji-EGFR cells (FIG. 45B), Raji-EpCAM cells (FIG. 45C), and Raji-EGFR/EpCAM cells (FIG. 45D) were tested with monospecifc COBRAs: Pro233 (EGFR/EGFR) and Pro566 (EpCAM/EpCAM) and with heteroCOBRAs Pro624 (EGFR/EpCAM) and Pro698 (EpCAM/EGFR). The results showed that hetero-specific COBRAs targeting both EGFR and EpCAM induced TDCC on Raji cells expressing one or both antigens (e.g., either EGFR alone, EpCAM alone, or both EGFR and EpCAM).

Pro624 comprises from N- to C-terminal: (sdABD-EGFR)-domain linker-aVH-CNCL-aVL-domain linker-(sdABD-EpCAM)-CL-iVL-CNCL-iVH-domain linker-(sdABD-HSA). Pro698 comprises from N- to C-terminal: (sdABD-EpCAM)-domain linker-aVH-CNCL-aVL-domain linker-(sdABD-EGFR)-CL-iVL-CNCL-iVH-domain linker-(sdABD-HSA). EGFR/EpCAM heteroCOBRAs were also able to induce TDCC on HT29 cells expressing one or both antigens.

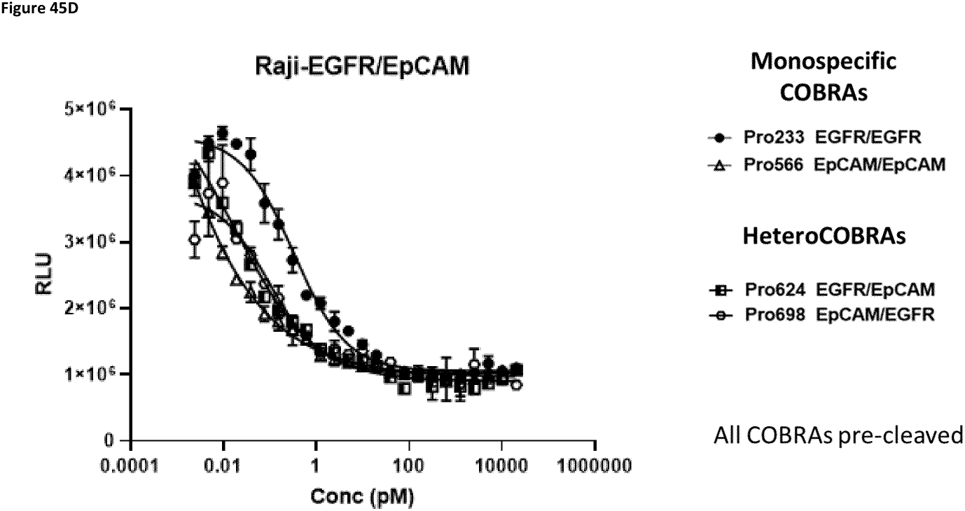

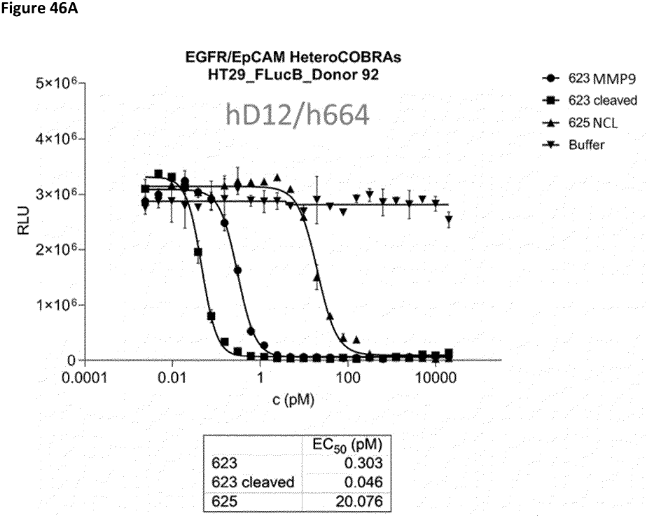

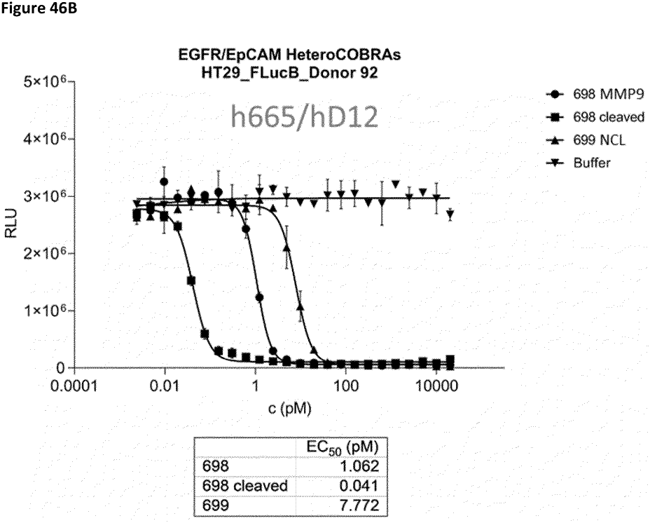

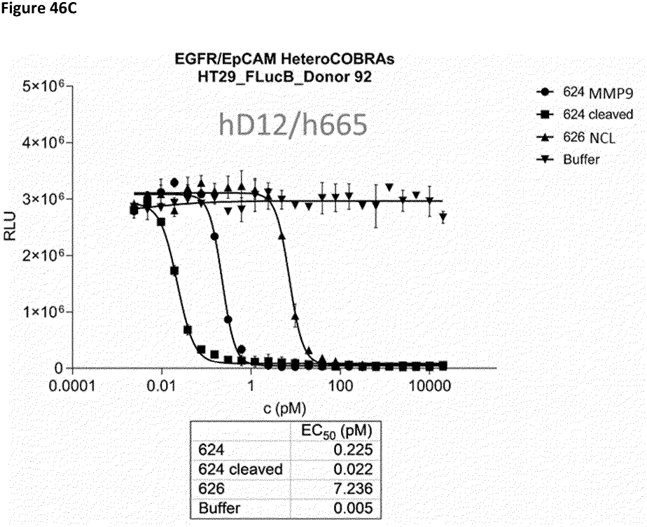

EGFR/EpCAM heteroCOBRAs comprising an aEGFR sdABD (aEGFR hD12) and an aEpCAM sdABD (aEpCAM h644) were tested with Pro623 MMP9, Pro623 cleaved, Pro625 NCL (FIG. 46A). Pro623 comprises from N- to C-terminal: (sdABD-EGFR)-domain linker-aVH-CNCL-aVL-domain linker-(sdABD-EpCAM)-CL-iVL-CNCL-iVH-domain linker-(sdABD-HSA).

EGFR/EpCAM heteroCOBRAs comprising an aEGFR sdABD (aEGFR hD12) and an aEpCAM sdABD (aEpCAM h665) were tested with Pro698 MMP9, Pro698 cleaved, 699 NCL (FIG. 46B). Pro699 comprises from N- to C-terminal: (sdABD-EpCAM)-domain linker-aVH-CNCL-aVL-domain linker-(sdABD-EGFR)-NCL-iVL-CNCL-iVH-domain linker-(sdABD-HSA). EGFR/EpCAM heteroCOBRAs comprising an aEGFR sdABD (aEGFR hD12) and an aEpCAM sdABD (aEpCAM h665) were tested with Pro624 MMP9, Pro624 cleaved, Pro699 NCL (FIG. 46C). EGFR/EpCAM heteroCOBRAs were able to induce TDCC in various cells expressing one or both antigens.

### Example 12: EGFR/FOLR1 HeteroCOBRAs Induced TDCC of Cells Expressing EGFR and FOLR1

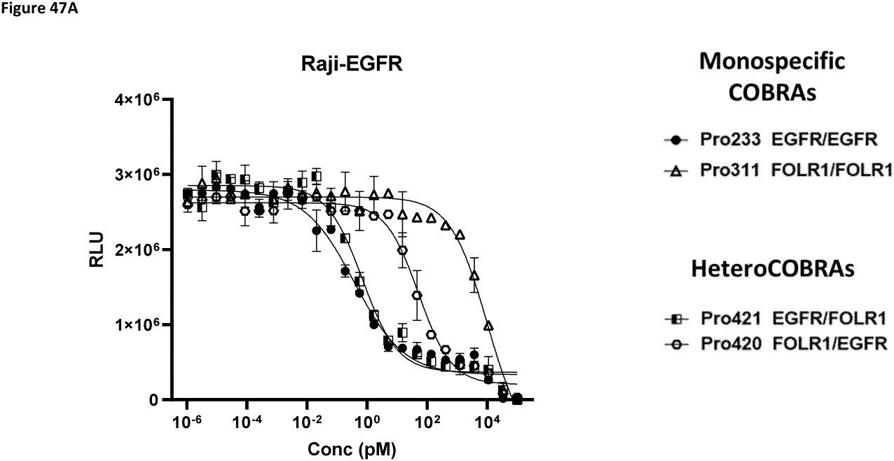

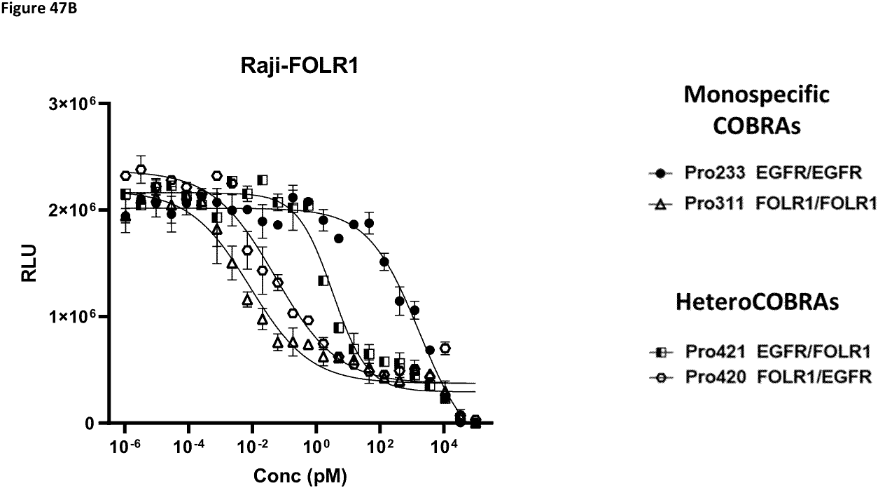

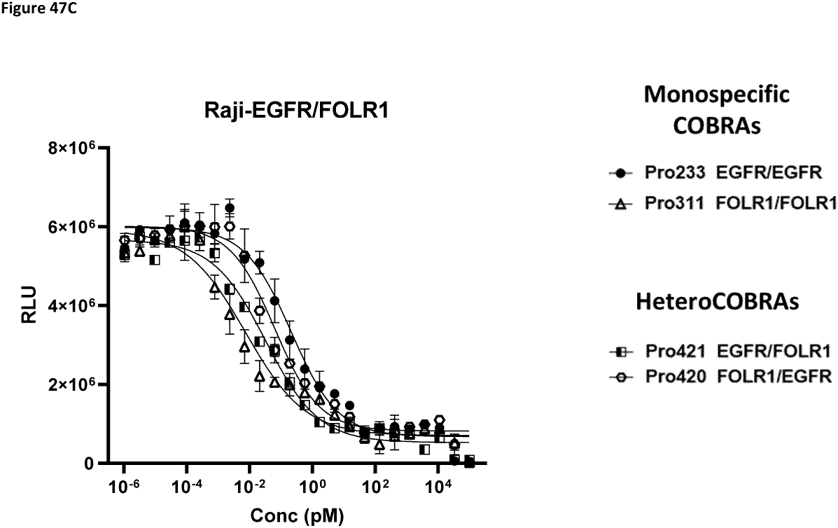

Raji-EGFR cells (FIG. 47A), Raji-FOLR1 cells (FIG. 47B), Raji-EGFR/FOLR1 cells (FIG. 47C) were tested with monospecifc COBRAs targeting either EGFR or FOLR1: Pro233 (EGFR/EGFR) and Pro311 (FOLR1/ FOLR1) and with heteroCOBRAs targeting both EGFR and FOLR1: Pro421 (EGFR/FOLR1) and Pro420 (FOLR1/EGFR). Pro421 comprises from N- to C-terminal: (sdABD-EGFR)-domain linker-aVH-CNCL-aVL-domain linker-(sdABD-FOLR1)-CL-iVL-CNCL-iVH-domain linker-(sdABD-HSA). Pro420 comprises from N- to C-terminal: (sdABD-FOLR1)-domain linker-aVH-CNCL-aVL-domain linker-(sdABD- EGFR)-CL-iVL-CNCL-iVH-domain linker-(sdABD-HSA). The results of these experiments demonstrated that EGFR/FOLR1 heteroCOBRAs (such as Pro421) induced TDCC on Raji cells expressing one or both antigens.

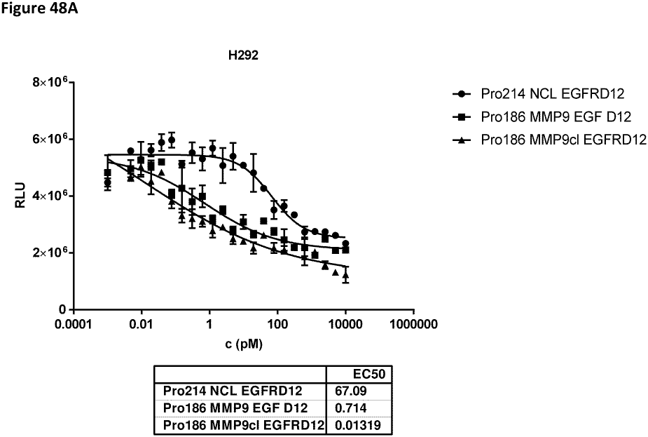

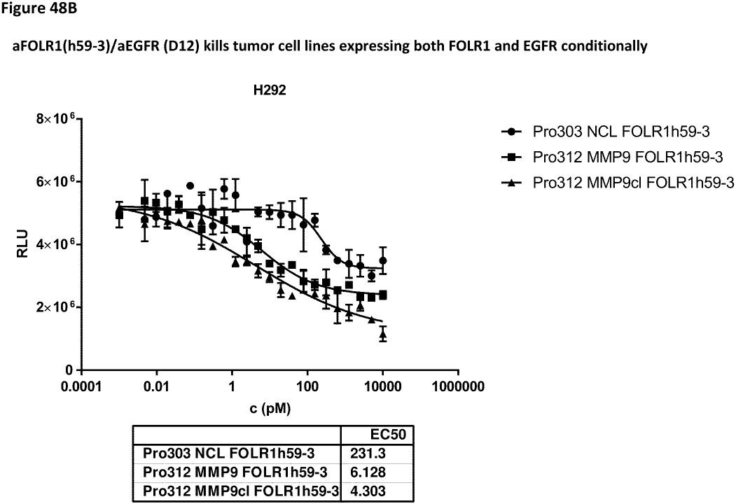

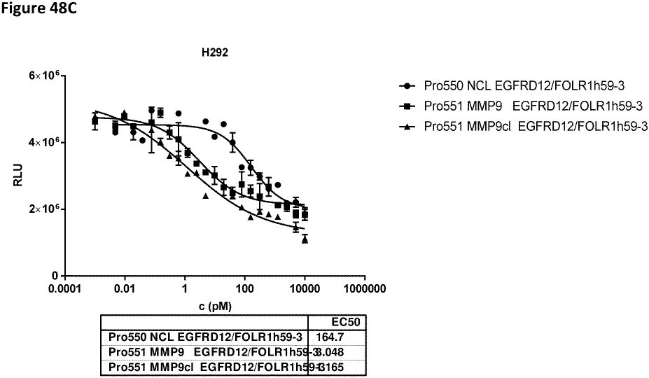

FIG. 48A: H292 cells were tested with Pro214 NCL (EGFR hD12), Pro186 MMP9 (EGFR hD12), and Pro186 MMP9cl (EGFR hD12). FIG. 48B: H292 cells were tested with Pro303 NCL (FOLR1 h59-3), Pro312 MMP9 (FOLR1 h59-3), and Pro312 MMP9cl (FOLR1 h59-3). FIG. 48C: H292 cells were tested with Pro550 NCL (EGFR/FOLR1 h59-3), Pro551 MMP9 (EGFR/FOLR1 h59-3), and Pro551 MMP9cl(EGFR/ FOLR1 h59-3). The results of these experiments demonstrated that aFOLR1(h59-3)/aEGFR (D12) was able to kill tumor cell lines expressing both FOLR1 and EGFR conditionally.

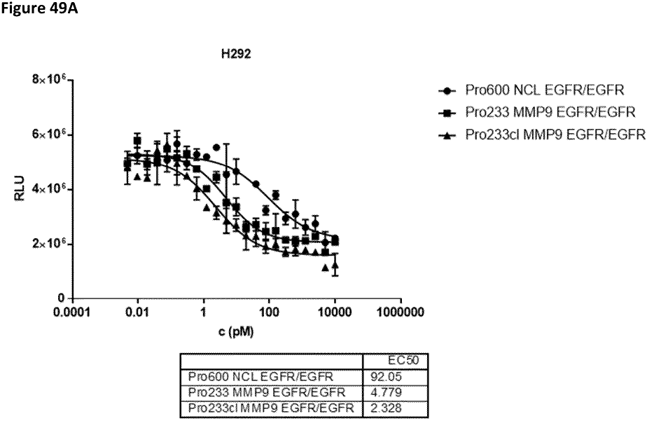

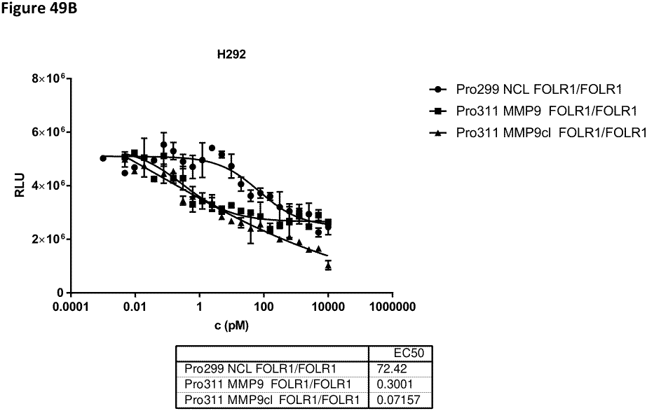

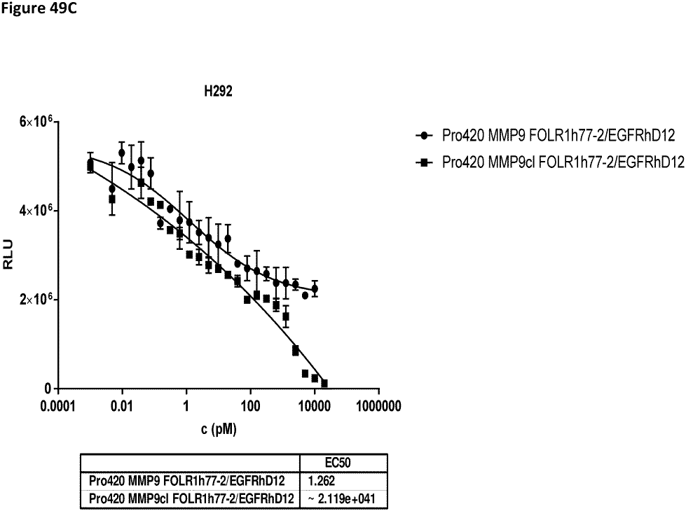

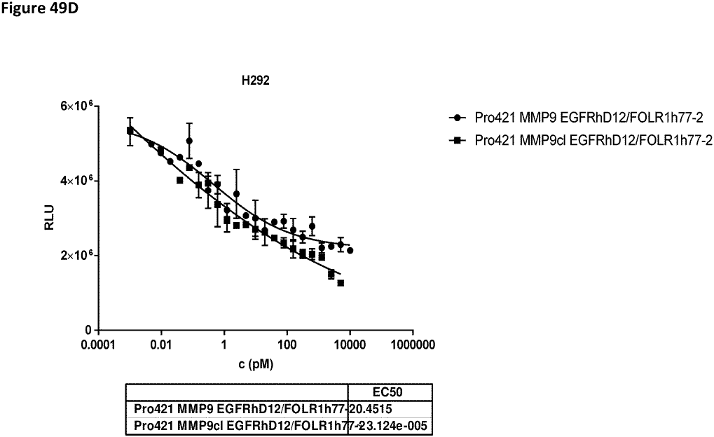

FIG. 49A: H292 cells were tested with Pro600 NCL EGFR/EGFR, Pro233 MMP9 EGFR/EGFR, and Pro233 MMP9cl EGFR/EGFR. FIG. 49B: H292 cells were tested with Pro299 NCL FOLR1/ FOLR1, Pro311 MMP9 FOLR1/FOLR1, and Pro311 MMP9cl FOLR1/FOLR1. FIG. 49C: H292 cells were tested with Pro420 MMP9 FOLR1/EGFR, and Pro420 MMP9cl FOLR1/EGFR. FIG. 49D: H292 cells were tested with Pro421 MMP9 EGFR/FOLR1, and Pro421 MMP9cl EGFR/FOLR1. The results of these experiments demonstrated that aFOLR1 (h77-2 or h57-3)/aEGFR (hD12) were able to kill tumor cell lines expressing both FOLR1 and EGFR conditionally.

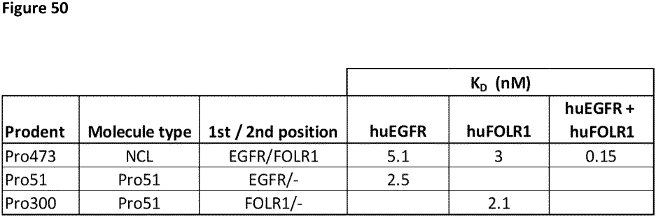

The affinities of EGFR/FOLR1 HeteroCOBRA vs Pro51 format molecules were assessed and are listed in FIG. 50.

### Example 13: Trop2/EpCAM HeteroCOBRA Induced TDCC of Cells Expressing Trop2 and EpCAM

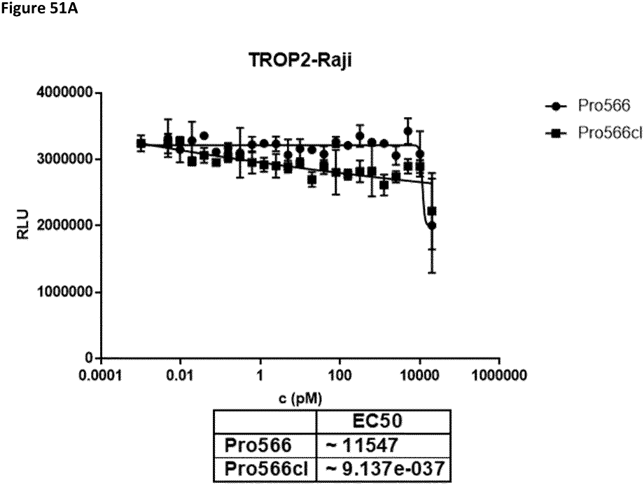

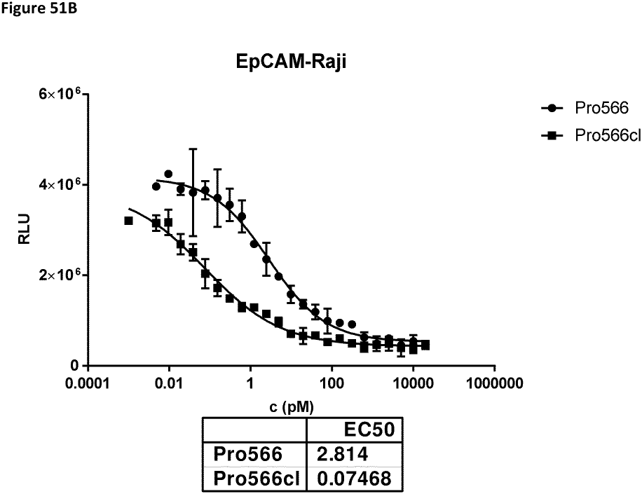

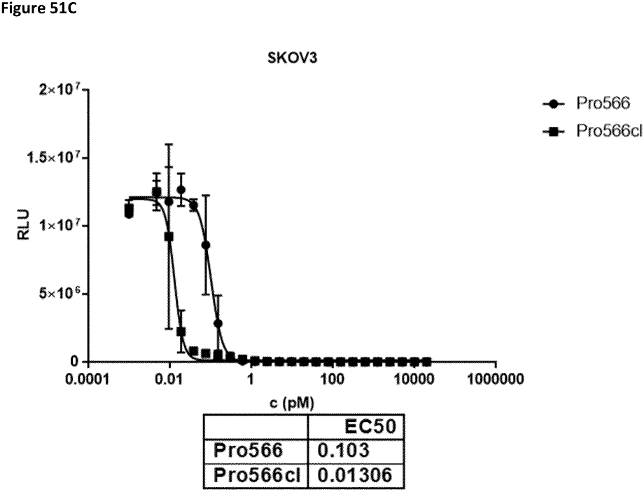

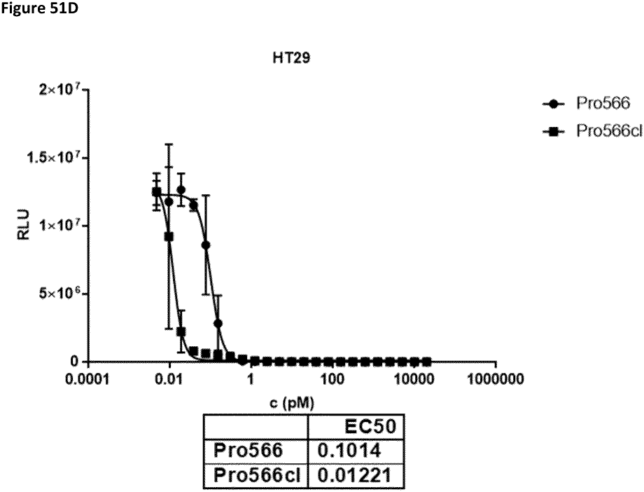

Raji-Trop2 cells (FIG. 51A), Raji-EpCAM (FIG. 51B), SKOV3 cells (FIG. 51C), and HT29 cells (FIG. 51D) were all tested with Pro566 and Pro566cl. These experiments demonstrated that Pro566 aEpCAM (h664) was able to kill Raji cells transfected to express EpCAM and tumor cell lines expressing EpCAM conditionally.

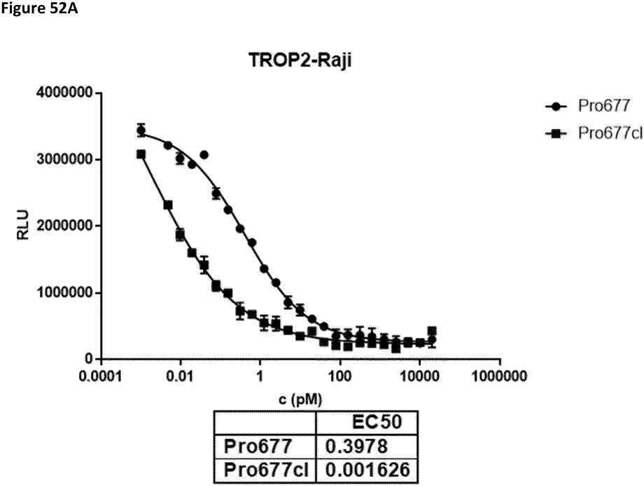

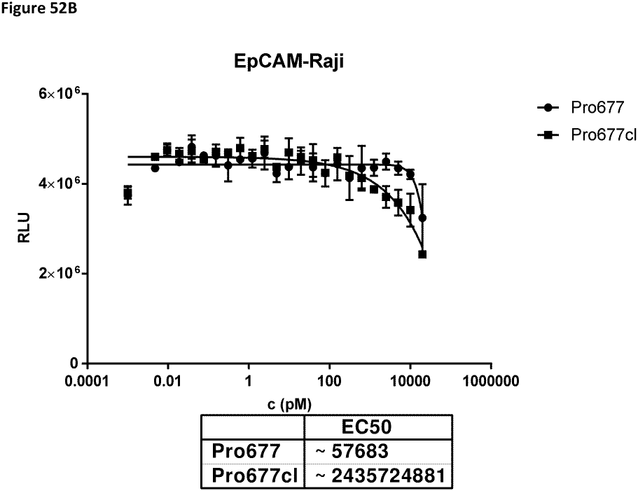

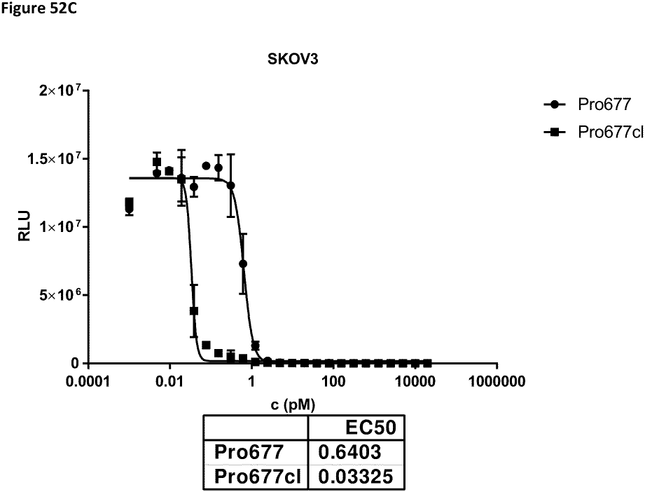

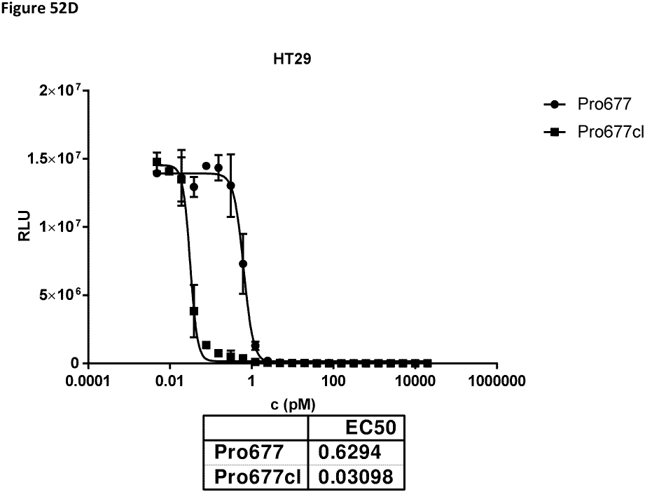

Raji-Trop2 cells (FIG. 52A), Raji-EpCAM cells (FIG. 52B), SKOV3 cells (FIG. 52C), and HT29 cells (FIG. 52D) were all tested with Pro677 and cleaved Pro677 (Pro677cl). These experiments demonstrated that Pro677 (aTrop2 h557) was able to kill Trop2 Raji cells transfected to express Trop2 and tumor cell lines expressing Trop2 conditionally.

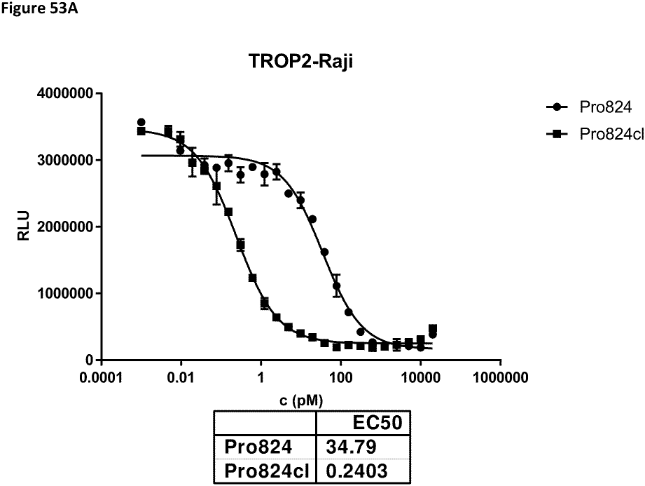

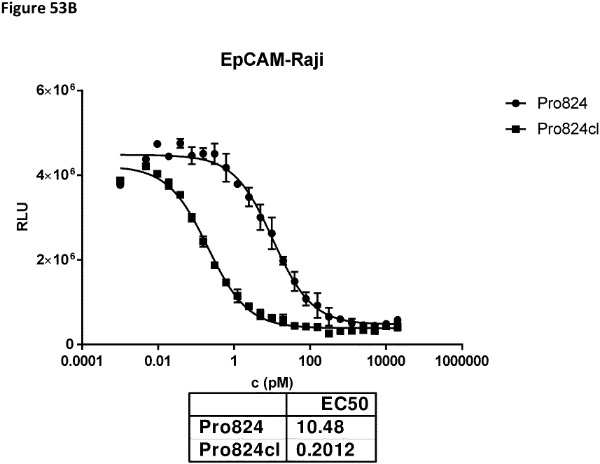

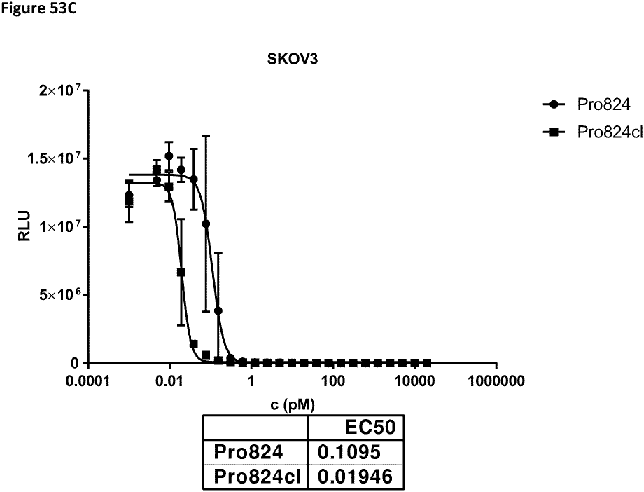

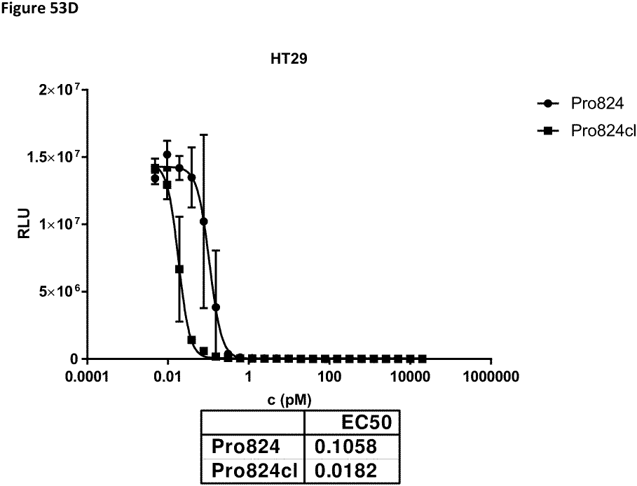

Raji-Trop2 cells (FIG. 53A), Raji-EpCAM cells (FIG. 53B), SKOV3 cells (FIG. 53C), and HT29 cells (FIG. 53D) were all tested with Pro824 and cleaved Pro824 (Pro824cl). These experiments demonstrated that Pro824 (aEpCAM h664/aTrop2 h557) was able to kill Raji transfected to express either EpCAM or Trop2 and tumor cell lines expressing both EpCAM and Trop2 conditionally.

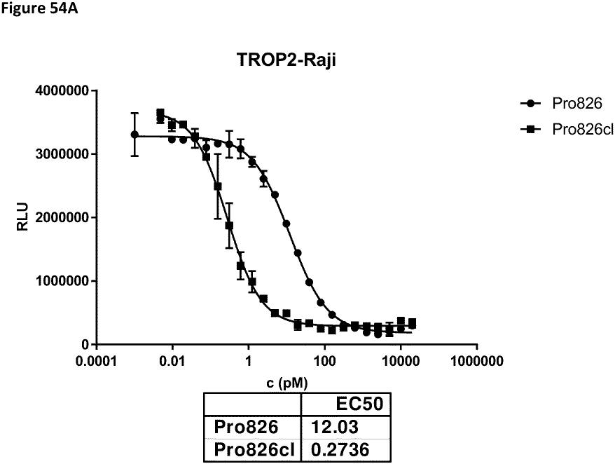

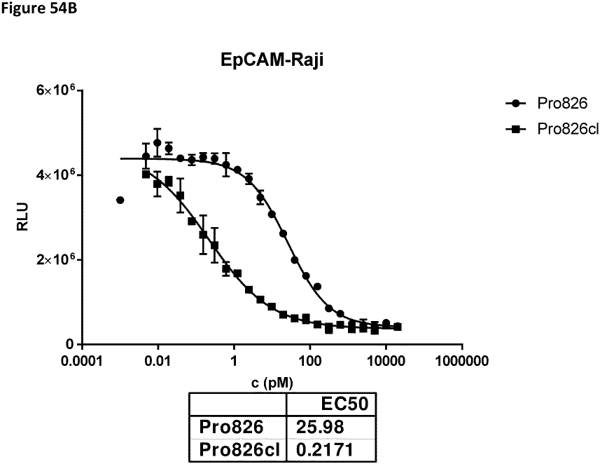

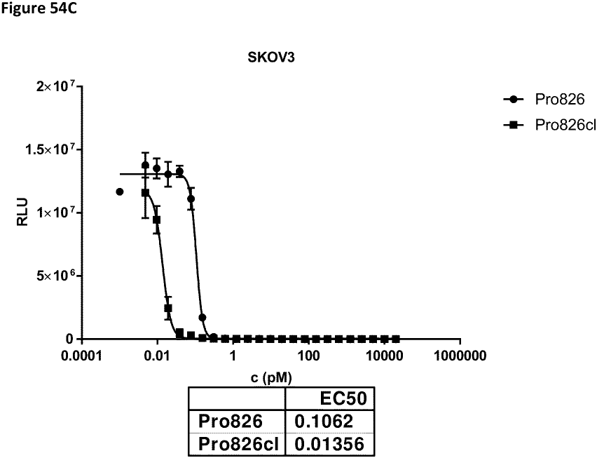

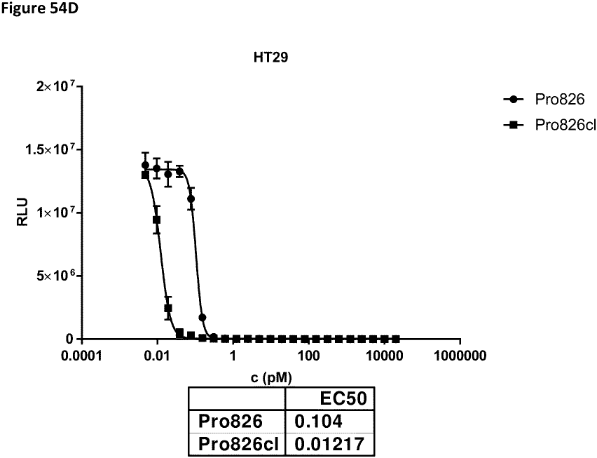

Raji-Trop2 cells (FIG. 54A), Raji-EpCAM cells (FIG. 54B), SKOV3 cells (FIG. 54C), and HT29 cells (FIG. 54D) were all tested with Pro826 and cleaved Pro826 (Pro826cl). These experiments demonstrated that Pro826 (aTROP2 h557/aEpCAM h664) was able kill to Raji transfected to express either Trop2 or EpCAM and tumor cell lines expressing both Trop2 and EpCAM conditionally.

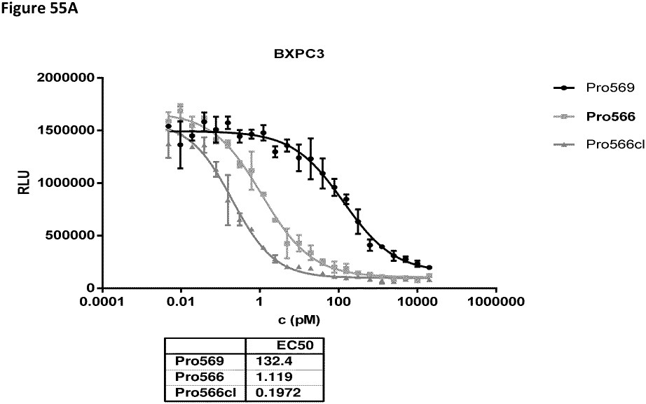

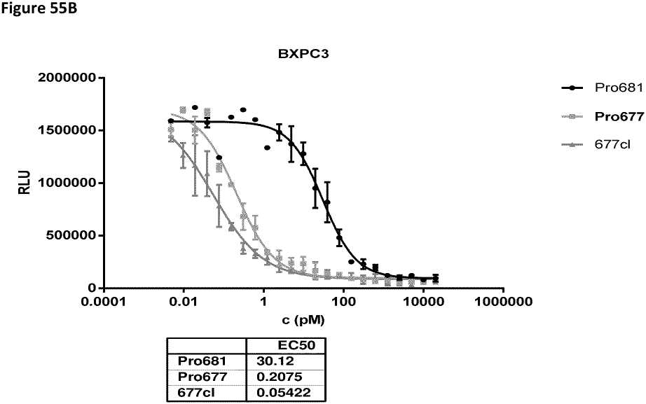

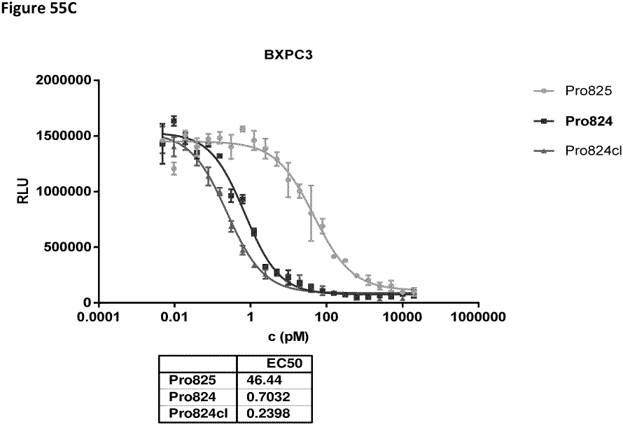

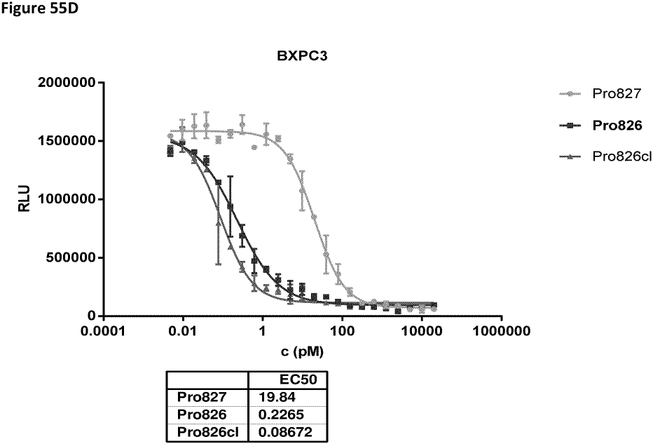

FIG. 55A: BXPC3 cells (human pancreatic cancer cell line) were tested with Pro569, Pro566 and Pro566cl. FIG. 55B: BXPC3 cells were tested with Pro681, Pro677 and Pro677cl. FIG. 55C: BXPC3 cells were tested with Pro825, Pro824 and Pro824cl. FIG. 55D: BXPC3 cells were tested with Pro827, Pro826 and Pro826cl. These experiments demonstrated that EpCAM monospecific COBRAs, Trop2 monospecific COBRAs and Trop2/EpCAM heterocobras all work well to conditionally kill BXPC3 cells.

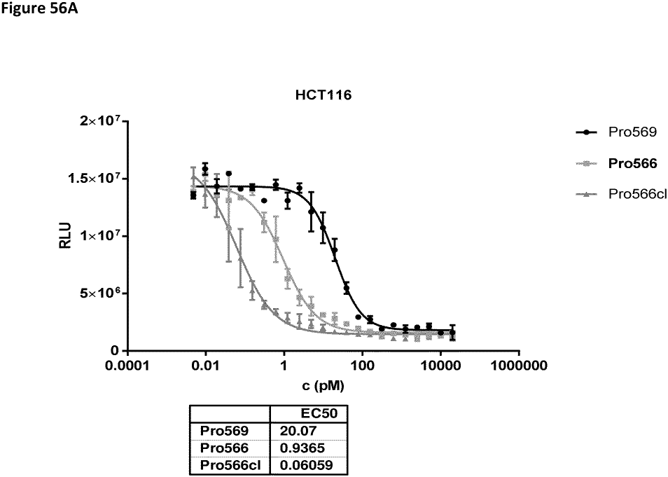

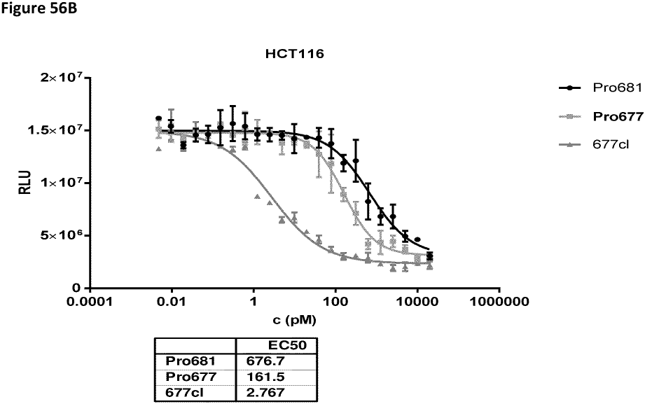

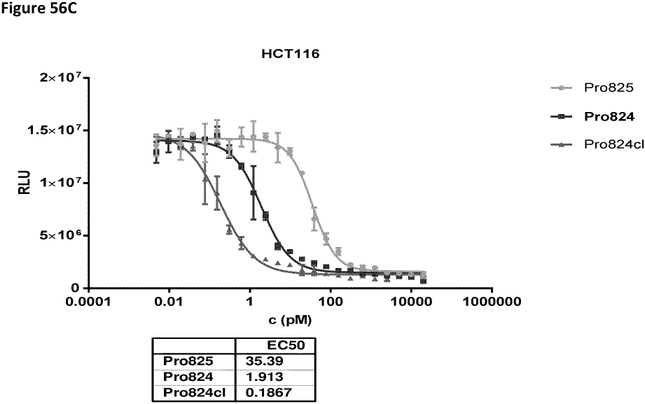

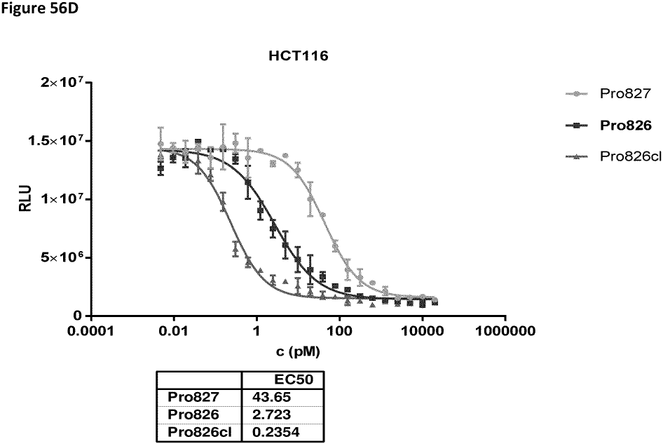

FIG. 56A: HCT116 cells (human colon cancer cell line) were tested with Pro569, Pro566 and Pro566cl. FIG. 56B: HCT116 cells were tested with Pro681 NCL, Pro677 MMP9, and Pro677MMP9cl. FIG. 56C: HCT116 cells were tested with Pro825, Pro824 and Pro824cl. FIG. 56D: HCT116 cells were tested with Pro827, Pro826 and Pro826cl. These experiments demonstrated that EpCAM monospecific COBRAs, Trop2 monospecific COBRAs and Trop2/EpCAM heterocobras all work well to conditionally kill HCT116 cells.

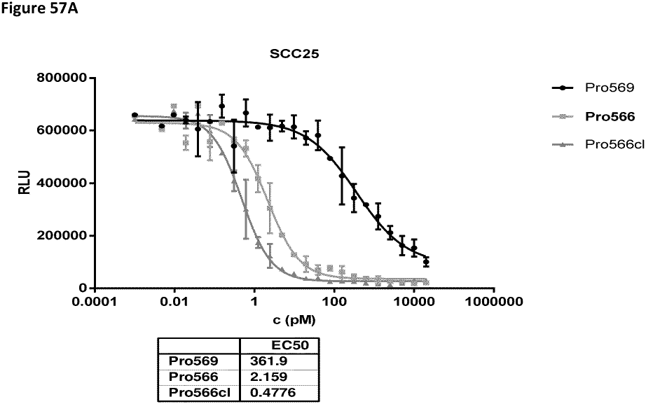

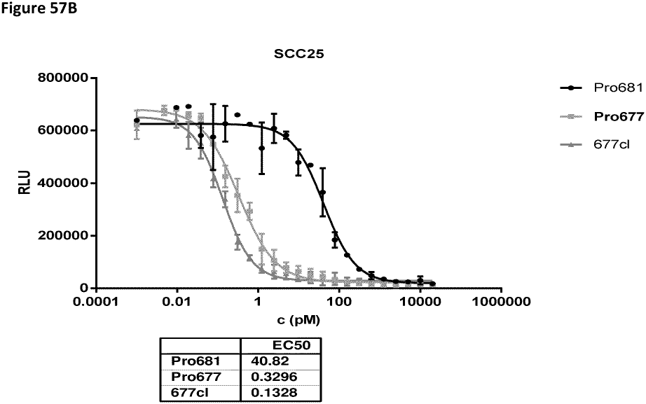

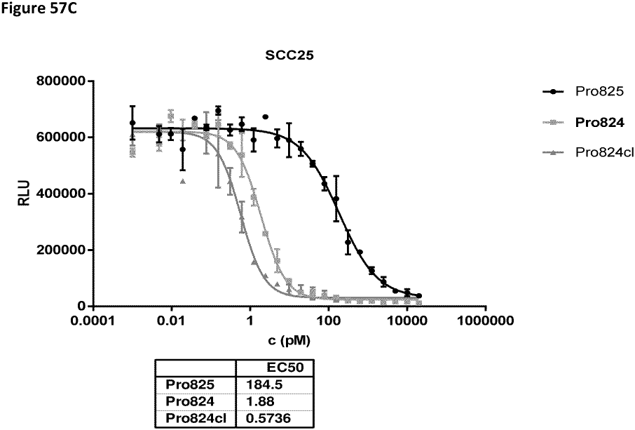

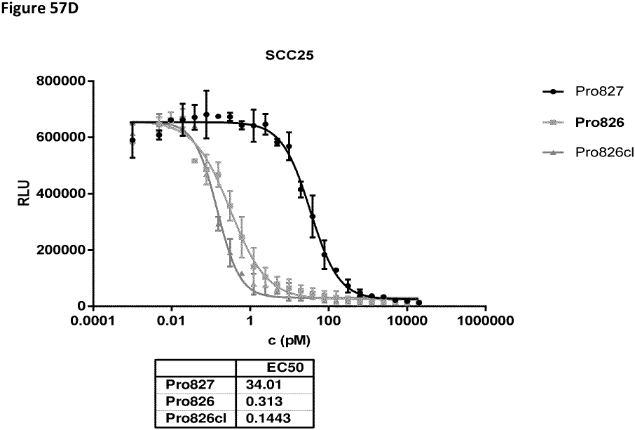

FIG. 57A: SCC25 cells (human squamous cell carcinoma cell line) were tested with Pro569, Pro566 and Pro566cl. FIG. 57B: SCC25 cells were tested with Pro681, Pro677 and Pro677cl. FIG. 57C: SCC25 cells were tested with Pro825, Pro824 and Pro824cl. FIG. 57D: SCC25 cells were tested with Pro827, Pro826 and Pro826cl. These experiments demonstrated that EpCAM monospecific COBRAs, Trop2 monospecific COBRAs and Trop2/EpCAM heterocobras all work well to conditionally kill SCC25 cells.

### Example 14: B7H3/EpCAM HeteroCOBRAs Induced TDCC of Cells Expressing B7H3 and EpCAM

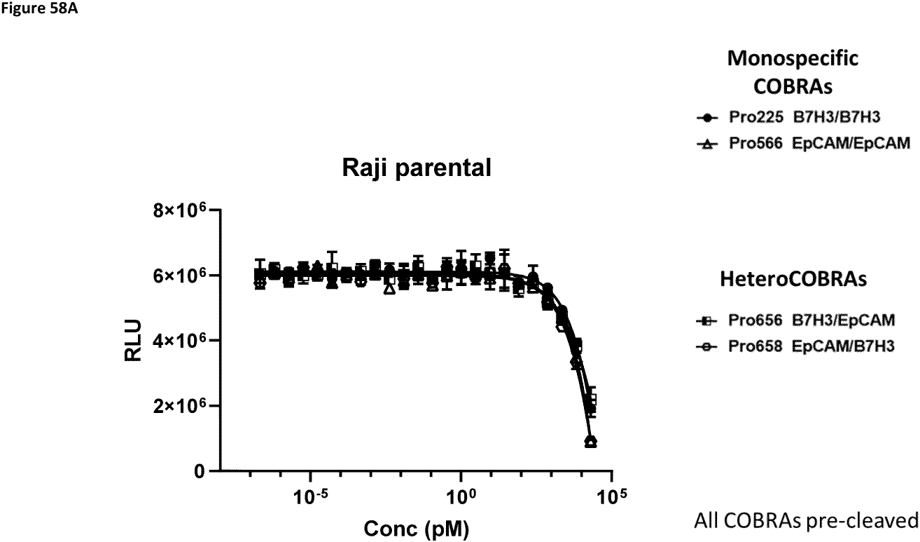

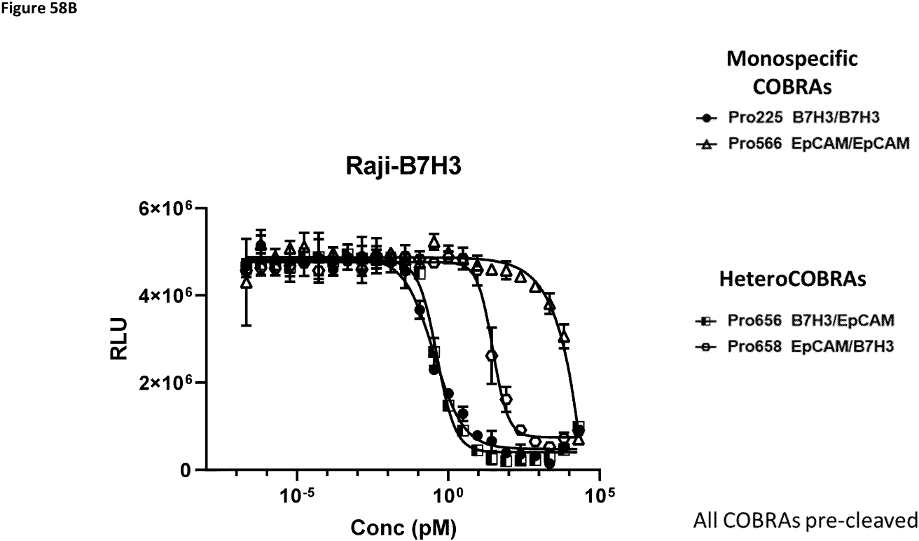

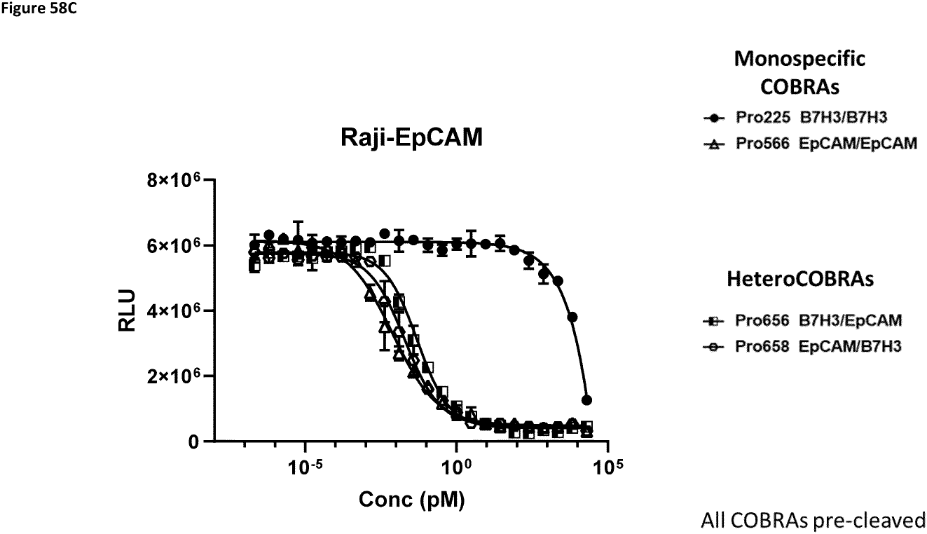

B7H3/EpCAM heteroCOBRAs were shown to induce TDCC on cells expressing one or both antigens. Raji-parental cells (FIG. 58A), Raji-B7H3 cells (FIG. 58B), Raji-EpCAM cells (FIG. 58C), and Raji-B7H3/EpCAM cells (FIG. 58D) were tested with monospecifc COBRAs: Pro225 (B7H3/B7H3 and Pro566 (EpCAM/EpCAM) and with heteroCOBRAs Pro656 (B7H3/EGFR) and Pro658 (EpCAM/B7H3).

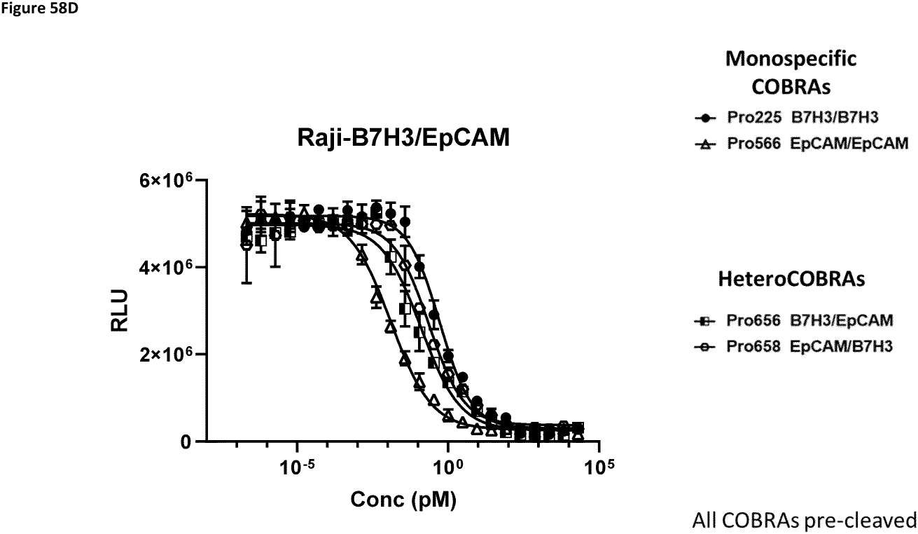

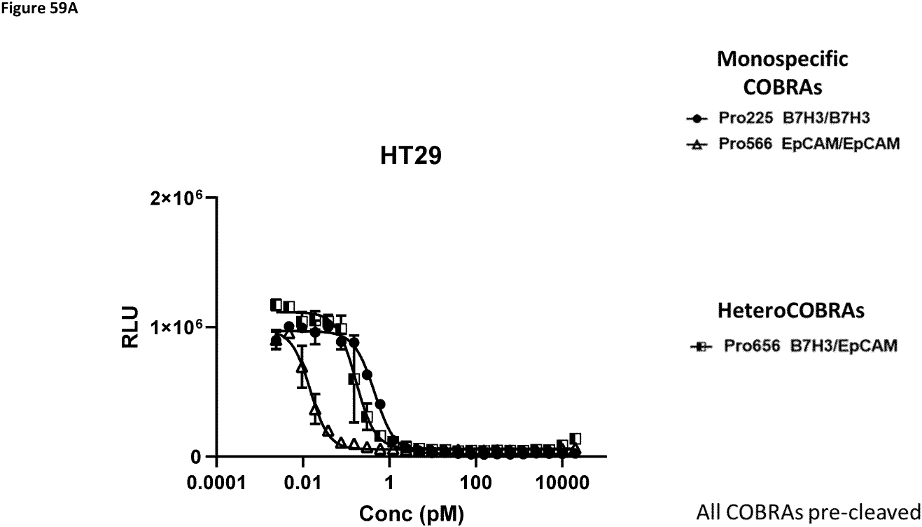

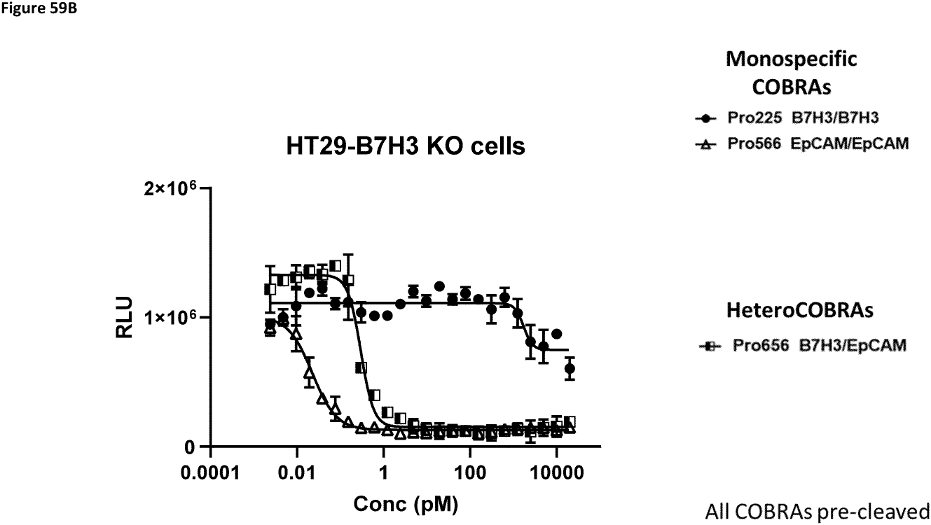

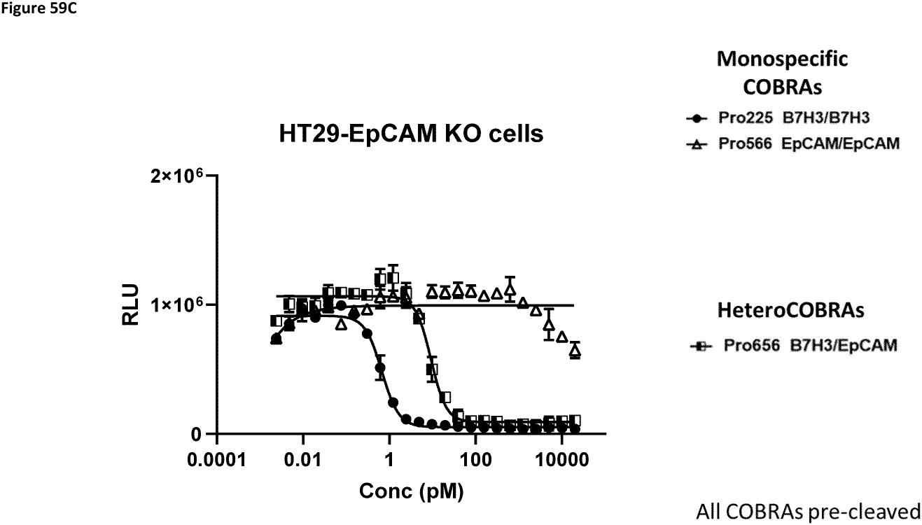

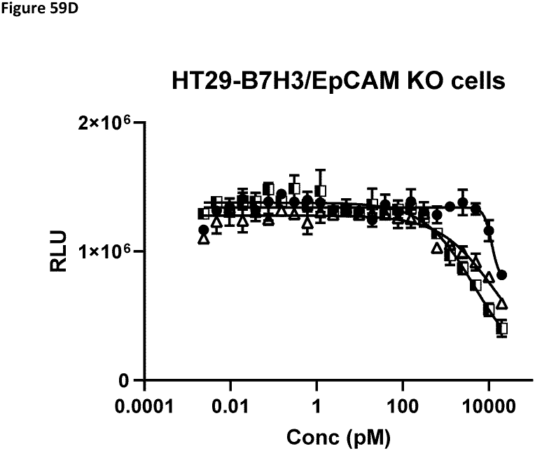

Experiments were performed in CRISPR knockout lines: HT29 cells (FIG. 59A), HT29-B7H3 KO cells (FIG. 59B), HT29-EpCAM KO cells (FIG. 59C), and HT29-B7H3/EpCAM KO cells (FIG. 59D) were all tested with monospecific COBRAs (Pro225 BN7H3/B7H3 and Pro566 EpCAM/EpCAM) and with heteroCOBRAS (Pro656 B7H3/EpCAM). All COBRAs were pre-cleaved.

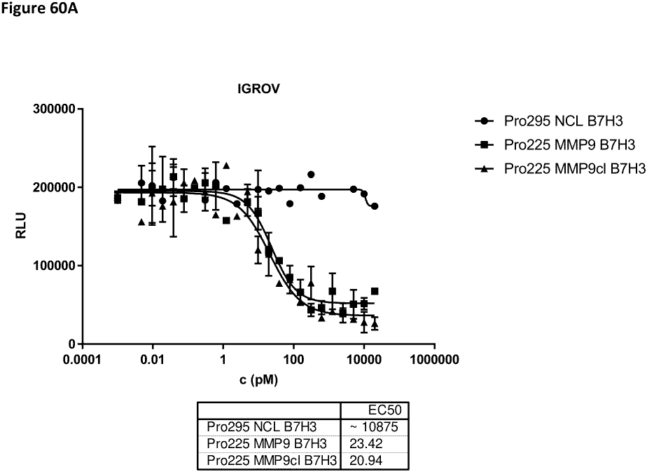

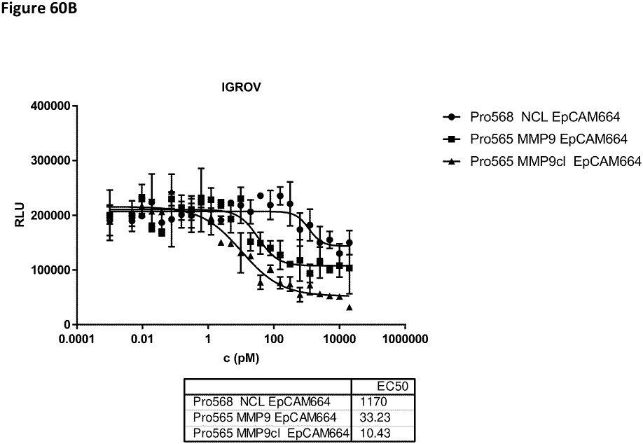

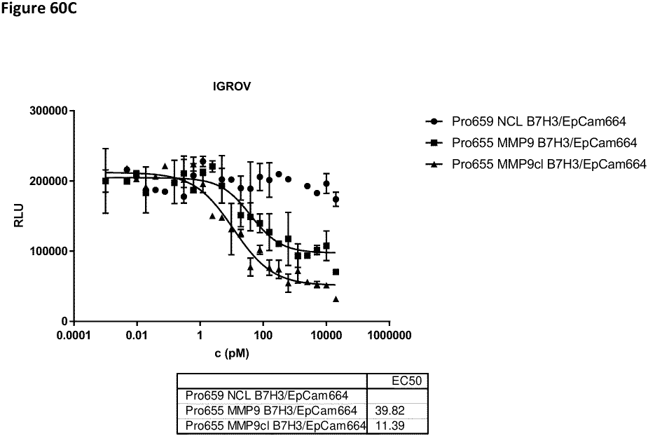

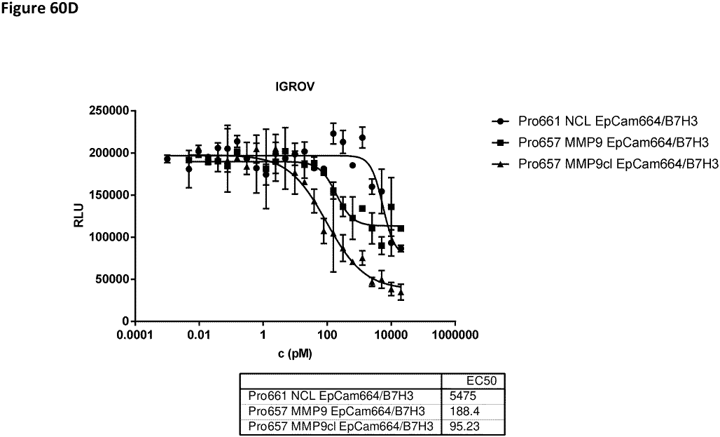

FIG. 60A: IGROV cells were tested with Pro295 NCL (B7H3 hF7), Pro225 MMP9 (B7H3 hF7) and Pro225 MMP9cl (B7H3 hF7). FIG. 60B: IGROV cells were tested with Pro568 NCL (EpCAM h664), Pro565 MMP9 (EpCAM h664), and Pro565 MMP9cl (EpCAM h664). FIG. 60C: IGROV cells were tested with Pro659 NCL (B7H3 hF7/EpCAM h664), Pro655 MMP9 (B7H3 hF7/EpCAM h664) and Pro655 MMP9cl (B7H3 hF7/EpCAM h664). FIG. 60D: IGROV cells were tested with Pro661 NCL (EpCAM h664/B7H3 hF7), Pro657 MMP9 (EpCAM h664/B7H3 hF7) Pro657 MMP9cl (EpCAM h664/B7H3 hF7). The results showed that aEpCAM (aEpCAM h664)/aB7H3 (aB7H3 hF7) heteroCOBRAs were able to kill tumor cell lines expressing both EpCAM and B7H3 conditionally.

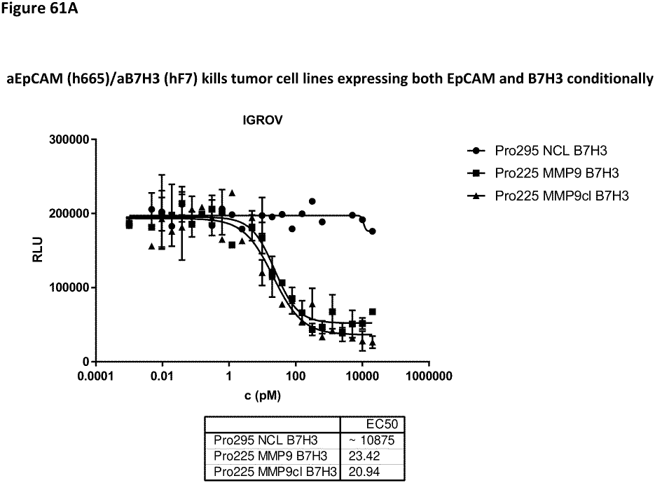

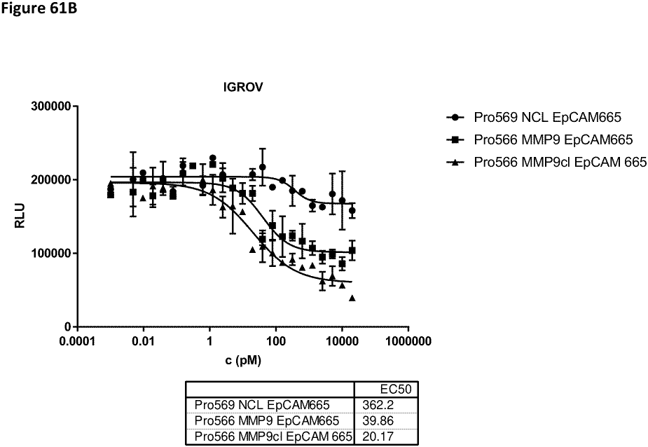

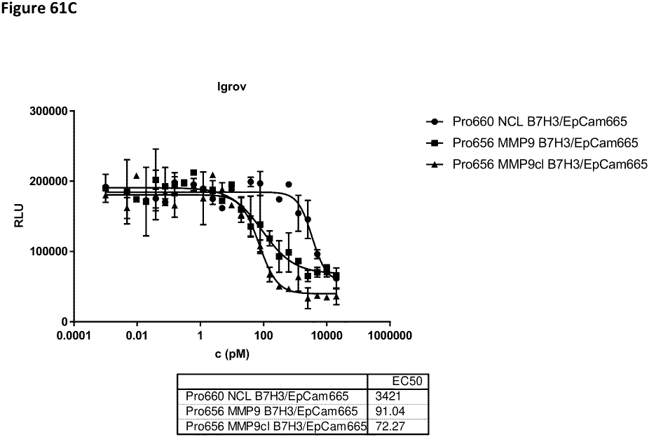

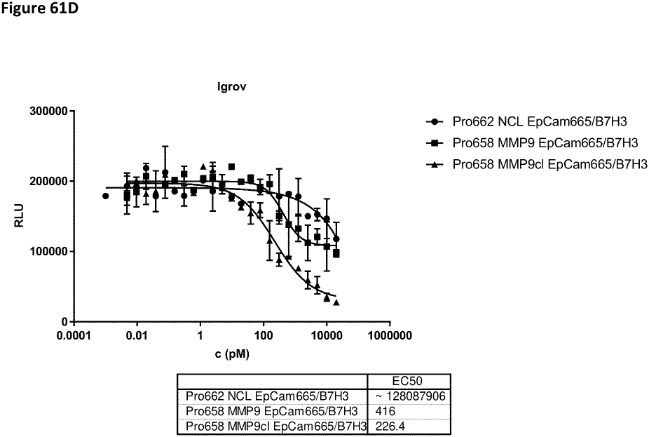

FIG. 61A: IGROV cells were tested with Pro295 NCL (B7H3 hF7), Pro225 MMP9 (B7H3 hF7) and Pro225 MMP9cl (B7H3 hF7). FIG. 61B: IGROV cells were tested with Pro569 NCL (EpCAM h665), Pro566 MMP9 (EpCAM h665), and Pro566 MMP9cl (EpCAM h665). FIG. 61C: IGROV cells were tested with Pro660 NCL (B7H3/EpCAM h665), Pro656 MMP9 (B7H3/EpCAM h665) and Pro656 MMP9cl (B7H3/EpCAM h665). FIG. 61D: IGROV cells were tested with Pro662 NCL (EpCAM h665/B7H3), Pro658 MMP9 (EpCAM h665/B7H3) and Pro658 (EpCAM h665/B7H3). The results showed that aEpCAM (aEPCAM h665)/aB7H3 (aB7H3 hF7) was able to kill tumor cell lines expressing both EpCAM and B7H3 conditionally.

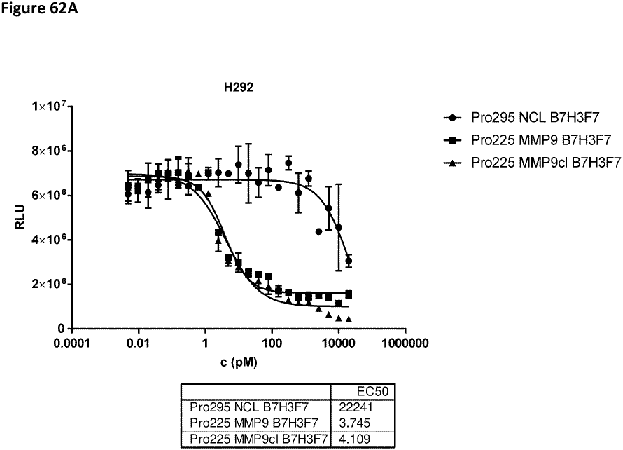

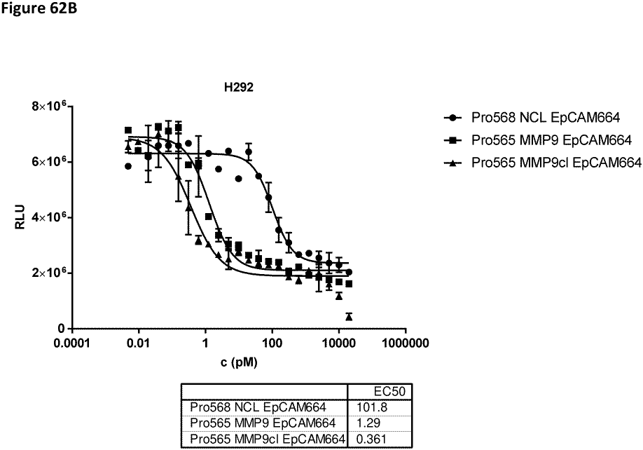

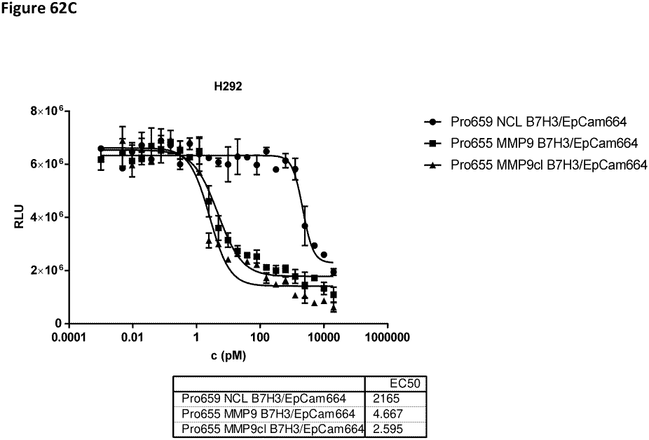

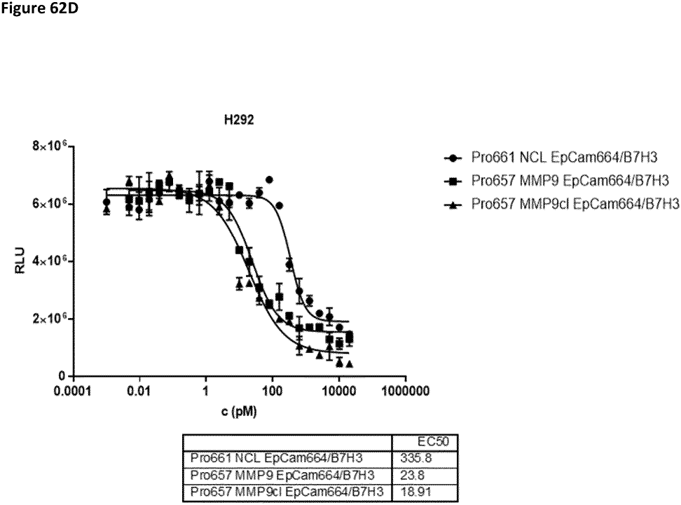

FIG. 62A: H292 cells were tested with Pro295 NCL (B7H3 hF7), Pro225 MMP9 (B7H3 hF7) and Pro225 MMP9cl (B7H3 hF7). FIG. 62B: H292 cells were tested with Pro568 NCL (EpCAM h664), Pro565 MMP9 (EpCAM h664), and Pro565 MMP9cl (EpCAM h664). FIG. 62C: H292 cells were tested with Pro659 NCL (B7H3/EpCAM h664), Pro655 MMP9 (B7H3/EpCAM h664) and Pro655 MMP9cl (B7H3/EpCAM h664). FIG. 62D: H292 cells were tested with Pro661 NCL (EpCAM h664/B7H3), Pro657 MMP9 (EpCAM h664/B7H3) and Pro657 MMP9cl (EpCAM h664/B7H3). The results showed that aEpCAM (aEpCAM h664)/aB7H3 (aB7H3 hF7) heteroCOBRAs were able to kill tumor cell lines expressing both EpCAM and B7H3 conditionally.

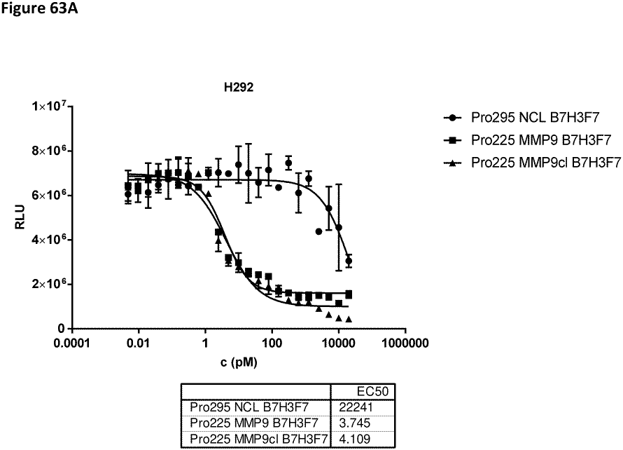

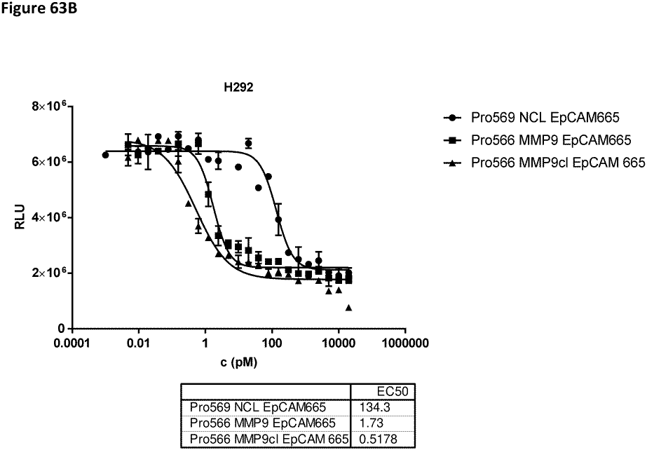

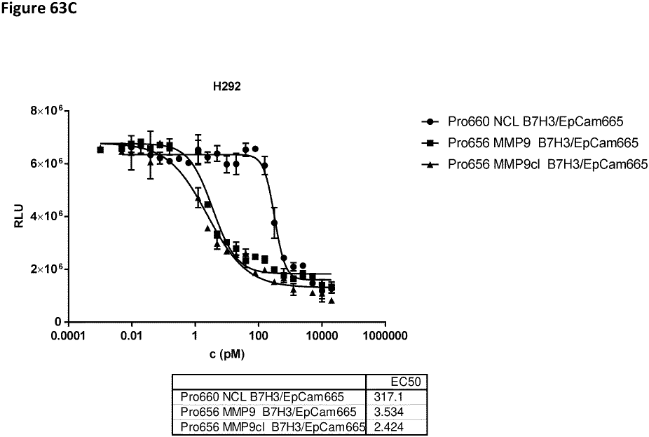

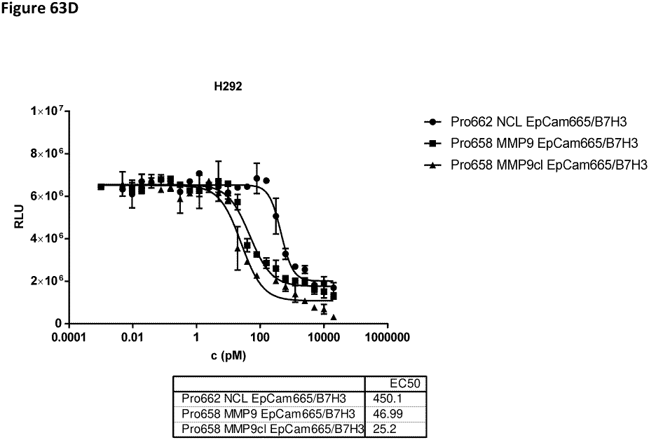

H292 cells were tested with Pro295 NCL (B7H3 hF7), Pro225 MMP9 (B7H3 hF7)and Pro225 MMP9cl (B7H3 hF7) (FIG. 63A); Pro569 NCL (EpCAM h665), Pro566 MMP9 (EpCAM h665), and Pro566 MMP9cl (EpCAM h665) (FIG. 63B); Pro660 NCL (B7H3/EpCAM h665), Pro656 MMP9 (B7H3/EpCAM h665) and Pro656 MMP9cl (B7H3/EpCAM h665) (FIG. 63C); and Pro662 NCL (EpCAM h665/B7H3), Pro658 MMP9 (EpCAM h665/B7H3) and Pro658 MMP9cl (EpCAM h665/B7H3) FIG. 63D). The results showed that aEpCAM (aEPCAM h665)/aB7H3 (aB7H3 hF7) heteroCOBRAs were able to kill tumor cell lines expressing both EpCAM and B7H3 conditionally.

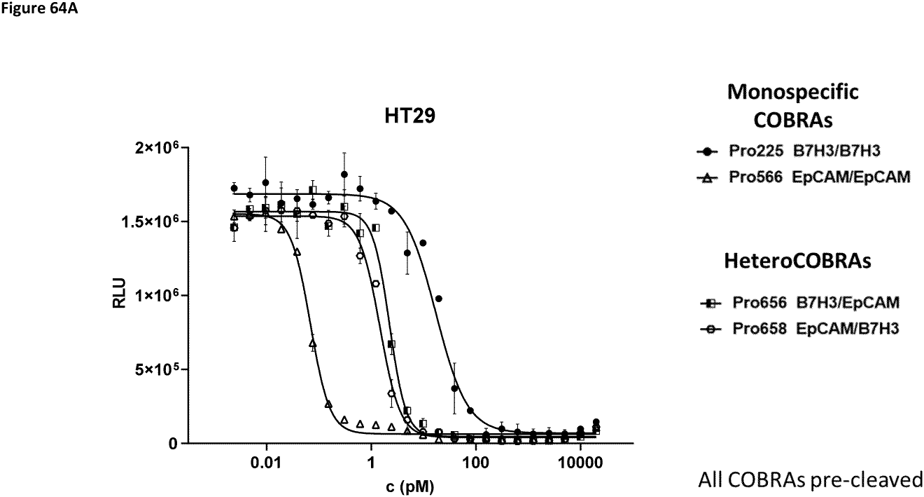

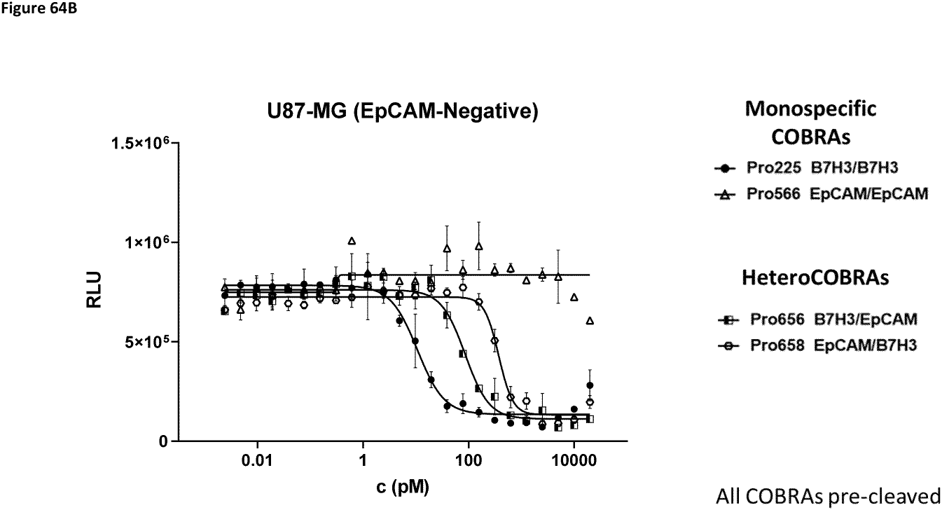

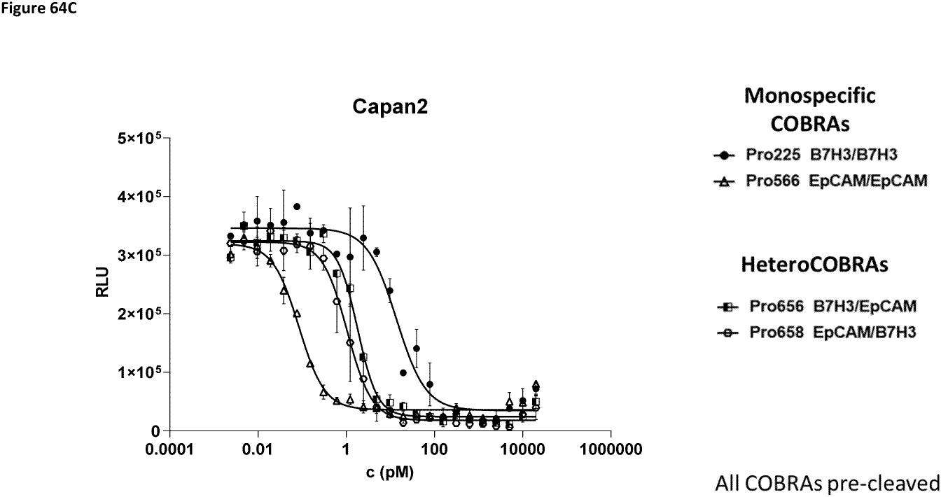

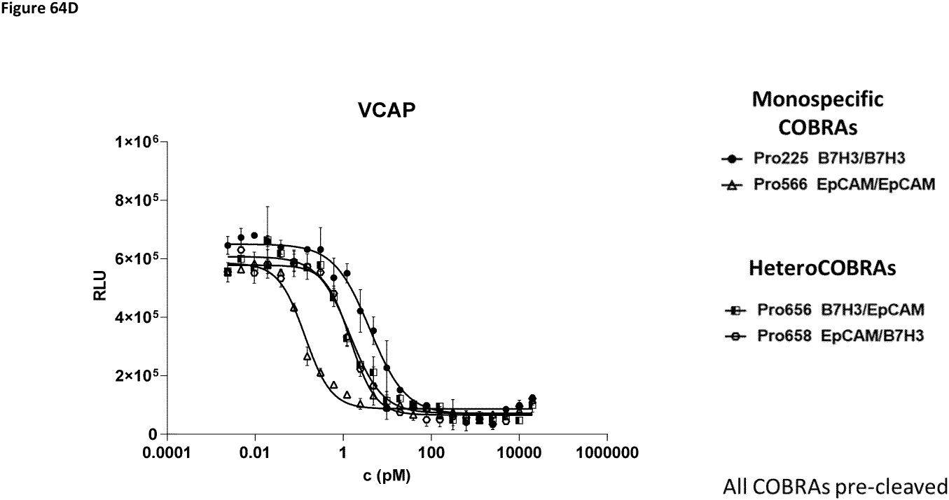

HT29 cells (FIG. 64A), U87-MG (EpCAM-negative) cells (FIG. 64B), Capan2 cells (FIG. 64C), and VCAP cells (FIG. 64D) were all tested with monospecific COBRAs: Pro225 (B7H3/B7H3 and Pro566 (EpCAM/EpCAM) and with heteroCOBRAS Pro656 (B7H3/EpCAM and Pro658 EpCAM/B7H3) to show the effect of TDCC on tumor cell lines.

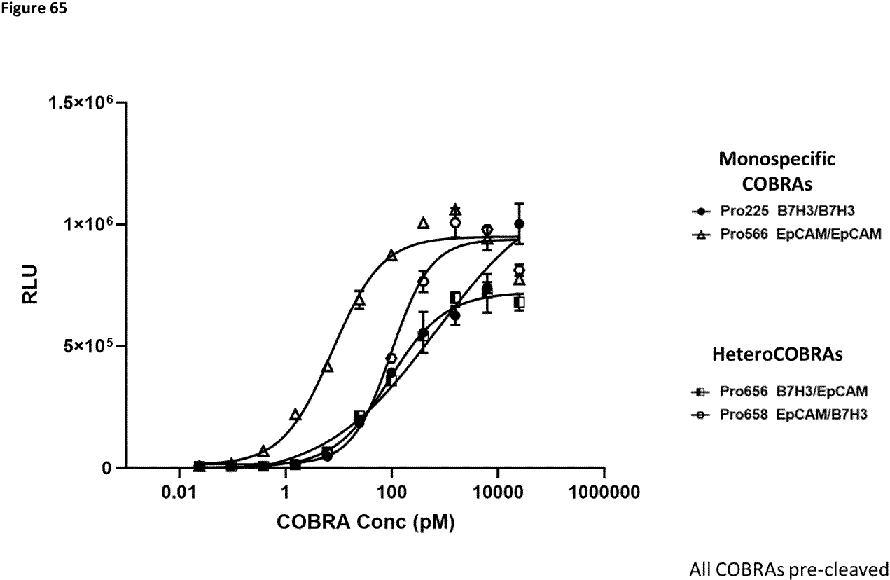

T cell activation in the presence of HT29 cells was determined using standard Jurkat luciferase assays known to those skilled in the art. The HT29 cells were tested with monospecific COBRAs: Pro225 (B7H3/B7H3) and Pro566(EpCAM/EpCAM) and with heteroCOBRAs: Pro656 (B7H3/EpCAM) and Pro658 (EpCAM/B7H3) (FIG. 65).

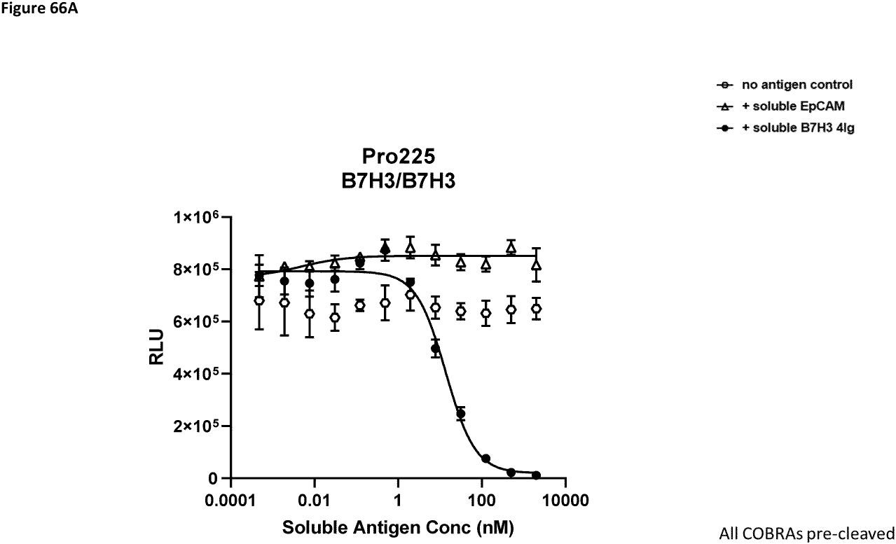

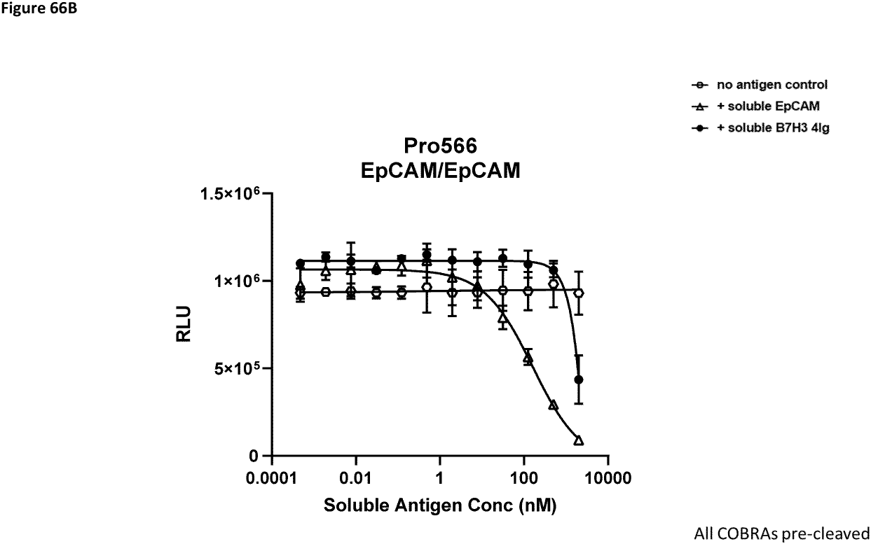

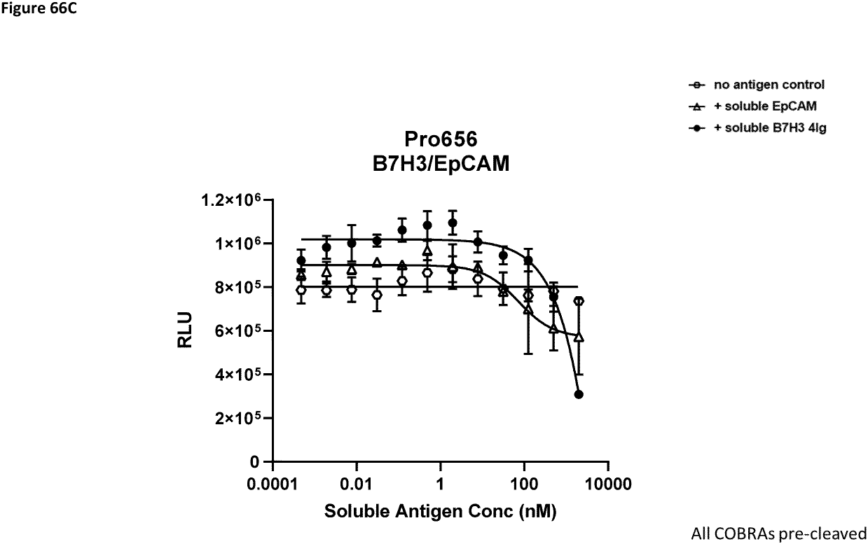

The activity of heteroCOBRAs in the Jurkat activation assay were shown to be less sensitive to inhibition by soluble antigen than with monospecific COBRAs on HT29 cells. The cells were assayed with soluble EpCAM, soluble B7H3-4Ig and with no antigen (control) together with monospecific COBRAs: Pro225 (B7H3/B7H3) (FIG. 66A) and Pro566 (EpCAM/EpCAM) (FIG. 66B) and with heteroCOBRAs: Pro656 B7H3/EpCAM (FIG. 66C) and Pro658 EpCAM/B7H3 (FIG. 66D). Pre-cleaved COBRAs were added at the EC₉₀ for each COBRA in the presence of varying concentrations of soluble antigen. A stronger inhibition of Jurkat activation was detected with the monospecific COBRAs.

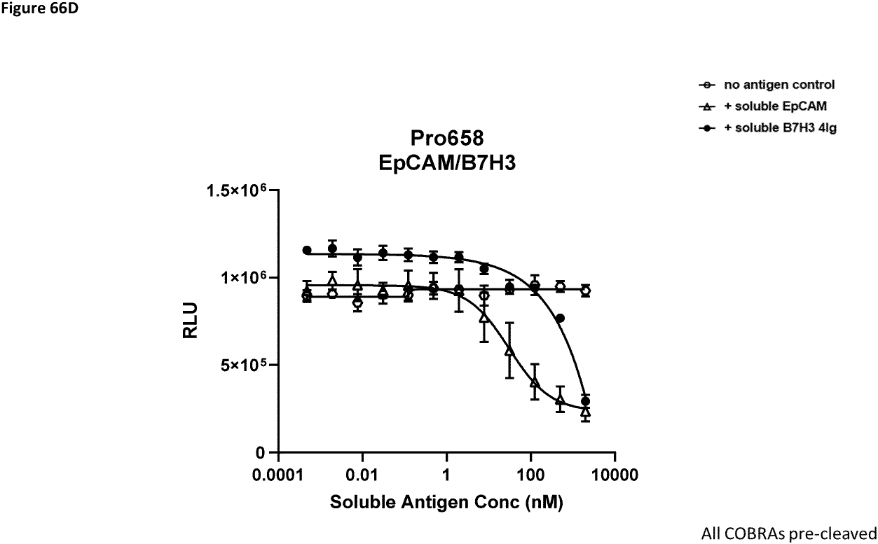

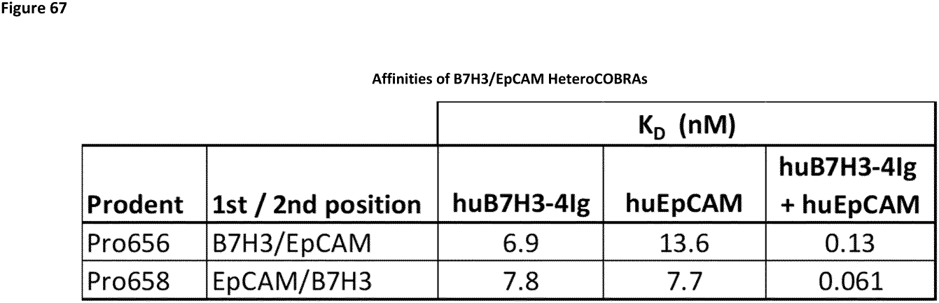

Antigens huB7H3-4Ig, huEpCAM and huB7H3-4Ig with huEpCAM were assayed with heteroCOBRAs: Pro656 B7H3/EpCAM and Pro658 EpCAM/B7H3 and FIG. 67 provides the list of the affinities of B7H3/EpCAM heteroCOBRAs.

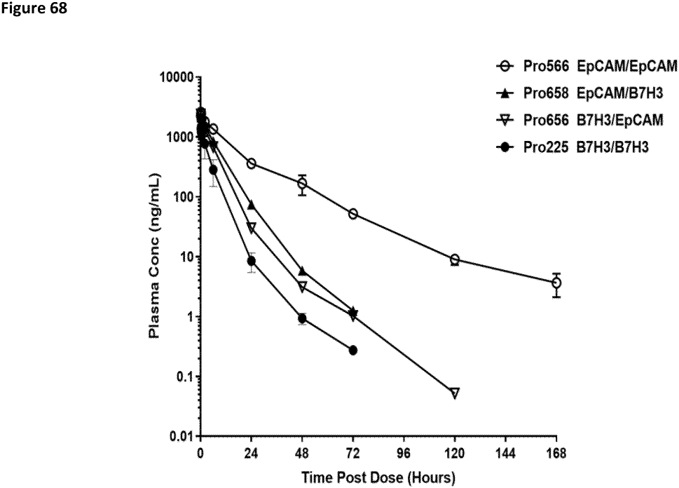

The pharmacokinetics of B7H3/EpCAM heteroCOBRAs (FIG. 68) showed that the EpCAM sdAb did not bind mouse B7H3. Pro566 which includes two EpCAM sdAbs (Pro 566 EpCAM/EpCAM) exhibited the highest circulating concentration. The B7H3 sdAb bound to mouse B7H3 proteins and Pro225 which includes two B7H3 sdAbs, (Pro225 B7H3/B7H3) exhibited the lowest exposure in circulation due to tissue-mediated drug disposition. The heteroCOBRAs, which have one B7H3 sdAB and one EpCAM sdAb (Pro656 B7H3/EpCAM and Pro658 EpCAM/B7H3) exhibited exposures between two parental monospecific COBRAs.

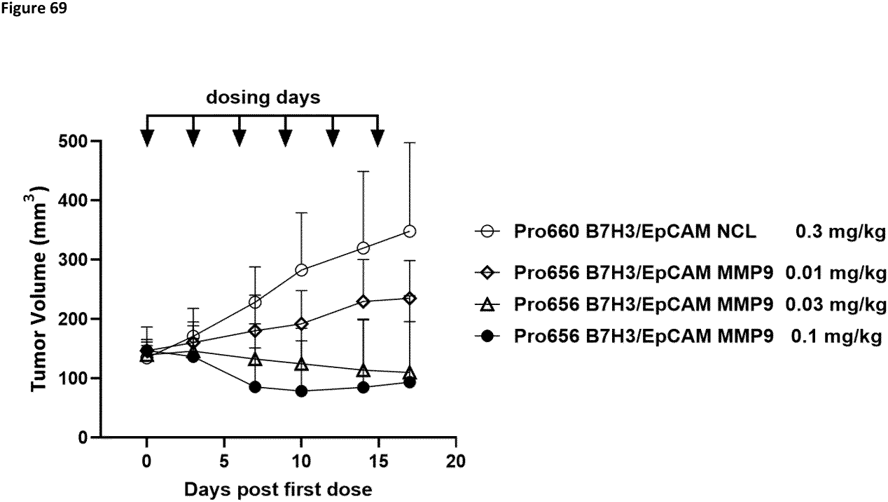

In vivo activity of the heteroCOBRAs were determined in the HT29 cell line xenograft model in mice. HeteroCOBRAs were administered at the following dosages: Pro660 NCL (B7H3/EpCAM; 0.3 mg/kg), Pro656 MMP9 (B7H3/EpCAM; 0.01 mg/kg), Pro656 MMP9 (B7H3/EpCAM; 0.03 mg/kg) and Pro656 MMP9 (B7H3/EpCAM; 0.1 mg/kg). The B7H3/EpCAM heteroCOBRAs were active in mice (FIG. 69).

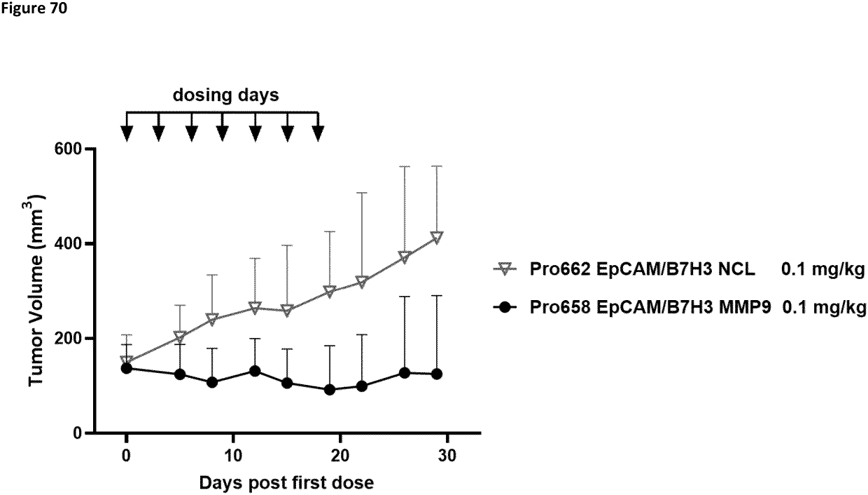

Additional heteroCOBRAs were tested in the HT29 cell line xenograft model and administered at the following dosages: Pro662 NCL (EpCAM/B7H3; 0.1 mg/kg) and Pro658 MMP9 (EpCAM/B7H3; 0.1 mg/kg). The B7H3/EpCAM heteroCOBRAs were active in mice (FIG. 70).

1. A fusion protein comprising, from N- to C-terminal:
a) a first sdABD that binds HER2 (sdABD-HER2);
b) a first domain linker;
c) a constrained Fv domain comprising:
i) a first variable heavy domain comprising a vhCDR1, vhCDR2 and vhCDR3;
ii) a constrained non-cleavable linker (CNCL); and
iii) a first variable light domain comprising vlCDR1, vlCDR2 and vlCDR3;
d) a second domain linker;
e) a second sdABD-HER2;
f) a cleavable linker (CL);
g) a constrained pseudo Fv domain comprising:
i) a first pseudo variable light domain;
ii) a non-cleavable linker (NCL); and
iii) a first pseudo variable heavy domain;
h) a third domain linker; and
i) a third sdABD that binds to human serum albumin (sdABD-HSA);
wherein the first variable heavy domain and the first variable light domain of the constrained Fv domain are capable of binding human CD3 but the constrained pseudo Fv domain does not bind CD3;
the first variable heavy domain and the first pseudo variable light domain intramolecularly associate to form an inactive Fv; and
the first variable light domain and the first pseudo variable heavy domain intramolecularly associate to form an inactive Fv; and
wherein the first and/or second sdABD-HER2 comprises an amino acid sequence comprising a set of CDRs selected from the group consisting of:
a) a sdCDR1 of SEQ ID NO:194 a sdCDR2 of SEQ ID NO:195 and a sdCDR3 of SEQ ID NO:196;
b) a sdCDR1 of SEQ ID NO:218, a sdCDR2 of SEQ ID NO:219 and a sdCDR3 of SEQ ID NO:220;
c) a sdCDR1 of SEQ ID NO:226, a sdCDR2 of SEQ ID NO:227 and a sdCDR3 of SEQ ID NO:228;
d) a sdCDR1 of SEQ ID NO:238, a sdCDR2 of SEQ ID NO:239 and a sdCDR3 of SEQ ID NO:240;
e) a sdCDR1 of SEQ ID NO:142, a sdCDR2 of SEQ ID NO:143 and a sdCDR3 of SEQ ID NO:144;
f) a sdCDR1 of SEQ ID NO146, a sdCDR2 of SEQ ID NO:147 and a sdCDR3 of SEQ ID NO:148;
g) a sdCDR1 of SEQ ID NO:150, a sdCDR2 of SEQ ID NO:151 and a sdCDR3 of SEQ ID NO:152;
h) a sdCDR1 of SEQ ID NO:154, a sdCDR2 of SEQ ID NO:155, and a sdCDR3 of SEQ ID NO:156;
i) a sdCDR1 of SEQ ID NO:158, a sdCDR2 of SEQ ID NO:159, and a sdCDR3 of SEQ ID NO:160;
j) a sdCDR1 of SEQ ID NO:162, a sdCDR2 of SEQ ID NO:163, and a sdCDR3 of SEQ ID NO:164;
k) a sdCDR1 of SEQ ID NO:166, a sdCDR2 of SEQ ID NO:167, and a sdCDR3 of SEQ ID NO:168;
1) a sdCDR1 of SEQ ID NO:170, a sdCDR2 of SEQ ID NO:171, and a sdCDR3 of SEQ ID NO:172;
m) a sdCDR1 of SEQ ID NO:174, a sdCDR2 of SEQ ID NO:175, and a sdCDR3 of SEQ ID NO:176;
n) a sdCDR1 of SEQ ID NO:178, a sdCDR2 of SEQ ID NO:179, and a sdCDR3 of SEQ ID NO:180;
o) a sdCDR1 of SEQ ID NO:182, a sdCDR2 of SEQ ID NO:183, and a sdCDR3 of SEQ ID NO:184;
p) a sdCDR1 of SEQ ID NO:186, a sdCDR2 of SEQ ID NO:187, and a sdCDR3 of SEQ ID NO:188;
q) a sdCDR1 of SEQ ID NO:190, a sdCDR2 of SEQ ID NO:191, and a sdCDR3 of SEQ ID NO:192;
r) a sdCDR1 of SEQ ID NO:194, a sdCDR2 of SEQ ID NO:195, and a sdCDR3 of SEQ ID NO:196;
s) a sdCDR1 of SEQ ID NO:198, a sdCDR2 of SEQ ID NO:199, and a sdCDR3 of SEQ ID NO:200;
t) a sdCDR1 of SEQ ID NO:202, a sdCDR2 of SEQ ID NO:203, and a sdCDR3 of SEQ ID NO:204;
u) a sdCDR1 of SEQ ID NO:206, a sdCDR2 of SEQ ID NO:207, and a sdCDR3 of SEQ ID NO:203;
v) a sdCDR1 of SEQ ID NO:210, a sdCDR2 of SEQ ID NO:211, and a sdCDR3 of SEQ ID NO:212;
w) a sdCDR1 of SEQ ID NO:214, a sdCDR2 of SEQ ID NO:215, and a sdCDR3 of SEQ ID NO:216;
x) a sdCDR1 of SEQ ID NO:218, a sdCDR2 of SEQ ID NO:219, and a sdCDR3 of SEQ ID NO:220;
y) a sdCDR1 of SEQ ID NO:222, a sdCDR2 of SEQ ID NO:223, and a sdCDR3 of SEQ ID NO:224;
z) a sdCDR1 of SEQ ID NO:226, a sdCDR2 of SEQ ID NO:227, and a sdCDR3 of SEQ ID NO:228;
aa) a sdCDR1 of SEQ ID NO:230, a sdCDR2 of SEQ ID NO:231, and a sdCDR3 of SEQ ID NO:232;
ab) a sdCDR1 of SEQ ID NO:234, a sdCDR2 of SEQ ID NO:235, and a sdCDR3 of SEQ ID NO:236;
ac) a sdCDR1 of SEQ ID NO:238, a sdCDR2 of SEQ ID NO:239, and a sdCDR3 of SEQ ID NO:240;
ad) a sdCDR1 of SEQ ID NO:242, a sdCDR2 of SEQ ID NO:243, and a sdCDR3 with SEQ ID NO:244; and
ae) a sdCDR1 of SEQ ID NO:500, a sdCDR2 of SEQ ID NO:501, and a sdCDR3 with SEQ ID NO:502;
af) a sdCDR1 of SEQ ID NO:504, a sdCDR2 of SEQ ID NO:505, and a sdCDR3 with SEQ ID NO:506;
ag) a sdCDR1 of SEQ ID NO:508, a sdCDR2 of SEQ ID NO:509, and a sdCDR3 with SEQ ID NO:510; and
ah) a sdCDR1 of SEQ ID NO:512, a sdCDR2 of SEQ ID NO:513, and a sdCDR3 with SEQ ID NO:5.

2. (canceled)

3. The fusion protein of claim 1, wherein the first and/or second sdABD-HER2 comprises an amino acid sequence selected from the group consisting of SEQ ID NO:193, SEQ ID NO:217, SEQ ID NO:225, SEQ ID NO:237, SEQ ID NO:141, SEQ ID NO:145, SEQ ID NO:149, SEQ ID NO:153, SEQ ID NO:157, SEQ ID NO:161, SEQ ID NO:165, SEQ ID NO:169, SEQ ID NO:173, SEQ ID NO:177, SEQ ID NO:181, SEQ ID NO:185, SEQ ID NO:189,, SEQ ID NO:197, SEQ ID NO:201, SEQ ID NO:205, SEQ ID NO:209, SEQ ID NO:213, SEQ ID NO:221, SEQ ID NO:229, SEQ ID NO:233, SEQ ID NO:241, SEQ ID NO:499, SEQ ID NO:503, SEQ ID NO:507, and SEQ ID NO:511.

4. The fusion protein of claim 1, wherein the first sdABD-HER2 and the second sdABD-HER2 are the same.

5. The fusion protein of claim 1, wherein the first sdABD-HER2 and the second sdABD-HER2 are different.

6. The fusion protein of claim 1, wherein the first variable heavy domain is N-terminal to the first variable light domain and the pseudo variable light domain is N-terminal to the pseudo variable heavy domain.

7. The fusion protein of claim 1, wherein the first variable heavy domain is N-terminal to the first variable light domain and the pseudo variable heavy domain is N-terminal to the pseudo variable light domain.

8. The fusion protein of claim 1, wherein the first variable light domain is N-terminal to the first variable heavy domain and the pseudo variable light domain is N-terminal to the pseudo variable heavy domain.

9. The fusion protein of claim 1, wherein the first variable light domain is N-terminal to the first variable heavy domain and the pseudo variable heavy domain is N-terminal to the pseudo variable light domain.

10-12. (canceled)

13. The fusion protein of claim 1, wherein the fusion protein comprises an amino acid sequence selected from group consisting of SEQ ID NOS:459-484 and 491-494.

14. A nucleic acid encoding a fusion protein of claim 1.

15. An expression vector comprising the nucleic acid of claim 14.

16. A host cell comprising the expression vector of claim 15.

17. A method of making a fusion protein comprising: (i) culturing the host cell of claim 16 under conditions wherein the fusion protein is expressed and (ii) recovering the fusion protein.

18. A method of treating cancer in a subject comprising administering the fusion protein of claim 1 to the subject.

19. A single domain antigen binding domain (sdABD) that binds human HER2 (sdABD-HER2) comprising
(i) an amino acid sequence selected from the group consisting of SEQ ID NO:141, SEQ ID NO:145, SEQ ID NO:149, SEQ ID NO:153, SEQ ID NO:157, SEQ ID NO:161, SEQ ID NO:165, SEQ ID NO:169, SEQ ID NO:173, SEQ ID NO:177, SEQ ID NO:181, SEQ ID NO:185, SEQ ID NO:189, SEQ ID NO:193, SEQ ID NO:197, SEQ ID NO:201, SEQ ID NO:205, SEQ ID NO:209, SEQ ID NO:213, SEQ ID NO:217, SEQ ID NO:221, SEQ ID NO:225, SEQ ID NO:229, SEQ ID NO:233, SEQ ID NO:237, SEQ ID NO:241, SEQ ID NO:499, SEQ ID NO:503, SEQ ID NO:507, and SEQ ID NO:511; or
(ii) an amino acid sequence comprising a set of CDRs selected from the group consisting of:
a) a sdCDR1 of SEQ ID NO:194 a sdCDR2 of SEQ ID NO:195 and a sdCDR3 of SEQ ID NO:196;
b) a sdCDR1 of SEQ ID NO:218, a sdCDR2 of SEQ ID NO:219 and a sdCDR3 of SEQ ID NO:220;
c) a sdCDR1 of SEQ ID NO:226, a sdCDR2 of SEQ ID NO:227 and a sdCDR3 of SEQ ID NO:228;
d) a sdCDR1 of SEQ ID NO:238, a sdCDR2 of SEQ ID NO:239 and a sdCDR3 of SEQ ID NO:240;
e) a sdCDR1 of SEQ ID NO:142, a sdCDR2 of SEQ ID NO:143 and a sdCDR3 of SEQ ID NO:144;
f) a sdCDR1 of SEQ ID NO146, a sdCDR2 of SEQ ID NO:147 and a sdCDR3 of SEQ ID NO:148;
g) a sdCDR1 of SEQ ID NO:150, a sdCDR2 of SEQ ID NO:151 and a sdCDR3 of SEQ ID NO:152; ]
h) a sdCDR1 of SEQ ID NO: 154, a sdCDR2 of SEQ ID NO: 155, and a sdCDR3 of SEQ ID NO: 156;
i) a sdCDR1 of SEQ ID NO: 158, a sdCDR2 of SEQ ID NO: 159, and a sdCDR3 of SEQ ID NO:160;
j) a sdCDR1 of SEQ ID NO:162, a sdCDR2 of SEQ ID NO:163, and a sdCDR3 of SEQ ID NO:164; k) a sdCDR1 of SEQ ID NO:166, a sdCDR2 of SEQ ID NO:167, and a sdCDR3 of SEQ ID NO:168;
l) a sdCDR1 of SEQ ID NO: 170, a sdCDR2 of SEQ ID NO:171, and a sdCDR3 of SEQ ID NO: 172;
m) a sdCDR1 of SEQ ID NO:174, a sdCDR2 of SEQ ID NO:175, and a sdCDR3 of SEQ ID NO:176;
n) a sdCDR1 of SEQ ID NO:178, a sdCDR2 of SEQ ID NO:179, and a sdCDR3 of SEQ ID NO:180;
o) a sdCDR1 of SEQ ID NO:182, a sdCDR2 of SEQ ID NO:183, and a sdCDR3 of SEQ ID NO:184;
p) a sdCDR1 of SEQ ID NO:186, a sdCDR2 of SEQ ID NO:187, and a sdCDR3 of SEQ ID NO:188;
q) a sdCDR1 of SEQ ID NO:190, a sdCDR2 of SEQ ID NO:191, and a sdCDR3 of SEQ ID NO:192;
r) a sdCDR1 of SEQ ID NO:194, a sdCDR2 of SEQ ID NO:195, and a sdCDR3 of SEQ ID NO:196;
s) a sdCDR1 of SEQ ID NO:198, a sdCDR2 of SEQ ID NO:199, and a sdCDR3 of SEQ ID NO:200;
t) a sdCDR1 of SEQ ID NO:202, a sdCDR2 of SEQ ID NO:203, and a sdCDR3 of SEQ ID NO:204;
u) a sdCDR1 of SEQ ID NO:206, a sdCDR2 of SEQ ID NO:207, and a sdCDR3 of SEQ ID NO:203;
v) a sdCDR1 of SEQ ID NO:210, a sdCDR2 of SEQ ID NO:211, and a sdCDR3 of SEQ ID NO:212;
w) a sdCDR1 of SEQ ID NO:214, a sdCDR2 of SEQ ID NO:215, and a sdCDR3 of SEQ ID NO:216;
x) a sdCDR1 of SEQ ID NO:218, a sdCDR2 of SEQ ID NO:219, and a sdCDR3 of SEQ ID NO:220;
y) a sdCDR1 of SEQ ID NO:222, a sdCDR2 of SEQ ID NO:223, and a sdCDR3 of SEQ ID NO:224;
z) a sdCDR1 of SEQ ID NO:226, a sdCDR2 of SEQ ID NO:227, and a sdCDR3 of SEQ ID NO:228;
aa) a sdCDR1 of SEQ ID NO:230, a sdCDR2 of SEQ ID NO:231, and a sdCDR3 of SEQ ID NO:232;
ab) a sdCDR1 of SEQ ID NO:234, a sdCDR2 of SEQ ID NO:235, and a sdCDR3 of SEQ ID NO:236;
ac) a sdCDR1 of SEQ ID NO:238, a sdCDR2 of SEQ ID NO:239, and a sdCDR3 of SEQ ID NO:240;
ad) a sdCDR1 of SEQ ID NO:242, a sdCDR2 of SEQ ID NO:243, and a sdCDR3 with SEQ ID NO:244;
ae) a sdCDR1 of SEQ ID NO:500, a sdCDR2 of SEQ ID NO:501, and a sdCDR3 with SEQ ID NO:502;
af) a sdCDR1 of SEQ ID NO:504, a sdCDR2 of SEQ ID NO:505, and a sdCDR3 with SEQ ID NO:506;
ag) a sdCDR1 of SEQ ID NO:508, a sdCDR2 of SEQ ID NO:509, and a sdCDR3 with SEQ ID NO:510; and
ah) a sdCDR1 of SEQ ID NO:512, a sdCDR2 of SEQ ID NO:513, and a sdCDR3 with SEQ ID NO: 514.

20. A fusion protein comprising, from N- to C-terminal:
a) a first sdABD that binds a tumor target antigen (sdABD-TTA);
b) a first domain linker;
c) a constrained Fv domain comprising:
i) a first variable heavy domain comprising a vhCDR1, vhCDR2 and vhCDR3;
ii) a constrained non-cleavable linker (CNCL); and
iii) a first variable light domain comprising vlCDR1, vlCDR2 and vlCDR3;
d) a second domain linker;
e) a second sdABD-TTA;
f) a cleavable linker (CL);
g) a constrained pseudo Fv domain comprising:
i) a first pseudo variable light domain;
ii) a non-cleavable linker (NCL); and
iii) a first pseudo variable heavy domain;
h) a third domain linker; and
i) a third sdABD that binds to human serum albumin (sdABD-HSA);
wherein the first variable heavy domain and the first variable light domain of the constrained Fv domain are capable of binding human CD3 but the constrained pseudo Fv domain does not bind CD3; the first variable heavy domain and the first pseudo variable light domain intramolecularly associate to form an inactive Fv; the first variable light domain and the first pseudo variable heavy domain intramolecularly associate to form an inactive Fv and
wherein either (1) the first sdABD-TTA is a sdABD-HER2 or a sdABD-LyPD3, and the second sdABD-TTA is selected from the group consisting of a sdABD-B7H3, a sdABD-CA9, a sdABD-EGFR, a sdABD-EpCAM, a sdABD-FOLR1, a sdABD-HER2, a sdABD-LyPD3 and a sdABD-Trop2; or (2) the first sdABD-TTA is selected from the group consisting of a sdABD-B7H3, a sdABD-CA9, a sdABD-EGFR, a sdABD-EpCAM, a sdABD-FOLR1, a sdABD-HER2, a sdABD-LyPD3 and a sdABD-Trop2, and the second sdABD-TTA is a sdABD-HER2 or a sdABD-LyPD3.

21-44. (canceled)

45. A single domain antigen binding domain that binds human LyPD3 (sdABD-LyPD3) comprising (i) an amino acid sequence selected from the group consisting of SEQ ID NO:117, SEQ ID NO:121, SEQ ID NO:125, SEQ ID NO:129, SEQ ID NO:133 and, SEQ ID NO: 137 or (ii) an amino acid sequence comprising a set of CDRs selected from the group consisting of: (a) a sdCDR1 of SEQ ID NO:118, a sdCDR2 of SEQ ID NO:119 and a sdCDR3 of SEQ ID NO:120; (b) a sdCDR1 of SEQ ID NO:122, a sdCDR2 of SEQ ID NO:123 and a sdCDR3 of SEQ ID NO: 124; (c) a sdCDR1 of SEQ ID NO: 126, a sdCDR2 of SEQ ID NO: 127 and a sdCDR3 of SEQ ID NO:128; (d) a sdCDR1 of SEQ ID NO: 130, a sdCDR2 of SEQ ID NO:131, and a sdCDR3 of SEQ ID NO:132; (e) a sdCDR1 of SEQ ID NO: 134, a sdCDR2 of SEQ ID NO:135, and a sdCDR3 of SEQ ID NO: 136; and (f) a sdCDR1 of SEQ ID NO: 138, a sdCDR2 of SEQ ID NO: 139, and a sdCDR3 of SEQ ID NO:140.

46-52. (canceled)

53. A fusion protein comprising, from N- to C-terminal:
a) a sdABD that binds HER2 (sdABD-HER2);
b) a first domain linker;
c) a constrained Fv domain comprising:
i) a first variable heavy domain comprising a vhCDR1, vhCDR2 and vhCDR3;
ii) a constrained non-cleavable linker (CNCL); and
iii) a first variable light domain comprising vlCDR1, vlCDR2 and vlCDR3;
d) a cleavable linker (CL);
e) a constrained pseudo Fv domain comprising:
i) a first pseudo variable light domain;
ii) a non-cleavable linker (NCL); and
iii) a first pseudo variable heavy domain;
f) a second domain linker; and
g) a second sdABD that binds to human serum albumin (sdABD-HSA);
wherein the first variable heavy domain and the first variable light domain of the constrained Fv domain are capable of binding human CD3 but the constrained pseudo Fv domain does not bind CD3;
the first variable heavy domain and the first pseudo variable light domain intramolecularly associate to form an inactive Fv; and
the first variable light domain and the first pseudo variable heavy domain intramolecularly associate to form an inactive Fv; and
wherein the sdABD-HER2 comprises an amino acid sequence comprising a set of CDRs selected from the group consisting of:
a) a sdCDR1 of SEQ ID NO:194 a sdCDR2 of SEQ ID NO:195 and a sdCDR3 of SEQ ID NO:196;
b) a sdCDR1 of SEQ ID NO:218, a sdCDR2 of SEQ ID NO:219 and a sdCDR3 of SEQ ID NO:220;
c) a sdCDR1 of SEQ ID NO:226, a sdCDR2 of SEQ ID NO:227 and a sdCDR3 of SEQ ID NO:228;
d) a sdCDR1 of SEQ ID NO:238, a sdCDR2 of SEQ ID NO:239 and a sdCDR3 of SEQ ID NO:240;
e) a sdCDR1 of SEQ ID NO:142, a sdCDR2 of SEQ ID NO:143 and a sdCDR3 of SEQ ID NO:144;
f) a sdCDR1 of SEQ ID NO146, a sdCDR2 of SEQ ID NO:147 and a sdCDR3 of SEQ ID NO:148;
g) a sdCDR1 of SEQ ID NO:150, a sdCDR2 of SEQ ID NO:151 and a sdCDR3 of SEQ ID NO: 152;
h) a sdCDR1 of SEQ ID NO: 154, a sdCDR2 of SEQ ID NO: 155, and a sdCDR3 of SEQ ID NO: 156;
i) a sdCDR1 of SEQ ID NO: 158, a sdCDR2 of SEQ ID NO: 159, and a sdCDR3 of SEQ ID NO:160;
j) a sdCDR1 of SEQ ID NO:162, a sdCDR2 of SEQ ID NO:163, and a sdCDR3 of SEQ ID NO:164;
k) a sdCDR1 of SEQ ID NO:166, a sdCDR2 of SEQ ID NO:167, and a sdCDR3 of SEQ ID NO:168;
l) a sdCDR1 of SEQ ID NO: 170, a sdCDR2 of SEQ ID NO:171, and a sdCDR3 of SEQ ID NO: 172;
m) a sdCDR1 of SEQ ID NO:174, a sdCDR2 of SEQ ID NO:175, and a sdCDR3 of SEQ ID NO:176;
n) a sdCDR1 of SEQ ID NO:178, a sdCDR2 of SEQ ID NO:179, and a sdCDR3 of SEQ ID NO:180;
o) a sdCDR1 of SEQ ID NO:182, a sdCDR2 of SEQ ID NO:183, and a sdCDR3 of SEQ ID NO:184;
p) a sdCDR1 of SEQ ID NO:186, a sdCDR2 of SEQ ID NO:187, and a sdCDR3 of SEQ ID NO:188;
q) a sdCDR1 of SEQ ID NO:190, a sdCDR2 of SEQ ID NO:191, and a sdCDR3 of SEQ ID NO:192;
r) a sdCDR1 of SEQ ID NO:194, a sdCDR2 of SEQ ID NO:195, and a sdCDR3 of SEQ ID NO:196;
s) a sdCDR1 of SEQ ID NO:198, a sdCDR2 of SEQ ID NO:199, and a sdCDR3 of SEQ ID NO:200;
t) a sdCDR1 of SEQ ID NO:202, a sdCDR2 of SEQ ID NO:203, and a sdCDR3 of SEQ ID NO:204;
u) a sdCDR1 of SEQ ID NO:206, a sdCDR2 of SEQ ID NO:207, and a sdCDR3 of SEQ ID NO:203;
v) a sdCDR1 of SEQ ID NO:210, a sdCDR2 of SEQ ID NO:211, and a sdCDR3 of SEQ ID NO:212;
w) a sdCDR1 of SEQ ID NO:214, a sdCDR2 of SEQ ID NO:215, and a sdCDR3 of SEQ ID NO:216;
x) a sdCDR1 of SEQ ID NO:218, a sdCDR2 of SEQ ID NO:219, and a sdCDR3 of SEQ ID NO:220;
y) a sdCDR1 of SEQ ID NO:222, a sdCDR2 of SEQ ID NO:223, and a sdCDR3 of SEQ ID NO:224;
z) a sdCDR1 of SEQ ID NO:226, a sdCDR2 of SEQ ID NO:227, and a sdCDR3 of SEQ ID NO:228;
aa) a sdCDR1 of SEQ ID NO:230, a sdCDR2 of SEQ ID NO:231, and a sdCDR3 of SEQ ID NO:232;
ab) a sdCDR1 of SEQ ID NO:234, a sdCDR2 of SEQ ID NO:235, and a sdCDR3 of SEQ ID NO:236;
ac) a sdCDR1 of SEQ ID NO:238, a sdCDR2 of SEQ ID NO:239, and a sdCDR3 of SEQ ID NO:240;
ad) a sdCDR1 of SEQ ID NO:242, a sdCDR2 of SEQ ID NO:243, and a sdCDR3 with SEQ ID NO:244; and
ae) a sdCDR1 of SEQ ID NO:500, a sdCDR2 of SEQ ID NO:501, and a sdCDR3 with SEQ ID NO:502;
af) a sdCDR1 of SEQ ID NO:504, a sdCDR2 of SEQ ID NO:505, and a sdCDR3 with SEQ ID NO:506;
ag) a sdCDR1 of SEQ ID NO:508, a sdCDR2 of SEQ ID NO:509, and a sdCDR3 with SEQ ID NO:510; and
ah) a sdCDR1 of SEQ ID NO:512, a sdCDR2 of SEQ ID NO:513, and a sdCDR3 with SEQ ID NO:5.

54. The fusion protein of claim 53, comprising an amino acid sequence selected from the group consisting of SEQ ID NO:193, SEQ ID NO:217, SEQ ID NO:225, SEQ ID NO:237, SEQ ID NO:141, SEQ ID NO:145, SEQ ID NO:149, SEQ ID NO:153, SEQ ID NO:157, SEQ ID NO:161, SEQ ID NO:165, SEQ ID NO:169, SEQ ID NO: 173, SEQ ID NO:177, SEQ ID NO:181, SEQ ID NO:185, SEQ ID NO: 189,, SEQ ID NO:197, SEQ ID NO:201, SEQ ID NO:205, SEQ ID NO:209, SEQ ID NO:213, SEQ ID NO:221, SEQ ID NO:229, SEQ ID NO:233, SEQ ID NO:241, SEQ ID NO:499, SEQ ID NO:503, SEQ ID NO:507, and SEQ ID NO:511.

55. A method of treating cancer in a subject comprising administering the fusion protein of claim 53 to the subject.
